(19) United States
(12) Patent Application Publication
(10) Pub. No.: US 2014/0315956 A1
(43) Pub. Date: Oct. 23, 2014

(54) SMALL MOLECULE INHIBITORS OF IL-6 AND USES THEREOF

(71) Applicants: James Fuchs, Columbus, OH (US); Chenglong Li, Dublin, OH (US); Pui Kai Li, Galloway, OH (US); Jiayuh Lin, Dublin, OH (US)

(72) Inventors: James Fuchs, Columbus, OH (US); Chenglong Li, Dublin, OH (US); Pui Kai Li, Galloway, OH (US); Jiayuh Lin, Dublin, OH (US)

(73) Assignees: NATIONWIDE CHILDREN'S HOSPITAL, Columbus, OH (US); OHIO STATE INNOVATION FOUNDATION, Columbus, OH (US)

(21) Appl. No.: 14/235,591

(22) PCT Filed: Jul. 29, 2012
(86) PCT No.: PCT/US12/48758

§ 371 Date: Jul. 10, 2014

Related U.S. Application Data
(60) Provisional application No. 61/513,351, filed on Jul. 29, 2011.
(60) Provisional application No. 61/513,360, filed on Jul. 29, 2011.

(51) Int. Cl. C07D 209/12; C07D 401/12; C07D 491/048

(52) U.S. Cl. CPC C07D 209/12; C07D 491/048; C07D 401/12
USPC 514/339; 548/364.4; 514/406; 548/431; 514/411; 548/510; 514/415; 548/494; 546/277.4

(57) ABSTRACT

In one aspect, the invention relates to substituted 2-(1H-indol-3-yl)ethanol analogs and substituted 3,3a,8,8a-tetrahydro-2H-furo[2,3-b]indole analogs, derivatives thereof, and related compounds, which are useful as inhibitors of IL-6 mediated activation of the Jak2/STAT3 pathway; synthetic methods for making the compounds; pharmaceutical compositions comprising the compounds; and methods of treating disorders of uncontrolled cellular proliferation associated with a IL6 dysfunction using the compounds and compositions. This abstract is intended as a scanning tool for purposes of searching in the particular art and is not intended to be limiting of the present invention.

### CROSS-REFERENCE TO RELATED APPLICATIONS

This application claims the benefit of U.S. Application Nos. 61/513,351 and 61/513,360, both of which were filed Jul. 29, 2011, which are hereby incorporated by reference in their entirety.

### STATEMENT REGARDING FEDERALLY SPONSORED RESEARCH

This invention was made with government support under grant numbers DOD Breast Cancer Research grant W81XWH-10-1-0996 and DOD Prostate Cancer Research grant W81XWH-10-1-0458 awarded by the United States Department of Defense. The United States government has certain rights in the invention.

### BACKGROUND

IL-6 is responsible for growth stimulation (or regulation) in a number of cancer cell types through the induction of various signaling pathways, including the critical Janus kinase/Signal Transducers and Activators of Transcription (JAK/STAT) pathway. The JAK2/STAT3 pathway mediates gene transcription and thereby directly influences growth, differentiation, and apoptosis in the cancer cells.¹⁰ Mounting evidence in numerous cancer types including prostate cancer indicates the importance of STAT3 in cancer progression and its dependence on IL-6.⁸ IL-6 initiates the JAK2/STAT3 signaling cascade via interaction with the extracellular domains of IL6-R and GP130 via a heterodimeric IL-6/IL-6R/GP130 complex (FIG. 1). This dimerization triggers activation of the Janus (JAK) kinases, which phosphorylate tyrosine residues in the cytoplasmic domain of GP130, leading to the tyrosine phosphorylation of STAT3 at the PY705 site.¹¹ Upon phosphorylation, STAT3 dissociates from GP130, undergoes dimerization, and translocates to the nucleus where it binds to DNA and activates gene transcription. Constitutive activation of STAT3 has been reported in more than 80% of prostate cancer tumor samples.¹² Persistent activation of STAT3 signaling has been demonstrated to contribute to oncogenesis by stimulating cell proliferation, mediating immune evasion, promoting angiogenesis, and resistance to apoptosis induced by conventional therapies.

IL-6 and its downstream target STAT3 have been recognized as promising molecular targets for the treatment of cancer.¹³⁻¹⁴ In previous studies IL-6 has been shown to up-regulate cell growth and enhance chemical resistance in PC-3 cells.¹⁵ Dominant-negative GP130 protein, anti-interleukin-6 monoclonal antibodies, and the IL-6 superantagonist Sant7 have demonstrated inhibition of cancer cell growth and sensitization of the cells to chemotherapeutic agents.¹⁶⁻¹⁷ To date, however, no small molecule capable of directly inhibiting the signaling of IL-6 has been evaluated in clinical trials for cancer patients. These reports support that the IL-6/gp130/STAT3 pathway is critical for prostate cancer progression and could serve as an attractive therapeutic target.

As described above, Interleukin-6 (IL-6) is a multifunctional cytokine that is important for immune responses, cell survival, apoptosis, and proliferation. IL-6 signals via a heterodimeric IL-6/IL-6R/gp130 complex, whose engagement triggers activation of Janus (JAK) kinases, and one of the major downstream effectors, STAT3. Previous studies implicated IL-6 and its major effector STAT3 as protumorigenic agents in many cancers, including breast, cancer. In breast cancer, STAT3 is tyrosine-phosphorylated mainly through the interleukin-6/glycoprotein 130/Janus kinase pathway. In fact, multiple studies have established IL-6 as a potent growth factor for several cancers including breast cancer. In addition, inhibition of gp130, the common signaling subunit of receptors used by IL-6 in breast cancer blocks constitutive activation of STAT3 and inhibits in vivo malignancy. Furthermore, IL-6 levels are significantly elevated in lung and breast cancer patients, which are associated with poor prognosis. A recent report establishes IL-6 as a potential regulator of breast tumor stem cell self renewal, implicating IL-6 as a critical factor in tumor mammosphere revival and resistance. Purohit et al. and Garcia-Tunon et al. found weak expression of IL-6 and its receptors in patients with benign lesions; however, in invasive breast tumors, the percentage of cases showing immunoreactivity for IL-6, gp130, and IL-6Ra was much higher than in non-malignant lesions, and the intensity of expression was two to three times higher. Knupfer and Preiss systematically tabulated IL-6 levels in a) healthy vs. breast cancer patients; b) patients in different tumor stages; c) patients at different severities of metastasis; d) link to clinical outcome in metastatic breast cancer patients; e) link to therapeutic success in metastatic and recurrent breast cancer patients and f) link to non-recurrent vs. recurrent breast cancer patients, all pointing to clear negative prognosticator as IL-6 elevates 3- to 40-fold increases. These reports support that IL-6/gp130/STAT3 pathway is critical for breast cancer progression and could serve as an attractive therapeutic target.

Thus, homodimerization of the IL-6/IL-6R/GP130 heterotrimer in various cancer types, resulting in IL-6/JAK2/STAT3 signaling, could be one of major causes of cancer proliferation, anti-apoptosis, metastasis, drug resistance and revival. Inhibition of this dimerization event and the resulting disruption of the downstream signal transduction pathway should provide an exciting new option for prostate cancer therapy.

Despite advances in understanding the role of IL-6 signaling in the development and progression of cancer, there is a scarcity of compounds effective in the treatment of cancer and other diseases, e.g. inflammatory diseases, associated with dysfunction in IL6 signaling, dysfunction in regulation of the Jak2/STAT3 pathway, or dysfunction in STAT3 regulation. These needs and other needs are satisfied by the present invention.

### SUMMARY

In accordance with the purpose(s) of the invention, as embodied and broadly described herein, the invention, in one aspect, relates to compounds useful in inhibiting IL6-mediated STAT3 phosphorylation, methods of making same, pharmaceutical compositions comprising same, methods of treating disorder of uncontrolled cellular proliferation, methods of treating an immune disorder, and using same. In various further aspects, the invention pertains to compounds useful in inhibiting homodimerization of IL6-IL6R-GP130 heterotrimers. In a further aspect, the invention pertains to compounds useful in therapeutically modulating a Jak2/STAT3 signaling pathway dysfunction.

Disclosed are compounds having a structure represented by a formula:

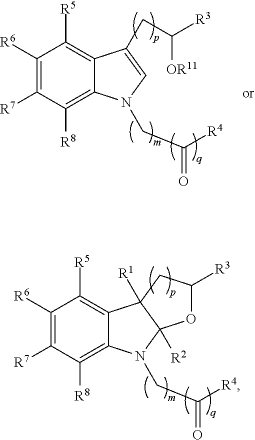

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

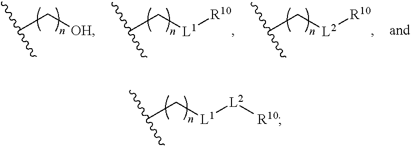

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O—Cy¹, and —NH—Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

Also disclosed are methods for the treatment of a disorder associated with an IL6 dysfunction in a mammal comprising the step of administering to the mammal a therapeutically effective amount of at least one compound having a structure represented by a formula:

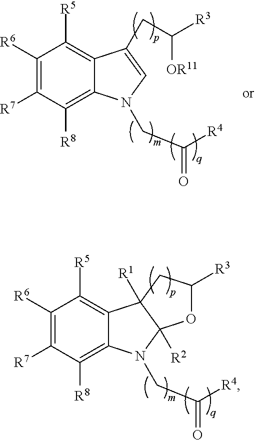

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

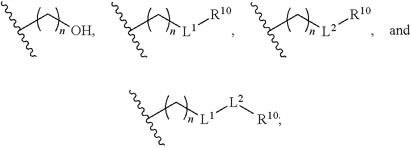

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

Also disclosed are methods for the treatment of a disorder of uncontrolled cellular proliferation associated with STAT3 dysfunction in a mammal comprising the step of administering to the mammal a therapeutically effective amount of at least one compound having a structure represented by a formula:

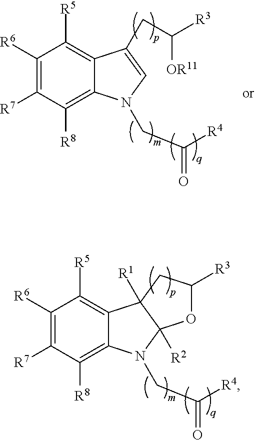

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

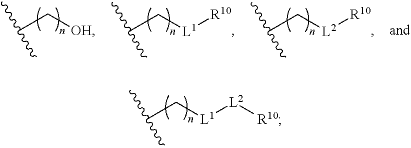

wherein L¹ is —O— or —NH—; wherein L² is —CH₂— or —(C═O) —; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

Also disclosed are methods for the treatment of an immune disorder associated with a STAT3 dysfunction in a mammal comprising the step of administering to the mammal a therapeutically effective amount of at least one compound having a structure represented by a formula:

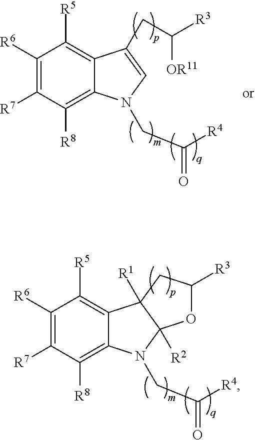

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen.

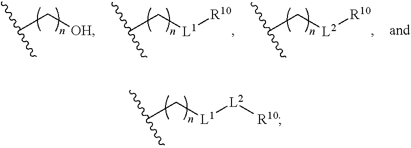

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

Also disclosed are methods for inhibition of IL6 mediated activation of the Jak2/STAT3 pathway in a mammal comprising the step of administering to the mammal a therapeutically effective amount of at least one compound having a structure represented by a formula:

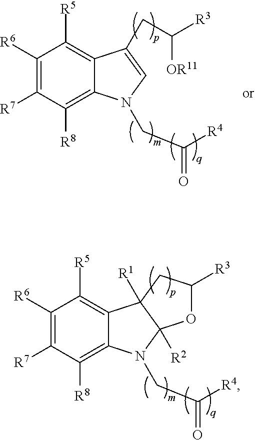

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH: wherein R³ is selected from: hydrogen,

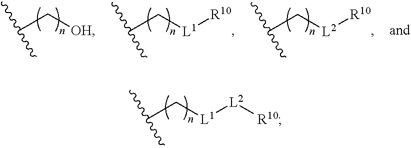

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

Also disclosed are methods inhibition of homodimerization of a IL6-IL6R-gp130 heterotrimer in a mammal comprising the step of administering to the mammal a therapeutically effective amount of at least one compound having a structure represented by a formula:

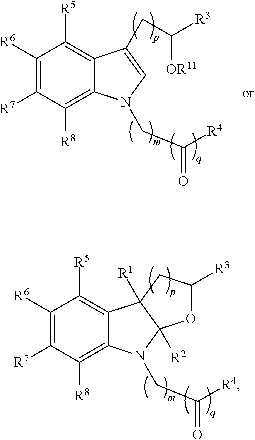

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

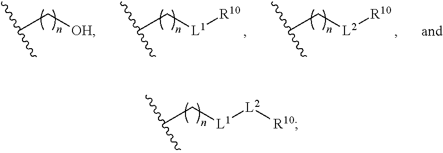

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

Also disclosed are methods for inhibition of IL6 mediated activation of the Jak2/STAT3 pathway in at least one cell, comprising the step of contacting the at least one cell with an effective amount of at least one compound having a structure represented by a formula:

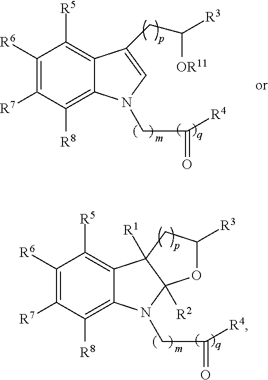

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

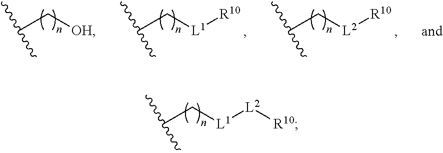

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

Also disclosed are methods for inhibition of homodimerization of a IL6-IL6R-gp130 heterotrimer in at least one cell, comprising the step of contacting the at least one cell with an effective amount of at least one compound having a structure represented by a formula:

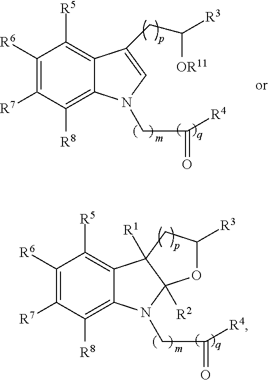

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

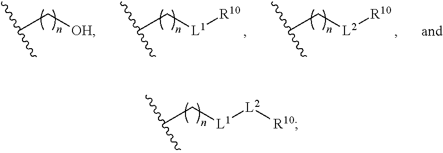

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

Also disclosed are kits comprising at least one compound having a structure represented by a formula:

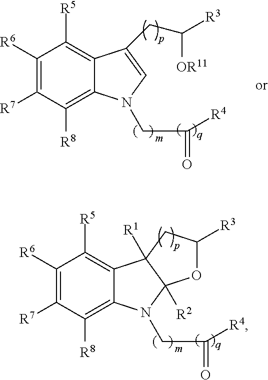

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

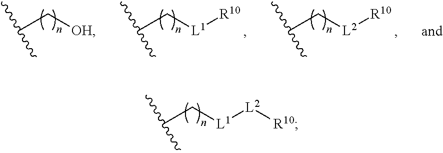

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof; and one or more of: (a) at least one agent known to increase IL6 activity; (b) at least one agent known to decrease IL6 activity; (c) at least one agent known to treat an immune disorder; (d) at least one agent known to treat a disease of uncontrolled cellular proliferation; or (e) instructions for treating a disorder associated with STAT3 dysfunction.

Also disclosed are pharmaceutical compositions comprising an effective amount of a disclosed compound or a product of a disclosed method of making and a pharmaceutically acceptable carrier. In various aspects, disclosed are pharmaceutical compositions comprising an effective amount of a disclosed compound and a pharmaceutically acceptable carrier.

Also disclosed are methods for manufacturing a medicament comprising combining at least one disclosed compound or a product of a disclosed method of making with a pharmaceutically acceptable carrier or diluent. In various aspects, disclosed are methods for manufacturing a medicament comprising combining at least one disclosed compound with a pharmaceutically acceptable carrier or diluent.

Also disclosed are uses of a disclosed compound or a disclosed product in the manufacture of a medicament for the treatment a disorder of uncontrolled cellular proliferation.

Also disclosed are uses of a disclosed compound or a disclosed product in the manufacture of a medicament for the treatment a disorder of uncontrolled cellular proliferation.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows the structure of the IL-6/IL-6R/GP130 hexameric complex (homodimer of a IL-6/IL-6R/GP130 heterotrimer) from Boulanger, et al. (See Ref. No. 24).

FIG. 2 shows the chemical structure of MDL-A ((+)-Madindoline A, CAS 184877-64-3).

FIG. 3 shows a schematic representation of a proposed mechanism for the inhibition of IL-6 mediated signaling by Madindoline A (MDL-A). Briefly, MDL-A binds to gp130 (site 2) and blocks the dimerization of the IL-6/IL-6R/GP130 heterotrimers. This binding interrupts the signaling in the Jak/STAT3 pathway and downstream gene expression.

FIG. 4 shows representative docking models of the present invention show the binding of MDL-A to the GP130 D1 domain using crystal structure data from Boulanger, et al. (See Ref. No. 24). Briefly, the binding of MDL-A prevents the IL-6/GP130 interaction, effectively disabling GP130 functional dimerization (as described in FIG. 3). (Panel A) D1 domain in ribbon representation and MDL-A in thick ball-and-stick. Asn92 side chain forms two hydrogen bonds with the both the upper and lower portion of MDL-A inhibitor, confirming biochemical studies that both portions of MDL-A are needed for GP130 binding and inhibition; the Gly95 main chain carbonyl forms one hydrogen bond with MDL-A and the Tyr94 has an aromatic interaction with the indoline portion of MDL-A. (Panel B) D1 domain in electrostatic surface representation with positively and negatively charged regions indicated, and any non-highlighted regions is hydrophobic in nature); IL-6 in ribbon representation. The figure shows two ellipses proximal to the highlighted Trp157 and Leu57 indicate two major binding “hot spots” between IL-6 and GP130. Both spots are disrupted by MDL-A: a) the first spot, Trp157, in the N-terminus of the last IL-6 helix is displaced by the indoline moiety of MDL-A; b) in the second spot, Leu57, the hydrophobic interaction between Leu57 on the first loop of IL-6 and the GP130 hydrophobic pocket is disrupted by the MDL-A aliphatic tail. A small ellipse (as indicated) highlights an extra empty polar subpocket that can be used to design more potent and specific inhibitors.

FIG. 5 shows a docking model showing a representative interaction of a representative disclosed compounds with the GP130 D1 domain. It should be noted that the aryl substituents (not present in MDL-A) are designed to increase potency through interaction with the “extra subpocket” described in FIG. 4.

FIG. 6 shows representative structural formula for representative disclosed substituted analogues of the present invention corresponding to benzyl and pyrazole analogues in the position corresponding to R⁴ of Formula II. In the figure R₁ and R₂ correspond to R⁶ and R³, respectively, of Formula II as disclosed herein.

FIG. 7 shows representative disclosed compounds of the present invention.

FIG. 8 shows the convergent synthesis strategy for pyrazole analogues of the present invention.

FIG. 9 shows structural fragments for the “Northern” hydroxylindoline portion of certain substituted analogues of the present invention that can be used in a convergent synthesis as shown in FIG. 8.

FIG. 10 shows structural fragments for the “Southern” benzyl or pyrazole derivative portion of certain substituted analogues of the present invention that can be used in a convergent synthesis as shown in FIG. 8.

FIG. 11 shows a flowchart representing an interactive cyclic approach to drug development for the disclosed compounds of the present invention, e.g. drug development of the disclosed compounds for use as inhibitors in prostate cancer.

FIG. 12 shows a schematic representation of the disruption of homodimerization of the IL-6/IL-6R/GP130 heterotrimers by disclosed compounds of the present invention.

FIG. 13 shows representative data for the purification of gp130 extracellular domain. (Panel A) A shows a representative Coomassie Blue-stained gel of purified gp130 (amino residue 18-615): lane M, molecular weight standard (Precision Plus Protein™ Standards, BIO-RAD); lane 1 and 2: purified gp130-Fc-HA protein. (Panel B) Anti-HA immunoblot of the purified gp130-Fc-HA protein.

FIG. 14 shows representative surface plasmon response data for MDL-A and representative disclosed compounds. The data show that in a direct binding assay, MDL-16, MDL-5,MDL-17, MDL-8, MDL-7, MDL-6 and MDL-3 show better binding than MDL-A. MDL-A shows dissociation constant (K_{D}) value about 290 μM.

FIG. 15 shows representative surface plasmon response data for benzyl analogues of the present invention (MDL-6, MDL-7 and MDL-8). These are examples of tail modification analogues and show better activity than MDL-A in this direct binding assay.

FIG. 16 shows representative surface plasmon response data for benzyl analogues of the present invention (MDL-5, MDL-16 and MDL-17). These are examples of tail modification analogues with additional modification for binding and show better activity than MDL-A in this direct binding assay.

FIG. 17 shows representative Western analysis data showing inhibition of Stat3 phosphorylation by Madindoline A (MDL-A) and representative disclosed compounds of the present invention.

FIG. 18 shows representative Western analysis data showing dose dependent inhibition of Stat3 phosphorylation by Madindoline A (MDL-A) and representative disclosed compounds of the present invention.

FIG. 19 shows representative Western analysis data showing inhibition of Stat3 phosphorylation and inhibition of apoptosis induction by Madindoline A (MDL-A) and representative disclosed compounds (MDL-5 and MDL-16) of the present invention in the presence of IL6.

FIG. 20 shows representative Western analysis data showing dose dependent inhibition of Stat3 phosphorylation by representative disclosed compounds of the present invention when tested on LNCaP cells and using 40 μM of the indicated compounds (treatment for 4 hours). IL6 was used at 12.5 ng/ml and cells were exposed for 30 min. The amount of sample loaded per lane was 5 μg total protein.

FIG. 21 shows representative Western analysis data showing dose dependent inhibition of Stat3 phosphorylation by representative disclosed compounds of the present invention when tested on LNCaP cells and using 40 μM of the indicated compounds (treatment for 4 hours). IL6 was used at 12.5 ng/ml and cells were exposed for 30 min. The amount of sample loaded per lane was 5 μg total protein.

FIG. 22 shows representative Western analysis data showing dose dependent inhibition of Stat3 phosphorylation by representative disclosed compounds of the present invention when tested on LNCaP cells and using 40 μM of the indicated compounds (treatment for 4 hours). IL6 was used at 12.5 ng/ml and cells were exposed for 30 min. The amount of sample loaded per lane was 5 μg total protein.

FIG. 23 shows representative immunoblot and cell proliferation data for representative disclosed compounds of the present invention. The cell-line and compound is as indicated in the panels. The lower two graphs show determination of IC₅₀ for cell proliferation using the indicated cell line. Cell proliferation was determined using a fluorescent assay with 5-carboxyfluorescein diacetate acetoxymethyl ester (CFDA-AM), which is cleaved to fluorescein in living cells.

FIG. 24 shows representative immunoblot and cell proliferation data for representative disclosed compounds of the present invention. The cell-line and compound is as indicated in the panels. The lower two graphs show determination of IC₅₀ for cell proliferation using the indicated cell line. Cell proliferation was determined using a fluorescent assay with 5-carboxyfluorescein diacetate acetoxymethyl ester (CFDA-AM), which is cleaved to fluorescein in living cells.

FIG. 25 shows representative data on the effect of representative disclosed compounds on MIA PaCa-2 pancreatic cell death. MIA PaCa-2 cells (n=8; 2 separate experiments) were exposed to various concentrations of MDL compounds for 4 hr in serum free media then 10% serum added back to media. Cells were photographed under brightfield phase contrast microscopy (40×) one day after drug exposure and 48 hr after exposure 25 μM CFDA-AM (InVitrogen, Eugene, Oreg.) was added to cells for 2 hr and fluorescence (485 NM EX; 520 NM EM) read on a plate reader as a marker of proliferation. IC50 values were calculated using non-linear regression analysis with Prism 5.0 software (GraphPad, San Diego, Calif.).

FIG. 26 shows representative data on the effect of representative disclosed compounds on PANC-1 pancreatic cell death. PANC-1 cells (n=8; 2 separate experiments) were exposed to various concentrations of MDL compounds for 4 hr in serum free media then 10% serum added back to media. Cells were photographed under brightfield phase contrast microscopy (40×) one day after drug exposure and 48 hr after exposure 25 μM CFDA-AM (InVitrogen, Eugene, Oreg.) was added to cells for 2 hr and fluorescence (485 NM EX; 520 NM EM) read on a plate reader as a marker of proliferation. IC50 values were calculated using non-linear regression analysis with Prism 5.0 software (GraphPad, San Diego, Calif.). Bottom panel. Effect of MDL compounds on p-STAT3 expression. U937 cells were serum starved overnight then exposed to 100 μM MDL compounds for 2 hr in serum free media followed by exposure to 20 ng/ml IL-6 for 10 min. Whole cell lysates were made and western blot against phospho STAT3 assessed. Data are representative of 3 samples.

FIG. 27 shows representative data on the effect of representative disclosed compounds on colorectal cancer cell death. HT-29 and COLO-205 cells (n=8; 2 separate experiments) were exposed to various concentrations of MDL compounds for 4 hr in serum free media then 10% serum added back to media. 48 hr after exposure 25 μM CFDA-AM (InVitrogen, Eugene, Oreg.) was added to cells for 2 hr and fluorescence (485 NM EX; 520 NM EM) read on a plate reader as a marker of proliferation. IC50 values were calculated using non-linear regression analysis with Prism 5.0 software (GraphPad, San Diego, Calif.).

FIG. 286 shows representative data on the effect of representative disclosed compounds on colorectal cancer cell death. T-84 cells (n=8; 2 separate experiments) were exposed to various concentrations of MDL compounds for 4 hr in serum free media then 10% serum added back to media. 48 hr after exposure 25 μM CFDA-AM (InVitrogen, Eugene, Oreg.) was added to cells for 2 hr and fluorescence (485 NM EX; 520 NM EM) read on a plate reader as a marker of proliferation. IC50 values were calculated using non-linear regression analysis with Prism 5.0 software (GraphPad, San Diego, Calif.).

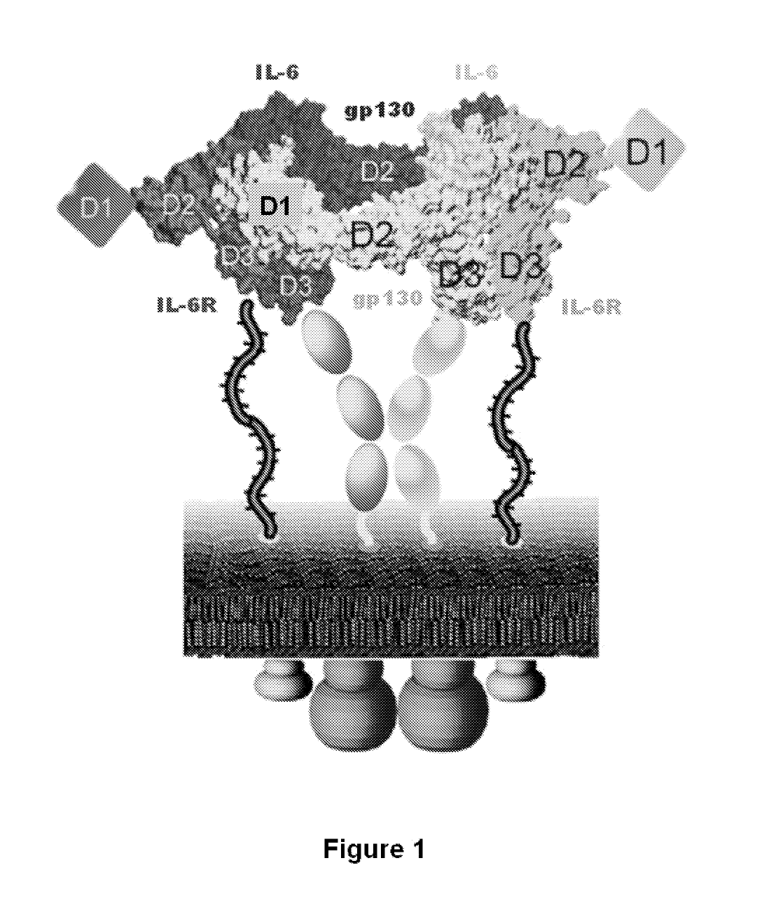

FIG. 29 shows representative data on the effect of representative disclosed compounds on breast cancer cell STAT3 inhibition (top panel) and death (bottom panel). HT-29 and COLO-205 cells (n=8; 2 separate experiments) were exposed to various concentrations of MDL compounds for 4 hr in serum free media then 10% serum added back to media. 48 hr after exposure 25 μM CFDA-AM (InVitrogen, Eugene, Oreg.) was added to cells for 2 hr and fluorescence (485 NM EX; 520 NM EM) read on a plate reader as a marker of proliferation. IC50 values were calculated using non-linear regression analysis with Prism 5.0 software (GraphPad, San Diego, Calif.).

FIG. 30 shows representative data on the effect of representative disclosed compounds on hepatocellular cancer cell death. Hep-G2 cells (n=8; 2 separate experiments) were exposed to various concentrations of MDL compounds for 4 hr in serum free media then 10% serum added back to media. 48 hr after exposure 25 μM CFDA-AM (InVitrogen, Eugene, Oreg.) was added to cells for 2 hr and fluorescence (485 NM EX; 520 NM EM) read on a plate reader as a marker of proliferation. IC50 values were calculated using non-linear regression analysis with Prism 5.0 software (GraphPad, San Diego, Calif.).

FIG. 31 shows representative data obtained from Western blot analysis of protein extracts from MCF-7 cell lines treated with the indicated levels of the indicated representative disclosed compound. The data in the figure shows that the representative disclosed compounds inhibit the phosphorylation of STAT3, but not the phosphorylation of ERK1/2. In addition, the data show selected for inhibition of phosphorylation of STAT3, but do not affect the phosphorylation of STAT1.

FIG. 32 shows representative data immunofluorescence data obtained in MCF-7 cell-lines treated as indicated with IL6 with and without the indicated representative compound, MDL-5. The data show that the representative compound, MDL-5, inhibits the nuclear translocation of STAT3 which is activated by the IL6.

FIG. 32 shows representative data immunofluorescence data obtained in MCF-7 cell-lines treated as indicated with IL6 with and without the indicated representative compound, MDL-16. The data show that the representative compound, MDL-16, inhibits the nuclear translocation of STAT3 which is activated by the IL6.

FIG. 34 shows representative data for the effect of a representative compound, MDL-16, on LIF versus IL6 induced phosphorylation of STAT3. The data show that MDL-16 inhibits IL6 mediated STAT3 phosphorylation, but not LIF mediated STAT3 phosphorylation.

FIG. 35 shows representative data obtained from an in vivo model of cancer, i.e. a tumor xenograft model, using SUM-159 breast cancer cells to establish the tumor xenograft. The effect of a representative compound, MDL-16, on tumor progression is compared to a group treated with vehicle (DMSO). The data show that a dose level of 100 mg/kg of MDL-16 has a significant effect on tumor progress compared to vehicle.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. DEFINITIONS

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

As used herein, the term “STAT3” and “signal transducer and activator of transcription 3” can be used interchangeably and refers the gene, mRNA, or protein encoded by the STAT3 gene. The STAT3 gene has a gene map locus of 17q21.31, 17q21.2, and 17q21 as described by, respectively, Entrez Gene cytogenetic band, band, Ensembl cytogenetic band, and the HGNC cytogenetic band. The term STAT3 refers to a native protein in human of 770 amino acids with a molecular weight of about 88,068 Da as described in UniProtKB/Swiss-Prot database, and is a member of the STAT family (signal transducers and activators of transcription), that is currently described as a family of 7 transcription factors that form part of the JAK-STAT signaling cascade. The term STAT3 is inclusive of the protein, gene product and/or gene referred to by such alternative designations as: signal transducer and activator of transcription 3 (acute-phase response factor), APRF, Acute-phase response factor, HIES, and DNA-binding protein APRF.

As used herein, the term “IL6” and “interleukin 6” can be used interchangeably and refers the gene, mRNA, or protein encoded by the IL6 gene. The IL6 gene has a gene map locus of 7p21, 7p15.3, and 7p21-p15 as described by, respectively, Entrez Gene cytogenetic band, band, Ensembl cytogenetic band, and the HGNC cytogenetic band. The term IL6 refers to a native protein in human of 212 amino acids with a molecular weight of about 23,718 Da as described in UniProtKB/Swiss-Prot database, and is a member of the IL6 family, that currently is described as including IL-6, IL-11, leukemia inhibitory factor (LIF), oncostatin M (OSM), cardiotrophin-1 (CT-1), ciliary neurotrophic factor (CNTF), and cardiotrophin-like cytokine (CLC). The term IL6 is inclusive of the protein, gene product and/or gene referred to by such alternative designations as: interleukin 6 (interferon, beta 2), IFNB2, IL-6, BSF2, HGF, HSF, Hybridoma growth factor, Interferon beta-2, BSF-2, CDF, IFN-beta-2, B-cell stimulatory factor 2, CTL differentiation factor, B-cell differentiation factor, and interleukin BSF-2.

As used herein, the term “IL6R” and “interleukin 6 receptor” can be used interchangeably and refers the gene, mRNA, or protein encoded by the IL6R gene. The IL6R gene has a gene map locus of 1q21, 1q21.3, and 1q21 as described by, respectively, Entrez Gene cytogenetic band, band, Ensembl cytogenetic band, and the HGNC cytogenetic band. The term IL6R refers to a native protein in human of 468 amino acids with a molecular weight of about 51,548 Da as described in UniProtKB/Swiss-Prot database, and is a member of the type I cytokine receptor family, that currently is described as comprising interleukin receptors (e.g. IL6R or IL27R), colony stimulating factor receptors (e.g. GM-CSF receptor or G-CSF receptor), hormone receptor/neuropeptide receptors (e.g. growth hormone receptor) and other cytokine receptors (e.g. leukemia inhibitory factor receptor). The type I cytokine receptor family are transmembrane receptors expressed on the surface of cells that recognize and respond to cytokines with four α-helical strands. The term IL6R is inclusive of the protein, gene product and/or gene referred to by such alternative designations as: CD126, membrane glycoprotein 80, IL-6RA, CD126 antigen, gp80, IL-6 receptor subunit alpha, IL-6R1, IL-6R-1, IL6RA, interleukin-6 receptor subunit alpha, IL-6R-alpha, and IL-6R subunit alpha.

As used herein, the term “gp130,” “interleukin 6 signal transducer,” “glycoprotein 130” and “membrane glycoprotein 130” can be used interchangeably and refers the gene, mRNA, or protein encoded by the IL6ST gene. The IL6ST gene has a gene map locus of 5q11.2 as described by Entrez Gene cytogenetic band, band, Ensembl cytogenetic band, and the HGNC cytogenetic band. The term gp130 refers to a native protein in human of 918 amino acids with a molecular weight of about 103,537 Da as described in UniProtKB/Swiss-Prot database, and is a transmembrane protein which is the founding member of the class of all cytokine receptors. It forms one subunit of type I cytokine receptors within the IL-6 receptor family. It is often referred to as the common gp130 subunit, and is important for signal transduction following cytokine engagement. The term gp130 is inclusive of the protein, gene product and/or gene referred to by such alternative designations as: IL6ST, interleukin 6 signal transducer (gp130, oncostatin M receptor), CDW130, CDw130, CD130, CD 130 antigen, gp130 of the rheumatoid arthritis antigenic peptide-bearing soluble form, GP130, interleukin receptor beta chain, interleukin-6 receptor subunit beta, oncostatin-M receptor subunit alpha, membrane glycoprotein gp130, IL-6RB, IL-6R-beta, IL-6 receptor subunit beta, and IL-6R subunit beta.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of a disorder of uncontrolled cellular proliferation. In further aspects, the subject has been diagnosed with a need for treatment of an immune disorder, e.g. an inflammatory disease. In some aspects of the disclosed methods, the subject has been diagnosed with a need for treatment of a disorder of uncontrolled cellular proliferation prior to the administering step. In various aspects of the disclosed methods, the subject has been diagnosed with a need for treatment of an immune disorder, e.g. an inflammatory disease, prior to the administering step. In some aspects of the disclosed methods, the subject has been diagnosed with a need for inhibition of the homodimerization of the IL6-IL6R-gp130 heterotrimer.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder of uncontrolled cellular proliferation” means having been subjected to a physical examination by a person of skill, for example, a physician, and found to have a condition that can be treated by a compound or composition that can ameliorate the disease pathology associated with uncontrolled cellular proliferation. Alternatively, “diagnosed with an immune disorder” means having been subjected to a physical examination by a person of skill, for example, a physician, and found to have a condition that can be treated by a compound or composition that can ameliorate the disease pathology associated with an immune disorder, e.g. an inflammatory disease. Such a diagnosis can be in reference to a disorder, such as a cancer or an inflammatory disease, and the like, as discussed herein. For example, the term “diagnosed with a need for inhibition of homodimerization of the IL6-IL6R-gp130 heterotrimer” refers to having been subjected to a physical examination by a person of skill, for example, a physician, and found to have a condition that can be diagnosed or treated by for inhibition of homodimerization of the IL6-IL6R-gp130 heterotrimer activity. For example, “diagnosed with a need for inhibition of STAT3 activity” means having been subjected to a physical examination by a person of skill, for example, a physician, and found to have a condition that can be diagnosed or treated by inhibition of STAT3 activity. For example, “diagnosed with a need for treatment of one or more neurological and/or psychiatric disorder associated with IL6 dysfunction” means having been subjected to a physical examination by a person of skill, for example, a physician, and found to have one or more neurological and/or psychiatric disorder associated with IL6 dysfunction.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to uncontrolled cellular proliferation activity) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target metabotropic glutamate receptor, or other biological entity together in such a manner that the compound can affect the activity of the target (e.g., spliceosome, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14 th edition), the Physicians' Desk Reference (64 th edition), and The Pharmacological Basis of Therapeutics (12 th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “EC₅₀,” is intended to refer to the concentration of a substance (e.g., a compound or a drug) that is required for 50% agonism or activation of a biological process, or component of a process, including a protein, subunit, organelle, ribonucleoprotein, etc. In one aspect, an EC₅₀ can refer to the concentration of a substance that is required for 50% agonism or activation in vivo, as further defined elsewhere herein. In a further aspect, EC₅₀ refers to the concentration of agonist or activator that provokes a response halfway between the baseline and maximum response. In a yet further aspect, the response is in vitro. In a still further aspect, the response is measured in a human cell or cell-line transfected with human gp130 and/or IL6R. Alternatively, the response is measured in a cell or cell-line that has native expression of gp130 and/or IL6R, and exhibit a response to IL6, e.g. DU145 or PC3 cells.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance. In a yet further aspect, the response is in vitro. In a still further aspect, the response is measured in a human cell or cell-line transfected with human gp130 and/or IL6R. Alternatively, the response is measured in a cell or cell-line that has native expression of gp130 and/or IL6R, and exhibit a response to IL6, e.g. DU145 or PC3 cells.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. It is understand that the alkyl group is acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The cycloalkyl group can be substituted or unsubstituted. The cycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The cycloalkenyl group can be substituted or unsubstituted. The cycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The cycloalkynyl group can be substituted or unsubstituted. The cycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2)π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is NH₂.

The term “alkylamino” as used herein is represented by the formulas —NH(-alkyl) and —N(-alkyl)₂, and where alkyl is as described herein. The alkyl group can be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like, up to and including a C1-C24 alkyl. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, N-ethyl-N-methylamino group, N-methyl-N-propylamino group, and N-ethyl-N-propylamino group. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group, and the like.

The term “monoalkylamino” as used herein is represented by the formula —NH(-alkyl), where alkyl is as described herein. The alkyl group can be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like, up to and including a C1-C24 alkyl. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂, where alkyl is as described herein. The alkyl group can be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like, up to and including a C1-C24 alkyl. It is understood that each alkyl group can be independently varied, e.g. as in the representative compounds such as N-ethyl-N-methylamino group, N-methyl-N-propylamino group, and N-ethyl-N-propylamino group. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group, and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted, and the heteroaryl group can be monocyclic, bicyclic or multicyclic aromatic ring. The heteroaryl group can be substituted with one or more groups including, but not limited to, alkyl, cycloalkyl, alkoxy, amino, ether, halide, hydroxy, nitro, silyl, sulfo-oxo, or thiol as described herein. It is understood that a heteroaryl group may be bound either through a heteroatom in the ring, where chemically possible, or one of carbons comprising the heteroaryl ring.

A variety of heteroaryl groups are known in the art and include, without limitation, oxygen-containing rings, nitrogen-containing rings, sulfur-containing rings, mixed heteroatom-containing rings, fused heteroatom containing rings, and combinations thereof. Non-limiting examples of heteroaryl rings include furyl, pyrrolyl, pyrazolyl, imidazolyl, triazolyl, pyridyl, pyridazinyl, pyrimidinyl, pyrazinyl, azepinyl, triazinyl, thienyl, oxazolyl, thiazolyl, oxadiazolyl, oxatriazolyl, oxepinyl, thiepinyl, diazepinyl, benzofuranyl, thionapthene, indolyl, benzazolyl, pyranopyrrolyl, isoindazolyl, indoxazinyl, benzoxazolyl, quinolinyl, isoquinolinyl, benzodiazonyl, naphthyridinyl, benzothienyl, pyridopyridinyl, acridinyl, carbazolyl and purinyl rings.

The term “monocyclic heteroaryl,” as used herein, refers to a monocyclic ring system which is aromatic and in which at least one of the ring atoms is a heteroatom. Monocyclic heteroaryl groups include, but are not limited, to the following exemplary groups: pyridine, pyrimidine, furan, thiophene, pyrrole, isoxazole, isothiazole, pyrazole, oxazole, thiazole, imidazole, oxadiazole, including, 1,2,3-oxadiazole, 1,2,5-oxadiazole and 1,3,4-thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, and the like. Monocyclic heteroaryl groups are numbered according to standard chemical nomenclature.

The term “bicyclic heteroaryl,” as used herein, refers to a ring system comprising a bicyclic ring system in which at least one of the two rings is aromatic and at least one of the two rings contains a heteroatom. Bicyclic heteroaryl encompasses ring systems wherein an aromatic ring is fused with another aromatic ring, or wherein an aromatic ring is fused with a non-aromatic ring. Bicyclic heteroaryl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Examples of bicyclic heteroaryl groups include without limitation indolyl, isoindolyl, indolyl, indolinyl, indolizinyl, quinolinyl, isoquinolinyl, benzofuryl, bexothiophenyl, indazolyl, benzimidazolyl, benzothiazinyl, benzothiazolyl, purinyl, quinolizyl, quinolyl, isoquinolyl, cinnolinyl, phthalazinyl, quinazolizinyl, quinoxalyl, naphthyridinyl, and pteridyl. Bicyclic heteroaryls are numbered according to standard chemical nomenclature.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The term heterocycloalkyl group can also be a C2 heterocycloalkyl, C2-C3 heterocycloalkyl, C2-C4 heterocycloalkyl, C2-C5 heterocycloalkyl, C2-C6 heterocycloalkyl, C2-C7 heterocycloalkyl, C2-C8 heterocycloalkyl, C2-C9 heterocycloalkyl, C2-C10 heterocycloalkyl, C2-C11 heterocycloalkyl, and the like up to and including a C2-C14 heterocycloalkyl. For example, a C2 heterocycloalkyl comprises a group which has two carbon atoms and at least one heteroatom, including, but not limited to, aziridinyl, diazetidinyl, oxiranyl, thiiranyl, and the like. Alternatively, for example, a C5 heterocycloalkyl comprises a group which has five carbon atoms and at least one heteroatom, including, but not limited to, piperidinyl, tetrahydropyranyl, tetrahydrothiopyranyl, diazepanyl, and the like. It is understood that a heterocycloalkyl group may be bound either through a heteroatom in the ring, where chemically possible, or one of carbons comprising the heterocycloalkyl ring. The heterocycloalkyl group can be substituted or unsubstituted. The heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O(CH₂)₀₋₄C(O)OR^{o}; —(CH₂)₀₋₄—CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; (CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; (CH₂)₀₋₄N(R^{o})C(O)NR^{o}₂; —N(R^{o})C(S)NRO₂; —(CH₂)₀₋₄N(R^{o})C(O)OR^{o}; —N(R^{o})N(R^{o})C(O)R^{o}; —N(R^{o})N(R^{o})C(O)NRO₂; —N(R^{o})N(R^{o})C(O)OR^{o}; —(CH₂)₀₋₄C(O)R^{o}; C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR, —SC(S)SR^{o}; —(CH₂)₀₋₄SC(O)R^{o}; —(CH₂)₀₋₄C(O)NRO₂; —C(S)NR^{o}₂; —C(S)SR^{o}; —(CH₂)₀₋₄OC(O)NRO₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NRO₂; —(CH₂)₀₋₄S(O)R^{o}; —N(R^{o})S(O)₂NRO₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; —SiR^{o}₃; —(C₁₋₄ straight or branched) alkylene)O—N(R^{o})₂; or (C₁₋₄ straight or branched)alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R^{*}₂))₂₋₃O—, or —S(C(R^{*}₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{*}₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono or bicyclic ring having 0-4 heteroatoms independently selected from nitrogen, oxygen, or sulfur.

Suitable substituents on the aliphatic group of R^{†} are independently halogen, R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure

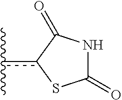

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound. For example, a compound prefixed with (−) or l meaning that the compound is levorotatory or a compound prefixed with (+) or d is dextrorotatory. For a given chemical structure, these compounds, called stereoisomers, are identical except that they are non-superimposable minor images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labelled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

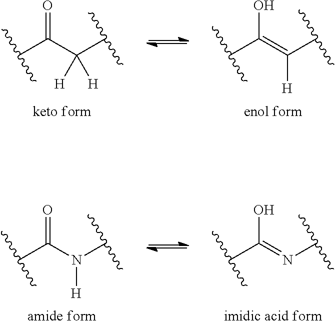 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyridinones can exist in two tautomeric forms, as shown below.

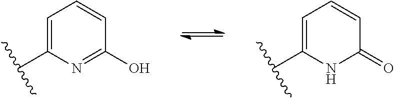 [SMILES: *c1cccc(O)n1 ; *c1cccc(=O)[nH]1]

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

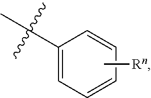

which is understood to be equivalent to a formula:

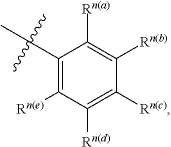

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

The following abbreviations are used herein. “ACN” means acetonitrile, “EtOAc” means ethyl acetate, “DCE” means 1,2-dichloroethane “DCM” means dichloromethane, “DIPE” means diisopropylether, “DMF” means N,N-dimethylformamide, “EtOH” means ethanol, “HATU” means 2-(7-aza-1H-benzotriazole-1-yl)-1,1,3,3-tetramethyluronium hexafluorophosphate, “HPLC” means high-performance liquid chromatography, “LCMS” means liquid chromatography/mass spectrometry, “MeOH” means methanol, “Ms” means methylsulfonyl, “NMR” means nuclear magnetic resonance, “RP” means reverse phase, “RT” means room temperature, “TEA” means triethylamine, and “THF” means tetrahydrofuran.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In accordance with the purpose(s) of the invention, as embodied and broadly described herein, the invention, in one aspect, relates to compounds useful in inhibiting IL6-mediated STAT3 phosphorylation, methods of making same, pharmaceutical compositions comprising same, methods of treating disorder of uncontrolled cellular proliferation, methods of treating an immune disorder, and using same. In various further aspects, the invention pertains to compounds useful in inhibiting homodimerization of IL6-IL6R-GP130 heterotrimers. In a further aspect, the invention pertains to compounds useful in therapeutically modulating a Jak2/STAT3 signaling pathway dysfunction. In a still further aspect, the disclosed compounds exhibit to GP130.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspect, the invention relates to a compound having a structure represented by a formula:

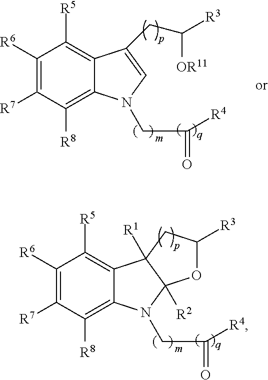

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

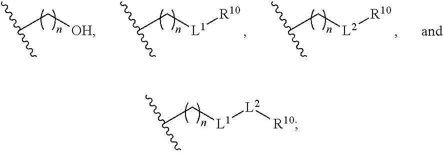

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —OCy¹, and —NH—Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

It should be noted that reference to compounds having the foregoing disclosed structural formulas can use “Formula I” to describe a compound having the structure represented by the formula:

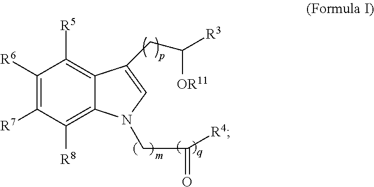

and “Formula II” to describe a compound having the structure represented by the formula:

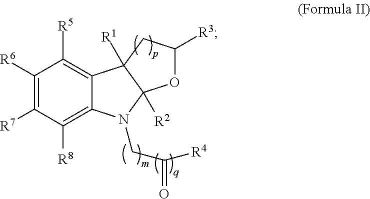

and wherein all other variables are as defined herein; or a pharmaceutically acceptable salt thereof.

In a further aspect, m is 1, 2, 3, 4, or 5. In a still further aspect, m is 1, 2, 3, or 4. In a yet further aspect, m is 1, 2, or 3. In an even further aspect, m is 1 or 2. In a still further aspect, m is 1. In a yet further aspect, m is 2. In an even further aspect, m is 3. In a still further aspect, m is 4. In a yet further aspect, m is 5. In an even further aspect, m is 6.

In a further aspect, n is 1, 2, 3, 4, or 5. In a still further aspect, n is 1, 2, 3, or 4. In a yet further aspect, n is 1, 2, or 3. In an even further aspect, n is 1 or 2. In a still further aspect, n is 1. In a yet further aspect, n is 2. In an even further aspect, n is 3. In a still further aspect, n is 4. In a yet further aspect, n is 5. In an even further aspect, n is 6.

In a further aspect, m is 1 and n is 1, 2, 3, 4, 5, or 6. In a still further aspect, m is 1 and n is 1, 2, 3, 4, or 5. In a yet further aspect, m is 1 and n is 1, 2, 3, or 4. In an even further aspect, m is 1 and n is 1, 2, or 3. In a still further aspect, m is 1 and n is for 2.

In a further aspect, m is 1 and n is 1. In an even further aspect, m is 1 and n is 2. In a still further aspect, m is 1 and n is 3. In a yet further aspect, m is 2 and n is 1. In an even further aspect, m is 2 and n is 2. In a still further aspect, m is 2 and n is 3. In a yet further aspect, m is 3 and n is 1. In an even further aspect, m is 3 and n is 2. In a still further aspect, m is 3 and n is 3.

In a further aspect, n is 1 and m is 1, 2, 3, 4, 5, or 6. In a still further aspect, n is 1 and m is 1, 2, 3, 4, or 5. In a yet further aspect, n is 1 and m is 1, 2, 3, or 4. In an even further aspect, n is 1 and m is 1, 2, or 3. In a still further aspect, n is 1 and m is for 2. In a further aspect, n is 1 and m is 2. In a still further aspect, n is 1 and m is 3.

In a further aspect, p is 1 or 2. In a still further aspect, p is 1 or 3. In a yet further aspect, p is 2 or 3. In an even further aspect, p is 1. In a still further aspect, p is 2. In a yet further aspect, p is 3.

In a further aspect, q is 0. In a still further aspect, q is 1.

In a various aspects, the compound has a structure represented by a formula:

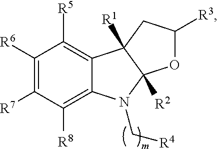

wherein n is 0-6; wherein m is 0-6; wherein each of R¹ and R², when present, is independently selected from H and —OH; wherein R³ is

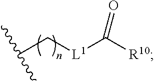

wherein L¹ is —O— or —NH—; wherein R⁴ is alkyl, alkoxy, O-alkyl, N-alkyl, aromatic, heteroaromatic, cyclic, or heterocyclic; wherein R⁵, R⁶, R⁷ and R⁸ are independently hydroxyl, alkyl, alkoxy, halogen, nitro (NO₂), amine (NH₂), or substituted amines; and wherein R¹⁰ is alkyl, alkoxy, O-alkyl, N-alkyl, aromatic, heteroaromatic, cyclic, or heterocyclic.

In a various aspects, the compound has a structure represented by a formula:

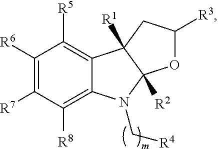

wherein n is 0-6; wherein m is 0-6; wherein each of R¹ and R², when present, is independently selected from H and —OH; wherein R³ is

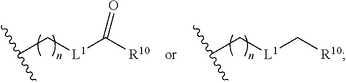

wherein L¹ is —O— or —NH—; wherein R⁴ is alkyl, alkoxy, O-alkyl, N-alkyl, aromatic, heteroaromatic, cyclic, or heterocyclic; wherein R⁵, R⁶, R⁷ and R⁸ are independently hydroxyl, alkyl, alkoxy, halogen, nitro (NO₂), amine (NH₂), or substituted amines; and wherein R¹⁰ is alkyl, alkoxy, O-alkyl, N-alkyl, aromatic, heteroaromatic, cyclic, or heterocyclic.

In a various aspects, the compound has a structure represented by a formula:

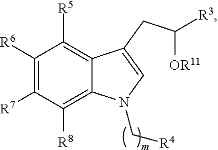

wherein n is 0-6; wherein m is 0-6; wherein R³ is

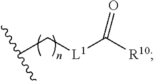

wherein L¹ is —O— or —NH—; wherein R⁴ is alkyl, alkoxy, O-alkyl, N-alkyl, aromatic, heteroaromatic, cyclic, or heterocyclic; wherein R⁵, R⁶, R⁷ and R⁸ are independently hydroxyl, alkyl, alkoxy, halogen, nitro (NO₂), amine (NH₂), or substituted amines; and wherein R¹⁰ is alkyl, alkoxy, O-alkyl, N-alkyl, aromatic, heteroaromatic, cyclic, or heterocyclic.

In a various aspects, the compound has a structure represented by a formula:

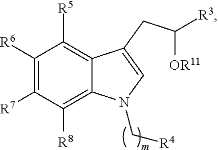

wherein n is 0-6; wherein m is 0-6; wherein R³ is

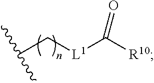

wherein L¹ is —O— or —NH—; wherein R⁴ is alkyl, alkoxy, O-alkyl, N-alkyl, aromatic, heteroaromatic, cyclic, or heterocyclic; wherein R⁵, R⁶, R⁷ and R⁸ are independently hydroxyl, alkyl, alkoxy, halogen, nitro (NO₂), amine (NH₂), or substituted amines; wherein R¹⁰ is alkyl, alkoxy, O-alkyl, N-alkyl, aromatic, heteroaromatic, cyclic, or heterocyclic; and wherein R¹¹ is hydrogen or alkyl.

In a various aspects, the compound has a structure represented by a formula:

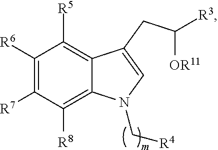

wherein n is 0-6; wherein m is 0-6; wherein R³ is

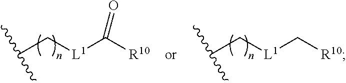

wherein L¹ is —O— or —NH—; wherein R⁴ is alkyl, alkoxy, O-alkyl, N-alkyl, aromatic, heteroaromatic, cyclic, or heterocyclic; wherein R⁵, R⁶, R⁷ and R⁸ are independently hydroxyl, alkyl, alkoxy, halogen, nitro (NO₂), amine (NH₂), or substituted amines; and wherein R¹⁰ is alkyl, alkoxy, O-alkyl, N-alkyl, aromatic, heteroaromatic, cyclic, or heterocyclic.

In a various aspects, the compound has a structure represented by a formula:

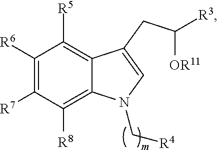

wherein n is 0-6; wherein m is 0-6; wherein R³ is

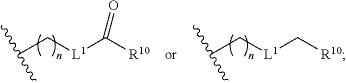

wherein L¹ is —O— or —NH—; wherein R⁴ is alkyl, alkoxy, O-alkyl, N-alkyl, aromatic, heteroaromatic, cyclic, or heterocyclic; wherein R⁵, R⁶, R⁷ and R⁸ are independently hydroxyl, alkyl, alkoxy, halogen, nitro (NO₂), amine (NH₂), or substituted amines; wherein R¹⁰ is alkyl, alkoxy, O-alkyl, N-alkyl, aromatic, heteroaromatic, cyclic, or heterocyclic; and wherein R¹¹ is hydrogen or alkyl.

In a further aspect, the compound has a structure represented by a formula:

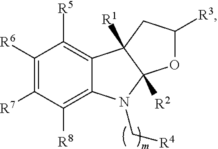

wherein n is 1; wherein m is 1; wherein each of R¹ and R² is hydrogen; wherein L¹ is —O—; wherein R³ is

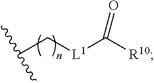

wherein R⁴ is:

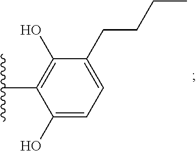 [SMILES: *c1c(O)ccc(CCCC)c1O]

and
wherein R¹⁰ is:

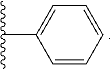 [SMILES: *c1ccccc1]

In a further aspect, the compound has a structure represented by a formula:

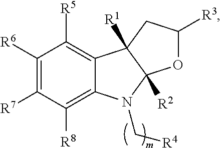

wherein n is 1; wherein m is 1; wherein each of R¹ and R² is hydrogen; wherein L¹ is —O—; wherein R³ is

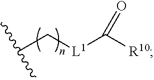

wherein R⁴ is:

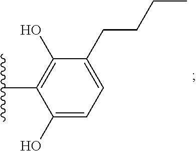

wherein R⁵, R⁶, R⁷ and R⁸ are independently hydroxyl, alkyl, alkoxy, halogen, nitro (NO₂), amine (NH₂), or substituted amines; and wherein R¹⁰ is

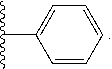 [SMILES: *c1ccccc1]

In a further aspect, the compound has a structure represented by a formula:

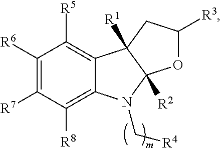

wherein n is 1; wherein m is 1; wherein each of R¹ and R² is hydrogen; wherein L¹ is —O—; wherein R³ is

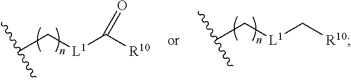

wherein R⁴ is:

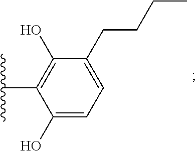 [SMILES: *c1c(O)ccc(CCCC)c1O]

and
wherein R¹⁰ is:

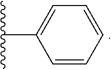 [SMILES: *c1ccccc1]

In a further aspect, the compound has a structure represented by a formula:

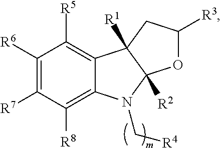

wherein n is 1; wherein m is 1; wherein each of R¹ and R² is hydrogen; wherein L¹ is —O—; wherein R³ is

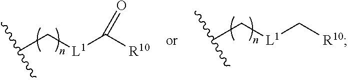

wherein R⁴ is:

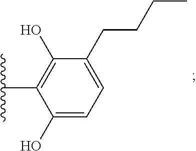

wherein R⁵, R⁶, R⁷ and R⁸ are independently hydroxyl, alkyl, alkoxy, halogen, nitro (NO₂), amine (NH₂), or substituted amines; and wherein R¹⁰ is:

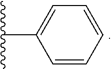 [SMILES: *c1ccccc1]

In a further aspect, the compound has a structure represented by a formula:

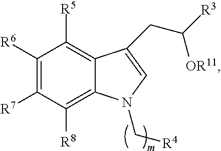

wherein n is 1; wherein m is 1; wherein L¹ is —O—; wherein R³ is

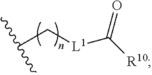

wherein R⁴ is:

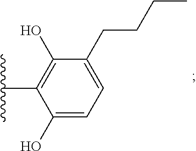 [SMILES: *c1c(O)ccc(CCCC)c1O]

and
wherein R¹⁰ is:

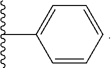 [SMILES: *c1ccccc1]

In a further aspect, the compound has a structure represented by a formula:

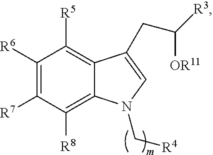

wherein n is 1; wherein m is 1; wherein L¹ is —O—; wherein R³ is

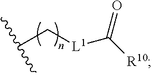

wherein R⁴ is:

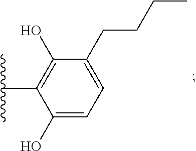

wherein R⁵, R⁶, R⁷ and R⁸ are independently hydroxyl, alkyl, alkoxy, halogen, nitro (NO₂), amine (NH₂), or substituted amines; and wherein R¹⁰ is:

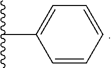 [SMILES: *c1ccccc1]

In a further aspect, the compound has a structure represented by a formula:

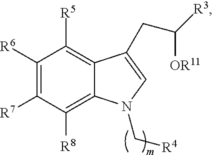

wherein n is 1; wherein m is 1; wherein L¹ is —O—; wherein R³ is

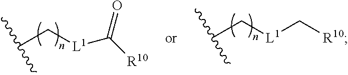

wherein R⁴ is:

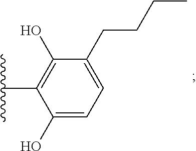 [SMILES: *c1c(O)ccc(CCCC)c1O]

and
wherein R¹⁰ is:

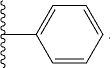 [SMILES: *c1ccccc1]

In a further aspect, the compound has a structure represented by a formula:

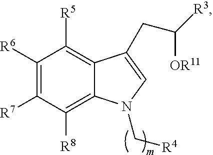

wherein n is 1; wherein m is 1; wherein L¹ is —O—; wherein R³ is

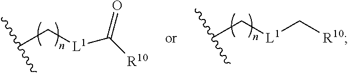

wherein R⁴ is:

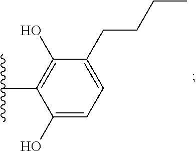

wherein R⁵, R⁶, R⁷ and R⁸ are independently hydroxyl, alkyl, alkoxy, halogen, nitro (NO₂), amine (NH₂), or substituted amines; and wherein R¹⁰ is

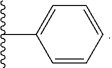 [SMILES: *c1ccccc1]

In a various aspects, the compound has a structure represented by a formula:

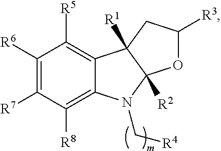

wherein n is 0-6; wherein m is 0-6; wherein each of R¹ and R², when present, is independently selected from H and —OH; wherein R³ is

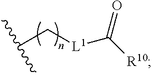

wherein L¹ is —O— or —NH—; wherein R⁴ is alkyl, alkoxy, O-alkyl, N-alkyl, aromatic, heteroaromatic, cyclic, or heterocyclic; wherein R⁵, R⁶, R⁷ and R⁸ are independently hydrogen, hydroxyl, alkyl, alkoxy, halogen, nitro (NO₂), amine (NH₂), or substituted amines; and wherein R¹⁰ is alkyl, alkoxy, O-alkyl, N-alkyl, aromatic, heteroaromatic, cyclic, or heterocyclic.

In a various aspects, the compound has a structure represented by a formula:

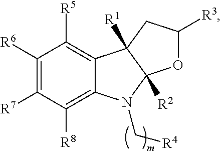

wherein n is 0-6; wherein m is 0-6; wherein each of R¹ and R², when present, is independently selected from H and —OH; wherein R³ is

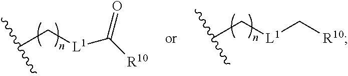

wherein L¹ is —O— or —NH—; wherein R⁴ is alkyl, alkoxy, O-alkyl, N-alkyl, aromatic, heteroaromatic, cyclic, or heterocyclic; wherein R⁵, R⁶, R⁷ and R⁸ are independently hydrogen, hydroxyl, alkyl, alkoxy, halogen, nitro (NO₂), amine (NH₂), or substituted amines; and wherein R¹⁰ is alkyl, alkoxy, O-alkyl, N-alkyl, aromatic, hetero aromatic, cyclic, or heterocyclic.

In a various aspects, the compound has a structure represented by a formula:

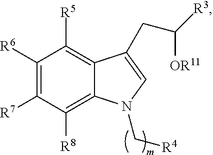

wherein n is 0-6; wherein m is 0-6; wherein R³ is

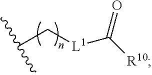

wherein L¹ is —O— or —NH—; wherein R⁴ is alkyl, alkoxy, O-alkyl, N-alkyl, aromatic, heteroaromatic, cyclic, or heterocyclic; wherein R⁵, R⁶, R⁷ and R⁸ are independently hydrogen, hydroxyl, alkyl, alkoxy, halogen, nitro (NO₂), amine (NH₂), or substituted amines; and wherein R¹⁰ is alkyl, alkoxy, O-alkyl, N-alkyl, aromatic, heteroaromatic, cyclic, or heterocyclic.

In a various aspects, the compound has a structure represented by a formula:

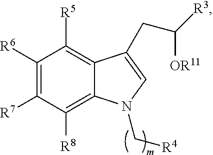

wherein n is 0-6; wherein m is 0-6; wherein R³ is

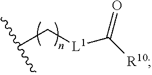

wherein L¹ is —O— or —NH—; wherein R⁴ is alkyl, alkoxy, O-alkyl, N-alkyl, aromatic, heteroaromatic, cyclic, or heterocyclic; wherein R⁵, R⁶, R⁷ and R⁸ are independently hydrogen, hydroxyl, alkyl, alkoxy, halogen, nitro (NO₂), amine (NH₂), or substituted amines; wherein R¹⁰ is alkyl, alkoxy, O-alkyl, N-alkyl, aromatic, heteroaromatic, cyclic, or heterocyclic; and wherein R¹¹ is hydrogen or alkyl.

In a various aspects, the compound has a structure represented by a formula:

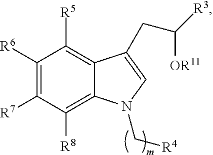

wherein n is 0-6; wherein m is 0-6; wherein R³ is

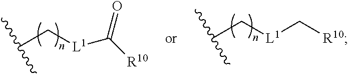

wherein L¹ is —O— or —NH—; wherein R⁴ is alkyl, alkoxy, O-alkyl, N-alkyl, aromatic, heteroaromatic, cyclic, or heterocyclic; wherein R⁵, R⁶, R⁷ and R⁸ are independently hydrogen, hydroxyl, alkyl, alkoxy, halogen, nitro (NO₂), amine (NH₂), or substituted amines; and wherein R¹⁰ is alkyl, alkoxy, O-alkyl, N-alkyl, aromatic, heteroaromatic, cyclic, or heterocyclic.

In a various aspects, the compound has a structure represented by a formula:

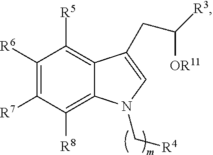

wherein n is 0-6; wherein m is 0-6; wherein R³ is

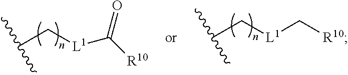

wherein L¹ is —O— or —NH—; wherein R⁴ is alkyl, alkoxy, O-alkyl, N-alkyl, aromatic, heteroaromatic, cyclic, or heterocyclic; wherein R⁵, R⁶, R⁷ and R⁸ are independently hydrogen, hydroxyl, alkyl, alkoxy, halogen, nitro (NO₂), amine (NH₂), or substituted amines; wherein R¹⁰ is alkyl, alkoxy, O-alkyl, N-alkyl, aromatic, heteroaromatic, cyclic, or heterocyclic; and wherein R¹¹ is hydrogen or alkyl.

In a further aspect, the compound has a structure represented by a formula:

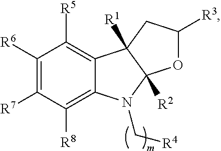

wherein n is 1; wherein m is 1; wherein each of R¹ and R² is hydrogen; wherein L¹ is —O—; wherein R³ is

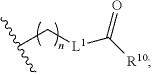

wherein R⁴ is:

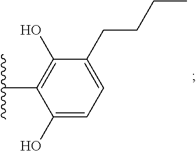

wherein R⁵, R⁶, R⁷ and R⁸ are independently hydrogen, hydroxyl, alkyl, alkoxy, halogen, nitro

(NO₂), amine (NH₂), or substituted amines; and wherein R¹⁰ is:

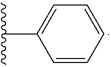 [SMILES: *c1ccccc1]

In a further aspect, the compound has a structure represented by a formula:

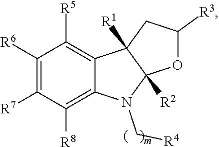

wherein n is 1; wherein m is 1; wherein each of R¹ and R² is hydrogen; wherein L¹ is —O—; wherein R³ is

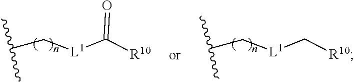

wherein R⁴ is:

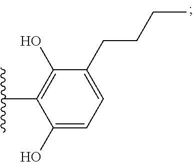

wherein R⁵, R⁶, R⁷ and R⁸ are independently hydrogen, hydroxyl, alkyl, alkoxy, halogen, nitro (NO₂), amine (NH₂), or substituted amines; and wherein R¹⁰ is:

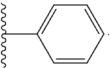 [SMILES: *c1ccccc1]

In a further aspect, the compound has a structure represented by a formula:

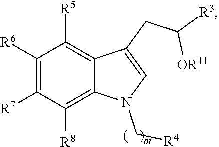

wherein n is 1; wherein m is 1; wherein L¹ is —O—; wherein R³ is

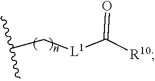

wherein R⁴ is:

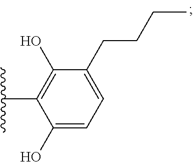

wherein R⁵, R⁶, R⁷ and R⁸ are independently hydrogen, hydroxyl, alkyl, alkoxy, halogen, nitro

(NO₂), amine (NH₂), or substituted amines; and wherein R¹⁰ is:

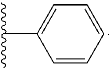 [SMILES: *c1ccccc1]

In a further aspect, the compound has a structure represented by a formula:

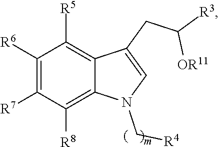

wherein n is 1; wherein m is 1; wherein L¹ is —O—; wherein R³ is

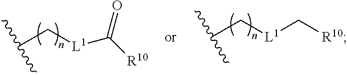

wherein R⁴ is:

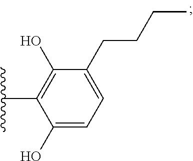

wherein R⁵, R⁶, R⁷ and R⁸ are independently hydrogen, hydroxyl, alkyl, alkoxy, halogen, nitro (NO₂), amine (NH₂), or substituted amines; and wherein R¹⁰ is:

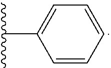 [SMILES: *c1ccccc1]

In a further aspect, the compound has a structure represented by a formula:

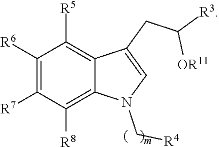

In a further aspect, the compound has a structure represented by a formula:

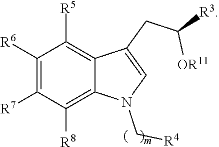

In a further aspect, the compound has a structure represented by a formula:

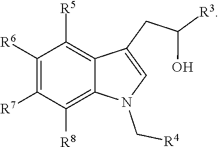

In a further aspect, the compound has a structure represented by a formula:

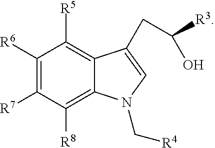

In a further aspect, the compound has a structure represented by a formula:

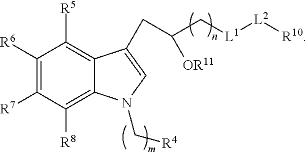

In a further aspect, the compound has a structure represented by a formula:

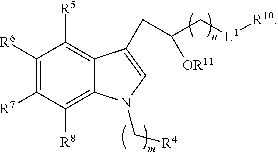

In a further aspect, the compound has a structure represented by a formula:

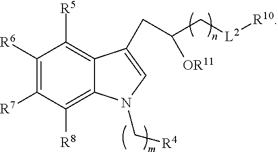

In a further aspect, the compound has a structure represented by a formula:

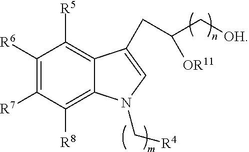

In a further aspect, the compound has a structure represented by a formula:

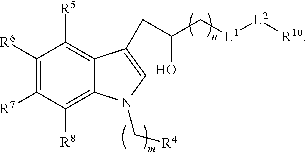

In a further aspect, the compound has a structure represented by a formula:

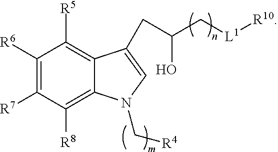

In a further aspect, the compound has a structure represented by a formula:

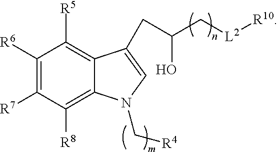

In a further aspect, the compound has a structure represented by a formula:

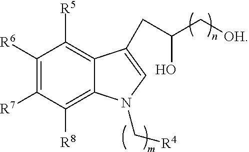

In a further aspect, the compound has a structure represented by a formula:

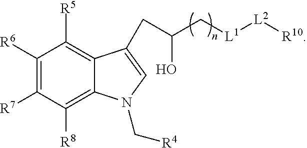

In a further aspect, the compound has a structure represented by a formula:

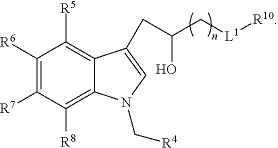

In a further aspect, the compound has a structure represented by a formula:

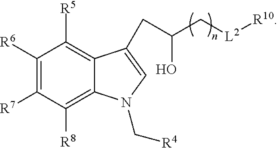

In a further aspect, the compound has a structure represented by a formula:

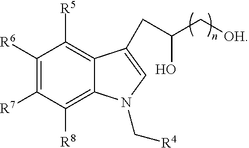

In a further aspect, the compound has a structure represented by a formula:

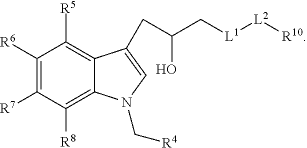

In a further aspect, the compound has a structure represented by a formula:

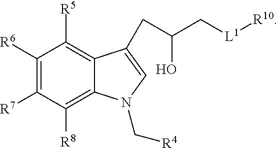

In a further aspect, the compound has a structure represented by a formula:

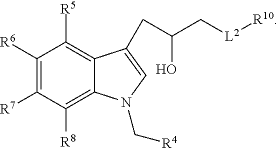

In a further aspect, the compound has a structure represented by a formula:

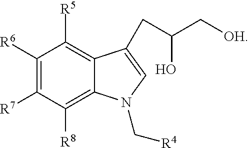

In a further aspect, the compound has a structure represented by a formula:

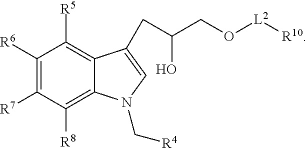

In a further aspect, the compound has a structure represented by a formula:

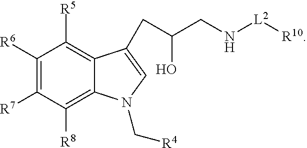

In a further aspect, the compound has a structure represented by a formula:

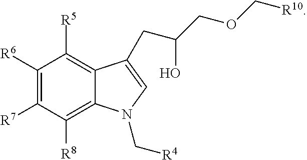

In a further aspect, the compound has a structure represented by a formula:

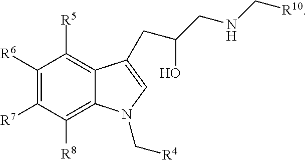

In a further aspect, the compound has a structure represented by a formula:

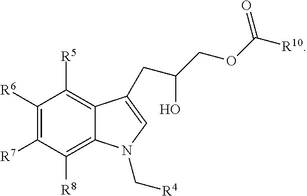

In a further aspect, the compound has a structure represented by a formula:

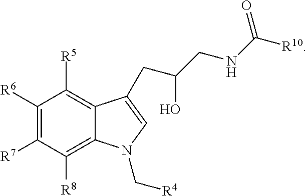

In a further aspect, the compound has a structure represented by a formula:

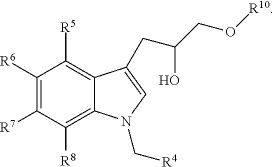

In a further aspect, the compound has a structure represented by a formula:

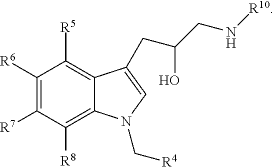

In a further aspect, the compound has a structure represented by a formula:

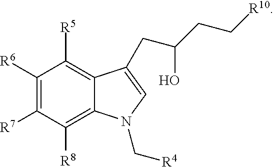

In a further aspect, the compound has a structure represented by a formula:

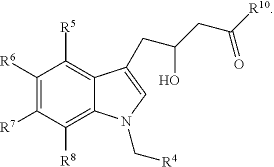

In a further aspect, the compound has a structure represented by a formula:

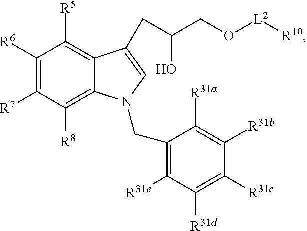

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

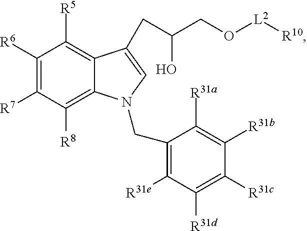

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

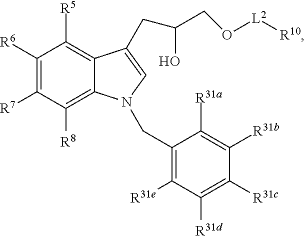

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

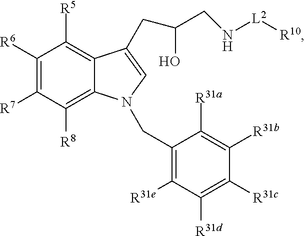

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

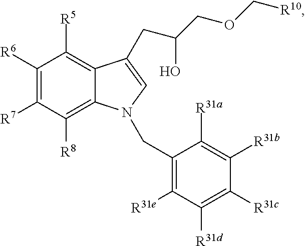

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

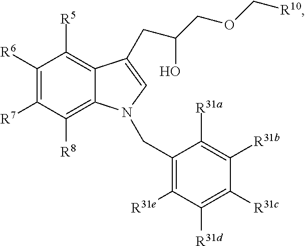

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

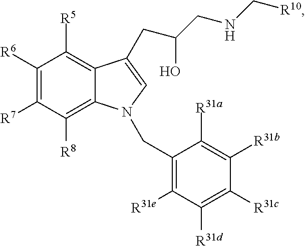

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

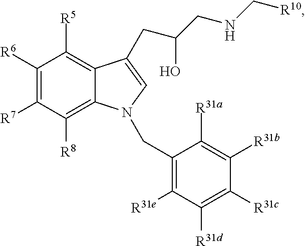

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

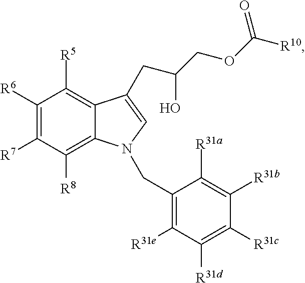

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

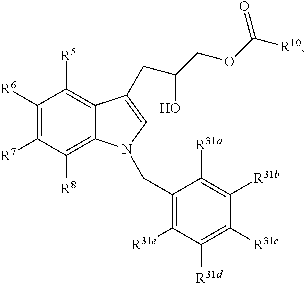

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

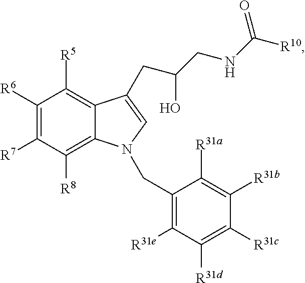

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

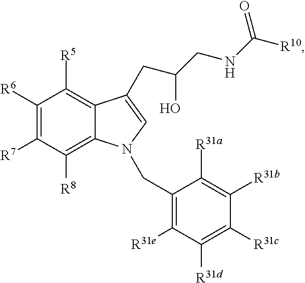

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

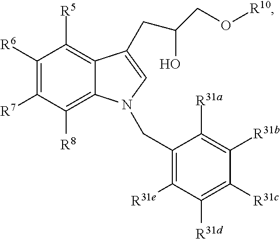

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

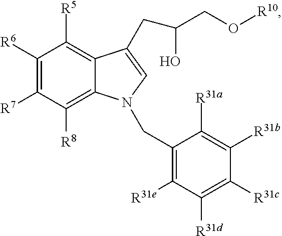

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

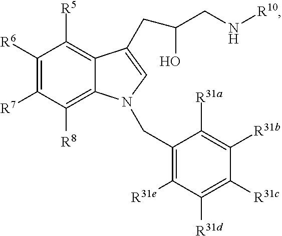

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

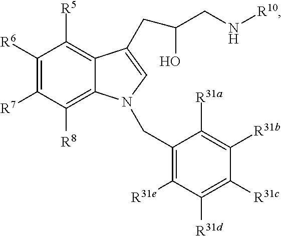

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

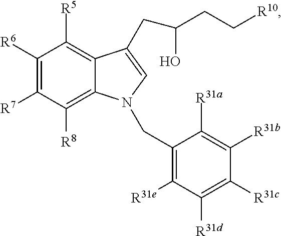

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

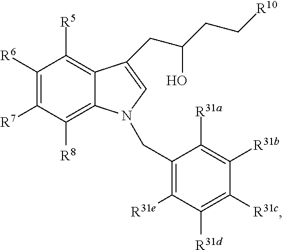

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

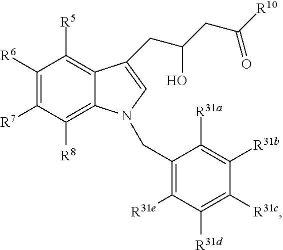

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

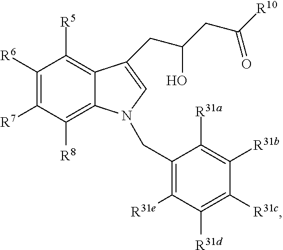

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

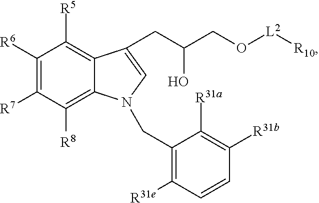

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

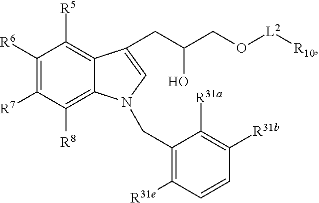

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

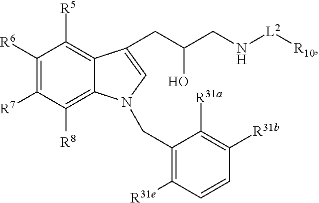

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

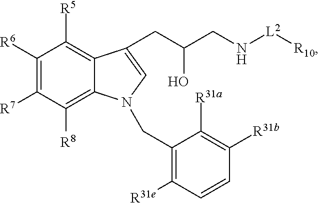

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

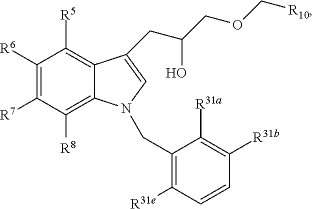

wherein each of R^{31a}, R^{31b}, and R^{31e} is independently selected from hydrogen, halogen, —OH, —NO₂, —NH₂, —NHCH₃, —N(CH₃)₂, NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

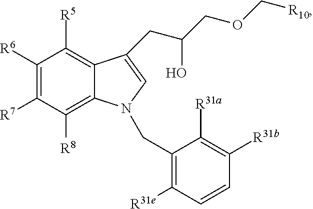

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

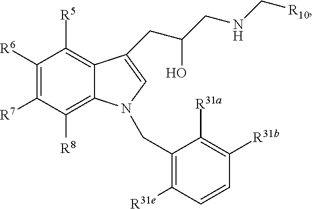

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

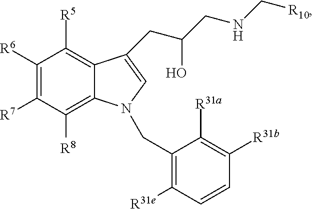

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

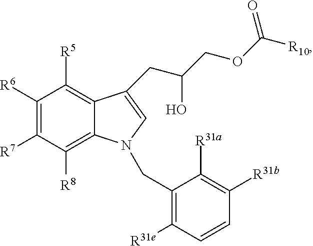

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

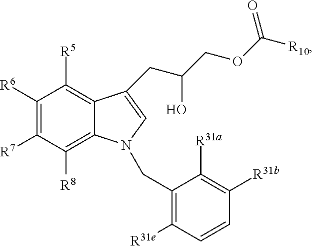

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

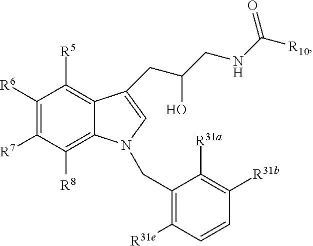

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

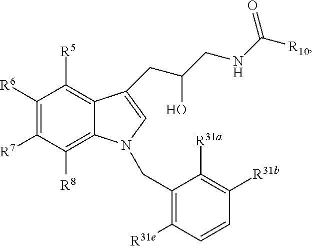

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

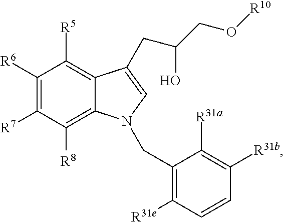

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

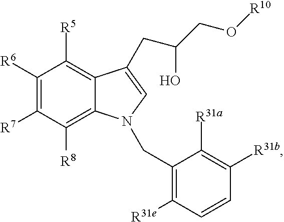

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

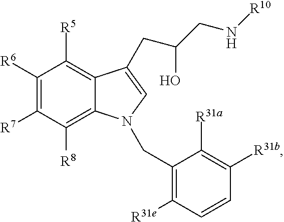

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

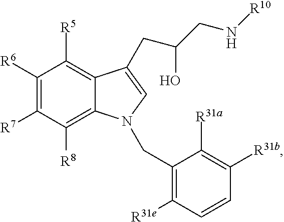

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

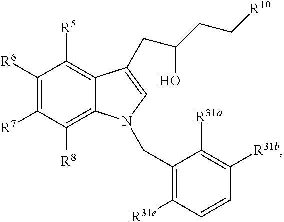

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

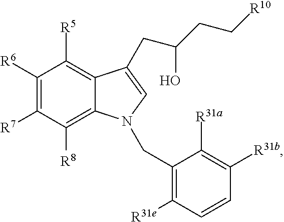

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

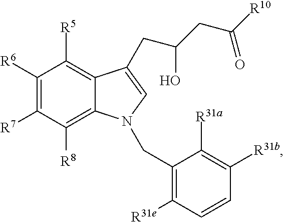

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

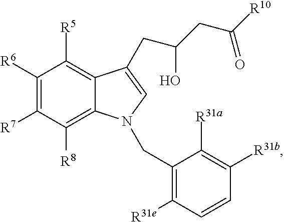

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

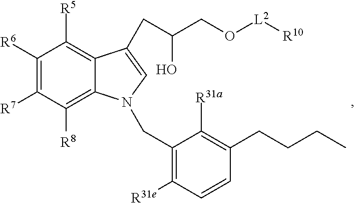

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

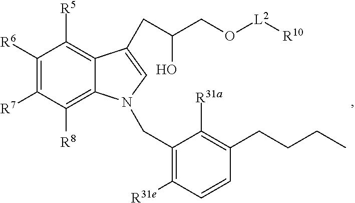

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

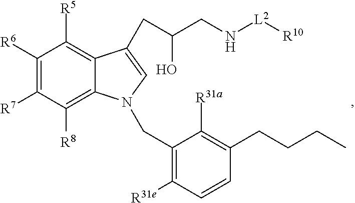

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

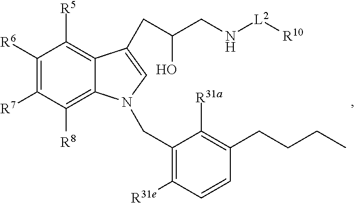

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

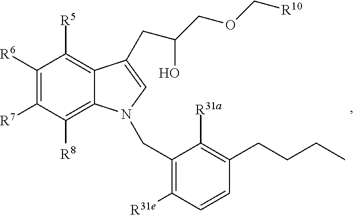

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

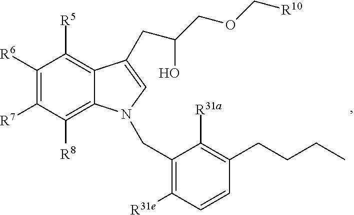

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

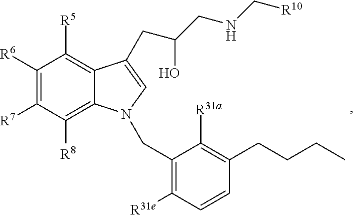

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

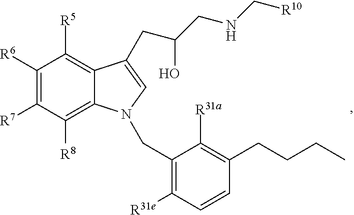

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

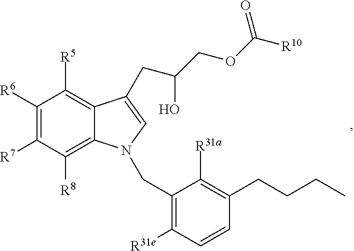

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

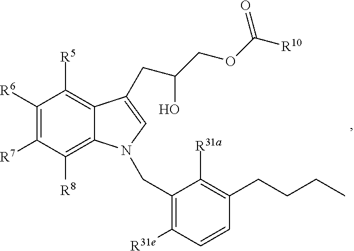

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

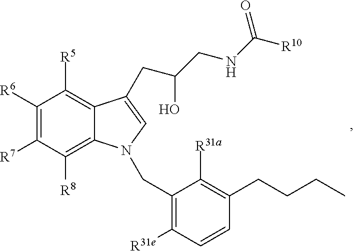

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

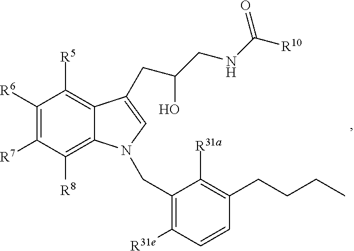

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

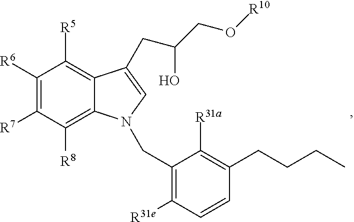

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

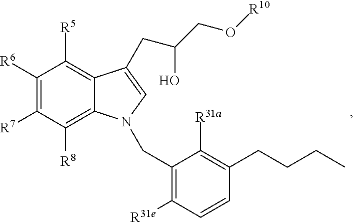

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

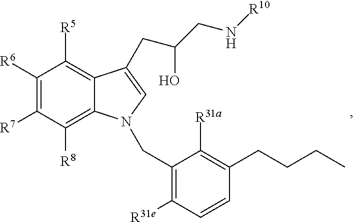

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

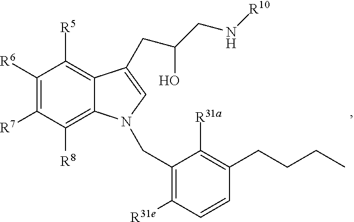

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

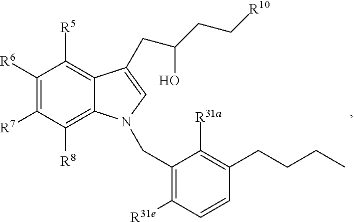

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

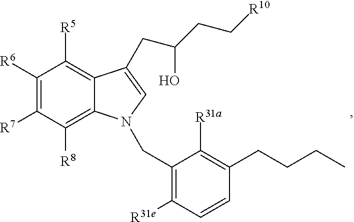

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

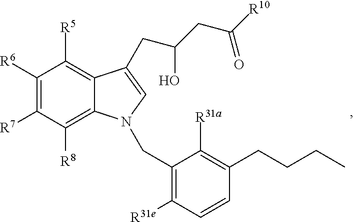

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

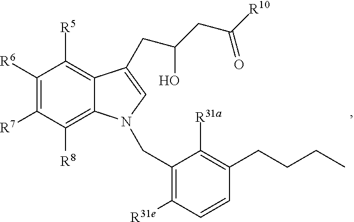

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

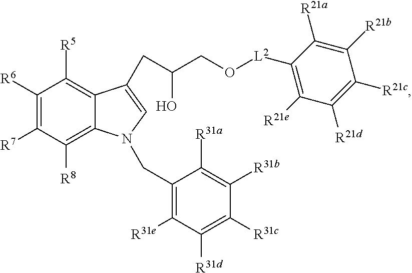

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d} and R^{31e} are hydrogen; and wherein all other variables are as defined herein; or a pharmaceutically acceptable salt thereof.

In a further aspect, the compound has a structure represented by a formula:

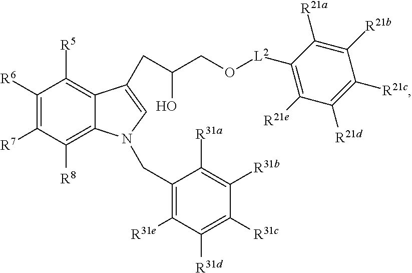

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

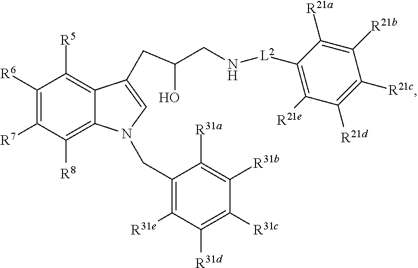

wherein each of R^{21a}, R^{21b}, R^{21c}, R^{21d} and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d} and R^{31e} are hydrogen; and wherein all other variables are as defined herein; or a pharmaceutically acceptable salt thereof.

In a further aspect, the compound has a structure represented by a formula:

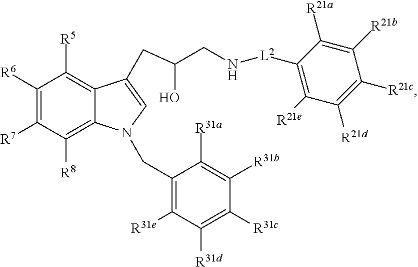

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

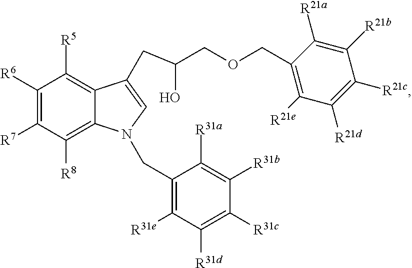

wherein each of R^{21a}, R^{21b}, R^{21c}, R^{21d} and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

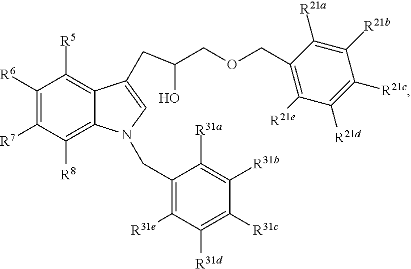

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

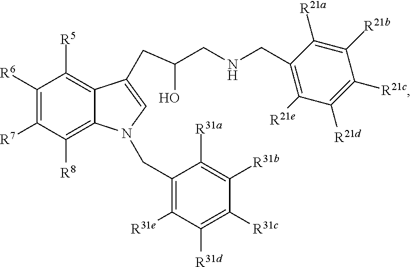

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d} and R^{31e} are hydrogen; and wherein all other variables are as defined herein; or a pharmaceutically acceptable salt thereof.

In a further aspect, the compound has a structure represented by a formula:

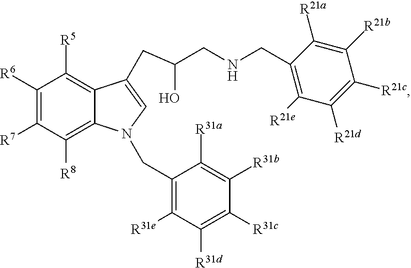

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

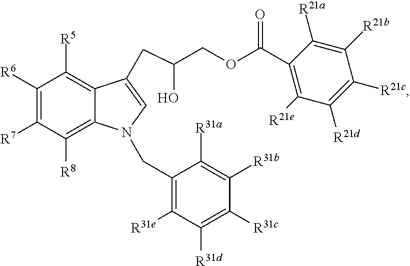

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31e}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

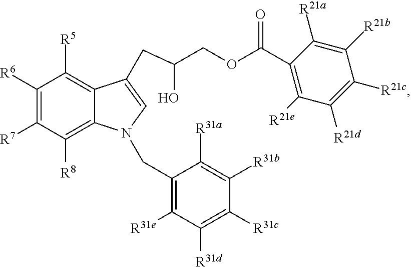

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

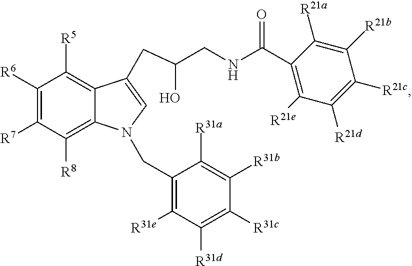

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d} and R^{31e} are hydrogen; and wherein all other variables are as defined herein; or a pharmaceutically acceptable salt thereof.

In a further aspect, the compound has a structure represented by a formula:

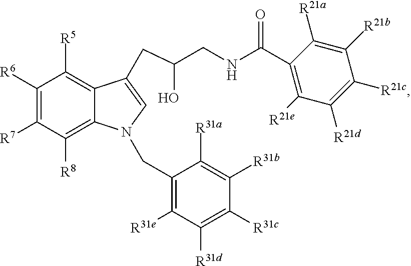

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

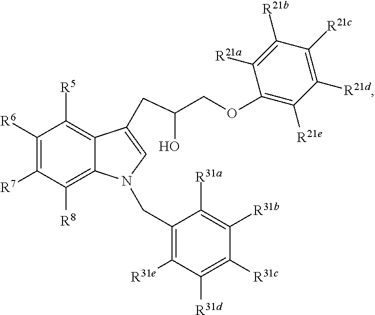

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31e}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

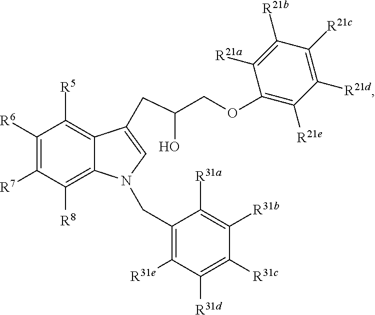

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

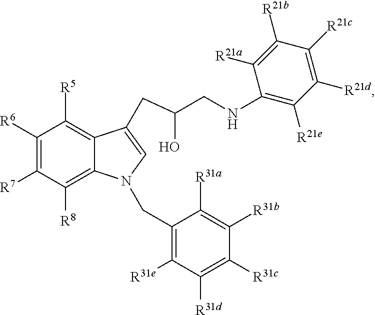

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

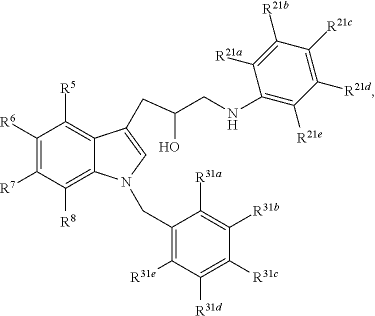

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

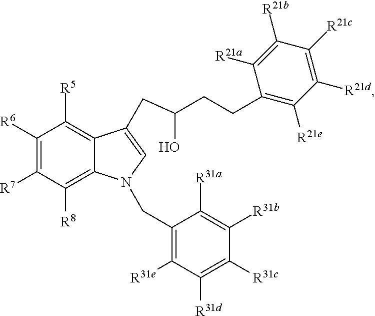

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

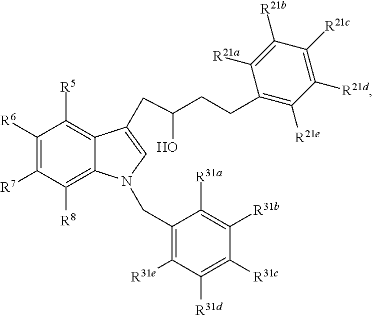

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

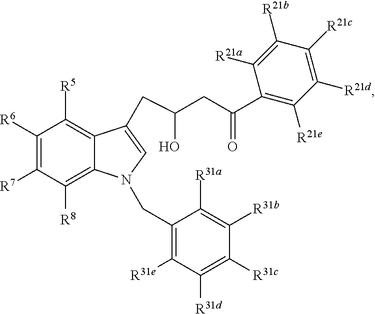

wherein each of R^{21a}, R^{21b}, R^{21c}, R^{21d} and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31e}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d} and R^{31e} are hydrogen; and wherein all other variables are as defined herein; or a pharmaceutically acceptable salt thereof.

In a further aspect, the compound has a structure represented by a formula:

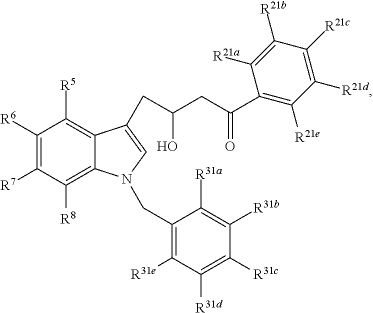

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

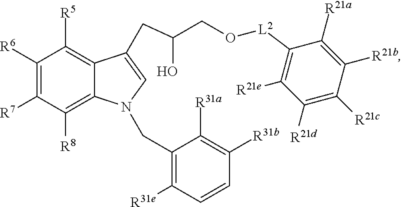

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

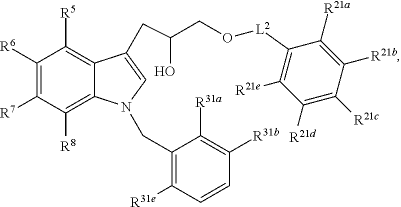

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

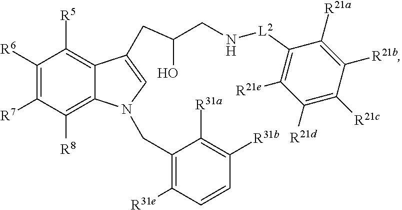

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

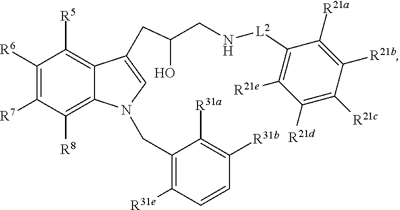

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

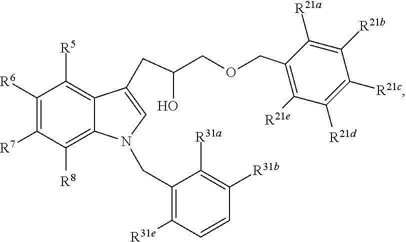

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

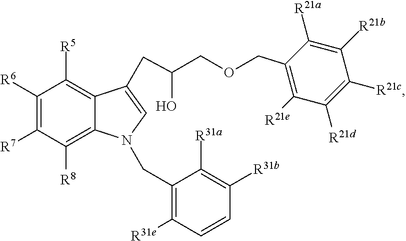

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

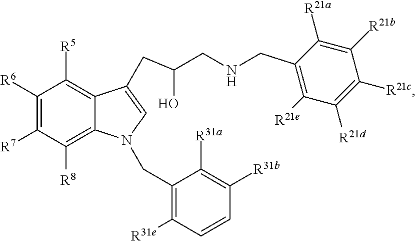

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

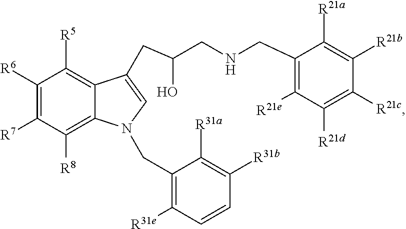

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

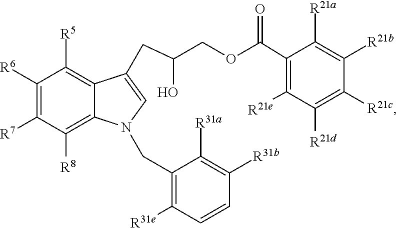

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

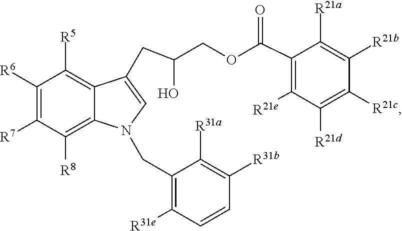

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

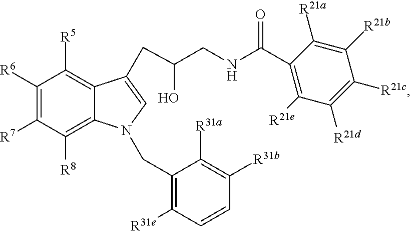

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

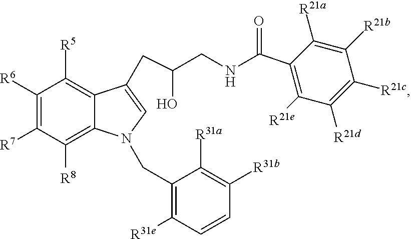

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

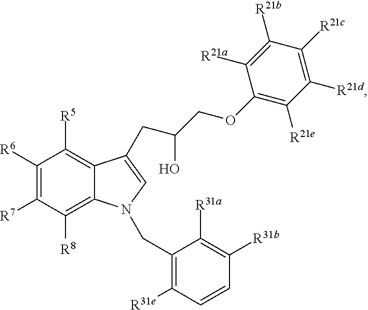

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

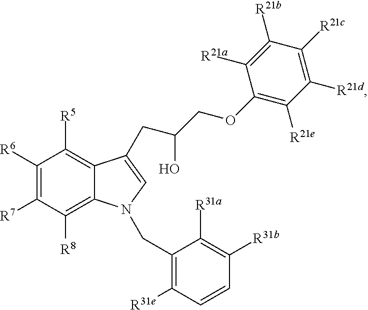

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

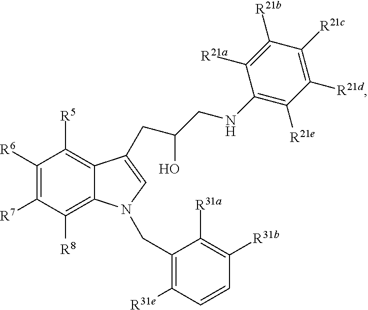

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

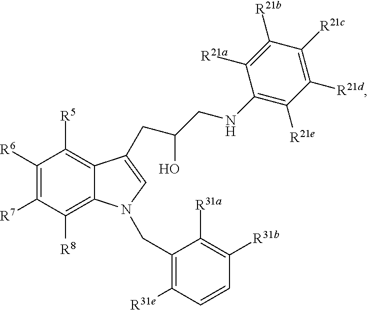

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

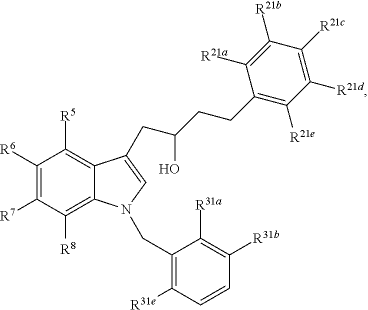

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

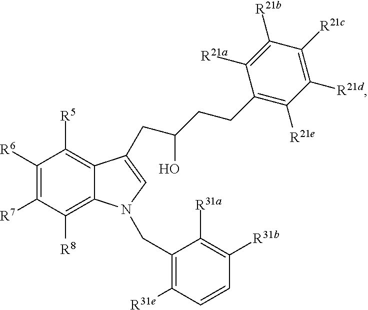

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

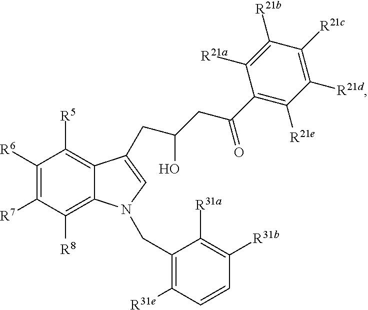

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

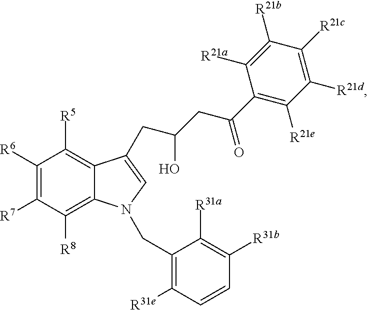

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

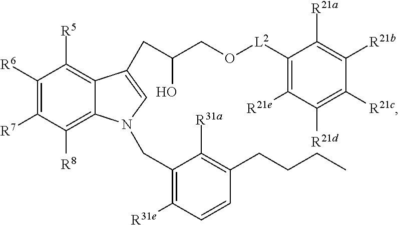

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

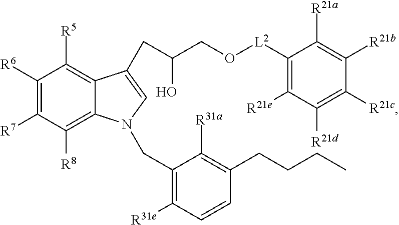

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

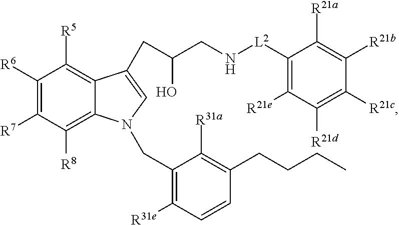

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

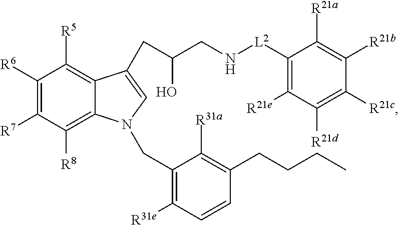

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

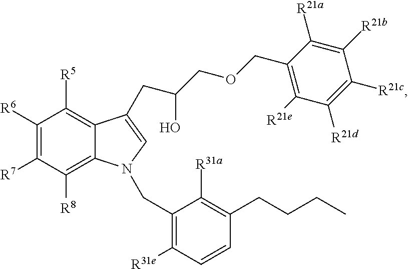

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

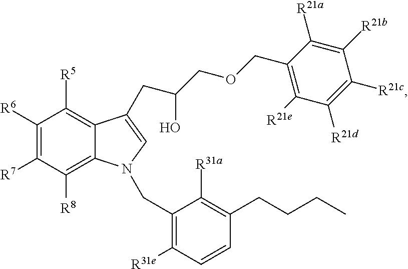

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

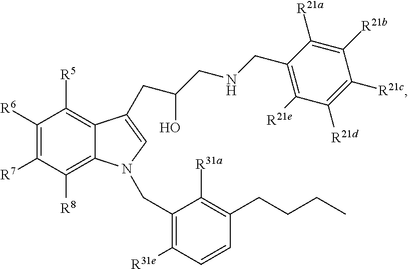

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

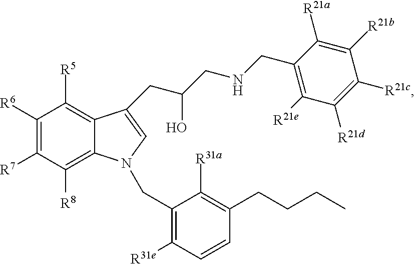

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

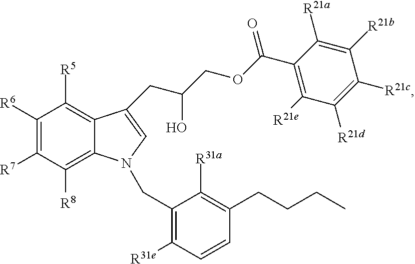

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

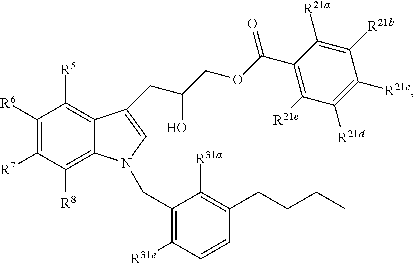

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

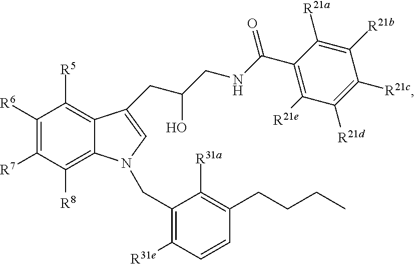

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

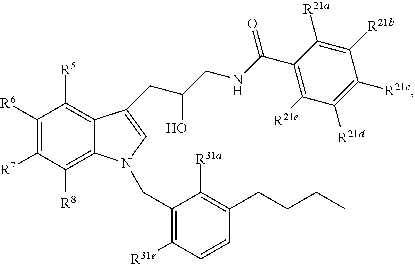

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

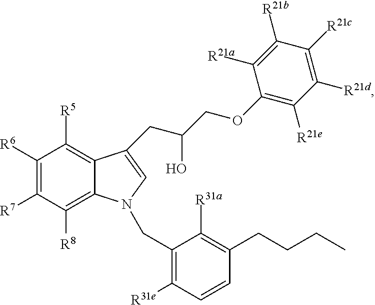

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

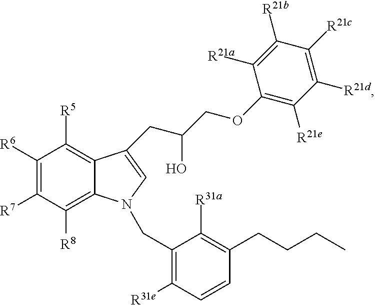

wherein each of R^{21a}, R^{21b}, R^{21c}, R^{21d}, and R²le is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

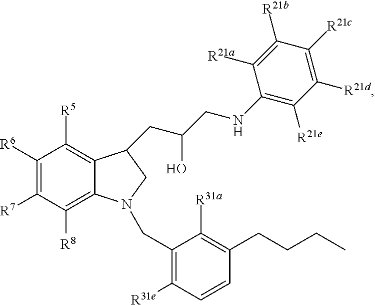

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

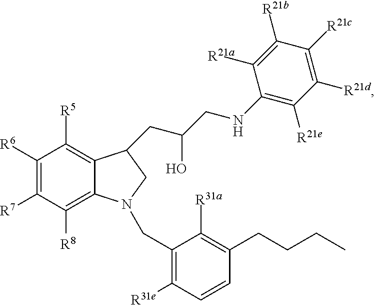

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

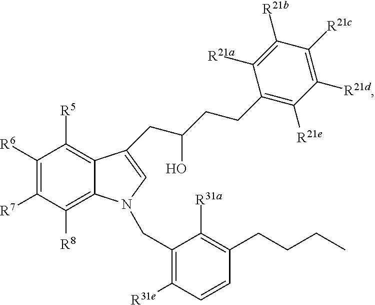

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

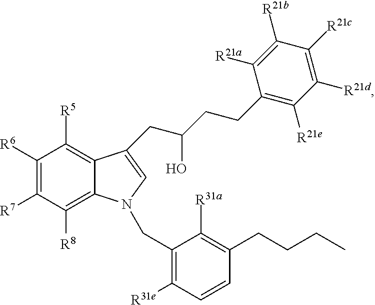

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

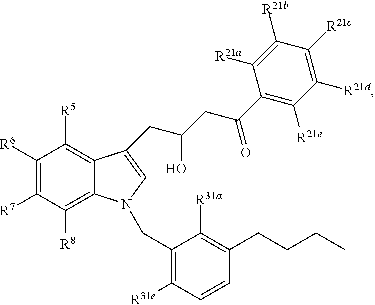

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

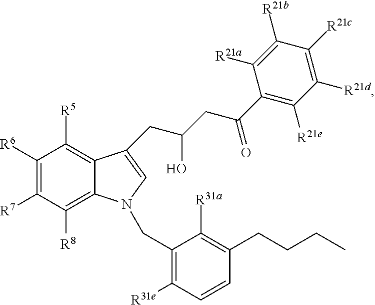

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

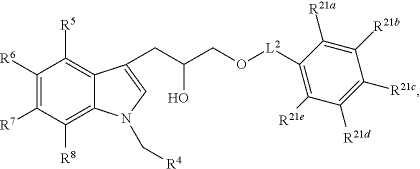

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

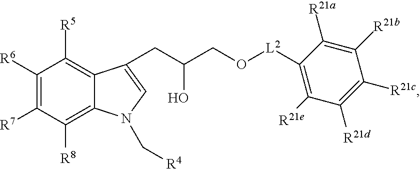

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

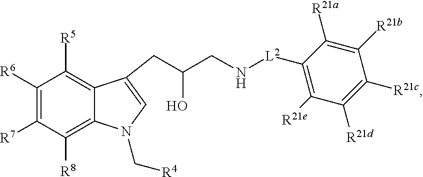

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

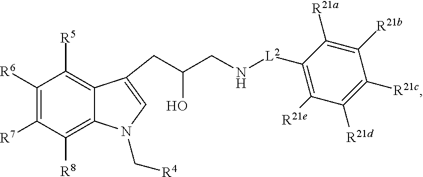

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

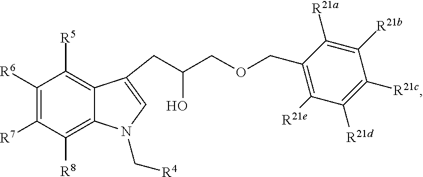

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

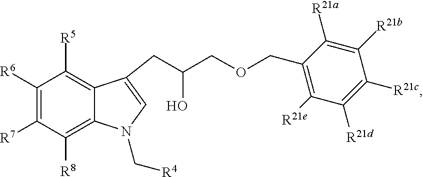

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

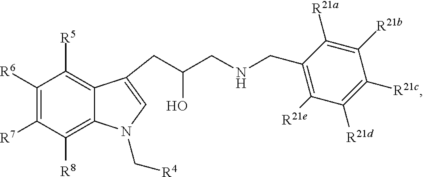

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

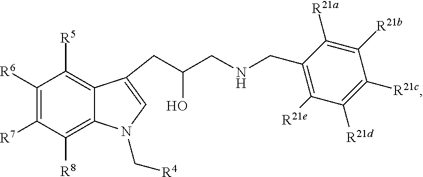

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

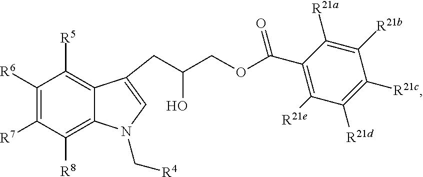

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

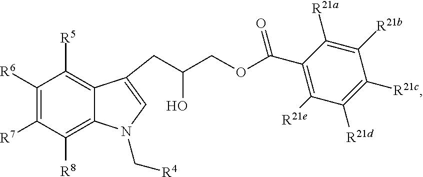

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

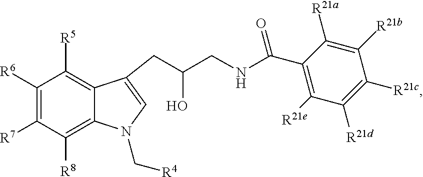

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

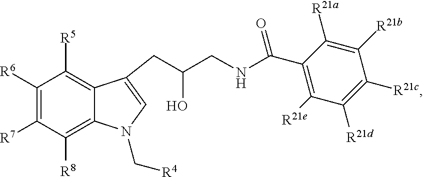

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

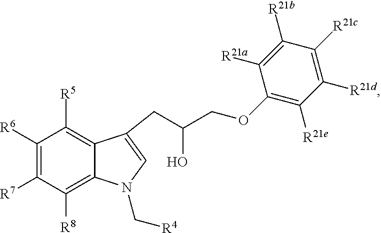

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

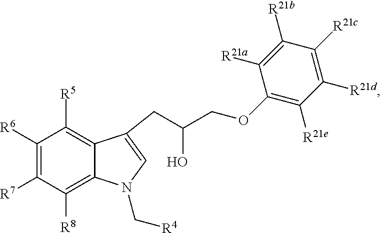

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

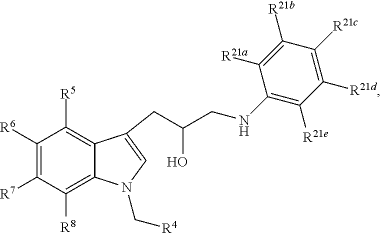

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

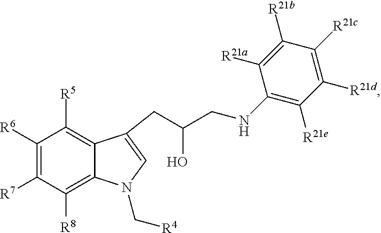

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

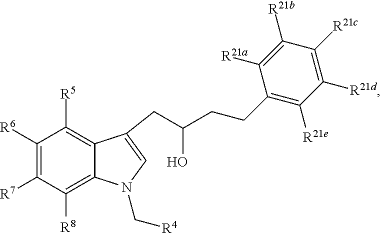

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

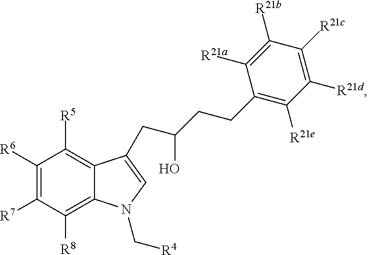

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

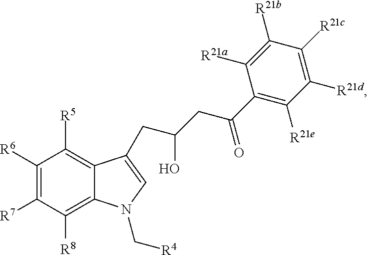

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

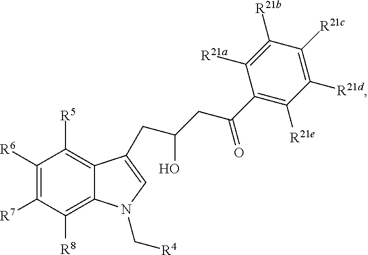

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

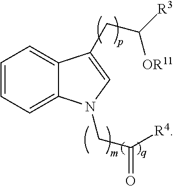

In a further aspect, the compound has a structure represented by a formula:

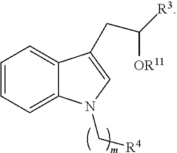

In a further aspect, the compound has a structure represented by a formula:

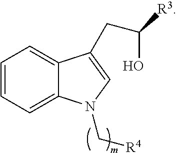

In a further aspect, the compound has a structure represented by a formula:

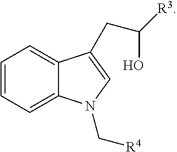

In a further aspect, the compound has a structure represented by a formula:

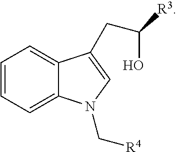

In a further aspect, the compound has a structure represented by a formula:

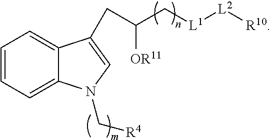

In a further aspect, the compound has a structure represented by a formula:

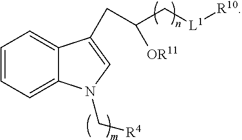

In a further aspect, the compound has a structure represented by a formula:

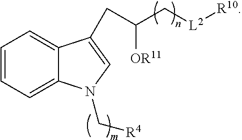

In a further aspect, the compound has a structure represented by a formula:

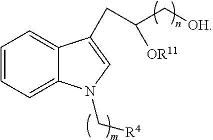

In a further aspect, the compound has a structure represented by a formula:

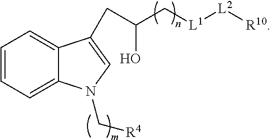

In a further aspect, the compound has a structure represented by a formula:

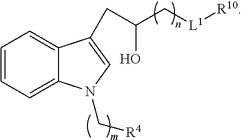

In a further aspect, the compound has a structure represented by a formula:

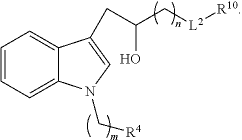

In a further aspect, the compound has a structure represented by a formula:

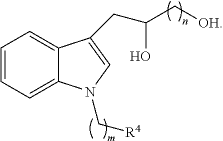

In a further aspect, the compound has a structure represented by a formula:

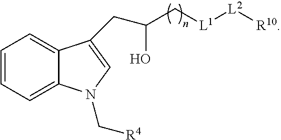

In a further aspect, the compound has a structure represented by a formula:

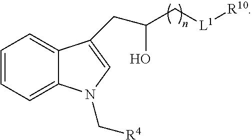

In a further aspect, the compound has a structure represented by a formula:

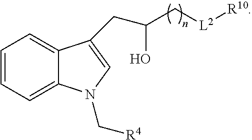

In a further aspect, the compound has a structure represented by a formula:

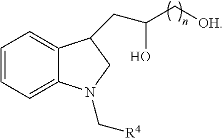

In a further aspect, the compound has a structure represented by a formula:

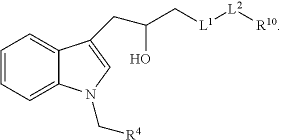

In a further aspect, the compound has a structure represented by a formula:

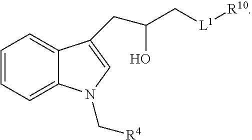

In a further aspect, the compound has a structure represented by a formula:

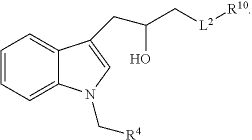

In a further aspect, the compound has a structure represented by a formula:

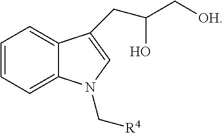

In a further aspect, the compound has a structure represented by a formula:

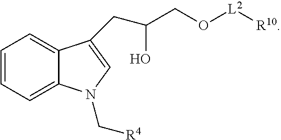

In a further aspect, the compound has a structure represented by a formula:

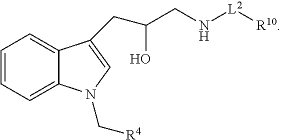

In a further aspect, the compound has a structure represented by a formula:

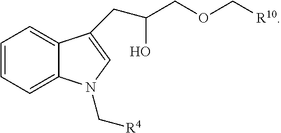

In a further aspect, the compound has a structure represented by a formula:

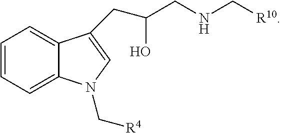

In a further aspect, the compound has a structure represented by a formula:

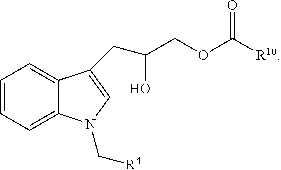

In a further aspect, the compound has a structure represented by a formula:

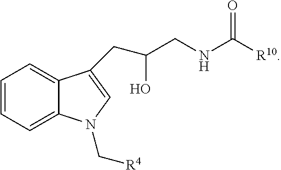

In a further aspect, the compound has a structure represented by a formula:

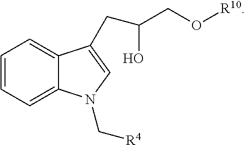

In a further aspect, the compound has a structure represented by a formula:

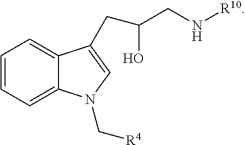

In a further aspect, the compound has a structure represented by a formula:

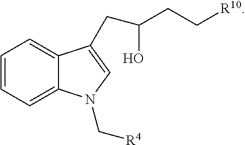

In a further aspect, the compound has a structure represented by a formula:

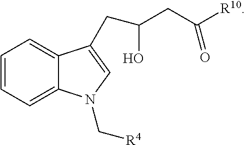

In a further aspect, the compound has a structure represented by a formula:

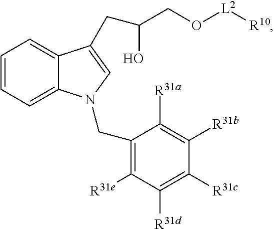

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

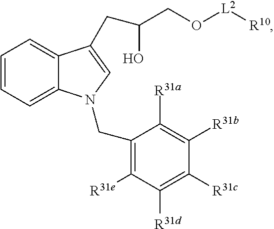

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

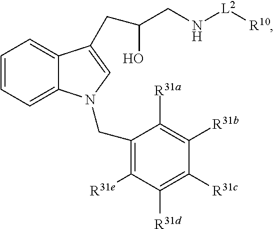

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

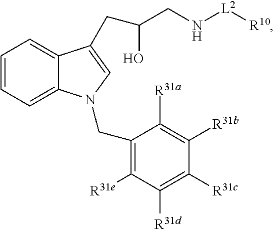

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

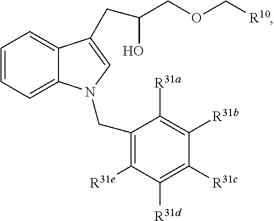

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

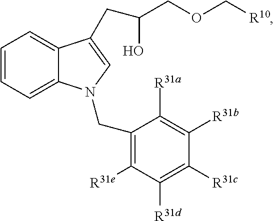

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

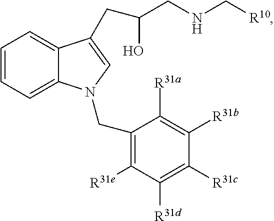

wherein each of R^{31a}, R^{31b}, R^{31e}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31e}, R^{31d}, and R^{31e} are hydrogen; and wherein all other variables are as defined herein; or a pharmaceutically acceptable salt thereof.

In a further aspect, the compound has a structure represented by a formula:

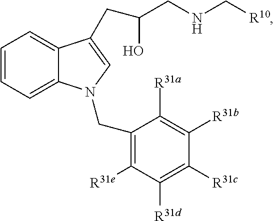

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

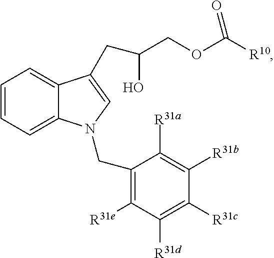

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

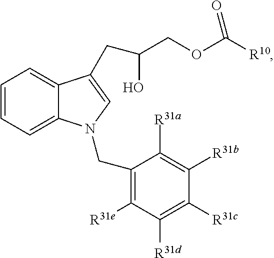

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

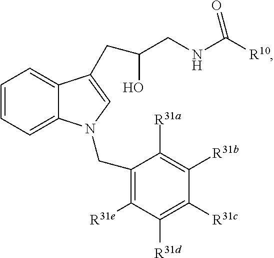

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

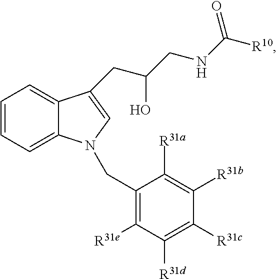

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

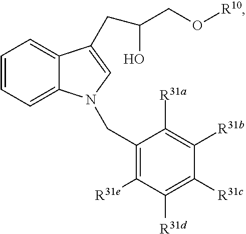

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

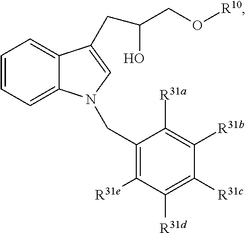

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

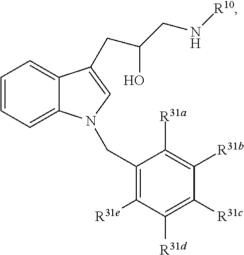

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

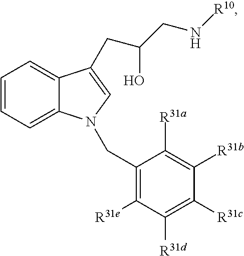

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

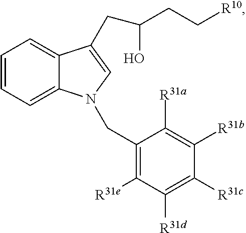

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

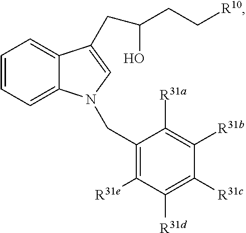

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

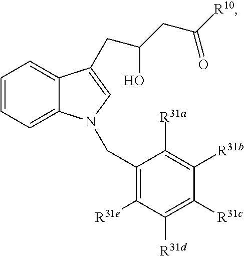

wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

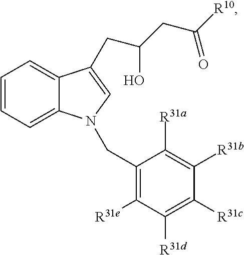

wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

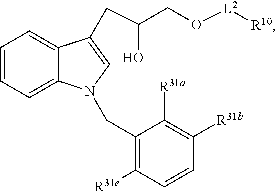

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

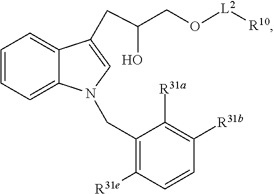

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

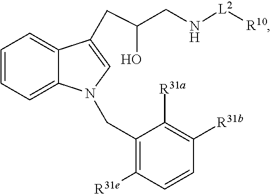

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

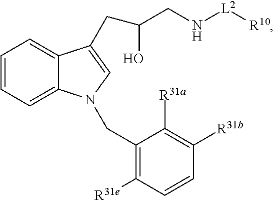

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

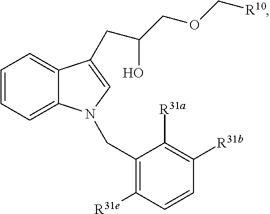

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

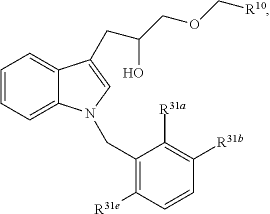

wherein each of R^{31a}, R^{31b}, and R^{31e} is independently selected from hydrogen, —F, —OH, —NH₂, —NHCH₃, —NHCH₂CH₃, methyl, —CH₂F, —CF₃, and —OCH₃; and wherein all other variables are as defined herein; or a pharmaceutically acceptable salt thereof.

In a further aspect, the compound has a structure represented by a formula:

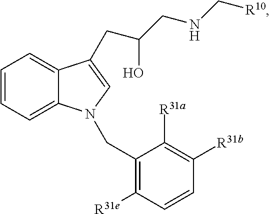

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

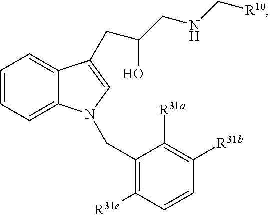

wherein each of R^{31a}, R^{31b}, and R^{31e} is independently selected from hydrogen, —F, —OH, NH₂, NHCH₃, NHCH₂CH₃, methyl, —CH₂F, CF₃, and OCH₃; and wherein all other variables are as defined herein; or a pharmaceutically acceptable salt thereof.

In a further aspect, the compound has a structure represented by a formula:

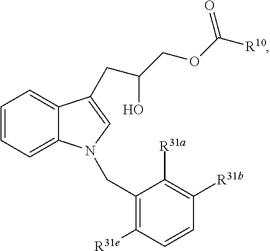

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

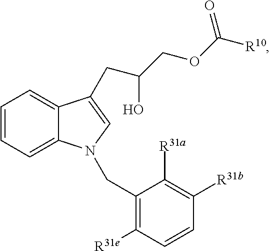

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

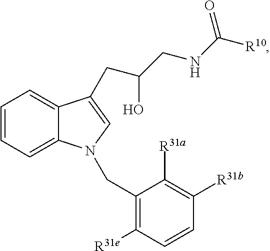

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

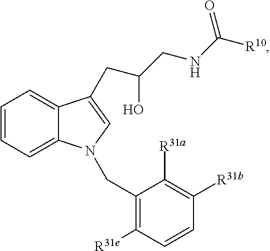

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

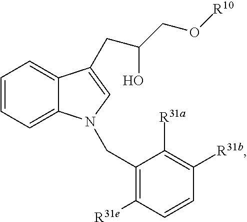

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

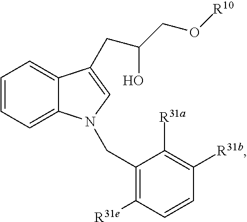

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

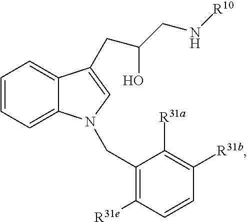

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

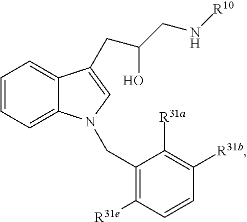

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

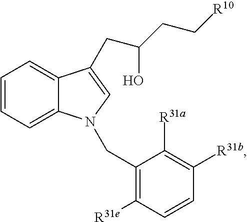

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

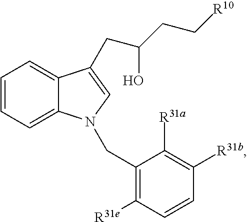

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

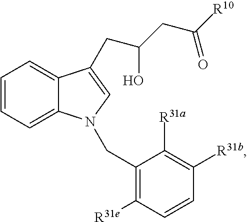

wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

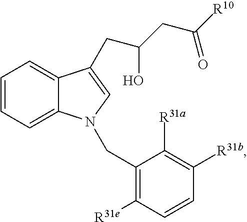

wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

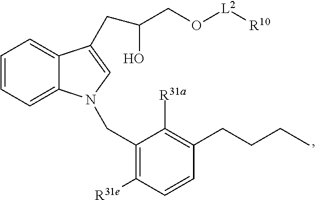

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

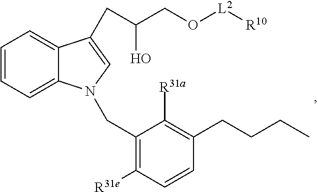

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

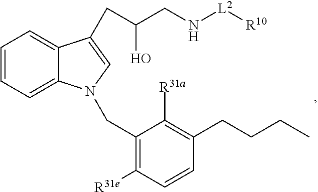

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

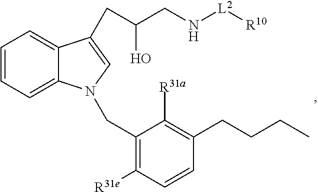

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

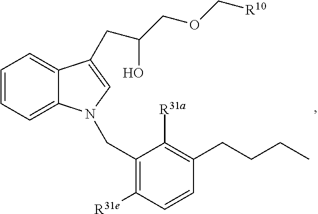

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

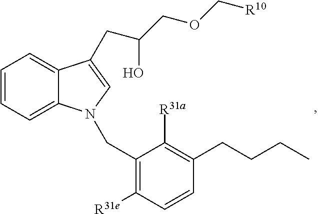

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

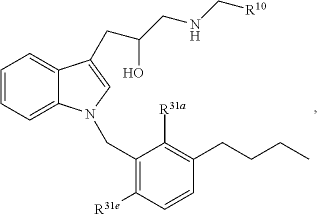

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

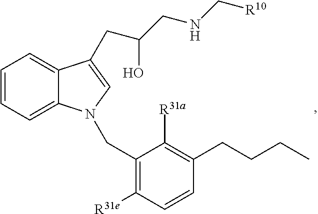

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

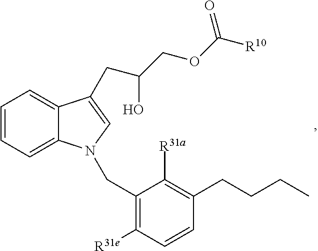

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

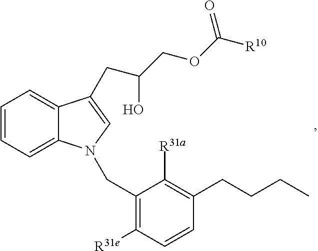

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

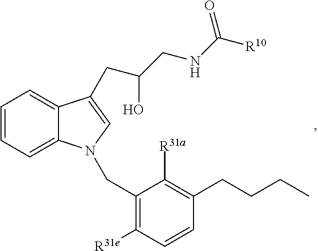

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

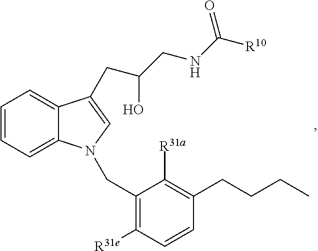

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

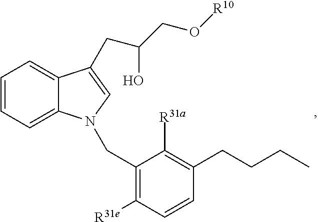

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

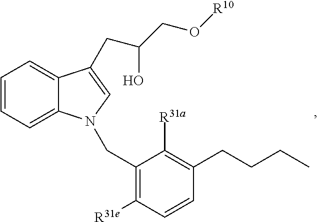

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

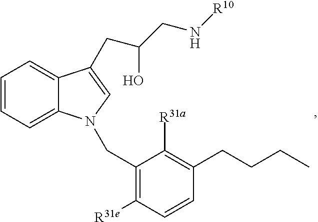

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

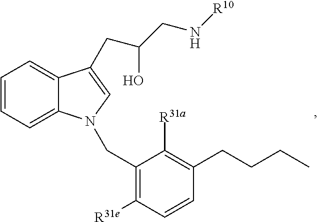

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

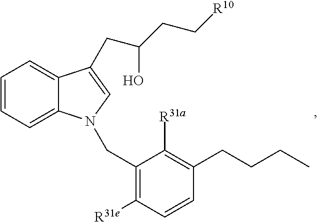

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

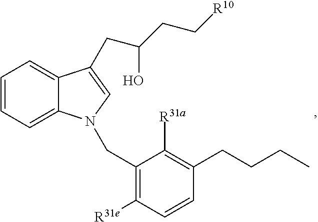

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

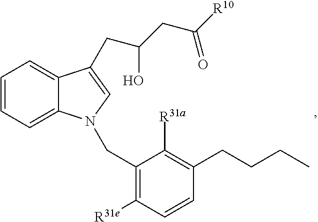

wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

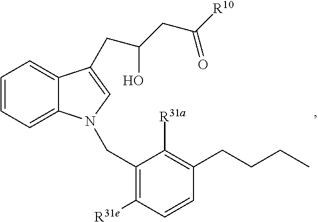

wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

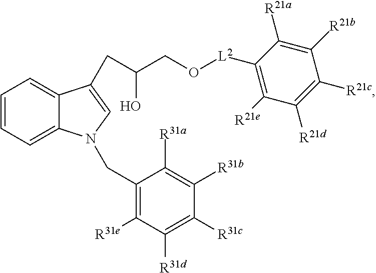

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d} and R^{31e} are hydrogen; and wherein all other variables are as defined herein; or a pharmaceutically acceptable salt thereof.

In a further aspect, the compound has a structure represented by a formula:

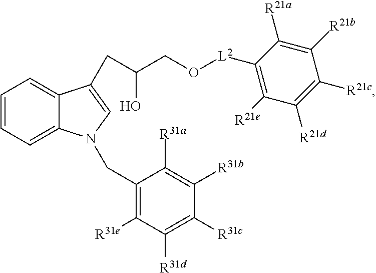

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

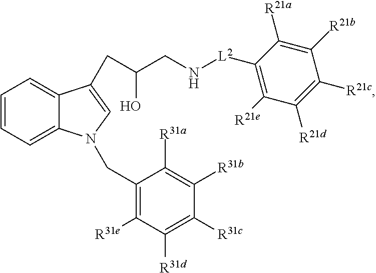

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31e}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

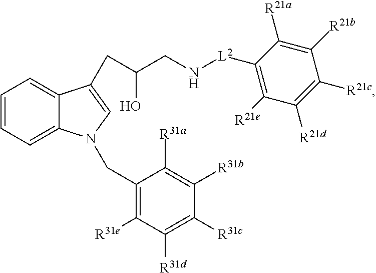

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

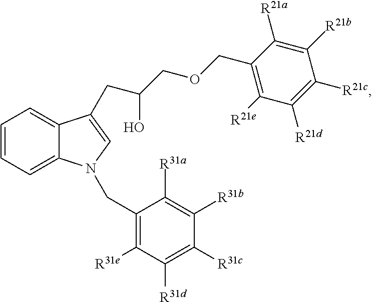

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d} and R^{31e} are hydrogen; and wherein all other variables are as defined herein; or a pharmaceutically acceptable salt thereof.

In a further aspect, the compound has a structure represented by a formula:

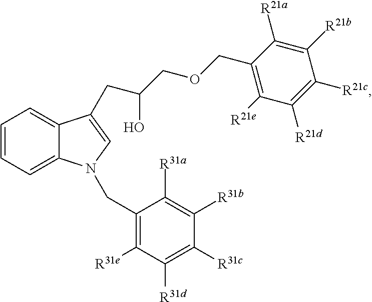

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

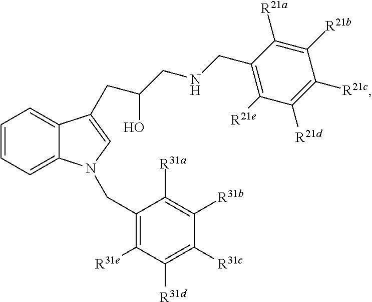

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d} and R^{31e} are hydrogen; and wherein all other variables are as defined herein; or a pharmaceutically acceptable salt thereof.

In a further aspect, the compound has a structure represented by a formula:

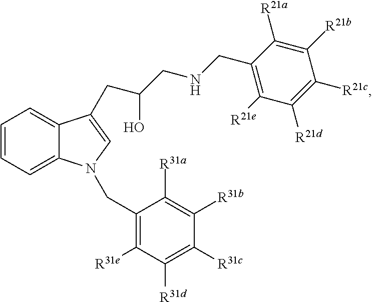

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

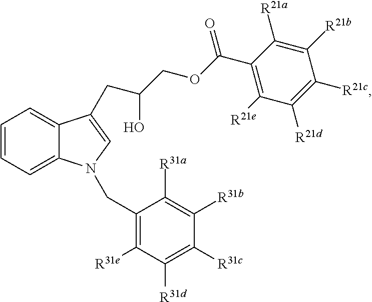

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31e}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d} and R^{31e} are hydrogen; and wherein all other variables are as defined herein; or a pharmaceutically acceptable salt thereof.

In a further aspect, the compound has a structure represented by a formula:

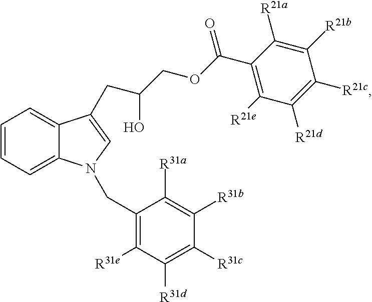

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d} and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

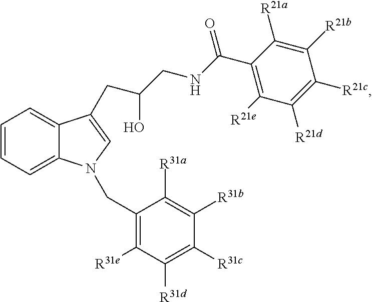

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d} and R^{31e} are hydrogen; and wherein all other variables are as defined herein; or a pharmaceutically acceptable salt thereof.

In a further aspect, the compound has a structure represented by a formula:

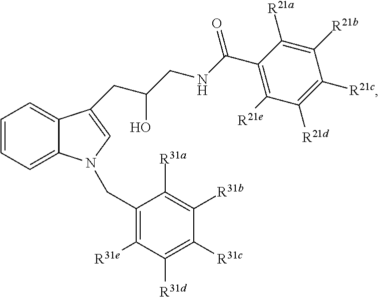

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

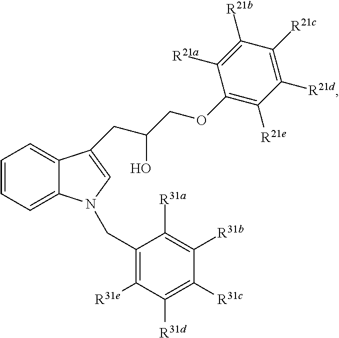

wherein each of R^{21a}, R^{21b}, R^{21c}, R^{21d}, and R^{21e} independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31e}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

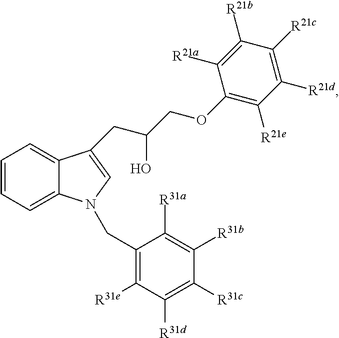

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

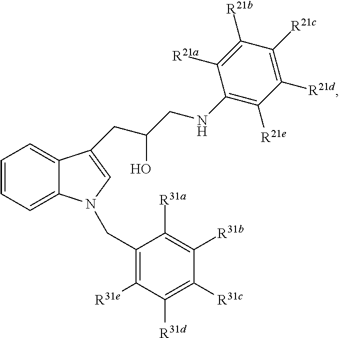

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

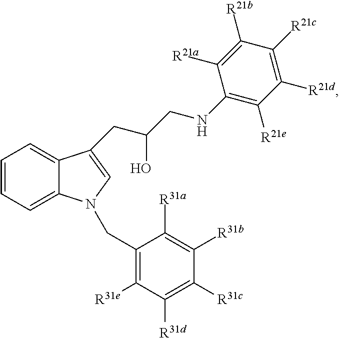

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

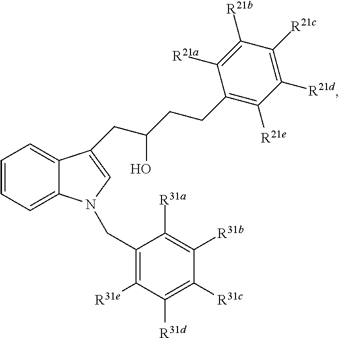

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

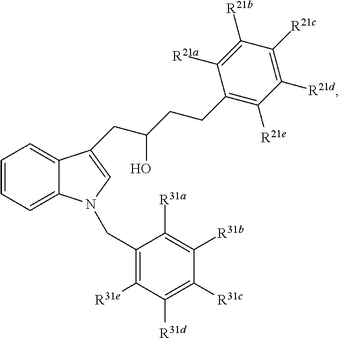

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

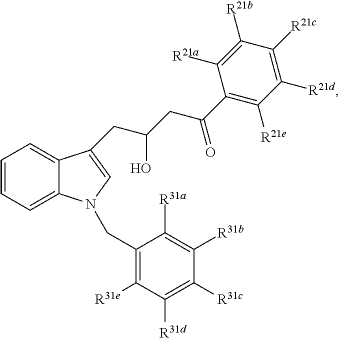

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, R^{31c}, R^{31d}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{31a}, R^{31b}, R^{31c}, R^{31d} and R^{31e} are hydrogen; and wherein all other variables are as defined herein; or a pharmaceutically acceptable salt thereof.

In a further aspect, the compound has a structure represented by a formula:

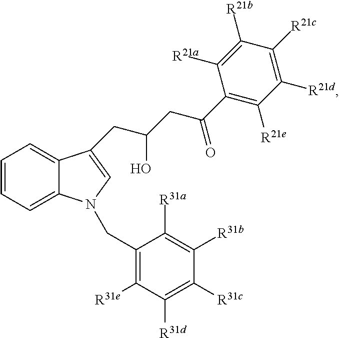

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R₂₁ₑ are hydrogen; wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen; and wherein all other variables are as defined herein; or a pharmaceutically acceptable salt thereof.

In a further aspect, the compound has a structure represented by a formula:

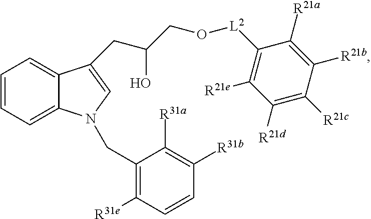

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

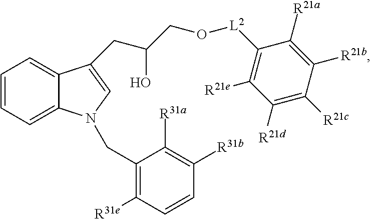

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

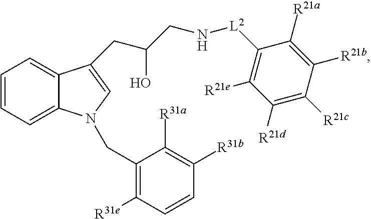

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

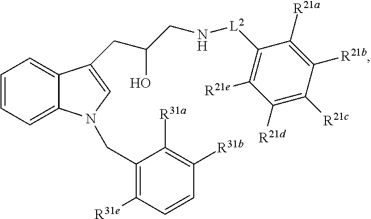

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

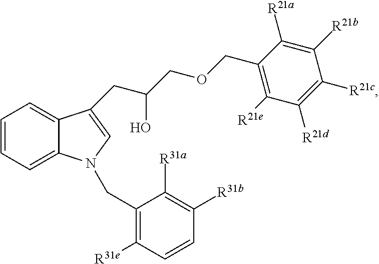

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

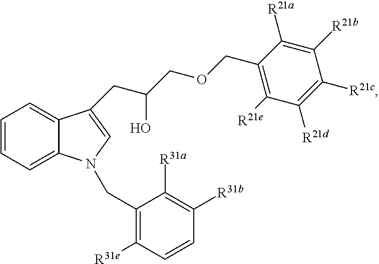

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

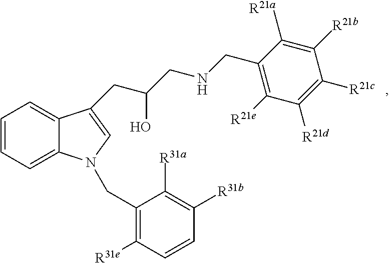

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

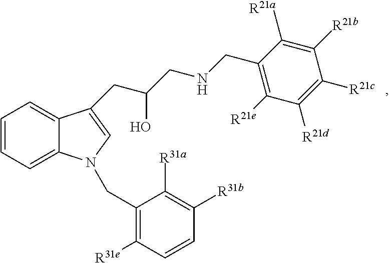

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

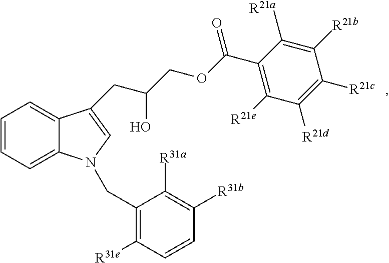

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

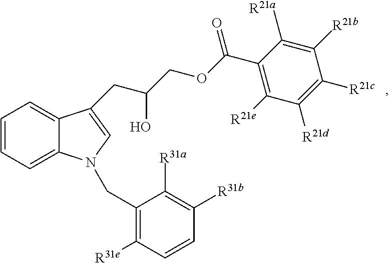

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

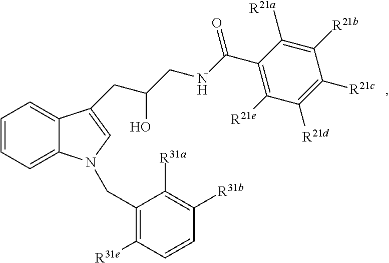

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

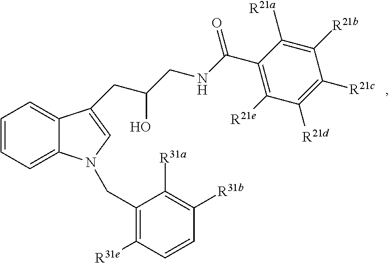

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

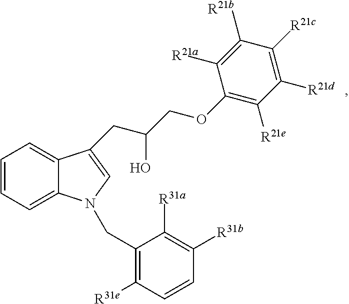

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

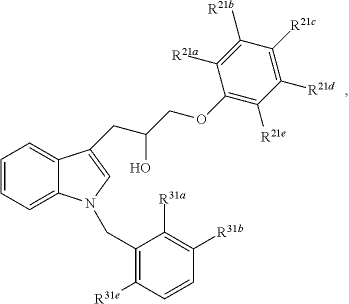

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

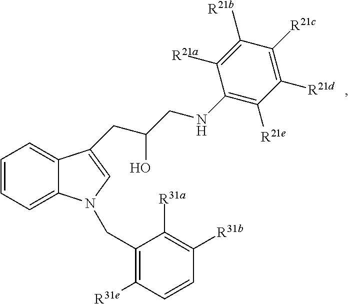

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

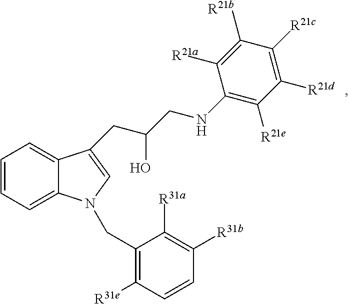

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

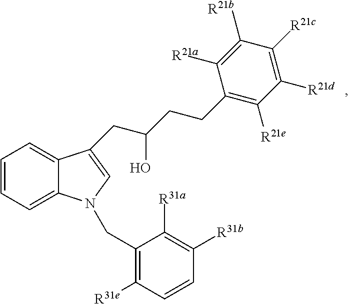

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

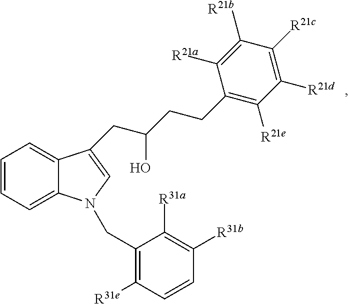

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

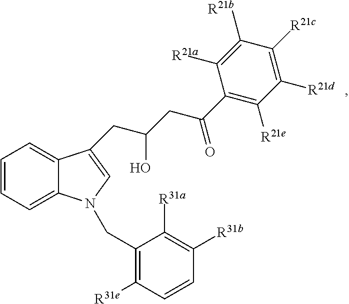

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a}, R^{31b}, and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

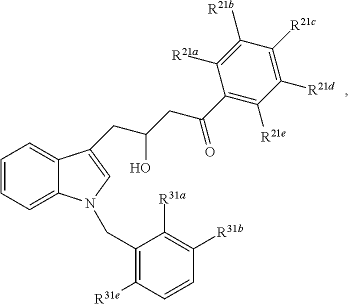

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a}, R^{31b}, and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

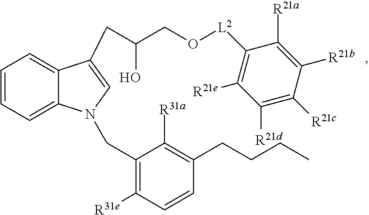

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

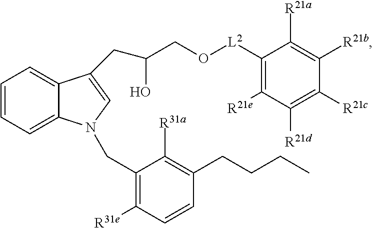

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

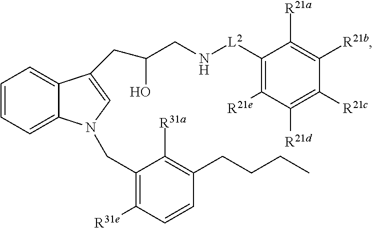

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

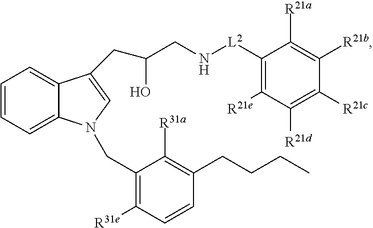

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

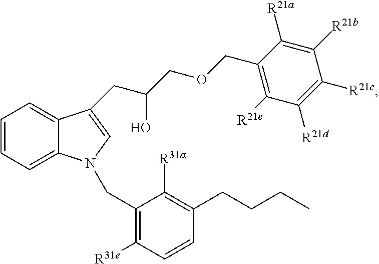

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

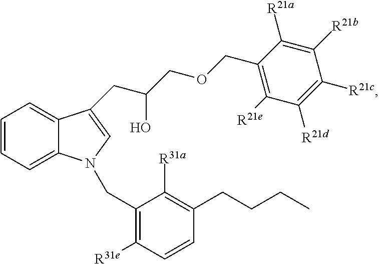

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

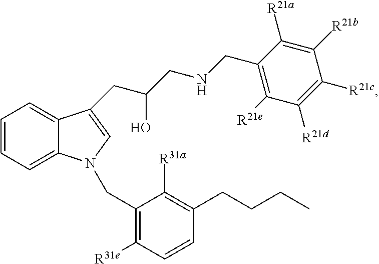

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

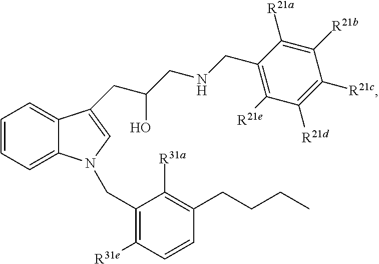

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

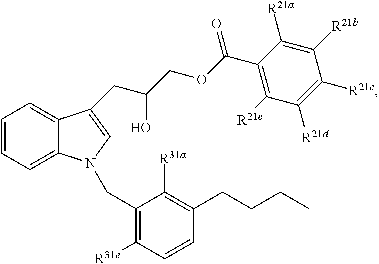

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

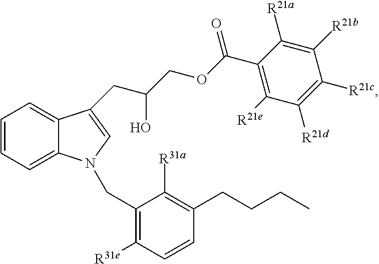

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

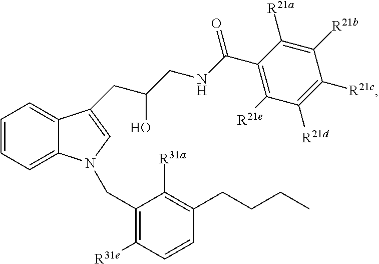

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

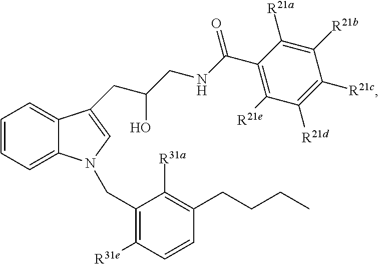

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

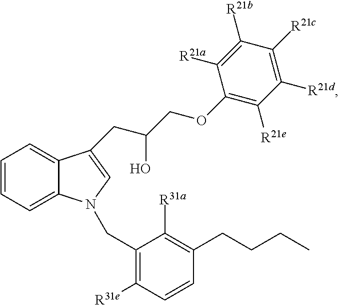

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

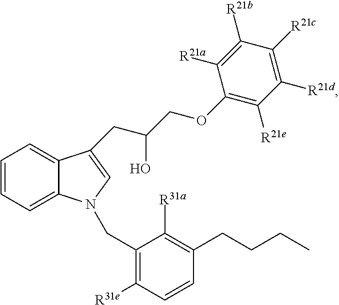

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

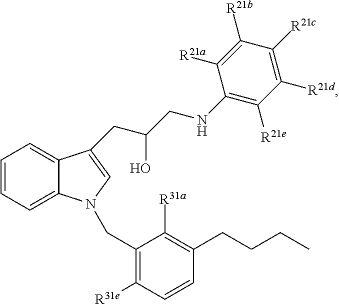

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

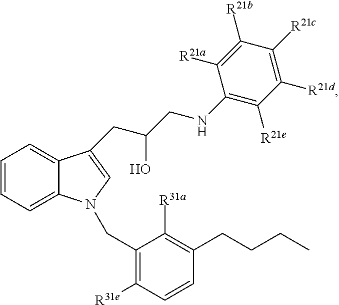

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

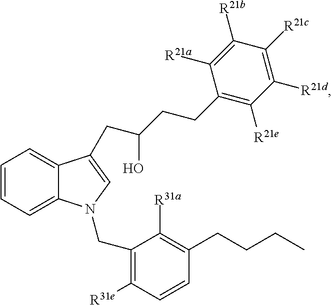

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

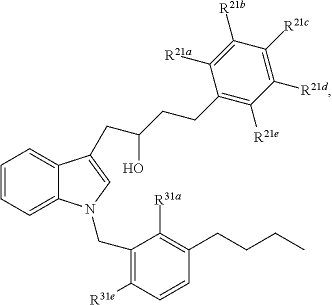

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

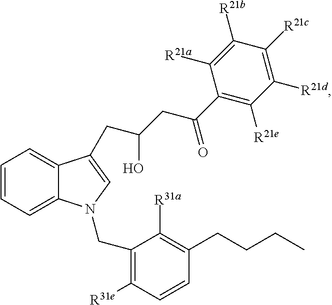

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; wherein each of R^{31a} and R^{31e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; and wherein all other variables are as defined herein; or a pharmaceutically acceptable salt thereof.

In a further aspect, the compound has a structure represented by a formula:

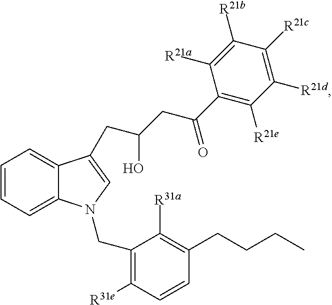

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃; and wherein all other variables are as defined herein; or a pharmaceutically acceptable salt thereof.

In a further aspect, the compound has a structure represented by a formula:

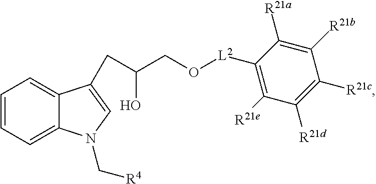

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

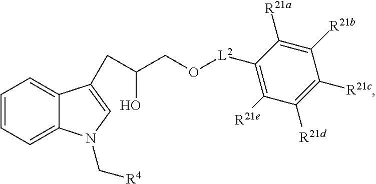

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

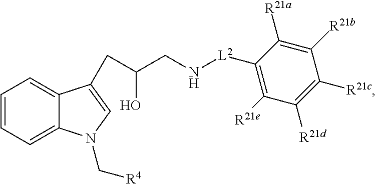

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

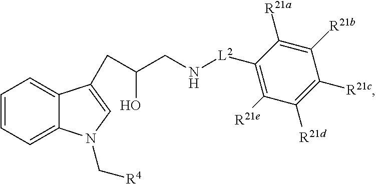

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

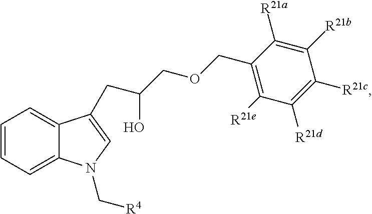

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

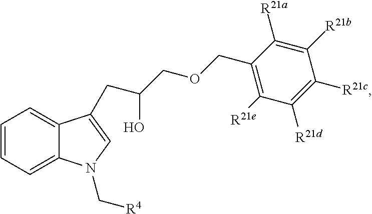

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

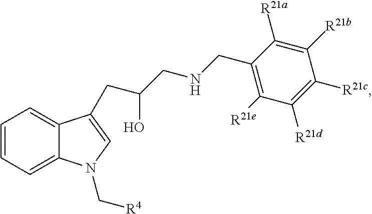

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

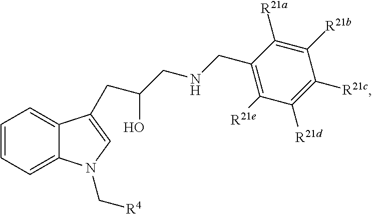

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

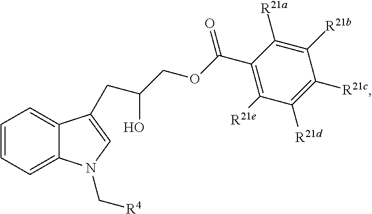

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

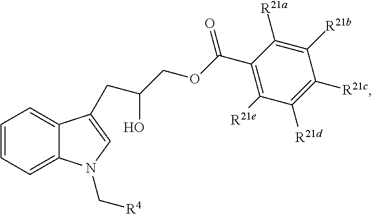

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

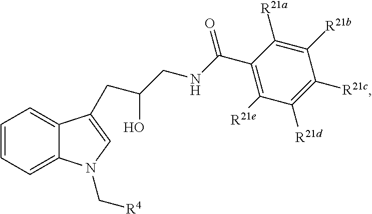

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

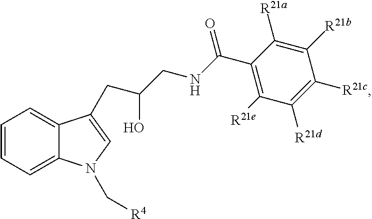

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

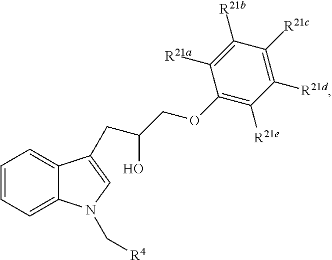

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

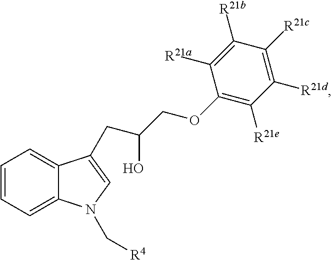

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

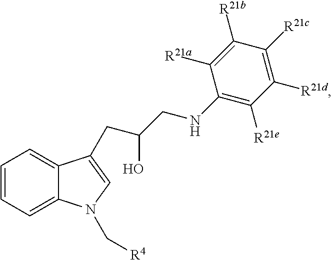

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

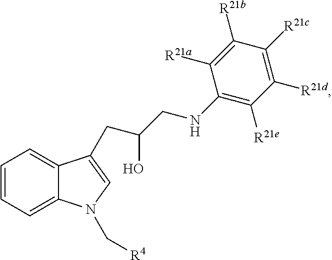

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

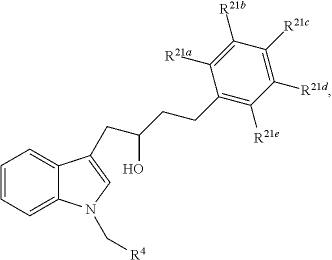

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

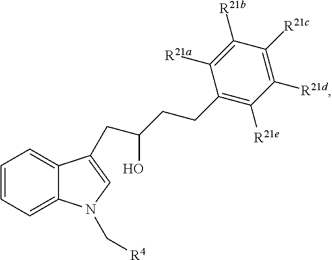

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

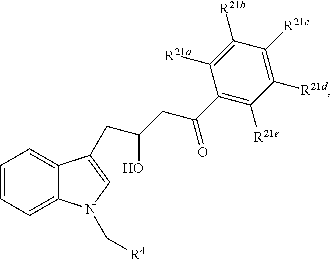

wherein each of R^{21a}, R^{21b}, R^{21c}, R^{21d}, and R^{21e} is independently selected from hydrogen, halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

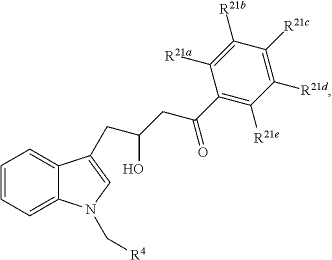

wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein all other variables are as defined herein; or a pharmaceutically acceptable salt thereof.

In a further aspect, the compound has a structure represented by a formula:

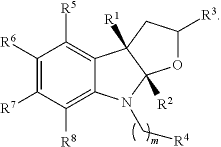

In a further aspect, the compound has a structure represented by a formula:

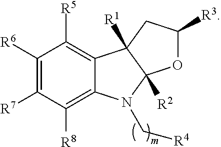

In a further aspect, the compound has a structure represented by a formula:

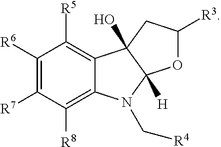

In a further aspect, the compound has a structure represented by a formula:

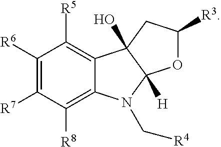

In a further aspect, the compound has a structure represented by a formula:

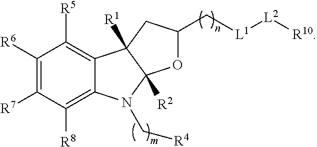

In a further aspect, the compound has a structure represented by a formula:

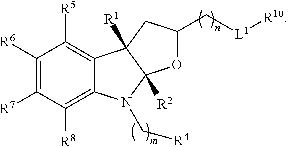

In a further aspect, the compound has a structure represented by a formula:

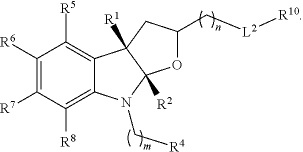

In a further aspect, the compound has a structure represented by a formula:

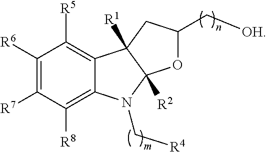

In a further aspect, the compound has a structure represented by a formula:

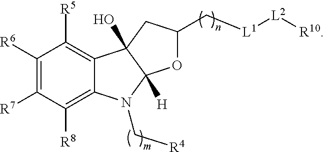

In a further aspect, the compound has a structure represented by a formula:

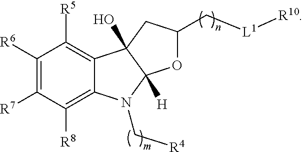

In a further aspect, the compound has a structure represented by a formula:

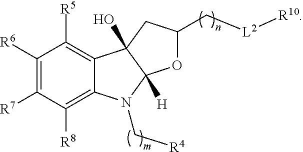

In a further aspect, the compound has a structure represented by a formula:

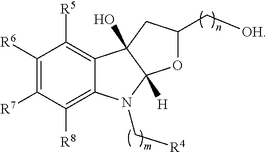

In a further aspect, the compound has a structure represented by a formula:

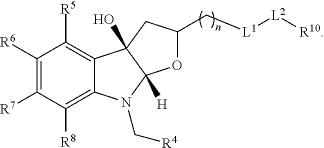

In a further aspect, the compound has a structure represented by a formula:

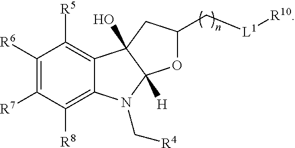

In a further aspect, the compound has a structure represented by a formula:

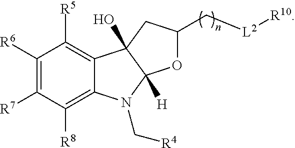

In a further aspect, the compound has a structure represented by a formula:

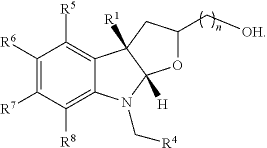

In a further aspect, the compound has a structure represented by a formula:

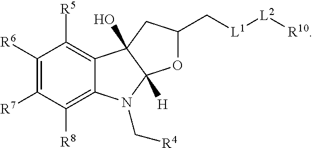

In a further aspect, the compound has a structure represented by a formula:

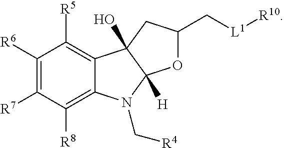

In a further aspect, the compound has a structure represented by a formula:

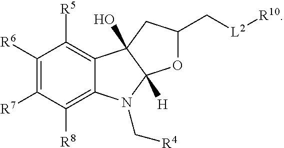

In a further aspect, the compound has a structure represented by a formula:

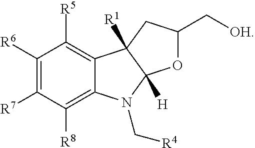

In a further aspect, the compound has a structure represented by a formula:

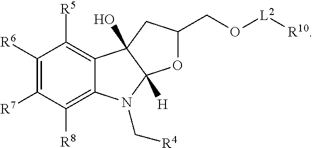

In a further aspect, the compound has a structure represented by a formula:

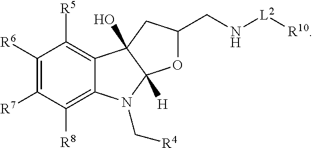

In a further aspect, the compound has a structure represented by a formula:

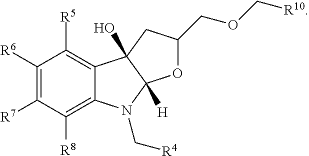

In a further aspect, the compound has a structure represented by a formula:

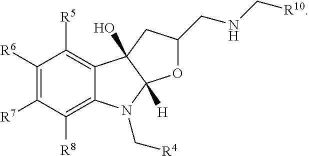

In a further aspect, the compound has a structure represented by a formula:

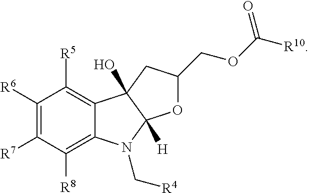

In a further aspect, the compound has a structure represented by a formula:

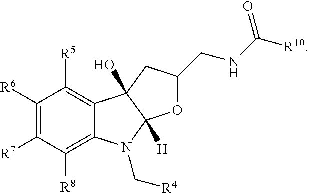

In a further aspect, the compound has a structure represented by a formula:

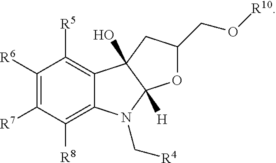

In a further aspect, the compound has a structure represented by a formula:

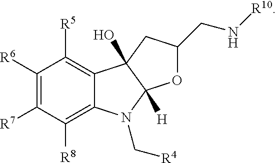

In a further aspect, the compound has a structure represented by a formula:

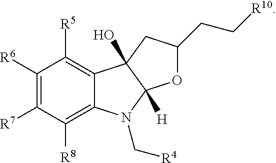

In a further aspect, the compound has a structure represented by a formula:

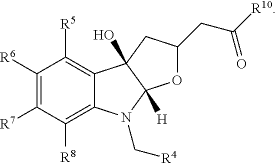

In a further aspect, the compound has a structure represented by a formula:

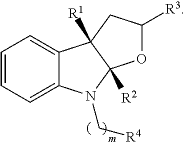

In a further aspect, the compound has a structure represented by a formula:

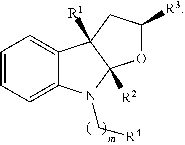

In a further aspect, the compound has a structure represented by a formula:

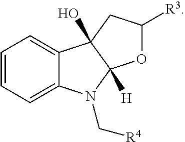

In a further aspect, the compound has a structure represented by a formula:

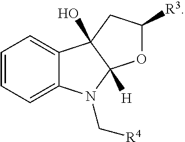

In a further aspect, the compound has a structure represented by a formula:

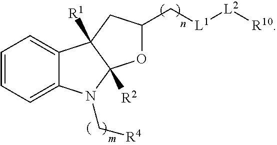

In a further aspect, the compound has a structure represented by a formula:

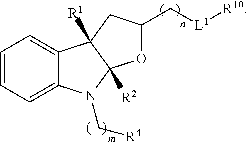

In a further aspect, the compound has a structure represented by a formula:

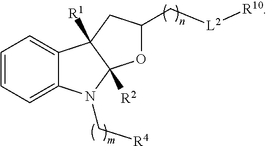

In a further aspect, the compound has a structure represented by a formula:

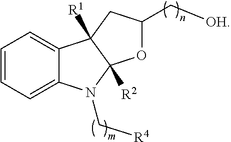

In a further aspect, the compound has a structure represented by a formula:

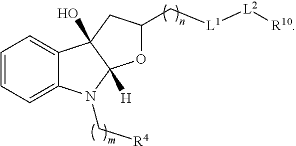

In a further aspect, the compound has a structure represented by a formula:

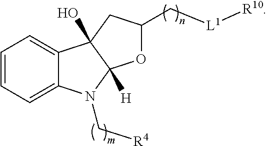

In a further aspect, the compound has a structure represented by a formula:

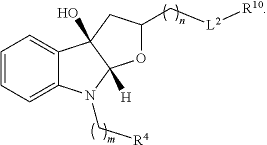

In a further aspect, the compound has a structure represented by a formula:

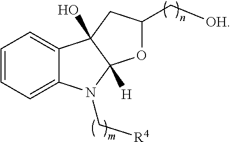

In a further aspect, the compound has a structure represented by a formula:

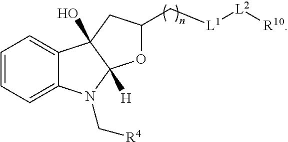

In a further aspect, the compound has a structure represented by a formula:

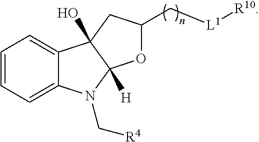

In a further aspect, the compound has a structure represented by a formula:

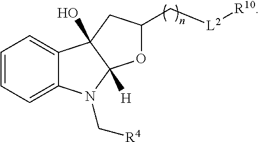

In a further aspect, the compound has a structure represented by a formula:

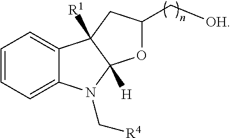

In a further aspect, the compound has a structure represented by a formula:

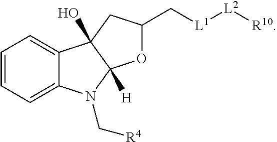

In a further aspect, the compound has a structure represented by a formula:

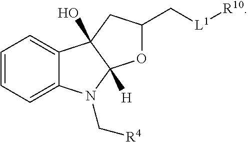

In a further aspect, the compound has a structure represented by a formula:

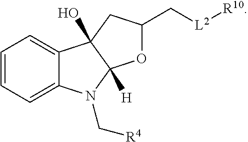

In a further aspect, the compound has a structure represented by a formula:

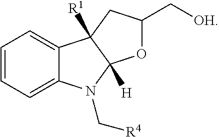

In a further aspect, the compound has a structure represented by a formula:

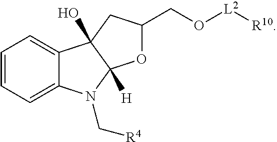

In a further aspect, the compound has a structure represented by a formula:

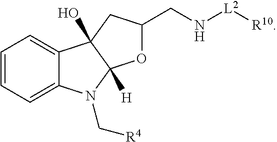

In a further aspect, the compound has a structure represented by a formula:

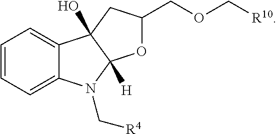

In a further aspect, the compound has a structure represented by a formula:

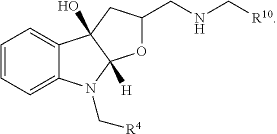

In a further aspect, the compound has a structure represented by a formula:

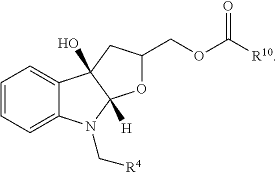

In a further aspect, the compound has a structure represented by a formula:

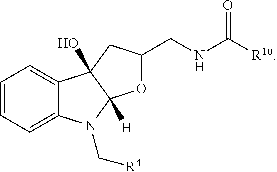

In a further aspect, the compound has a structure represented by a formula:

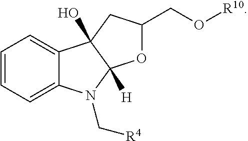

In a further aspect, the compound has a structure represented by a formula:

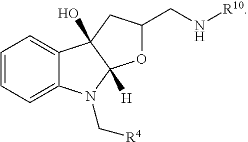

In a further aspect, the compound has a structure represented by a formula:

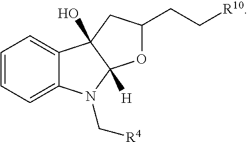

In a further aspect, the compound has a structure represented by a formula:

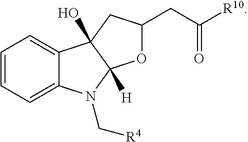

a. Ar¹ Groups

In various aspects, Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy.

b. Cy¹ Groups

In various aspects, Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy.

c. Ar² Groups

In various aspects, Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy.

d. Cy² Groups

In various aspects, Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy.

e. L¹ Groups

In various aspects, L¹ is —O— or —NH—. In a further aspect, L¹ is —O—. In a still further aspect, L¹ is —NH—.

f. L² Groups

In various aspects, L² is —CH₂— or —(C═O)—. In a further aspect, L² is —CH₂—. In a still further aspect, L² is —(C═O)—.

g. R¹ and R² Groups

In various aspects, each of R¹ and R², when present, is independently selected from H and —OH. In a further aspect, each of R¹ and R², when present, is hydrogen.

In a further aspect, R¹, when present, is —OH and R², when present, is selected from H and —OH. In a further aspect, R¹, when present, is —OH and R², when present, is hydrogen. In a further aspect, R¹, when present, is —OH and R², when present, is —OH.

In a further aspect, R¹, when present, is hydrogen and R², when present, is selected from H and —OH. In a further aspect, R¹, when present, is hydrogen and R², when present, is —OH.

h. R³ Groups

In one aspect, R³ is selected from hydrogen,

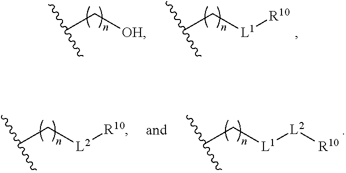

In a further aspect, R³ is hydrogen.

In a further aspect, R³ is selected from

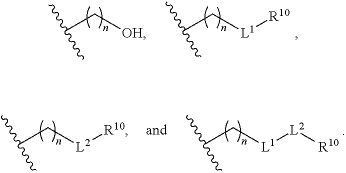

In a still further aspect, R³ is selected from

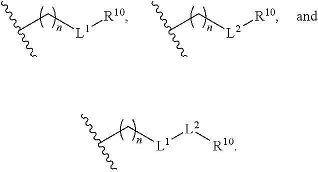

In a yet further aspect, R³ is selected from

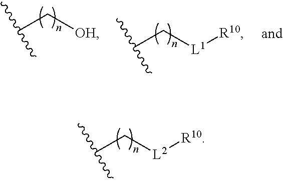

In a further aspect, R³ is

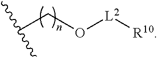

In a further aspect, R³ is

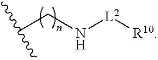

In a further aspect, R³ is

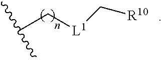

In a further aspect, R³ is

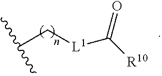

In a further aspect, R³ is selected from

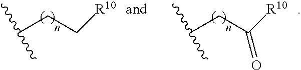

In a further aspect, R³ is selected from

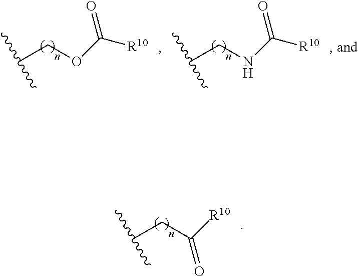

In a further aspect, R³ is selected from

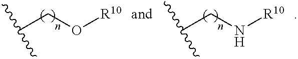

In a further aspect, R³ is selected from

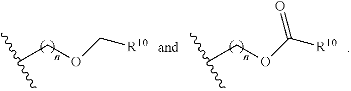

In a further aspect, R³ is selected from

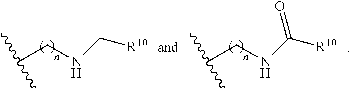

In a further aspect, R³ is selected from

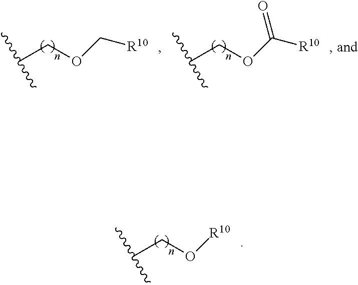

In a further aspect, R³ is selected from

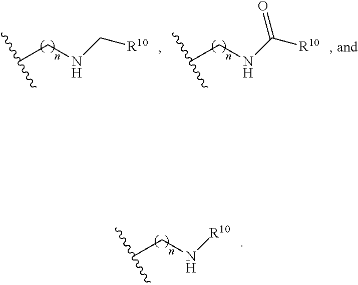

In a further aspect, R³ is

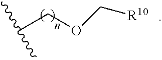

In a further aspect, R³ is

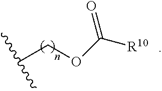

In a further aspect, R³ is

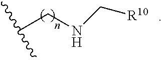

In a further aspect, R³ is

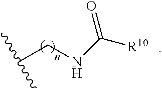

In a further aspect, R³ is

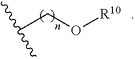

In a further aspect, R³ is

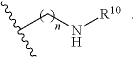

In a further aspect, R³ is

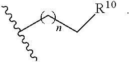

In a further aspect, R³ is

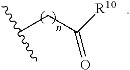

In a further aspect, R³ is

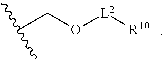

In a further aspect, R³ is

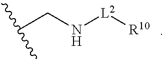

In a further aspect, R³ is

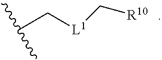

In a further aspect, R³ is

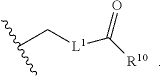

In a further aspect, R³ is selected from

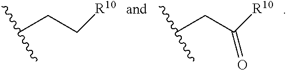

In a further aspect, R³ is selected from

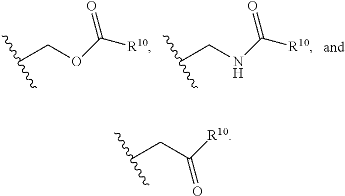

In a further aspect, R³ is selected from

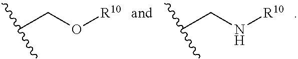

In a further aspect, R³ is selected from

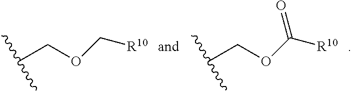

In a further aspect, R³ is selected from

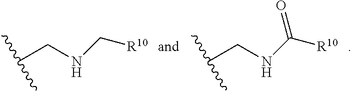

In a further aspect, R³ is selected from

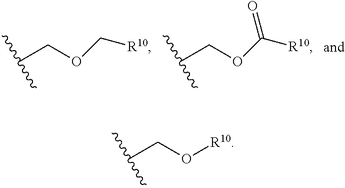

In a further aspect, R³ is selected from

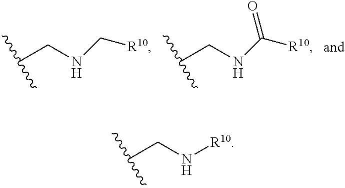

In a further aspect, R³ is

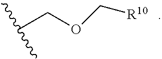

In a further aspect, R³ is

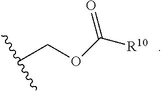

In a further aspect, R³ is

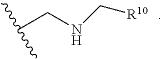

In a further aspect, R³ is

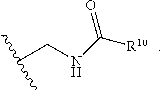

In a further aspect, R³ is

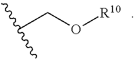

In a further aspect, R³ is

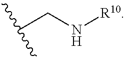

In a further aspect, R³ is

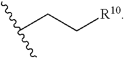

In a further aspect, R³ is

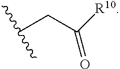

i. R⁴ Groups

In one aspect, R⁴ is selected from C1-C8 alkyl, C1-C8 alkoxy, —NR²³R²⁴, —O—Ar², —NH—Ar², —O-Cy², and —NH-Cy². In a further aspect, R⁴ is hydrogen.

In a further aspect, R⁴ is selected from hydrogen, C2-C8 alkyl, C2-C8 alkoxy, and —NR²³R²⁴. In a still further aspect, R⁴ is selected from hydrogen, methyl, ethyl, propyl, isopropyl, tert-butyl, sec-butyl, isobutyl, tert-butyl, —OCH₃, —OCH₂CH₃, —O(CH₂)₂CH₃, —OCH(CH₃)₂, —OCH(CH₂CH₃)(CH₃), —NHCH₃, —NHCH₂CH₃, —NH(CH₂)₂CH₃, —NHCH(CH₃)₂, —NH(CH₂)₃CH₃, —NH(CH₂)₄—CH₃, —N(CH₃)₂, —N(CH₃)CH₂CH₃, —N(CH₃)(CH₂)₂CH₃, —N(CH₃)CH(CH₃)₂, —N(CH₂CH₃)₂, —N(CH₂CH₃)((CH₂)₂CH₃), and —N(CH₂CH₃)(CH(CH₃)₂). In a yet further aspect, R⁴ is selected from hydrogen, methyl, ethyl, propyl, isopropyl, —OCH₃, —OCH₂CH₃, —O(CH₂)₂CH₃, —OCH(CH₃)₂, —NHCH₃, —NHCH₂CH₃, —N(CH₃)₂, —N(CH₃)CH₂CH₃, and —N(CH₂CH₃)₂. In a yet further aspect, R⁴ is selected from hydrogen, methyl, —OCH₃, —NHCH₃, and —N(CH₃)₂.

In a further aspect, R⁴ is selected from hydrogen, —O—Ar², —NH—Ar², —O-Cy², and —NH-Cy². In a still further aspect, R⁴ is —O—Ar². In a yet further aspect, R⁴ is —NH—Ar². In an even further aspect, R⁴ is —O-Cy². In a still further aspect, R⁴ is —NH-Cy².

In a further aspect, R⁴ is selected from C2-C8 alkyl, C2-C8 alkoxy, and —NR²³R²⁴. In a still further aspect, R⁴ is selected from methyl, ethyl, propyl, isopropyl, tert-butyl, sec-butyl, isobutyl, tert-butyl, —OCH₃, —OCH₂CH₃, —O(CH₂)₂CH₃, —OCH(CH₃)₂, —OCH(CH₂CH₃)(CH₃), —NHCH₃, —NHCH₂CH₃, —NH(CH₂)₂CH₃, —NHCH(CH₃)₂, —NH(CH₂)₃CH₃, —NH(CH₂)₄—CH₃, —N(CH₃)₂, —N(CH₃)CH₂CH₃, —N(CH₃)(CH₂)₂CH₃, —N(CH₃)CH(CH₃)₂, —N(CH₂CH₃)₂, —N(CH₂CH₃)((CH₂)₂CH₃), and —N(CH₂CH₃)(CH(CH₃)₂). In a yet further aspect, R⁴ is selected from methyl, ethyl, propyl, isopropyl, —OCH₃, —OCH₂CH₃, —O(CH₂)₂CH₃, —OCH(CH₃)₂, —NHCH₃, —NHCH₂CH₃, —N(CH₃)₂, —N(CH₃)CH₂CH₃, and —N(CH₂CH₃)₂. In a yet further aspect, R⁴ is selected from methyl, —OCH₃, —NHCH₃, and —N(CH₃)₂.

In a further aspect, R⁴ is selected from —O—Ar², —NH—Ar², —O-Cy², and —NH-Cy².

J. R⁵, R⁶, R⁷, and R⁸ Groups

In one aspect, wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy. In a further aspect, each of R⁵, R⁶, R⁷, and R⁸ is hydrogen.

In a further aspect, each of R⁵, R⁶, R⁷, and R⁸ is independently selected from hydrogen, halogen, —OH, —NO₂, —NR²⁵R²⁶, C1-C3 alkyl, C1-C3 haloalkyl, —(C1-C3 alkyl)-OH, and C1-C3 alkoxy. In a still further aspect, each of R⁵, R⁶, R⁷, and R⁸ is independently selected from hydrogen, —F, —Cl, —OH, —NO₂, methyl, ethyl, propyl, isopropyl, tert-butyl, sec-butyl, isobutyl, tert-butyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃, —(CH₂)₂CHF₂, —(CH₂)₂CF₃, —(CH₂)₂CHCl₂, —(CH₂)₂CCl₃, —CH₂OH, —(CH₂)₂OH, —(CH₂)₃OH, —(CH₂)₄OH, —(CHOH)CH₃, —(CHOH)CH₂CH₃, —(CHOH)(CH₂)₂CH₃, —CH₂(CHOH)CH₃, —CH₂(CHOH)CH₂CH₃, —(CH₂)₂(CHOH)CH₃, —(CHOH)CH(CH₃)₂, —OCH₃, —OCH₂CH₃, —O(CH₂)₂CH₃, —OCH(CH₃)₂, —OCH(CH₂CH₃)(CH₃), —NHCH₃, —NHCH₂CH₃, —NH(CH₂)₂CH₃, —NHCH(CH₃)₂, —NH(CH₂)₃CH₃, —NH(CH₂)₄—CH₃, —N(CH₃)₂, —N(CH₃)CH₂CH₃, —N(CH₃)(CH₂)₂CH₃, —N(CH₃)CH(CH₃)₂, —N(CH₂CH₃)₂, —N(CH₂CH₃)((CH₂)₂CH₃), and —N(CH₂CH₃)(CH(CH₃)₂). In a yet further aspect, each of R⁵, R⁶, R⁷, and R⁸ is independently selected from hydrogen, —F, —Cl, —OH, —NO₂, methyl, ethyl, propyl, isopropyl, —CH₂F, —CHF₂, —CF₃, —CHCl₂, —CH₂Cl, —CCl₃, —CH₂OH, —(CH₂)₂OH, —(CHOH)CH₃, —OCH₃, —OCH₂CH₃, —O(CH₂)₂CH₃, —OCH(CH₃)₂, —NHCH₃, —NHCH₂CH₃, —NH(CH₂)₂CH₃, —NHCH(CH₃)₂, —N(CH₃)₂, —N(CH₃)CH₂CH₃, and —N(CH₂CH₃)₂. In a yet further aspect, each of R⁵, R⁶, R⁷, and R⁸ is independently selected from hydrogen, —F, —Cl, —CH₂F, —CHF₂, —CF₃, —CH₂OH, —OH, —NO₂, methyl, —OCH₃, —NHCH₃, and —N(CH₃)₂.

In a further aspect, each of R⁵, R⁶, R⁷, and R⁸ is independently selected from hydrogen, —F, —OH, —NO₂, methyl, —OCH₃, —NHCH₃, and —N(CH₃)₂. In a still further aspect, each of R⁵, R⁶, R⁷, and R⁸ is independently selected from hydrogen, —F, —OH, methyl, —OCH₃, —NHCH₃, and —N(CH₃)₂. In a yet further aspect, each of R⁵, R⁶, R⁷, and R⁸ is independently selected from hydrogen, —F, —OH, —OCH₃, and —NHCH₃.

In a further aspect, each of R⁵, R⁶, and R⁷ is hydrogen and R⁸ is selected from hydrogen, —F, —OH, —OCH₃, and —NHCH₃. In a still further aspect, each of R⁵, R⁶, and R⁸ is hydrogen and R⁷ is selected from hydrogen, —F, —OH, —OCH₃, and —NHCH₃. In a yet further aspect, each of R⁵, R⁷, and R⁸ is hydrogen and R⁶ is selected from hydrogen, —F, —OH, —OCH₃, and —NHCH₃. In an even further aspect, each of R⁶, R⁷, and R⁸ is hydrogen and R⁵ is selected from hydrogen, —F, —OH, —OCH₃, and —NHCH₃.

In a further aspect, each of R⁵, R⁶, R⁷, and R⁸ is independently selected from hydrogen, —F, —OH, —CH₂OH, —NO₂, methyl, —OCH₃, —NHCH₃, and —N(CH₃)₂. In a still further aspect, each of R⁵, R⁶, R⁷, and R⁸ is independently selected from hydrogen, —F, —CH₂OH, —OH, methyl, —OCH₃, —NHCH₃, and —N(CH₃)₂. In a yet further aspect, each of R⁵, R⁶, R⁷, and R⁸ is independently selected from hydrogen, —F, —CH₂OH, —OH, —OCH₃, and —NHCH₃.

In a further aspect, each of R⁵, R⁶, and R⁷ is hydrogen and R⁸ is selected from hydrogen, —F, —CH₂OH, —OH, —OCH₃, and —NHCH₃. In a still further aspect, each of R⁵, R⁶, and R⁸ is hydrogen and R⁷ is selected from hydrogen, —F, —CH₂OH, —OH, —OCH₃, and —NHCH₃. In a yet further aspect, each of R⁵, R⁷, and R⁸ is hydrogen and R⁶ is selected from hydrogen, —F, —CH₂OH, —OH, —OCH₃, and —NHCH₃. In an even further aspect, each of R⁶, R⁷, and R⁸ is hydrogen and R⁵ is selected from hydrogen, —F, —CH₂OH, —OH, —OCH₃, and —NHCH₃.

k. R¹⁰ GROUPS

In one aspect, R¹⁰ is selected from hydrogen, C1-C8 alkyl, C1-C8 alkoxy, —NR²¹R²², —O—Ar¹, —NH—Ar¹, —O-Cy¹, and —NH-Cy¹. In a further aspect, R¹⁰ is hydrogen.

In a further aspect, R¹⁰ is selected from hydrogen, C1-C8 alkyl, C1-C8 alkoxy, and —NR²¹R²². In a still further aspect, R¹⁰ is selected from hydrogen, methyl, ethyl, propyl, isopropyl, tert-butyl, sec-butyl, isobutyl, tert-butyl, —OCH₃, —OCH₂CH₃, —O(CH₂)₂CH₃, —OCH(CH₃)₂, —OCH(CH₂CH₃)(CH₃), —NHCH₃, —NHCH₂CH₃, —NH(CH₂)₂CH₃, —NHCH(CH₃)₂, —NH(CH₂)₃CH₃, —NH(CH₂)₄—CH₃, —N(CH₃)₂, —N(CH₃)CH₂CH₃, —N(CH₃)(CH₂)₂CH₃, —N(CH₃)CH(CH₃)₂, —N(CH₂CH₃)₂, —N(CH₂CH₃)((CH₂)₂CH₃), and —N(CH₂CH₃)(CH(CH₃)₂). In a yet further aspect, R¹⁰ is selected from hydrogen, methyl, ethyl, propyl, isopropyl, —OCH₃, —OCH₂CH₃, —O(CH₂)₂CH₃, —OCH(CH₃)₂, —NHCH₃, —NHCH₂CH₃, —N(CH₃)₂, —N(CH₃)CH₂CH₃, and —N(CH₂CH₃)₂. In a yet further aspect, R¹⁰ is selected from hydrogen, methyl, —OCH₃, —NHCH₃, and —N(CH₃)₂.

In a further aspect, R¹⁰ is selected from hydrogen, —O—Ar¹, —NH—Ar¹, —O-Cy¹, and —NH-Cy¹. In a still further aspect, R¹⁰ is —O—Ar¹. In a yet further aspect, R¹⁰ is —NH—Ar¹. In an even further aspect, R¹⁰ is —O-Cy¹. In a still further aspect, R¹⁰ is —NH-Cy¹.

In a further aspect, R¹⁰ is selected from C1-C8 alkyl, C1-C8 alkoxy, and —NR²¹R²². In a still further aspect, R¹⁰ is selected from methyl, ethyl, propyl, isopropyl, tert-butyl, sec-butyl, isobutyl, tert-butyl, —OCH₃, —OCH₂CH₃, —O(CH₂)₂CH₃, —OCH(CH₃)₂, —OCH(CH₂CH₃)(CH₃), —NHCH₃, —NHCH₂CH₃, —NH(CH₂)₂CH₃, —NHCH(CH₃)₂, —NH(CH₂)₃CH₃, —NH(CH₂)₄—CH₃, —N(CH₃)₂, —N(CH₃)CH₂CH₃, —N(CH₃)(CH₂)₂CH₃, —N(CH₃)CH(CH₃)₂, —N(CH₂CH₃)₂, —N(CH₂CH₃)((CH₂)₂CH₃), and —N(CH₂CH₃)(CH(CH₃)₂). In a yet further aspect, R¹⁰ is selected from methyl, ethyl, propyl, isopropyl, —OCH₃, —OCH₂CH₃, —O(CH₂)₂CH₃, —OCH(CH₃)₂, —NHCH₃, —NHCH₂CH₃, —N(CH₃)₂, —N(CH₃)CH₂CH₃, and —N(CH₂CH₃)₂. In a yet further aspect, R¹⁰ is selected from methyl, —OCH₃, —NHCH₃, and —N(CH₃)₂.

In a further aspect, R¹⁰ is selected from —O—Ar¹, —NH—Ar¹, —O-Cy¹, and —NH-Cy¹.

l. R¹¹ Groups

In one aspect, R¹¹, when present, is selected from hydrogen and C1-C8 alkyl. In a further aspect, R¹¹, when present, is hydrogen. In a still further aspect, R¹¹, when present, is methyl.

In a further aspect, R¹¹, when present, is selected from hydrogen and C1-C6 alkyl. In a further aspect, R¹¹, when present, is selected from hydrogen and C1-C3 alkyl. In a still further aspect, R¹¹, when present, is selected from hydrogen, methyl, ethyl, propyl, isopropyl, tert-butyl, sec-butyl, isobutyl, and tert-butyl. In a yet further aspect, R¹¹, when present, is selected from hydrogen, methyl, ethyl, propyl, and isopropyl. In an even further aspect, R¹¹, when present, is selected from hydrogen and methyl.

M. R²¹ and R²² Groups

In one aspect, each of R²¹ and R²² is independently selected from hydrogen and C1-C6 alkyl. In a further aspect, each of R²¹ and R²² is hydrogen.

In a further aspect, each of R²¹ and R²² is independently selected from hydrogen and C1-C3 alkyl. In a still further aspect, each of R²¹ and R²² is independently selected from hydrogen, methyl, ethyl, propl, isopropyl, tert-butyl, sec-butyl, isobutyl, and tert-butyl. In a yet further aspect, each of R²¹ and R²² is independently selected from hydrogen, methyl, ethyl, propyl, and isopropyl. In an even further aspect, each of R²¹ and R²² is independently selected from hydrogen and methyl.

In a further aspect, R²¹ is hydrogen and R²² is selected from hydrogen and C1-C3 alkyl. In a still further aspect, R²¹ is hydrogen and R²² is selected from hydrogen, methyl, ethyl, propyl, isopropyl, tert-butyl, sec-butyl, isobutyl, and tert-butyl. In a yet further aspect, R²¹ is hydrogen and R²² is selected from hydrogen, methyl, ethyl, propyl, and isopropyl. In an even further aspect, R²¹ is hydrogen and R²² is selected from hydrogen and methyl.

In a further aspect, R²¹ is hydrogen and R²² is C1-C3 alkyl. In a still further aspect, R²¹ is hydrogen and R²² is selected from methyl, ethyl, propyl, isopropyl, tert-butyl, sec-butyl, isobutyl, and tert-butyl. In a yet further aspect, R²¹ is hydrogen and R²² is selected from methyl, ethyl, propyl, and isopropyl. In an even further aspect, R²¹ is hydrogen and R²² is methyl.

N. R²³ and R²⁴ Groups

In one aspect, each of R²³ and R²⁴ is independently selected from hydrogen and C1-C6 alkyl. In a further aspect, each of R²³ and R²⁴ is hydrogen.

In a further aspect, each of R²³ and R²⁴ is independently selected from hydrogen and C1-C3 alkyl. In a still further aspect, each of R²³ and R²⁴ is independently selected from hydrogen, methyl, ethyl, propyl, isopropyl, tert-butyl, sec-butyl, isobutyl, and tert-butyl. In a yet further aspect, each of R²³ and R²⁴ is independently selected from hydrogen, methyl, ethyl, propyl, and isopropyl. In an even further aspect, each of R²³ and R²⁴ is independently selected from hydrogen and methyl.

In a further aspect, R²³ is hydrogen and R²⁴ is selected from hydrogen and C1-C3 alkyl. In a still further aspect, R²³ is hydrogen and R²⁴ is selected from hydrogen, methyl, ethyl, propyl, isopropyl, tert-butyl, sec-butyl, isobutyl, and tert-butyl. In a yet further aspect, R²³ is hydrogen and R²⁴ is selected from hydrogen, methyl, ethyl, propyl, and isopropyl. In an even further aspect, R²³ is hydrogen and R²⁴ is selected from hydrogen and methyl.

In a further aspect, R²³ is hydrogen and R²⁴ is C1-C3 alkyl. In a still further aspect, R²³ is hydrogen and R²⁴ is selected from methyl, ethyl, propyl, isopropyl, tert-butyl, sec-butyl, isobutyl, and tert-butyl. In a yet further aspect, R²³ is hydrogen and R²⁴ is selected from methyl, ethyl, propyl, and isopropyl. In an even further aspect, R²³ is hydrogen and R²⁴ is methyl.

o. R²⁵ and R²⁶ Groups

In one aspect, each of R²⁵ and R²⁶ is independently selected from hydrogen and C1-C6 alkyl. In a further aspect, each of R²⁵ and R²⁶ is hydrogen.

In a further aspect, each of R²⁵ and R²⁶ is independently selected from hydrogen and C1-C3 alkyl. In a still further aspect, each of R²⁵ and R²⁶ is independently selected from hydrogen, methyl, ethyl, propyl, isopropyl, tert-butyl, sec-butyl, isobutyl, and tert-butyl. In a yet further aspect, each of R²⁵ and R²⁶ is independently selected from hydrogen, methyl, ethyl, propyl, and isopropyl. In an even further aspect, each of R²⁵ and R²⁶ is independently selected from hydrogen and methyl.

In a further aspect, R²⁵ is hydrogen and R²⁶ is selected from hydrogen and C1-C3 alkyl. In a still further aspect, R²⁵ is hydrogen and R²⁶ is selected from hydrogen, methyl, ethyl, propyl, isopropyl, tert-butyl, sec-butyl, isobutyl, and tert-butyl. In a yet further aspect, R²⁵ is hydrogen and R²⁶ is selected from hydrogen, methyl, ethyl, propyl, and isopropyl. In an even further aspect, R²⁵ is hydrogen and R²⁶ is selected from hydrogen and methyl.

In a further aspect, R²⁵ is hydrogen and R²⁶ is C1-C3 alkyl. In a still further aspect, R²⁵ is hydrogen and R²⁶ is selected from methyl, ethyl, propyl, isopropyl, tert-butyl, sec-butyl, isobutyl, and tert-butyl. In a yet further aspect, R²⁵ is hydrogen and R²⁶ is selected from methyl, ethyl, propyl, and isopropyl. In an even further aspect, R²⁵ is hydrogen and R²⁶ is methyl.

p. Halogen(X)

In one aspect, halogen is fluoro, chloro, bromo or iodo. In a still further aspect, halogen is fluoro, chloro, or bromo. In a yet further aspect, halogen is fluoro or chloro. In a further aspect, halogen is fluoro. In an even further aspect, halogen is chloro or bromo. In an even further aspect, halogen is chloro. In a yet further aspect, halogen is iodo. In a still further aspect, halogen is bromo.

It is also contemplated that pseudohalogens (e.g. triflate, mesylate, brosylate, etc.) can be used as leaving groups in place of halogens in certain aspects.

2. Example Compounds

In one aspect, a compound can be present as:

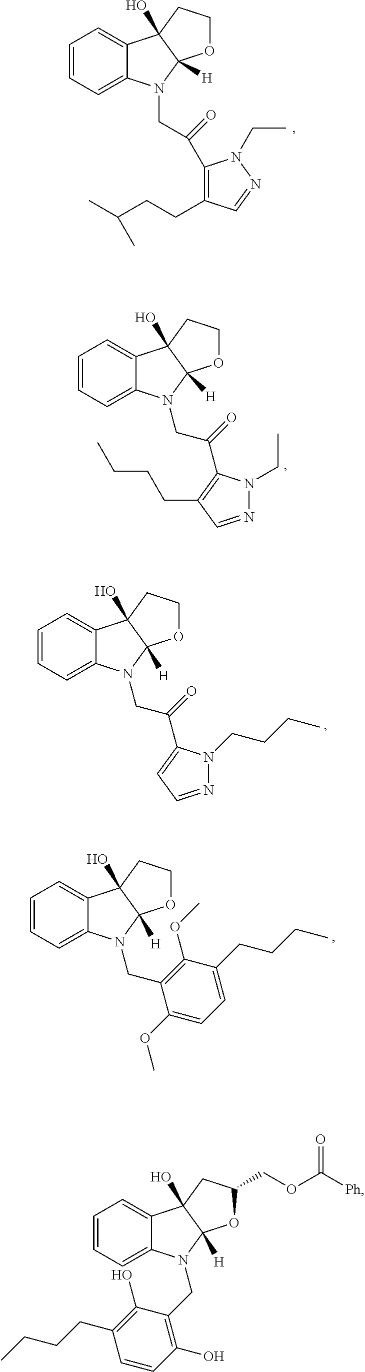

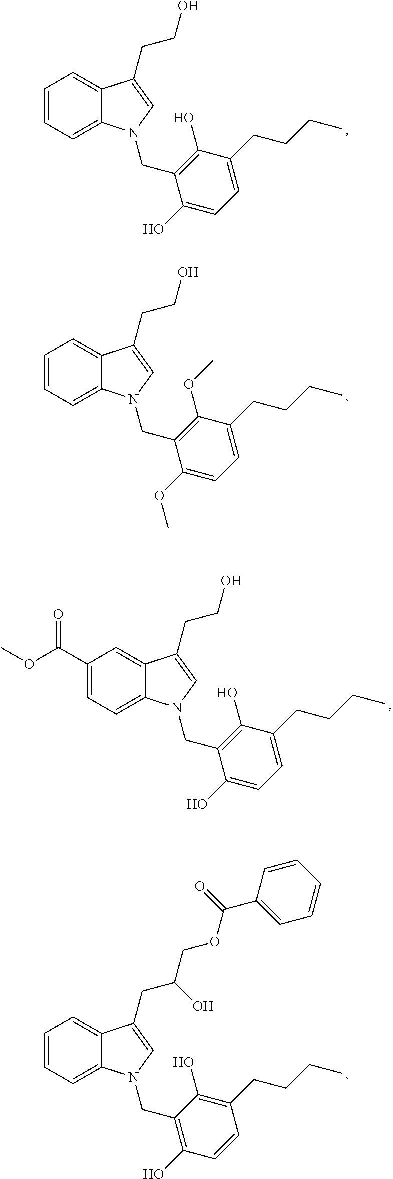

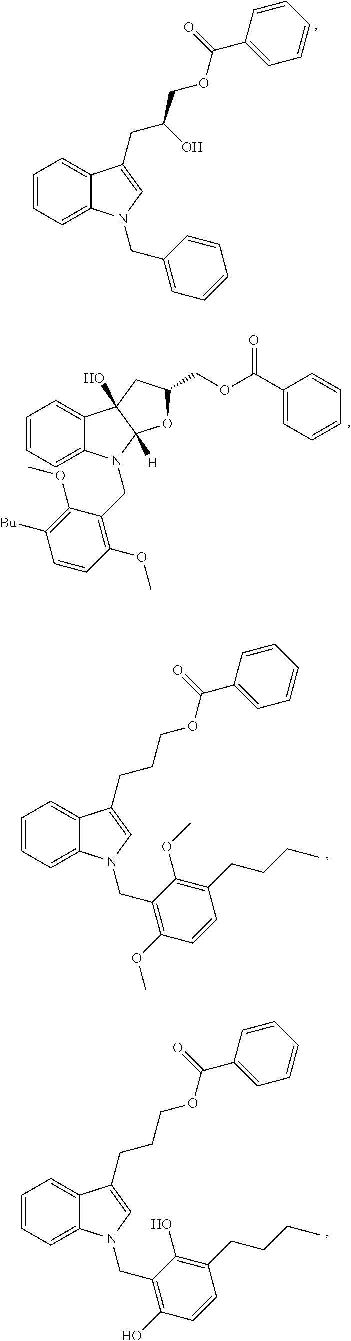

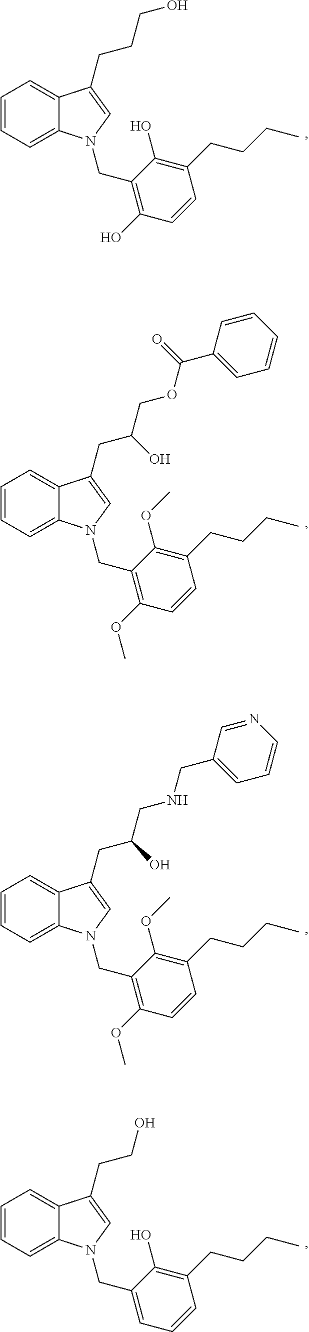 [SMILES: CCCCc1ccc(O)c(Cn2cc(CCCO)c3ccccc32)c1O ; CCCCc1ccc(OC)c(Cn2cc(CC(O)COC(=O)c3ccccc3)c3ccccc32)c1OC ; CCCCc1ccc(OC)c(Cn2cc(C[C@H](O)CNCc3cccnc3)c3ccccc32)c1OC ; CCCCc1cccc(Cn2cc(CCO)c3ccccc32)c1O]

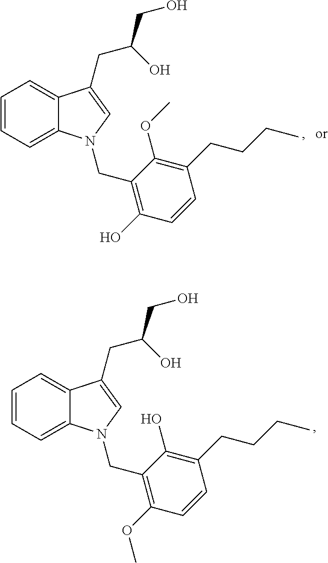 [SMILES: CCCCc1ccc(O)c(Cn2cc(C[C@H](O)CO)c3ccccc32)c1OC ; CCCCc1ccc(OC)c(Cn2cc(C[C@H](O)CO)c3ccccc32)c1O]

or subgroup thereof.

In one aspect, a compound can be present as:

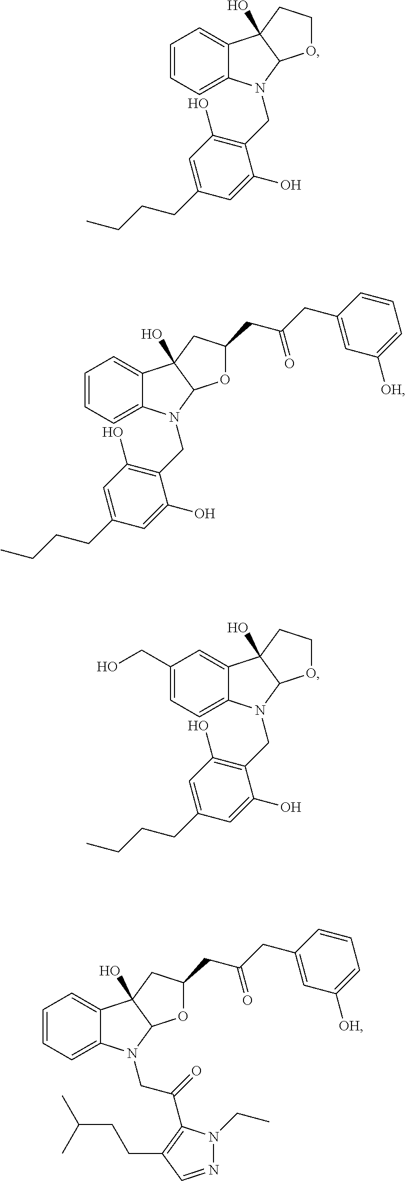 [SMILES: CCCCc1cc(O)c(CN2c3ccccc3[C@]3(O)CCOC23)c(O)c1 ; CCCCc1cc(O)c(CN2c3ccccc3[C@]3(O)C[C@@H](CC(=O)Cc4cccc(O)c4)OC23)c(O)c1 ; CCCCc1cc(O)c(CN2c3ccc(CO)cc3[C@]3(O)CCOC23)c(O)c1 ; CCn1ncc(CCC(C)C)c1C(=O)CN1c2ccccc2[C@]2(O)C[C@@H](CC(=O)Cc3cccc(O)c3)OC12]

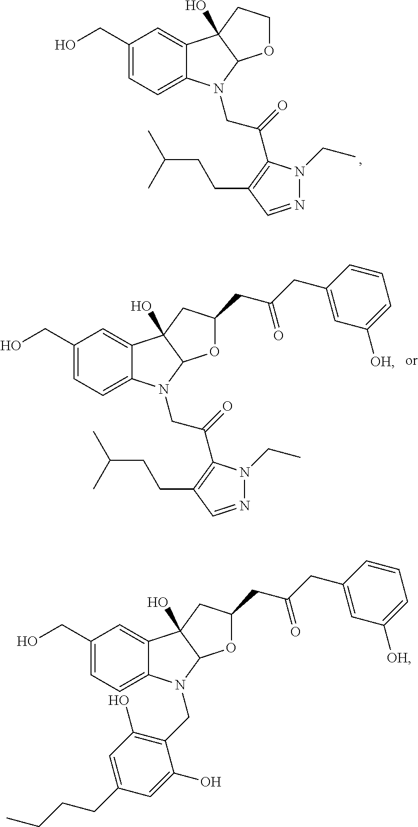

or a subgroup thereof.

In one aspect, a compound can be present as:

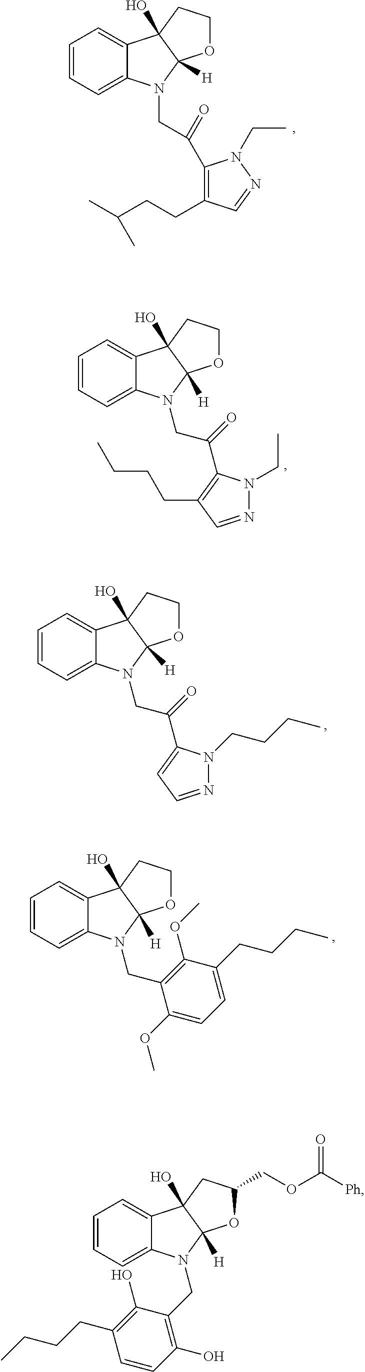

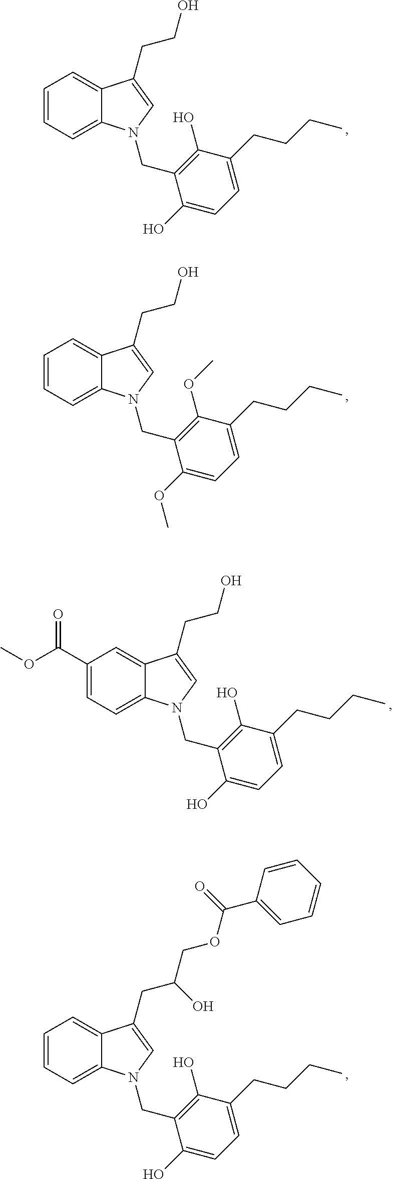

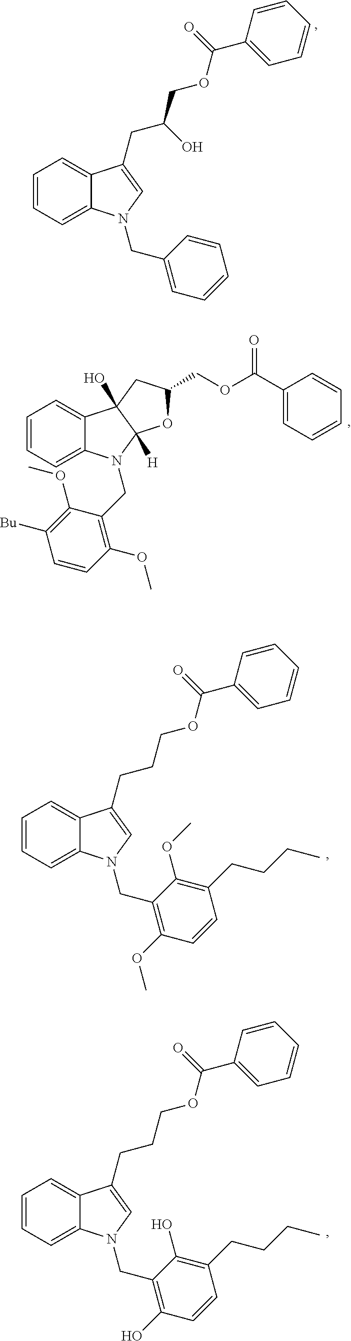

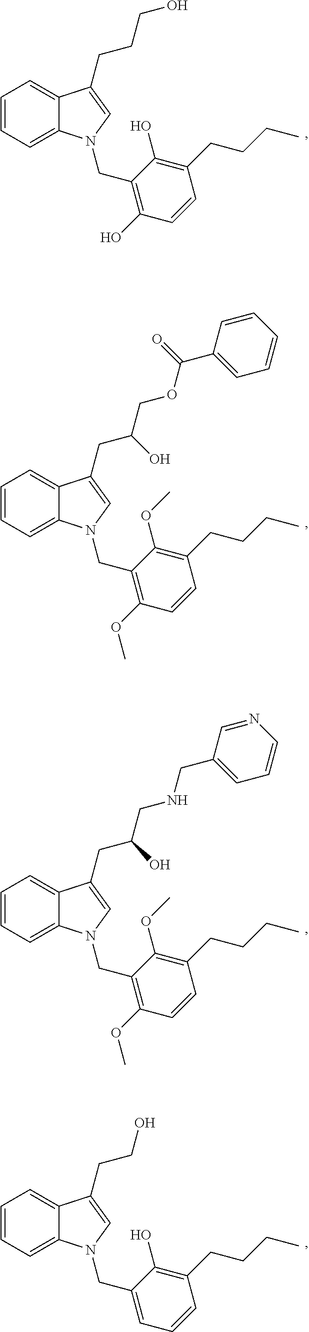 [SMILES: CCCCc1ccc(O)c(Cn2cc(CCCO)c3ccccc32)c1O ; CCCCc1ccc(OC)c(Cn2cc(CC(O)COC(=O)c3ccccc3)c3ccccc32)c1OC ; CCCCc1ccc(OC)c(Cn2cc(C[C@H](O)CNCc3cccnc3)c3ccccc32)c1OC ; CCCCc1cccc(Cn2cc(CCO)c3ccccc32)c1O]

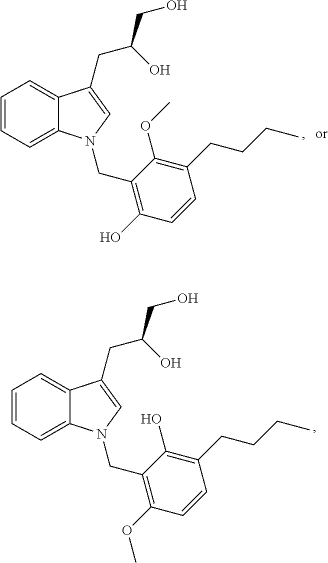 [SMILES: CCCCc1ccc(O)c(Cn2cc(C[C@H](O)CO)c3ccccc32)c1OC ; CCCCc1ccc(OC)c(Cn2cc(C[C@H](O)CO)c3ccccc32)c1O]

or a subgroup thereof.

In one aspect, a compound can be present as:

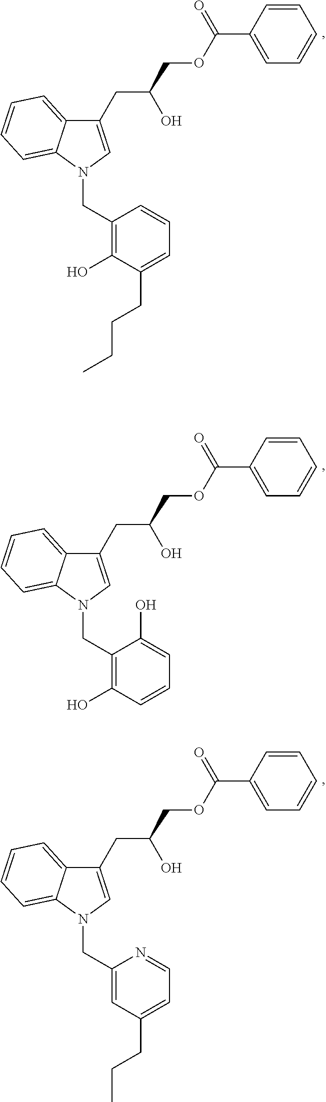 [SMILES: CCCCc1cccc(Cn2cc(C[C@H](O)COC(=O)c3ccccc3)c3ccccc32)c1O ; O=C(OC[C@@H](O)Cc1cn(Cc2c(O)cccc2O)c2ccccc12)c1ccccc1 ; CCCc1ccnc(Cn2cc(C[C@H](O)COC(=O)c3ccccc3)c3ccccc32)c1]

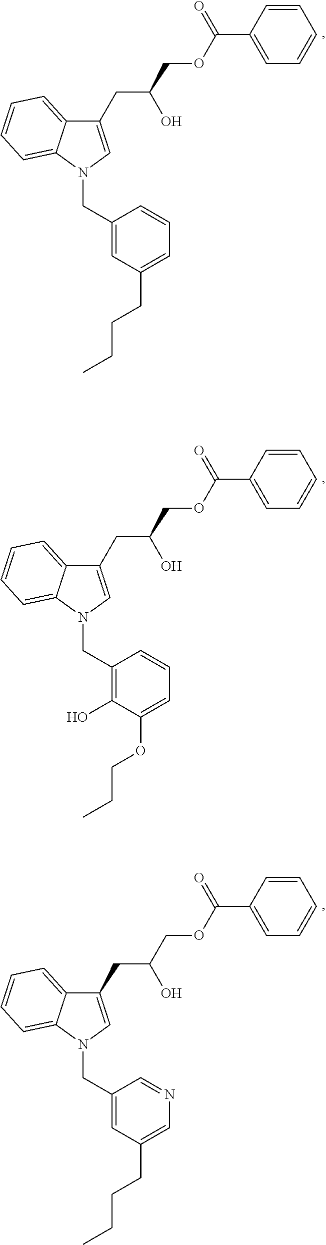

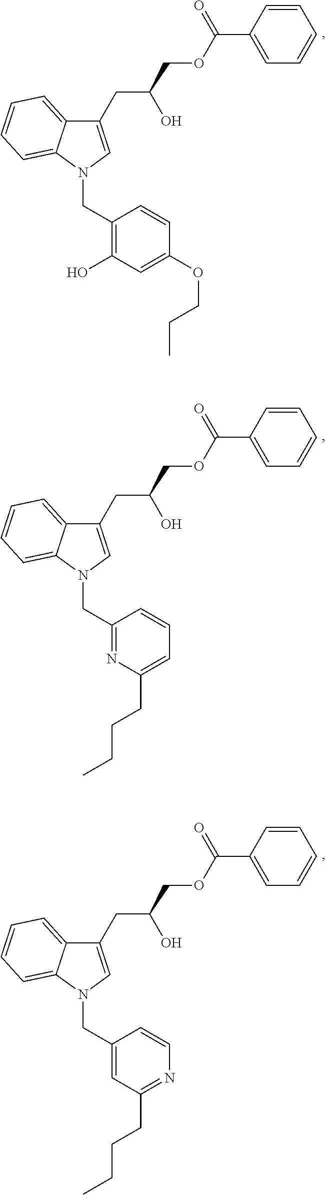 [SMILES: CCCOc1ccc(Cn2cc(C[C@H](O)COC(=O)c3ccccc3)c3ccccc32)c(O)c1 ; CCCCc1cccc(Cn2cc(C[C@H](O)COC(=O)c3ccccc3)c3ccccc32)n1 ; CCCCc1cc(Cn2cc(C[C@H](O)COC(=O)c3ccccc3)c3ccccc32)ccn1]

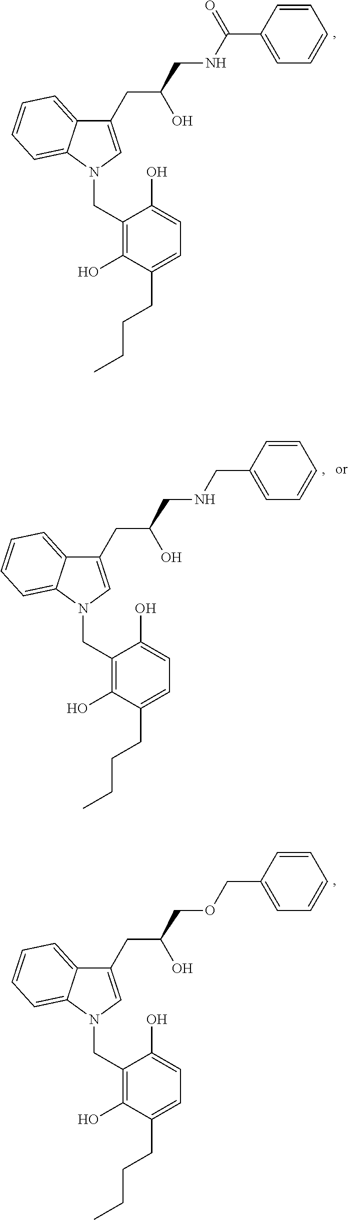 [SMILES: CCCCc1ccc(O)c(Cn2cc(C[C@H](O)CNC(=O)c3ccccc3)c3ccccc32)c1O ; CCCCc1ccc(O)c(Cn2cc(C[C@H](O)CNCc3ccccc3)c3ccccc32)c1O ; CCCCc1ccc(O)c(Cn2cc(C[C@H](O)COCc3ccccc3)c3ccccc32)c1O]

or a subgroup thereof.

In one aspect, a compound can be present as:

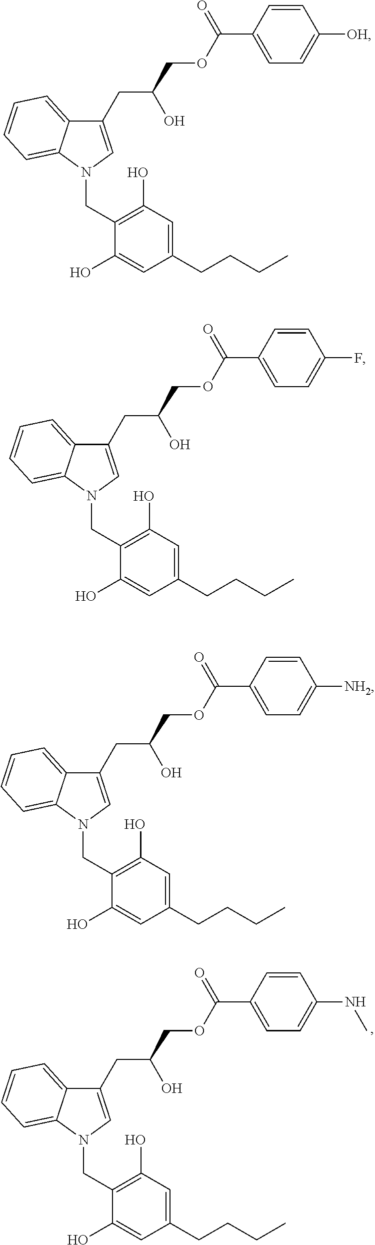 [SMILES: CCCCc1cc(O)c(Cn2cc(C[C@H](O)COC(=O)c3ccc(O)cc3)c3ccccc32)c(O)c1 ; CCCCc1cc(O)c(Cn2cc(C[C@H](O)COC(=O)c3ccc(F)cc3)c3ccccc32)c(O)c1 ; CCCCc1cc(O)c(Cn2cc(C[C@H](O)COC(=O)c3ccc(N)cc3)c3ccccc32)c(O)c1 ; CCCCc1cc(O)c(Cn2cc(C[C@H](O)COC(=O)c3ccc(NC)cc3)c3ccccc32)c(O)c1]

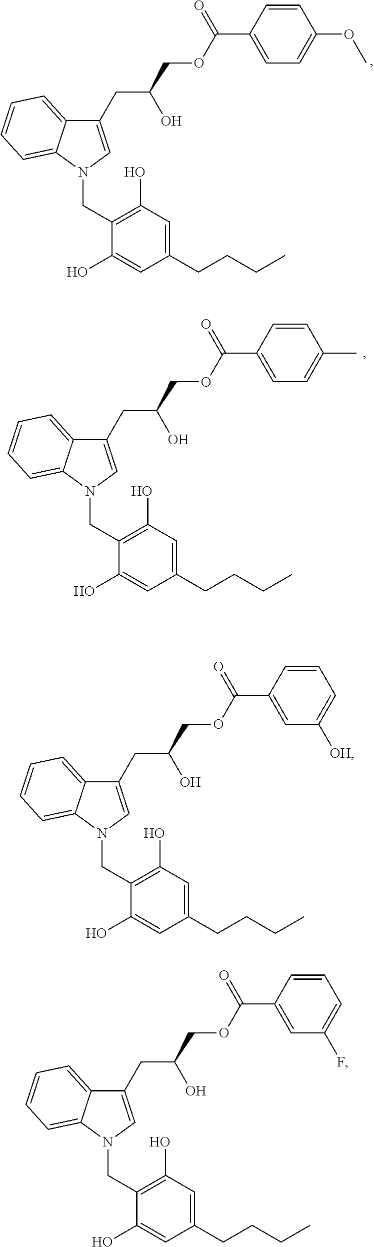

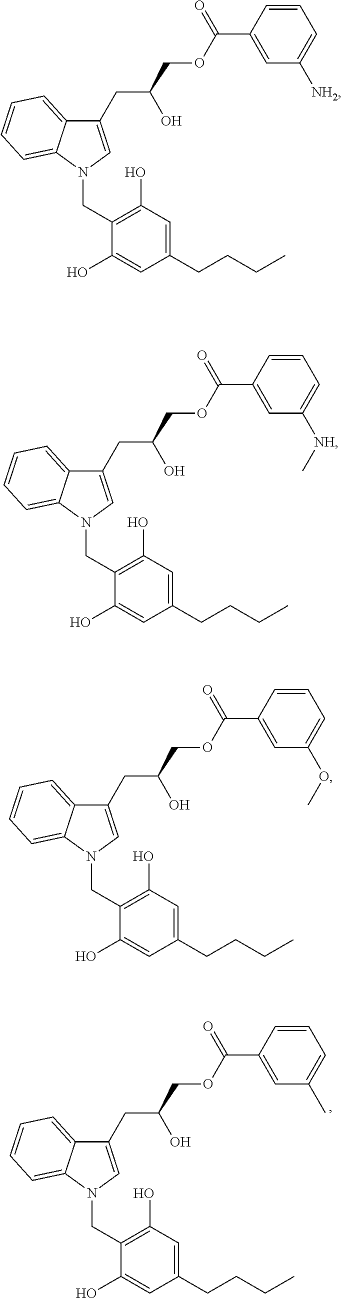 [SMILES: CCCCc1cc(O)c(Cn2cc(C[C@H](O)COC(=O)c3cccc(N)c3)c3ccccc32)c(O)c1 ; CCCCc1cc(O)c(Cn2cc(C[C@H](O)COC(=O)c3cccc(NC)c3)c3ccccc32)c(O)c1 ; CCCCc1cc(O)c(Cn2cc(C[C@H](O)COC(=O)c3cccc(OC)c3)c3ccccc32)c(O)c1 ; CCCCc1cc(O)c(Cn2cc(C[C@H](O)COC(=O)c3cccc(C)c3)c3ccccc32)c(O)c1]

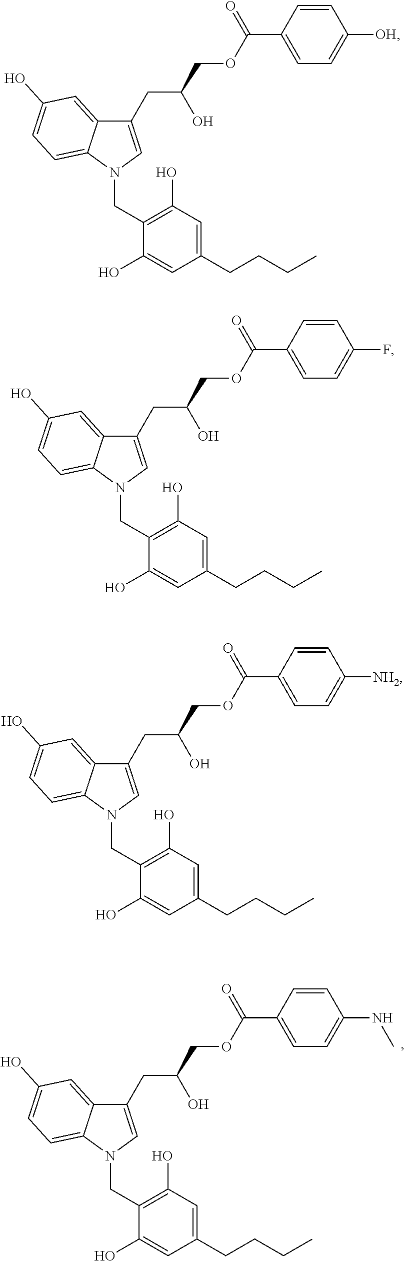 [SMILES: CCCCc1cc(O)c(Cn2cc(C[C@H](O)COC(=O)c3ccc(O)cc3)c3cc(O)ccc32)c(O)c1 ; CCCCc1cc(O)c(Cn2cc(C[C@H](O)COC(=O)c3ccc(F)cc3)c3cc(O)ccc32)c(O)c1 ; CCCCc1cc(O)c(Cn2cc(C[C@H](O)COC(=O)c3ccc(N)cc3)c3cc(O)ccc32)c(O)c1 ; CCCCc1cc(O)c(Cn2cc(C[C@H](O)COC(=O)c3ccc(NC)cc3)c3cc(O)ccc32)c(O)c1]

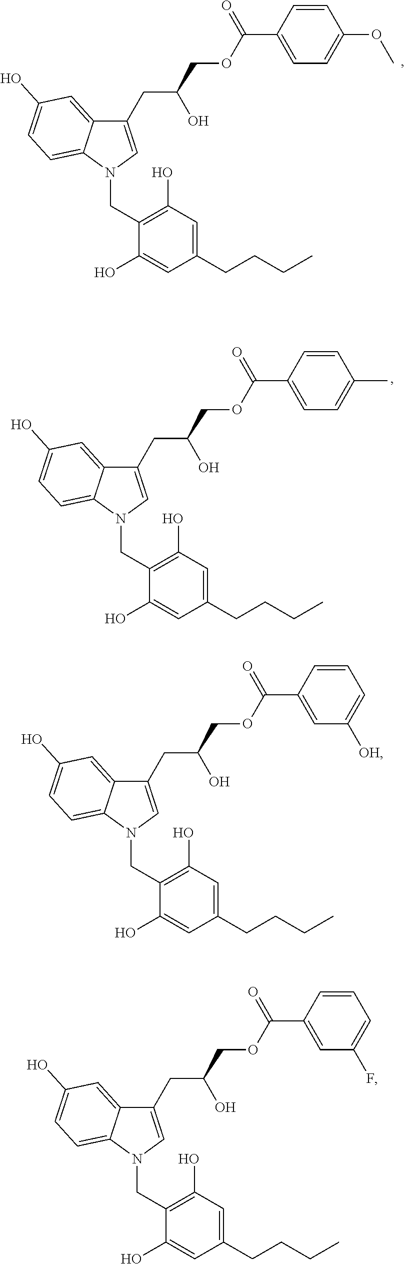 [SMILES: CCCCc1cc(O)c(Cn2cc(C[C@H](O)COC(=O)c3ccc(OC)cc3)c3cc(O)ccc32)c(O)c1 ; CCCCc1cc(O)c(Cn2cc(C[C@H](O)COC(=O)c3ccc(C)cc3)c3cc(O)ccc32)c(O)c1 ; CCCCc1cc(O)c(Cn2cc(C[C@H](O)COC(=O)c3cccc(O)c3)c3cc(O)ccc32)c(O)c1 ; CCCCc1cc(O)c(Cn2cc(C[C@H](O)COC(=O)c3cccc(F)c3)c3cc(O)ccc32)c(O)c1]

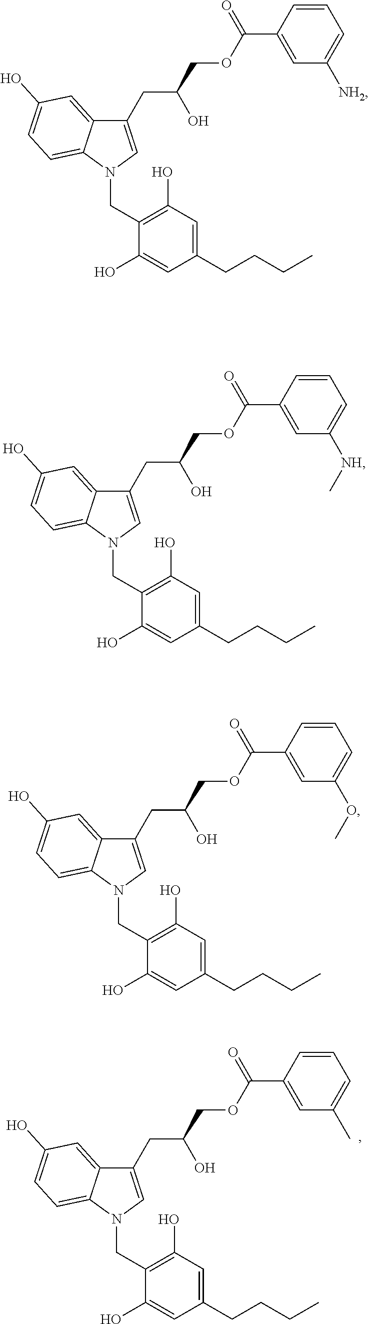 [SMILES: CCCCc1cc(O)c(Cn2cc(C[C@H](O)COC(=O)c3cccc(N)c3)c3cc(O)ccc32)c(O)c1 ; CCCCc1cc(O)c(Cn2cc(C[C@H](O)COC(=O)c3cccc(NC)c3)c3cc(O)ccc32)c(O)c1 ; CCCCc1cc(O)c(Cn2cc(C[C@H](O)COC(=O)c3cccc(OC)c3)c3cc(O)ccc32)c(O)c1 ; CCCCc1cc(O)c(Cn2cc(C[C@H](O)COC(=O)c3cccc(C)c3)c3cc(O)ccc32)c(O)c1]

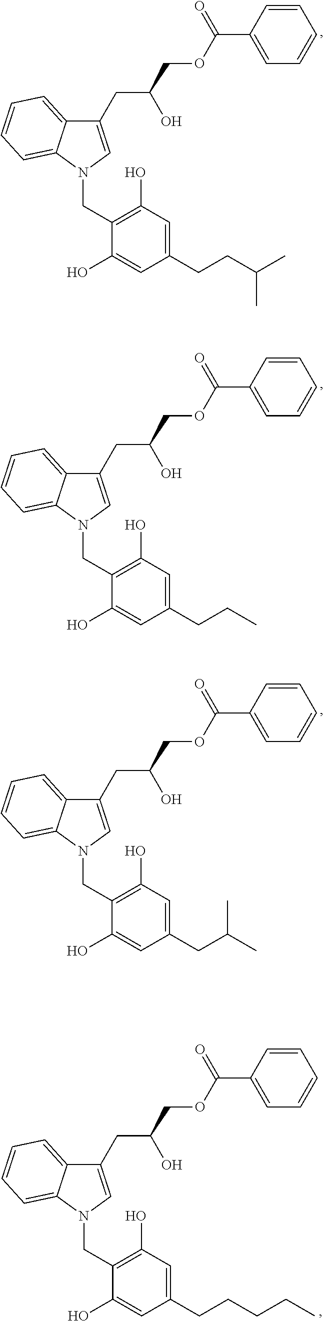 [SMILES: CC(C)CCc1cc(O)c(Cn2cc(C[C@H](O)COC(=O)c3ccccc3)c3ccccc32)c(O)c1 ; CCCc1cc(O)c(Cn2cc(C[C@H](O)COC(=O)c3ccccc3)c3ccccc32)c(O)c1 ; CC(C)Cc1cc(O)c(Cn2cc(C[C@H](O)COC(=O)c3ccccc3)c3ccccc32)c(O)c1 ; CCCCCc1cc(O)c(Cn2cc(C[C@H](O)COC(=O)c3ccccc3)c3ccccc32)c(O)c1]

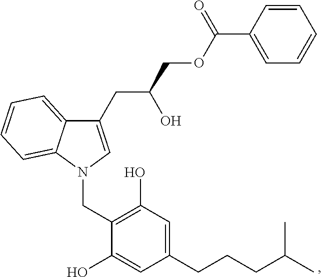 [SMILES: CC(C)CCCc1cc(O)c(Cn2cc(C[C@H](O)COC(=O)c3ccccc3)c3ccccc32)c(O)c1]

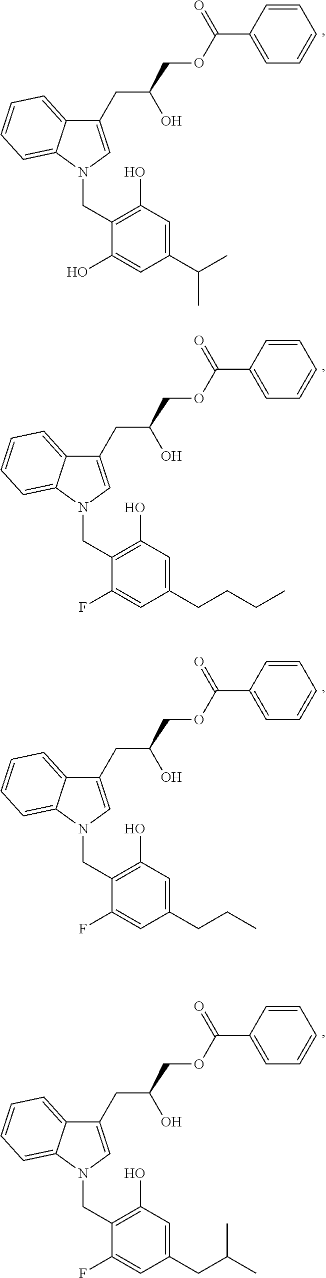

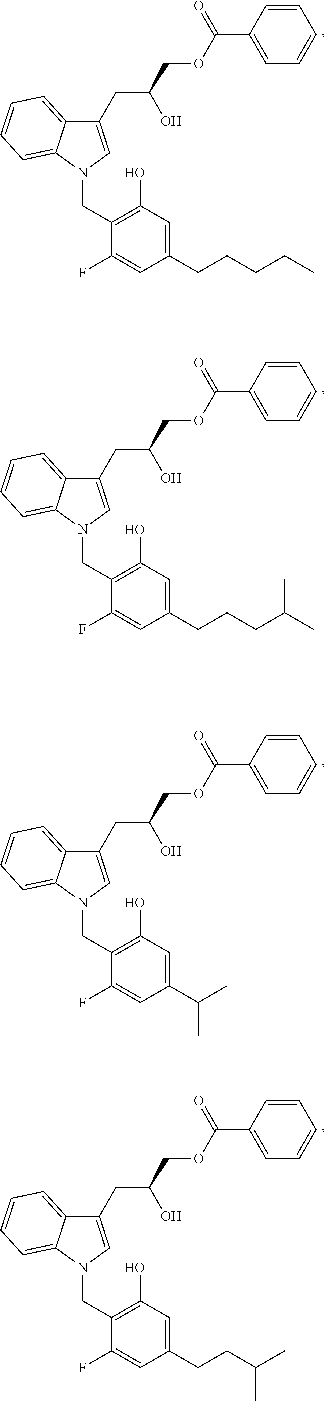 [SMILES: CCCCCc1cc(O)c(Cn2cc(C[C@H](O)COC(=O)c3ccccc3)c3ccccc32)c(F)c1 ; CC(C)CCCc1cc(O)c(Cn2cc(C[C@H](O)COC(=O)c3ccccc3)c3ccccc32)c(F)c1 ; CC(C)c1cc(O)c(Cn2cc(C[C@H](O)COC(=O)c3ccccc3)c3ccccc32)c(F)c1 ; CC(C)CCc1cc(O)c(Cn2cc(C[C@H](O)COC(=O)c3ccccc3)c3ccccc32)c(F)c1]

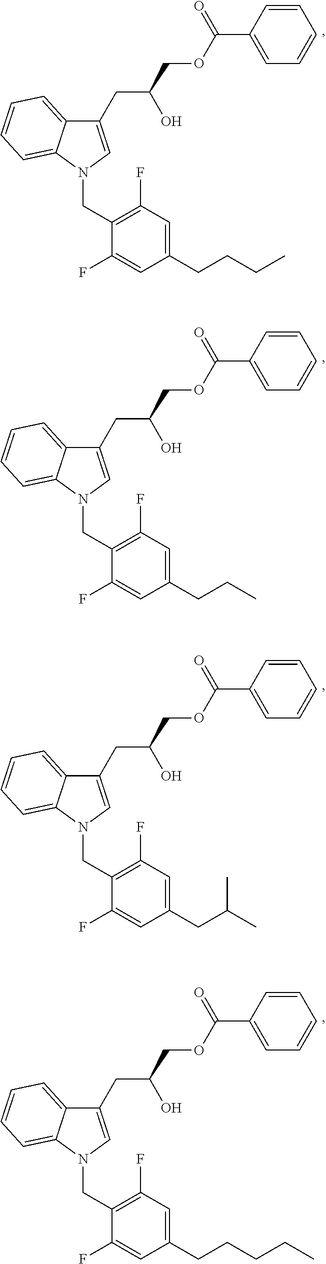 [SMILES: CCCCc1cc(F)c(Cn2cc(C[C@H](O)COC(=O)c3ccccc3)c3ccccc32)c(F)c1 ; CCCc1cc(F)c(Cn2cc(C[C@H](O)COC(=O)c3ccccc3)c3ccccc32)c(F)c1 ; CC(C)Cc1cc(F)c(Cn2cc(C[C@H](O)COC(=O)c3ccccc3)c3ccccc32)c(F)c1 ; CCCCCc1cc(F)c(Cn2cc(C[C@H](O)COC(=O)c3ccccc3)c3ccccc32)c(F)c1]

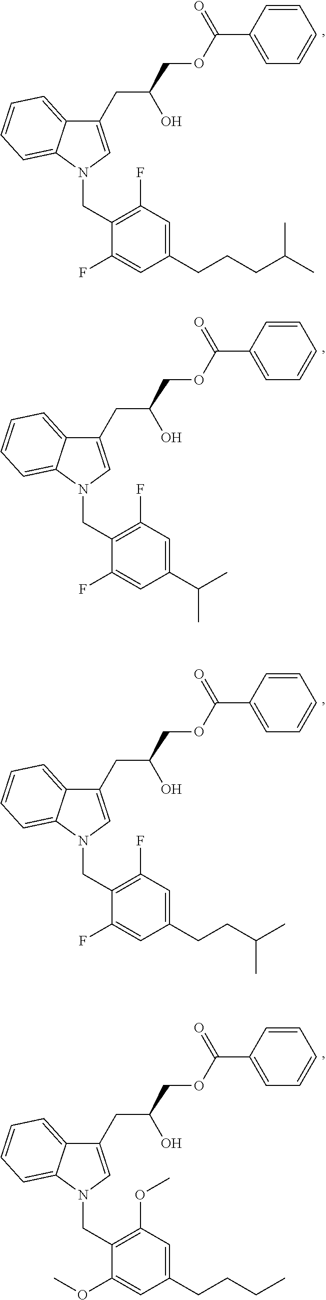 [SMILES: CC(C)CCCc1cc(F)c(Cn2cc(C[C@H](O)COC(=O)c3ccccc3)c3ccccc32)c(F)c1 ; CC(C)c1cc(F)c(Cn2cc(C[C@H](O)COC(=O)c3ccccc3)c3ccccc32)c(F)c1 ; CC(C)CCc1cc(F)c(Cn2cc(C[C@H](O)COC(=O)c3ccccc3)c3ccccc32)c(F)c1 ; CCCCc1cc(OC)c(Cn2cc(C[C@H](O)COC(=O)c3ccccc3)c3ccccc32)c(OC)c1]

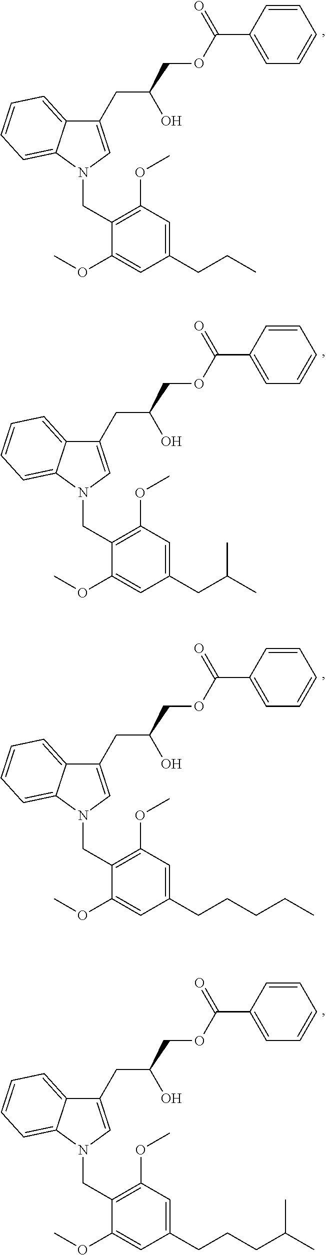 [SMILES: CCCc1cc(OC)c(Cn2cc(C[C@H](O)COC(=O)c3ccccc3)c3ccccc32)c(OC)c1 ; COc1cc(CC(C)C)cc(OC)c1Cn1cc(C[C@H](O)COC(=O)c2ccccc2)c2ccccc21 ; CCCCCc1cc(OC)c(Cn2cc(C[C@H](O)COC(=O)c3ccccc3)c3ccccc32)c(OC)c1 ; COc1cc(CCCC(C)C)cc(OC)c1Cn1cc(C[C@H](O)COC(=O)c2ccccc2)c2ccccc21]

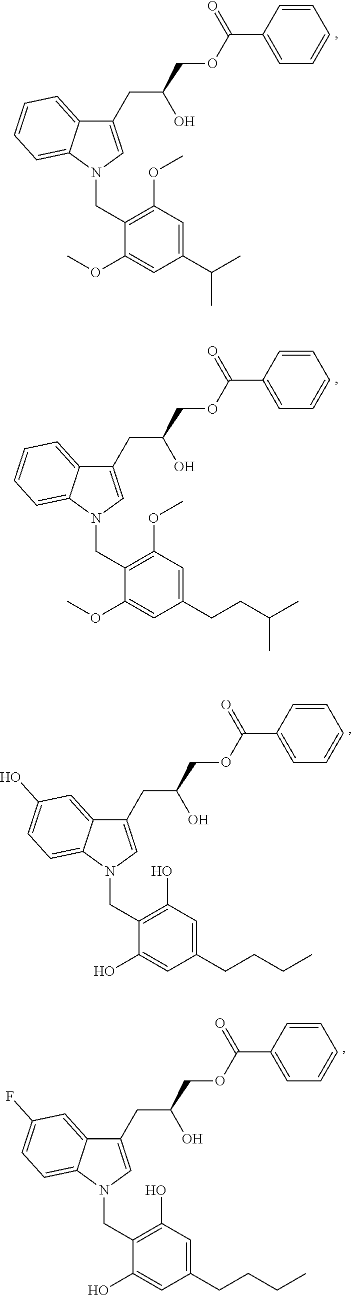

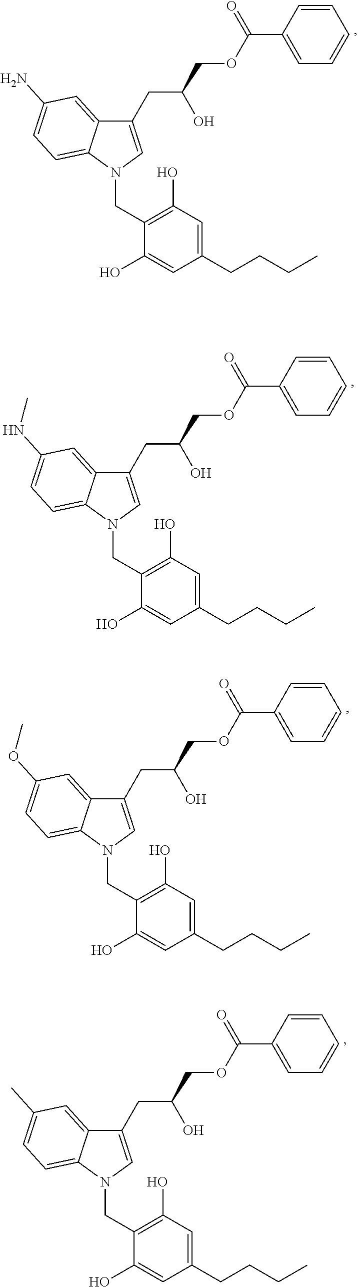 [SMILES: CCCCc1cc(O)c(Cn2cc(C[C@H](O)COC(=O)c3ccccc3)c3cc(N)ccc32)c(O)c1 ; CCCCc1cc(O)c(Cn2cc(C[C@H](O)COC(=O)c3ccccc3)c3cc(NC)ccc32)c(O)c1 ; CCCCc1cc(O)c(Cn2cc(C[C@H](O)COC(=O)c3ccccc3)c3cc(OC)ccc32)c(O)c1 ; CCCCc1cc(O)c(Cn2cc(C[C@H](O)COC(=O)c3ccccc3)c3cc(C)ccc32)c(O)c1]

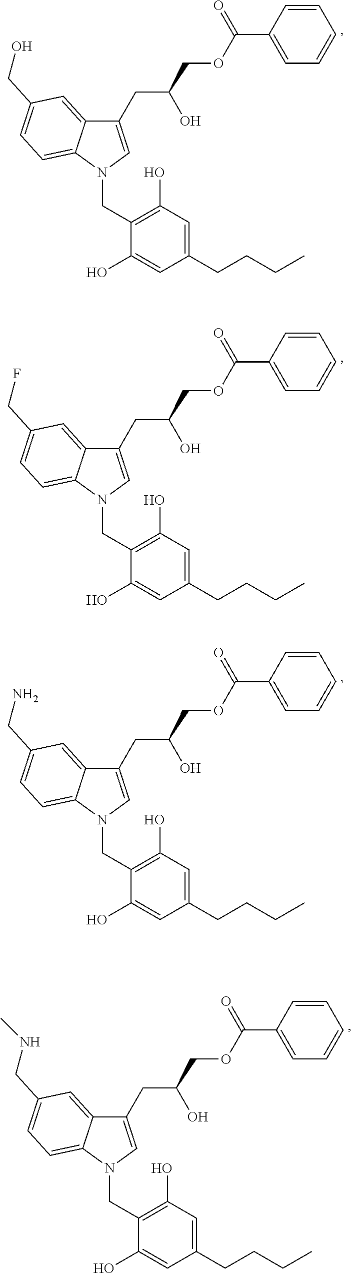 [SMILES: CCCCc1cc(O)c(Cn2cc(C[C@H](O)COC(=O)c3ccccc3)c3cc(CO)ccc32)c(O)c1 ; CCCCc1cc(O)c(Cn2cc(C[C@H](O)COC(=O)c3ccccc3)c3cc(CF)ccc32)c(O)c1 ; CCCCc1cc(O)c(Cn2cc(C[C@H](O)COC(=O)c3ccccc3)c3cc(CN)ccc32)c(O)c1 ; CCCCc1cc(O)c(Cn2cc(C[C@H](O)COC(=O)c3ccccc3)c3cc(CNC)ccc32)c(O)c1]

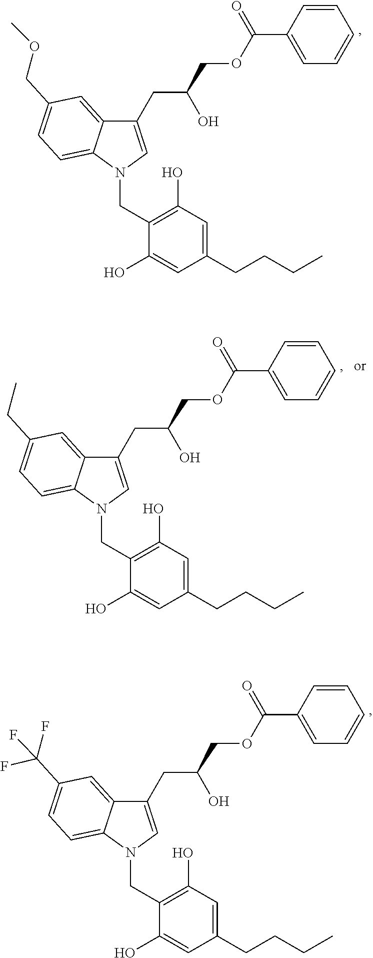 [SMILES: CCCCc1cc(O)c(Cn2cc(C[C@H](O)COC(=O)c3ccccc3)c3cc(COC)ccc32)c(O)c1 ; CCCCc1cc(O)c(Cn2cc(C[C@H](O)COC(=O)c3ccccc3)c3cc(CC)ccc32)c(O)c1 ; CCCCc1cc(O)c(Cn2cc(C[C@H](O)COC(=O)c3ccccc3)c3cc(C(F)(F)F)ccc32)c(O)c1]

or a subgroup thereof.

In one aspect, a compound can be present as:

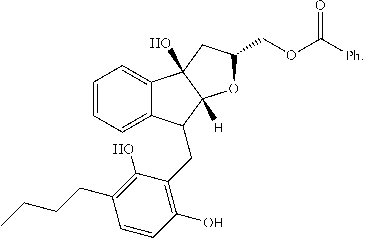

In one aspect, a compound can be present as:

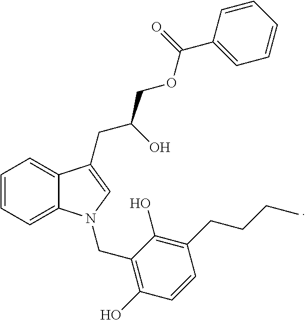 [SMILES: CCCCc1ccc(O)c(Cn2cc(C[C@H](O)COC(=O)c3ccccc3)c3ccccc32)c1O ; "."]

In a further aspect, the compound exhibits binding to gp130 with an equilibrium dissociation binding constant (K_{D}) of less than about 200 μM. In a still further aspect, the compound exhibits binding to gp130 with an equilibrium dissociation binding constant (K_{D}) of less than about 100 μM. In a yet further aspect, the compound exhibits binding to gp130 with an equilibrium dissociation binding constant (K_{D}) of less than about 50 μM. In an even further aspect, the compound exhibits binding to gp130 with an equilibrium dissociation binding constant (K_{D}) of less than about 30 μM. In a still further aspect, the compound exhibits binding to gp130 with an equilibrium dissociation binding constant (K_{D}) of less than about 10 μM.

In a further aspect, the K_{D} is determined by surface Plasmon resonance using gp130-Fc-HA cross-linked to the flow cell on a carboxymethylated dextran matrix via amino coupling.

In a further aspect, the disclosed compounds are inhibitors of homodimerization of a IL6-IL6R-gp130 heterotrimer. In a still further aspect, the disclosed compounds inhibit activation of the Jak2/STAT3 pathway by IL6.

In a further aspect, the disclosed compounds are inhibitors of STAT3 activation.

It is contemplated that one or more compounds can optionally be omitted from the disclosed invention.

3. gp130 Binding Activity

Generally, the disclosed compounds exhibit binding to gp130. In various aspects, the disclosed compounds exhibit binding to gp130 with an equilibrium dissociation binding constant (K_{D}) of less than about 200 μM. In a further aspect, the disclosed compounds exhibit binding to gp130 with an equilibrium dissociation binding constant (K_{D}) of less than about 100 μM. In a still further aspect, the disclosed compounds exhibit binding to gp130 with an equilibrium dissociation binding constant (K_{D}) of less than about 50 μM. In a yet further aspect, the disclosed compounds exhibit binding to gp130 with an equilibrium dissociation binding constant (K_{D}) of less than about 40 μM. In an even further aspect, the disclosed compounds exhibit binding to gp130 with an equilibrium dissociation binding constant (K_{D}) of less than about 30 μM. In a still further aspect, the disclosed compounds exhibit binding to gp130 with an equilibrium dissociation binding constant (K_{D}) of less than about 20 μM. In a yet further aspect, the disclosed compounds exhibit binding to gp130 with an equilibrium dissociation binding constant (K_{D}) of less than about 10 μM.

In a further aspect, the K_{D} is determined by surface Plasmon resonance using gp130-Fc-HA cross-linked to the flow cell on a carboxymethylated dextran matrix via amino coupling.

### C. METHODS OF MAKING THE COMPOUNDS

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, in addition to other standard manipulations known in the literature or to one skilled in the art. The following examples are provided so that the invention might be more fully understood, are illustrative only, and should not be construed as limiting.

In one aspect, the disclosed compounds comprise the products of the synthetic methods described herein. In a further aspect, the disclosed compounds comprise a compound produced by a synthetic method described herein. In a still further aspect, the invention comprises a pharmaceutical composition comprising a therapeutically effective amount of the product of the disclosed methods and a pharmaceutically acceptable carrier. In a still further aspect, the invention comprises a method for manufacturing a medicament comprising combining at least one compound of any of disclosed compounds or at least one product of the disclosed methods with a pharmaceutically acceptable carrier or diluent.

In one aspect, the invention relates to methods of making compounds useful as inhibitors of gp130, which can be useful in the treatment of hyperproliferation disorders, immune disorders, and other diseases in which interleukin-6 or STAT3 is involved.

1. Synthesis of “Northern” Moiety

In one aspect, compounds of the present invention can be prepared as shown below.

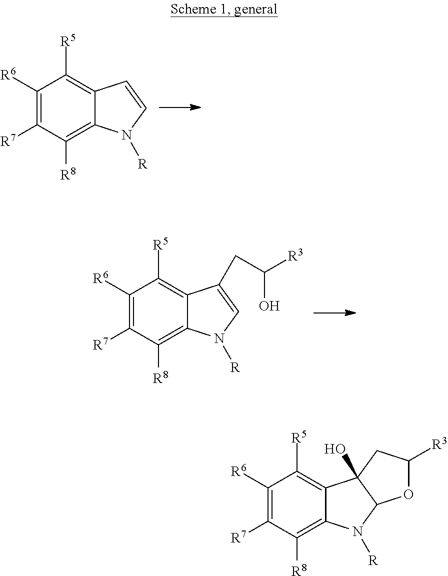

In one aspect of the disclosed convergent synthesis, the “Northern” portion can be prepared by, for example, addition of an electrophile to indole ring to provide a 2-(1H-indol-3-yl)ethanol structure. In a further aspect, the hydroxyl functionality can be used to effect ring closure, thereby providing a 3,3a,8,8a-tetrahydro-2H-furo[2,3-b]indole structure. It is appreciated but either the open-ring form for the closed-ring form can be carried forward in the synthetic route. In this Scheme, compounds are represented in generic form, with substituents as noted in compound descriptions elsewhere herein. More specific examples are set forth below.

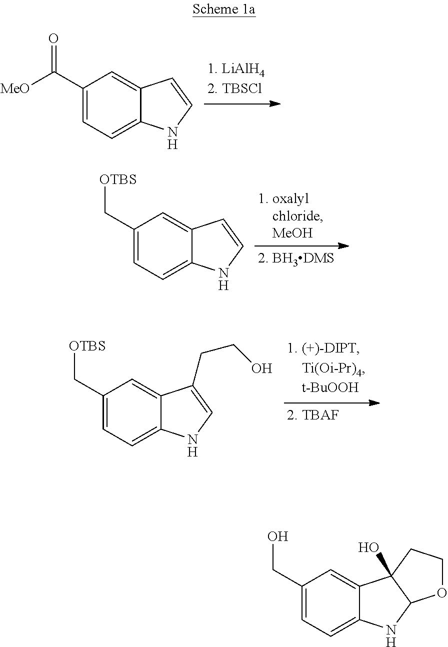

In this example, substituents are manipulated using conventional reactions. Addition of a hydroxyethyl functionality via oxalyl chloride, followed by reduction, provides the open ring form. Asymmetric epoxidation and intramolecular written closure yields the closed-ring form. The resultant compound can then be optionally deprotected.

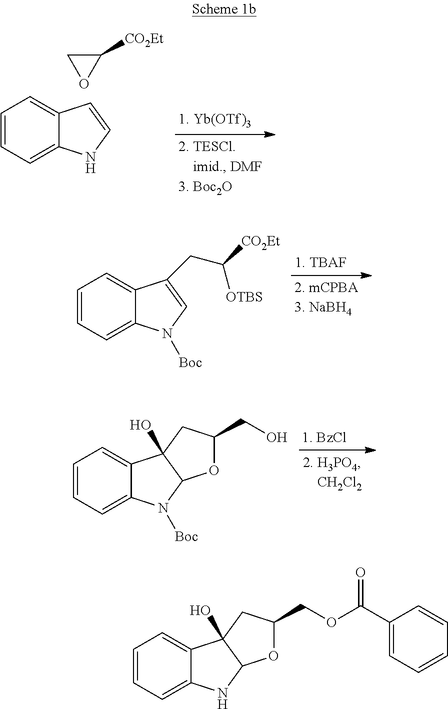

As another example, electrophilic addition of a substituted chiral epoxide provides the open-ring form. The indole nitrogen and hydroxyl are then optionally protected. Selective deprotection of the hydroxyl group, followed by oxidation and intramolecular ring closure, yields the closed-ring form. Again, substituents can be manipulated using conventional reactions.

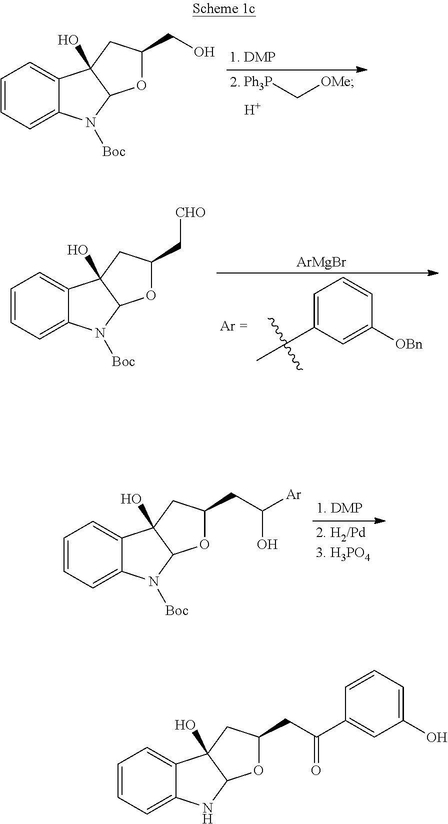

In yet another example, the hydroxyethyl substituent can be homologated via oxidation and Wittig reaction. Subsequent reaction with a Grignard reagent further elaborates the side-chain. Finally, oxidation provides the desired ketone. It is contemplated that the various substituents can be protected and deprotected as necessary during the synthetic sequence.

2. Synthesis of “Southern” Moiety

In a further aspect of the disclosed convergent synthesis, the “Southern” portion can be obtained commercially or can be prepared by, for example, conventional reactions. Suitable compounds for use as the “Southern” portion include electrophiles that can form a covalent bond with the indole nitrogen atom. Specific examples are set forth below.

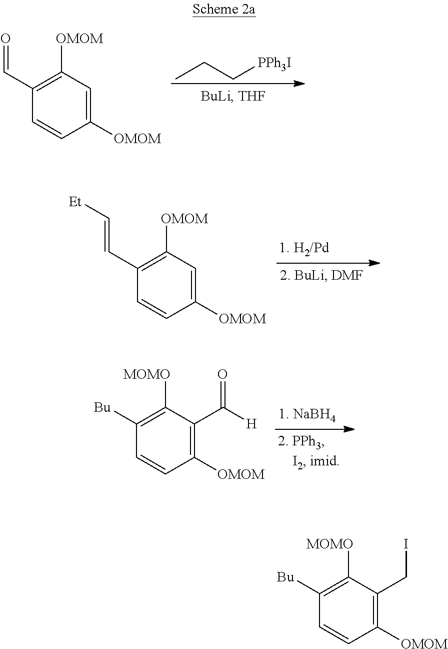

In this example, the aromatic moiety is elaborated by Wittig reaction, hydrogenation, Villsmeyer formylation, reduction, and halogenation to provide the desired compound.

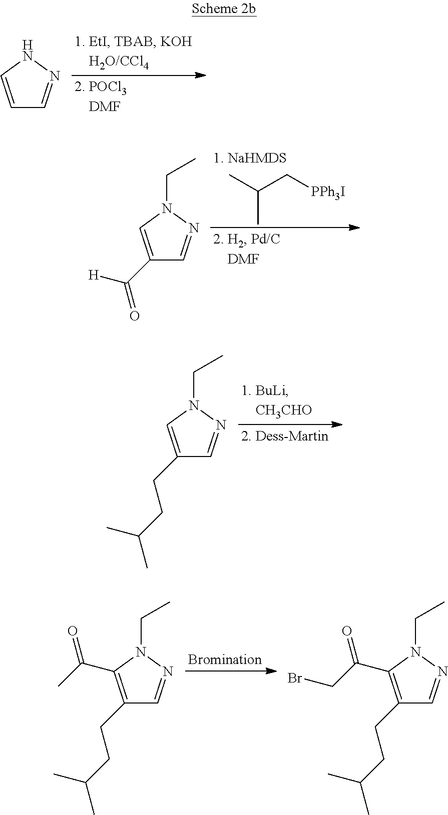

In a further example, pyrazole is functionalized by alkylation and formylation, and the side chain is elaborated by Wittig reaction. Subsequent lithiation and treatment with acetaldehyde, followed by oxidation and halogenation, provides the desired compound.

3. Joining of ‘Northern” and “Southern” Moieties

In a yet further aspect of the disclosed convergent synthesis, the “Northern” portion can be joined to the “Southern” portion by, for example, conventional reactions. Suitable reactions for use in this aspect include nucleophilic substitution reactions between an electrophile and the indole nitrogen atom.

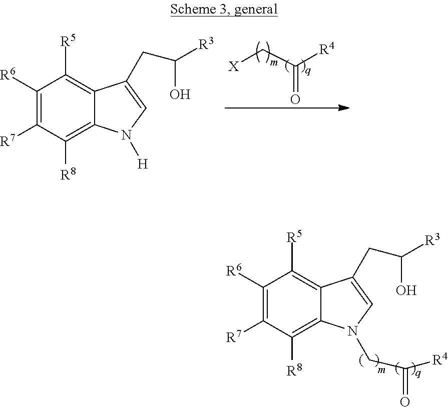

In one aspect, the open-ring form can be reacted with a suitable halogenated compound in a nucleophilic substitution reaction to form a covalent bond at the indole nitrogen atom.

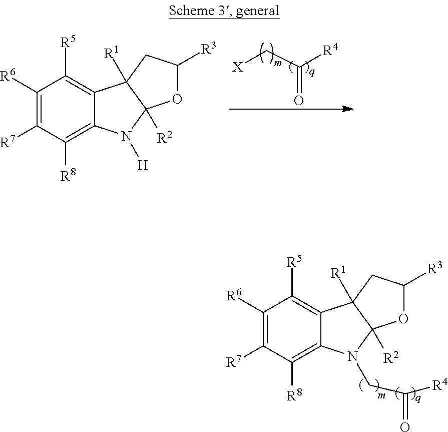

In a further aspect, the closed-ring form can be reacted with a suitable halogenated compound in a nucleophilic substitution reaction to form a covalent bond at the indole nitrogen atom. In these general Schemes, compounds are represented in generic form, with substituents as noted in compound descriptions elsewhere herein. More specific examples are set forth below.

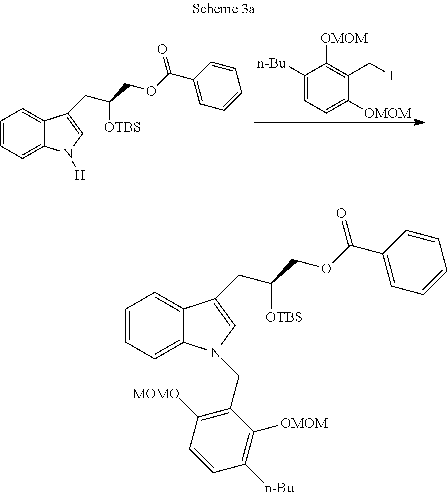

In one example, an open-ring compounds can be used as the “Northern” portion and coupled to the “Southern’ portion from Scheme 2a via a nucleophilic substitution reaction.

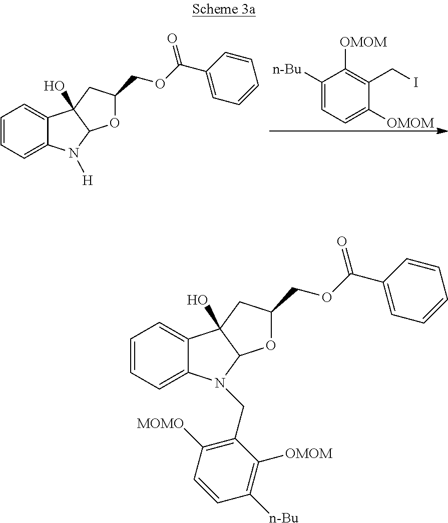

In a further example, the closed-ring “Northern” portion from Scheme 1b and the “Southern” portion from Scheme 2a can be joined via nucleophilic substitution reaction.

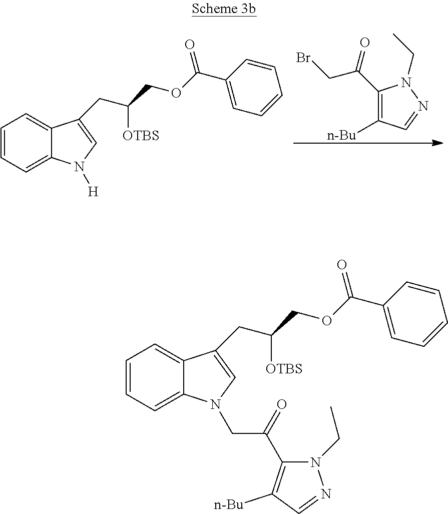

In a further example, an open-ring compounds can be used as the “Northern” portion and coupled to the “Southern’ portion from Scheme 2b via a nucleophilic substitution reaction.

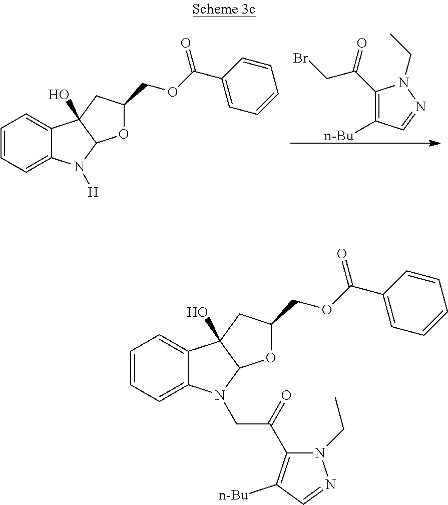

In a further example, the closed-ring “Northern” portion from Scheme 1b and the “Southern” portion from Scheme 2b can be joined via nucleophilic substitution reaction.

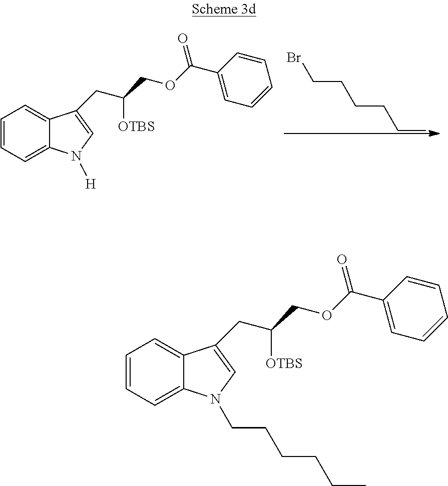

In a yet further example, an open-ring compounds can be used as the “Northern” portion and coupled via a nucleophilic substitution reaction to the commercially available hexyl bromide as the “Southern’ portion.

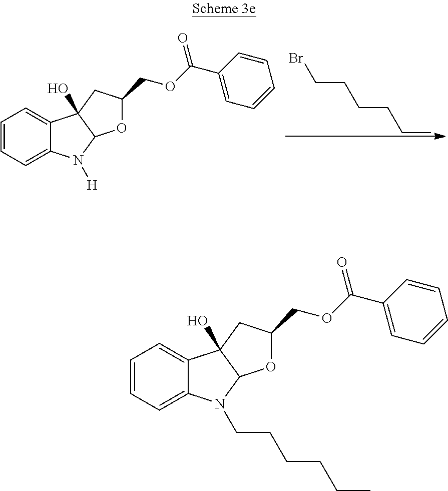

In a yet further example, the closed-ring “Northern” portion from Scheme 1b can be coupled via a nucleophilic substitution reaction to the commercially available hexyl bromide as the “Southern’ portion.

### D. COMPUTATIONAL MODELING

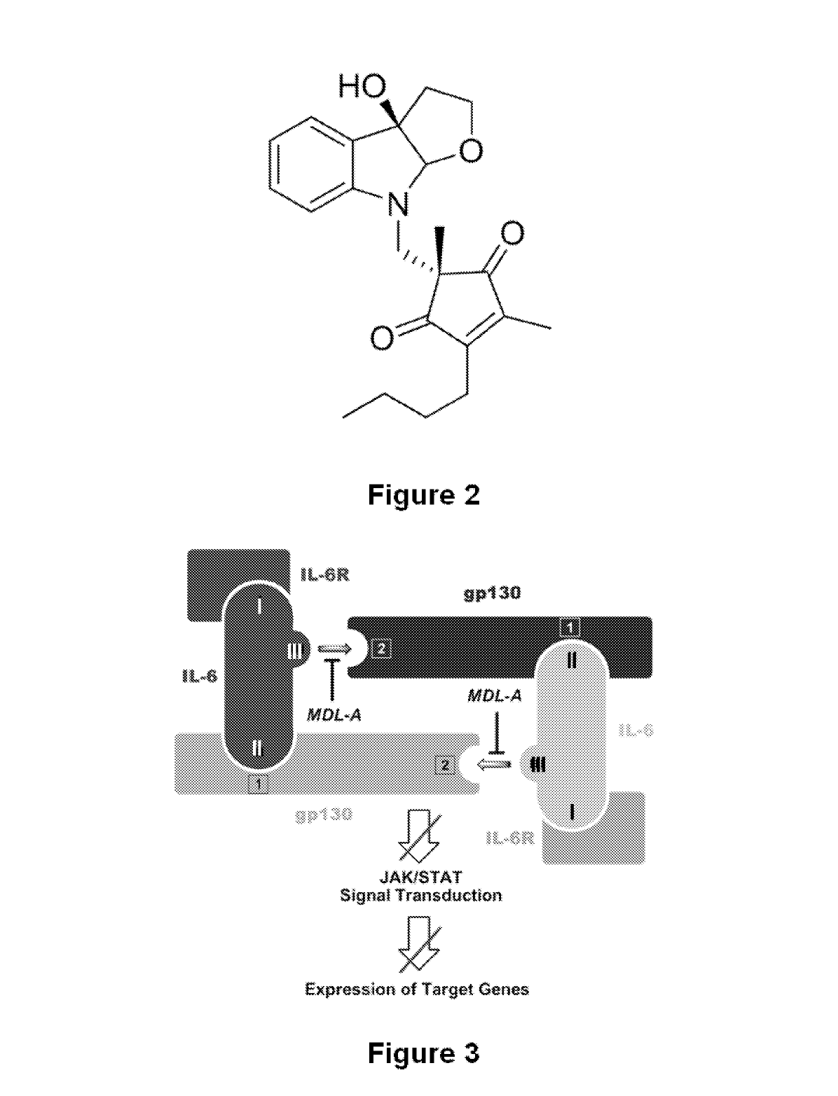

In 1996, Hayashi et al. discovered the novel natural product, madindoline A (MDL-A or MadA, FIG. 2), from the fermentation broth of Streptomyces nitrosporeus K93-0711 as a nonpeptide antagonist of GP130.¹⁸ The nontoxic compound was found to induce osteoclastogenesis in vitro and bone resorption in an experimental model of postmenopausal osteoporosis in vivo through inhibition of GP130.¹⁹ Madindoline A does not disrupt the formation of the IL-6/IL-6R/GP130 heterotrimer but rather prevents its homodimerization, suppressing the IL-6/JAK/STAT signaling cascade (FIG. 3). Omura and coworkers showed that the compound specifically inhibited the growth of the IL-6-dependent murine hepatoma cell line MH60, while the IL-6-independent MH60 cells were unaffected.²⁰ Saleh and coworkers²¹ subsequently confirmed that the compound binds to the extracellular domain of GP130 and inhibits IL-6-dependent STAT3 tyrosine phosphorylation in HepG2 cells. However, the natural product itself cannot practically be used as a drug, because madindoline A is no longer available from its natural source²⁰ and its GP130 binding affinity is very low^{21 (K}_{D} of 288 μM). Several synthetic approaches to the preparation of the natural product have been reported (ref. 22 and references therein), but due to the length and complexity of the syntheses cannot be applied economically in a pharmaceutical process. A structure-based approach to the synthesis of madindoline A analogues has not yet been reported.

Without wishing to be bound by a particular theory, homodimerization of the IL-6/IL-6R/GP130 heterotrimer in androgen independent prostate cancer, resulting in IL-6/JAK2/STAT3 signaling, could be one of major causes of cancer proliferation, anti-apoptosis, metastasis, drug resistance and revival. Thus, inhibition of this dimerization event and the resulting disruption of the downstream signal transduction pathway should provide an exciting new option for prostate cancer therapy. Novel drug-like small molecules will be designed and synthesized based on the general structure of madindoline A to effectively disrupt the dimerization of the IL-6/IL-6R/GP130 heterotrimers. The focus of this strategy will be on simplification of the structure of madindoline A to increase synthetic feasibility and on structural modification in order to increase potency.

To verify the direct binding of MDL-A and optimized synthetic analogues, the crystal structures of the D1 domain/inhibitor complexes can be deduced. Multiple GP130 extracellular domain structures have been solved over the years. As described herein, the D1 domain can be cloned, expressed and purified with sequence range of Leu2-Ser100 following the established protocol.²⁴ Inhibitors can be soaked into native crystals or co-crystallized with D1 protein solution sample. The complex structures can be solved through molecular replacement using native D1 structure as search model. The complex can then be used as a structural template to further optimize additional inhibitors in the iterative design cycle. ITC (isothermal titration calorimetry) measurements can also be carried out to determine the experimental D1/inhibitor binding free energy in order to cross-validate with the computational results.

Computational modeling and design can also be carried out continually and iteratively to provide additional design options through in silico library screening, fragment replacement and attachment, dynamics simulation to probe binding induced-fit effects, etc. Several additional designs for compound optimization in different fragment replacement choices have been identified. See flow chart (FIG. 11).

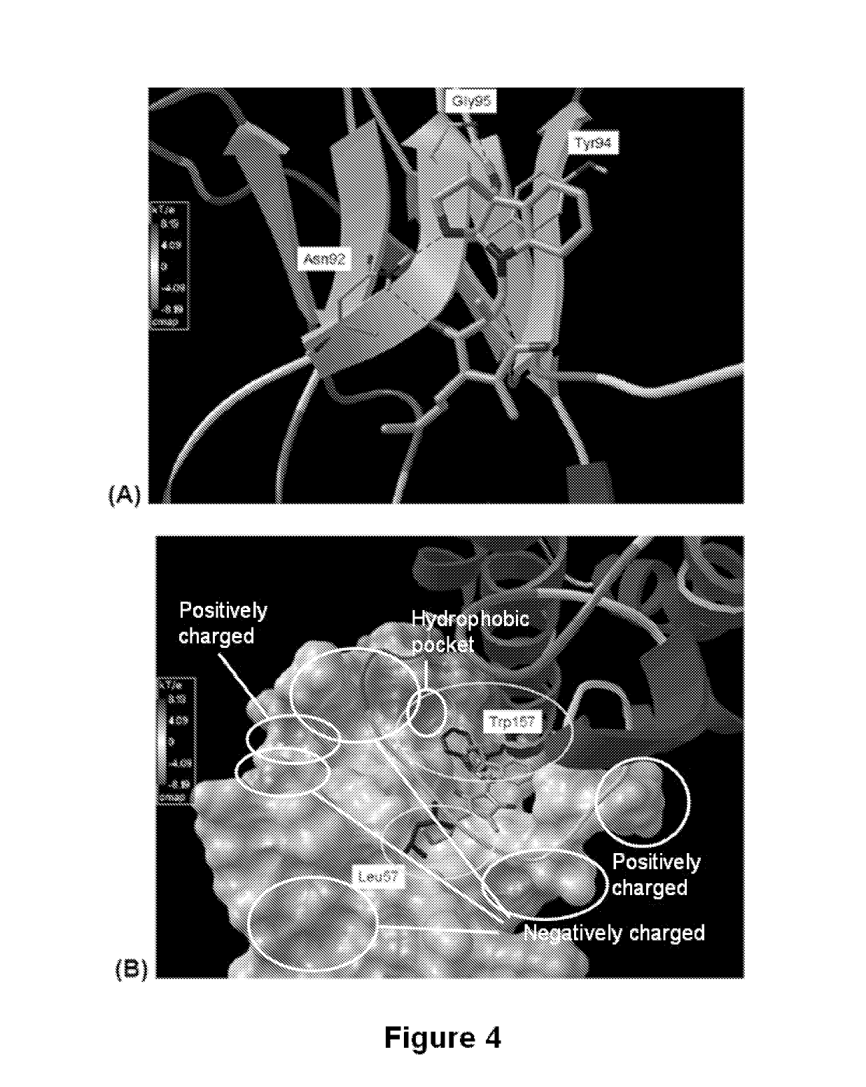

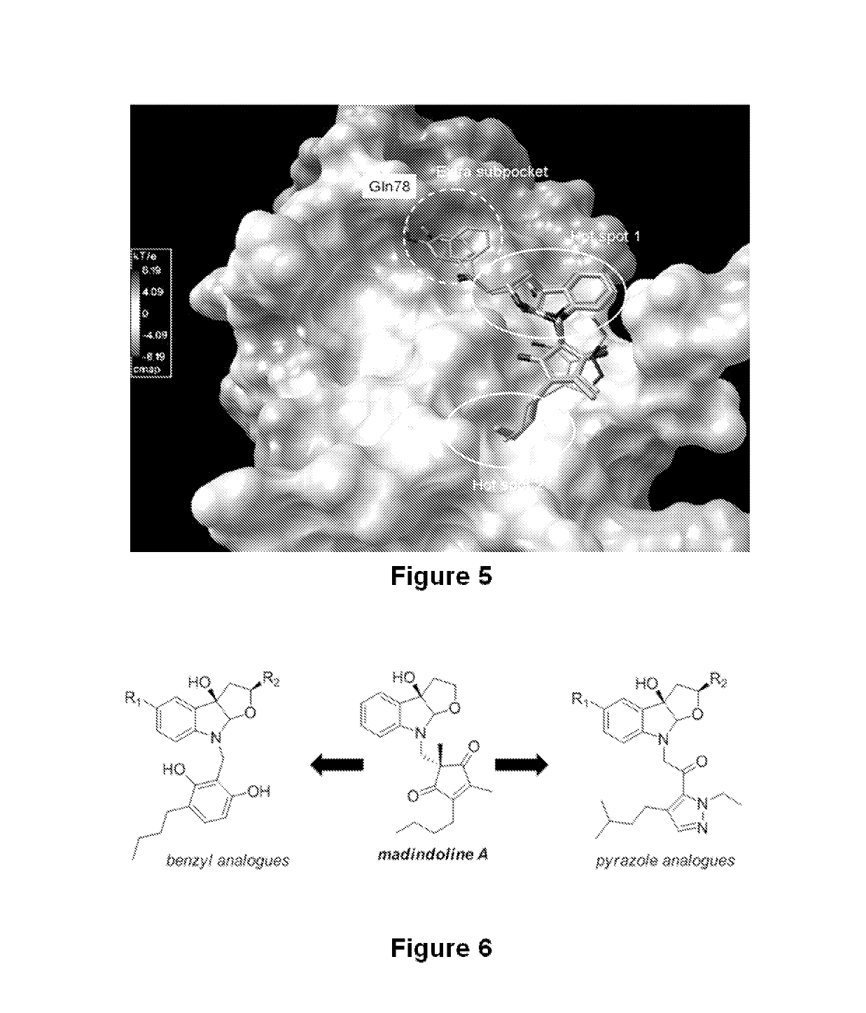

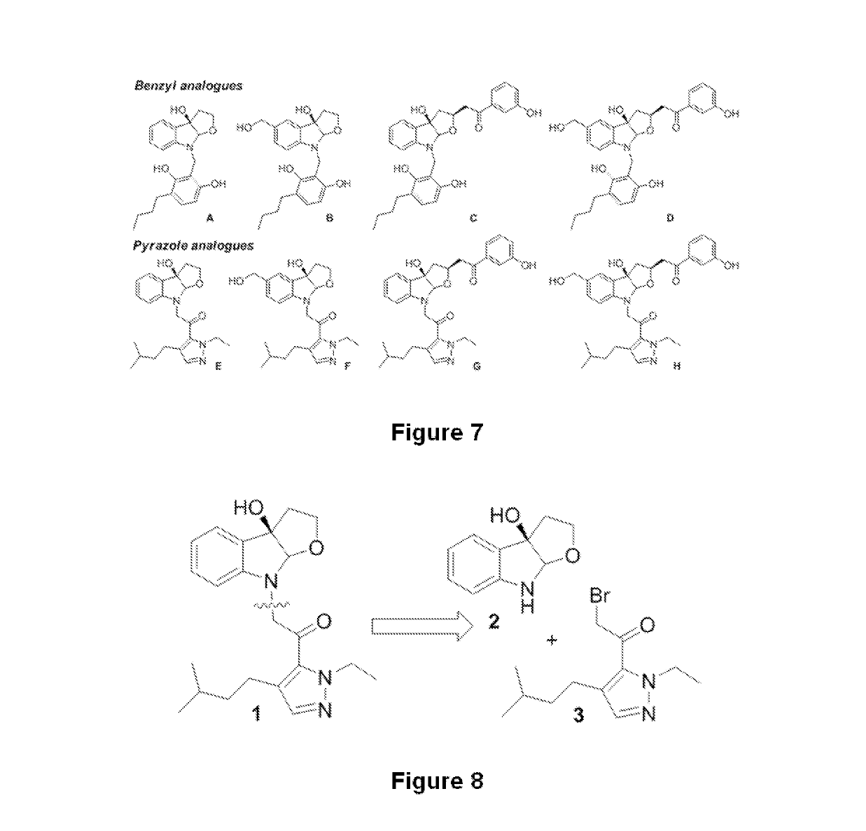

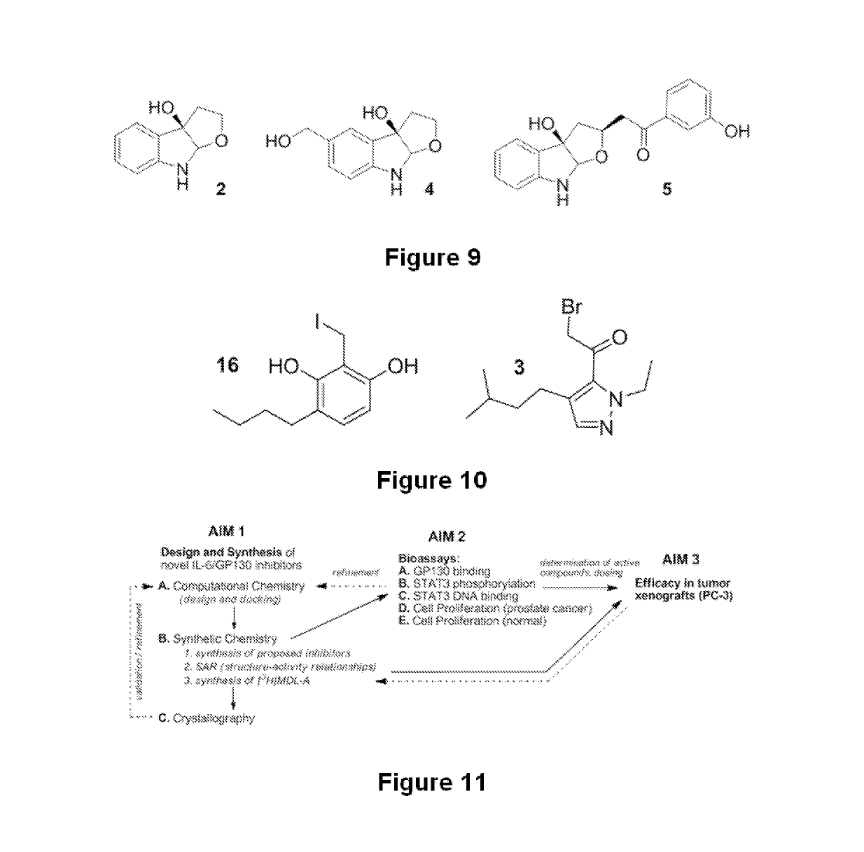

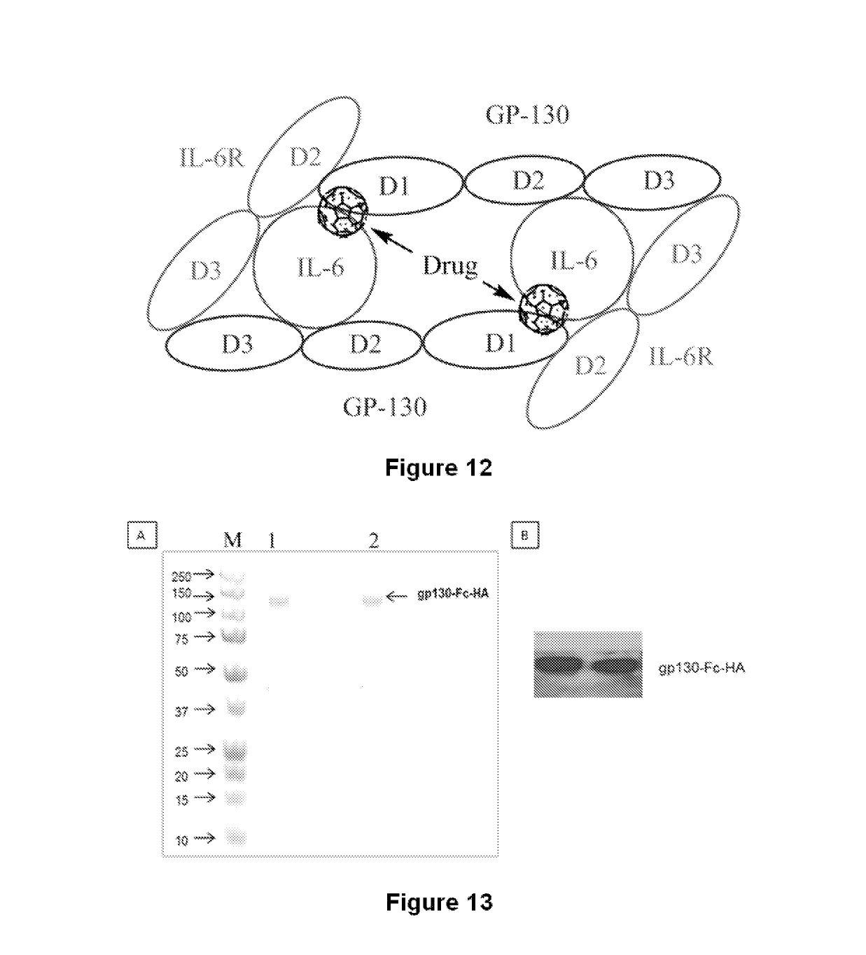

Analysis of the structure of madindoline A (MDL-A) and the computational model of its binding to the gp130 D1 extracellular domain has highlighted key structural features. To design novel derivatives with increased potency and selectivity, modifications through structure-based strategy can be used. For the start, two optimizations were addressed: a) improved synthetic efficiency. Fragment-based design methods were used to search for new fragments to replace the pentendione ring. With AlleGrow²⁵, hydroxylbenzyl and pyrazole rings were identified (see FIG. 6). b) improved potency/selectivity via targeting additional D1 domain binding subpocket. As shown in both FIGS. 4 and 5, additional fragments can be designed to bind to the extra subpocket. CombiGlide²⁶ was used to search a fragment library with 6000 fragments and came up with several options. FIGS. 5 and 7 show two possible choices. As shown in FIG. 5, the optimized analogues bind exactly as MDL-A with all its binding features preserved, except that the “southern” half of the molecules is easier to be synthesized and the extra subpocket is occupied plus additional hydrogen bond to Gln78 side chain. With hydroxybenzyl binding to the extra subpocket and the benzyl- and pyrazole-substituted “southern” half (see FIG. 7, compounds C and G), the binding free energies are −8.2 Kcal/mol and −8.6 Kcal/mol, respectively. These translate to 21- and 41-fold stronger affinity to GP130 compared to MDL-A, respectively. The analogues, therefore, can feature the addition of functional groups to the “northern” hydroxyfuroindoline portion of the molecule and/or replacement of the “southern” pentendione ring with benzyl or 5-acylpyrazole derivatives.

To evaluate the possible drug-likeness of these inhibitors, QikProp (Schrodinger LLC) can be used to compute fifty drug-likeness parameters. For MDL-A and re-designed analogues (FIG. 7), all analogues showed drug-like properties. For example, (1) composite log P values in range of 2.7 to 4.2; (2) predicted Caco-2 and MCDK cell permeability values are acceptable (Caco-2 range 207-476; MCDK range: 188-245); (3) predicted brain/blood partition coefficients are between −1.4 to −0.2; (4) predicted index of binding to human serum albumin ranges from −0.2 to 0.7, well within recommended range of −1.5-1.5; (5) predicted human oral absorption percentage is around 90%. Compared to existing drugs, they are 85% similar to Tretoquinol, Fexofenadine, Almitrine, Raloxifene, Cyclovalone and Eprozinol. As such, the compounds can possess high potentials to be developed into nontoxic, orally available drug without worrying about blood serum binding.

### E. BIOLOGICAL ASSAY METHODS

In order to confirm that the disclosed compounds can bind to the same receptor subunits as MDL-A, immunoblotting and autoradiography can be performed using a procedure similar to that reported by Hayashi and coworkers.¹⁹ An androgen-independent prostate cancer cell line (PC-3) can be used as the source of the receptor. IL-6 receptor and gp130 transducer have been reported to be expressed in all prostate cancer cell lines including PC-3.⁷ [³H]MDL-A, for the autoradiography study, can be synthesized according to published procedures.

In various aspects, the ability of IL-6 inhibitors to inhibit STAT3 phosphorylation at tyrosine residue 705 in PC-3 and DU-145 cancer cell lines expressing elevated levels of IL-6 and STAT3 phosphorylation can be examined using Western blot. Further, the inhibition of the stimulation of STAT3 phosphorylation by IL-6 in LNCap cells, which express very low levels of IL-6 can be performed. Since IL-6 stimulates STAT3 phosphorylation through JAK1 and JAK2, the possible inhibition of the stimulation of JAK1 and JAK2 phosphorylation by IL-6 inhibitors can also be investigated in PC-3 cells.

In a further aspect, The ability of IL-6 inhibitors to inhibit STAT3 phosphorylation at tyrosine residue 705 in MDA-MB-231, SUM-159, and SK-BR-3 human breast cancer cell lines expressing elevated levels of IL-6 and STAT3 phosphorylation can be examined. Further, the inhibition of the stimulation of STAT3 phosphorylation by IL-6 in MDA-MB-453 cells, which express very low levels of IL-6 and STAT3 phosphorylation can be examined. Since IL-6 stimulates STAT3 phosphorylation through JAK1 and JAK2, the possible inhibition of the stimulation of JAK1 and JAK2 phosphorylation by IL-6 inhibitors can also be investigated in MDA-MB-453 cells.

To confirm the inhibition of STAT3 activity, inhibition of STAT3 DNA binding activity by IL-6 inhibitors can be determined. PC-3 and DU-145 prostate cancer cell lines can be treated with different concentrations of IL-6 inhibitors for 12-24 hours using untreated and DMSO treated cells as negative controls. The nuclear extracts can be analyzed for STAT3 DNA binding activity using STAT3 Transcription Factor Assay kits (Upstate/Millipore Corporation and Active Motif Company). To determine the ability of IL-6 inhibitors to inhibit transcription of STAT3 downstream targets, Bcl-2, survivin, VEGF, cyclin D1, MMP-9, and Bcl-xL expression that are involved in cell cycle regulation, anti-apoptosis, and angiogenesis³³⁻³⁸ can be determined. The genes downstream of STAT3 can be examined by Western blots or Reverse Transcriptase-Polymerase Chain Reaction (RT-PCR) assay.

To confirm the inhibition of STAT3 activity, the inhibition of STAT3 DNA binding activity by IL-6 inhibitors can be examined. MDA-MB-231, SUM-159, and SK-BR-3 breast cancer cell lines can be treated with different concentrations of IL-6 inhibitors for 12-24 hours using untreated and DMSO treated as negative controls. The nuclear extracts can be analyzed for STAT3 DNA binding activity using STAT3 Transcription Factor Assay kits (Upstate/Millipore Corporation and Active Motif Company). To determine the ability of IL-6 inhibitors to inhibit transcription of STAT3 downstream targets, Bcl-2, survivin, VEGF, cyclin D1, MMP-9, and Bcl-xL expression that are involved in cell cycle regulation, anti-apoptosis, and angiogenesis²⁸⁻³³ can be examined. The STAT3's downstream genes can be examined by Western blots and/or Reverse Transcriptase-Polymerase Chain Reaction (RT-PCR) assay.

Inhibition of STAT3 phosphorylation and STAT3 DNA binding activity would block STAT3 oncogenic function in prostate cancer cells, which can result in the inhibition of cell proliferation and induction of apoptosis in androgen-independent cell lines. The cell viability and Half-Maximal inhibitory concentrations (IC₅₀) using the MTT assays can be determined. IC₅₀ can be determined using Sigma Plot 9.0 software (Systat Software Inc., San Jose, Calif.). Apoptosis can be assessed by flow cytometry using annexinV/propidium iodide staining and immunoblot analysis of relevant proteins (PARP and Caspase-3) following treatment of prostate cancer cells at various doses and time points of IL-6 inhibitors. The affect of the IL-6 inhibitors can be examined using LNCaP cells.

Inhibition of STAT3 phosphorylation and STAT3 DNA binding activity would block STAT3 oncogenic function in breast cancer cells, which can result in the inhibition of cell proliferation and induction of apoptosis. The cell viability can also be determined as well as Half-Maximal inhibitory concentrations (IC₅₀) using the MTT assays. IC₅₀ can be determined using Sigma Plot 9.0 software (Systat Software Inc., San Jose, Calif.). Apoptosis can be assessed by flow cytometry using annexinV/propidium iodide staining and immunoblot analysis of relevant proteins (PARP and Caspase-3) following treatment of breast cancer cells at various doses and time points of IL-6 inhibitors.

Toxicity of IL-6 inhibitors in normal human cells including normal human hepatocytes, skeletal muscle, bladder cells, and mammary epithelial cells (from Cambrex Corp.) without constitutively active STAT3 can be examined. Apoptosis can be assessed by flow cytometry using annexinV/propidium iodide staining and immunoblot analysis of relevant proteins (PARP and Caspase-3) following treatment with various doses and time points of IL-6 inhibitors. The possible induction of G1 cell cycle growth arrest can be assayed using BrdU labeling (Becton Dickinson) and flow cytometry. Cell viability can be examined and IC₅₀ can be determined using the MTT assays.

IL-6 inhibitors can exhibit potent activity in prostate cancer cell lines with elevated levels of IL-6 and STAT3 phosphorylation. Since many normal cells are not dependent upon IL-6/STAT3 pathway for survival, IL-6 inhibitors can have less or little toxicity to normal human cells.

The in vivo anti-tumor activity of the madindoline A (MDL-A) analogues can be examined in a PC-3 prostate tumor xenograft model which expresses elevated levels of IL-6 and GP130.

Prior to evaluating the in vivo efficacies of the optimal disclosed compounds, as well as the parental compound, in the PC-3 tumor xenograft model, the MTD of each agent can be determined in a 14-day, repeat-dose tolerance study in tumor-free athymic nude mice. These results can guide the dose-range selection for the subsequent efficacy studies. Briefly, five- to seven-week old male NCr athymic nu/nu mice can be randomly assigned to experimental groups representing different doses of three optimized MDL-A derivatives (Inhibitors A, B, and C), each to be administered at 0, 5, 10, 25, 50, and 100 mg/kg (6 mice/treatment group). Compounds can be administered by i.p. injection, once per day for 14 days. Body weights, to be measured twice per week, and direct observations of general health and behavior, to be recorded daily, can provide the primary indicators of tolerance to the drug. MTD will be the maximum dose tested that does not cause limiting toxicity as defined by a loss of ≧10% of starting body weight, inactivity/lethargy (≧2 days), inability or unwillingness to eat and/or drink (≧2 days), hunched posture, or other signs indicating moribundity. In addition, complete necropsies can be performed and tissues with grossly visible lesions can be fixed in formalin, paraffin-embedded and stained with hematoxylin-eosin for microscopic evaluation by a veterinary pathologist at the OSU Veterinary Biosciences Mouse Phenotyping Shared Resource.

Based on the results of the MTD determination, a dose range for each compound with three levels can be selected using the MTD as the highest dose level (H), along with intermediate (I) and low (L) dose levels. For example, subcutaneous PC-3 prostate cancer xenografts can be established in male NCr athymic nu/nu mice as described below. When tumor volumes reach approximately 100 mm³, mice can be randomly assigned to experimental groups for initiation of treatments. Treatments can include three optimized disclosed compounds, each administered at L, I and H dose levels, and docetaxel as positive control at 20 mg/kg. Control groups can receive vehicle only. Experimental compounds can be administered as described below with mice receiving one dose per day (i.p., 7 days/week) for the duration of the study. Body weights can be measured once per week and dosing volumes adjusted accordingly. Tumors can be measured weekly with microcalipers and tumor volumes calculated as the primary endpoint parameter of in vivo efficacy. When control tumors reach a mean volume of 1000 mm³, mice can be sacrificed. Tumors can be collected, weighed and portions fixed in formalin or snap-frozen in liquid nitrogen for subsequent assessment of biomarkers of drug activity by immunohistochemistry and immunoblotting as described below (Table 7). Three mice from each treatment group can be submitted to for evaluation of gross and histological pathology to identify potential treatment-related toxicities. Also, blood samples can be collected into heparinized tubes for evaluation of serum chemistry and hematological parameters. Treatment groups and numbers of animals/group are summarized in Table 1.

TABLE 1

| Control | | IL-6/G P130 inhibitor^{a} | | |
|---|---|---|---|---|
| | Docetaxel | (Inhibitors A, B, and C) | | |
| Vehicle | (20 mg/kg) | L | I | H |
| N = 10 | 10 | 10 | 10 | 10 |

^{a}Three different inhibitors will be assessed at three different dose levels based on pilot studies to determine MTDs for each compound. H, high dose equivalent to MTD; I, intermediate dose; L, low dose.
Total number of mice: 150 athymic nude mice (50 mice/inhibitor; 3 inhibitors)

In various aspects, the an in vivo assessment of a disclosed compound can be carried out as described here. Briefly, five- to seven week old male NCr athymic mice (NCI/Charles River Animal Facility, Frederick, Md.) can receive human prostate cancer xenografts by s.c. injection of PC-3 cells suspended in equal volumes of serum-free medium and Matrigel basement membrane matrix (1.0×10⁶ cells/0.1 ml/mouse). When tumor volumes reach approximately 100 mm³, mice can be randomly assigned to experimental groups for initiation of treatments as described above. Autoclaved water and manufacturer-sterilized food (Diet 7912, Harlan Teklad, Madison, Wis.) can be provided ad libitum. Animals can be group-housed in rooms maintained at 22±2° C. with 12 hrs of fluorescent lighting per 24-hour period.

IL-6/GP130 inhibitors can be prepared for i.p. administration in 50% DMSO (in physiological saline; 50 μL/mouse) as vehicle (50 μL/mouse). All agents can be administered to mice in single daily treatments by i.p. injection (1 cc syringe, 25 G needle) for the duration of the study at three dose levels based on pilot studies to determine the MTD of each compound. As a positive control, mice can also be treated with docetaxel i.p. every 3-4 days for a total of four treatments at 20 mg/kg. This dose level, dosing schedule, and route of administration follows that reported to be safe and effective in nude mouse xenograft models.⁵¹

Immunohistochemical and immunoblotting techniques will be employed to characterize in vivo intratumoral biomarkers of drug activity with the goal of providing correlations for the activities and mechanisms established in our in vitro studies that may elucidate in vivo mechanisms of action (Table 2).

TABLE 2

| | |
|---|---|
| Proliferation | PCNA (proliferating cell nuclear antigen) is a co-factor for |
| index | DNA-polymerase in both the S-phase and in DNA |
| | synthesis associated with DNA repair. |
| | Ki67 is expressed throughout the cell cycle (G1, S, G2, M) |
| | but not in G0. |
| Apoptosis | The ApopTag in situ detection kit will be used to identify |
| index | apoptotic cells, which uses the terminal |
| | deoxynucleotidyltransferase (TdT)-mediated TUNEL |
| | procedure. |

The effective number of athymic nude mice in each group can exclude those that do not survive to the designated study endpoint and those whose tissues are lost to evaluation for reasons of cannibalism or autolysis. Inter-group comparisons of mean tumor size can be made using one-way ANOVA, provided that assumptions are appropriately met, followed the Tukey's HSD method for pairwise comparisons. Tumor suppressive activity is defined as a statistically significant (P<0.05) reduction in tumor size in a drug-treated group as compared to the control group.

### F. PHARMACEUTICAL COMPOSITIONS

In one aspect, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in moulds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment conditions which require negative allosteric modulation of metabotropic glutamate receptor activity an appropriate dosage level will generally be about 0.01 to 500 mg per kg patient body weight per day and can be administered in single or multiple doses. Preferably, the dosage level will be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the from of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900 and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The present invention is further directed to a method for the manufacture of a medicament for modulating glutamate receptor activity (e.g., treatment of one or more neurological and/or psychiatric disorder associated with glutamate dysfunction) in mammals (e.g., humans) comprising combining one or more disclosed compounds, products, or compositions with a pharmaceutically acceptable carrier or diluent. Thus, in one aspect, the invention relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

### G. METHODS OF USING THE COMPOUNDS AND COMPOSITIONS

Early stage prostate cancer occurs as an androgen-dependent tumor, thus making androgen-deprivation a common therapeutic strategy even in advanced cases. Although tumors tend to respond well to this initial treatment, over time the prostate cancer typically recurs in a hormone-refractory or androgen-independent state. This progression is associated with a poor prognosis, ultimately leading to death. Although the precise mechanisms through which androgen-independence occur are not yet clear, non-androgen receptor (AR) dependent growth factors are believed to play a key role in this type of prostate cancer proliferation.¹

TABLE 3

| Cell Line | Cell Type | IL-6 level^{a} |
|---|---|---|
| PC3 | androgen-independent | 1,965 |
| DU145 | androgen-independent | 453 |
| TSU | androgen-independent | 987 |
| LNCaP | androgen-dependent | undetectable |

One of the key growth factors in prostate cancer is the multifunctional cytokine interleukin-6 (IL-6), which plays a key role in immune response, cell survival, apoptosis, and proliferation.² The expression of IL-6 and its receptor [both interleukin-6 receptor (IL-6R, also called GP80) and glycoprotein 130 (GP130)] has been widely observed in both benign and malignant prostate cell tissues,³⁻⁶ although levels of both the cytokine and receptor increase during carcinogensesis. It was also determined that IL-6 levels in culture supernatants are much higher in androgen-independent prostate cancer cells (e.g., PC-3, DU-145, and TSU cells, Table 3 which shows the levels of IL-6 in conditioned medium of prostate cancer cell lines, see Gao and co-works, Ref. No. 6; the data are shown in pg IL6 per 10⁶ cells per 24 hr) and that IL-6 is linked to increased cell proliferation.⁷ Based on these observations IL-6 is frequently associated with a poor prognosis in prostate cancer, despite the fact that IL-6 plays a key role in cell proliferation and differentiation in all prostate cells.⁸ AR-positive LNCaP cells have been reported to be IL-6 negative, although both IL-6R and GP130 are expressed.⁷ This has led to numerous studies which indicate either growth stimulation or growth inhibition in LNCap cells upon treatment with IL-6. Gao and coworkers, however, have recently explained these apparently conflicting results by demonstrating that IL-6 transitions from a paracrine growth inhibitor to an autocrine growth stimulator in LNCaP cells.⁹ These results act to further reinforce the role of IL-6 in prostate-cancer progression.

Interleukin-6 (IL-6) is a key signaling molecule in prostate cancer cells. It is responsible for many cellular responses in both cancer and normal cells, including immune response, cell survival, cell death, and proliferation. IL-6 may also play a key role in the progression of prostate cancer from an androgen-dependent to androgen-independent cancer (typically associated with a poor prognosis among prostate cancer patients). This change to an androgen-independent cancer is associated with significantly increased levels of IL-6, which is believed to affect the subsequent proliferation and metastasis of the tumor cells by initiating a complex series of molecular signal pathways, specifically the IL-6/JAK/STAT pathway. Therefore, a new strategy to combat androgen-independent prostate cancers by disrupting the initiation of the IL-6 signaling using small synthetic molecules using the natural product madindoline A as a starting point is described herein. As described herein, Madindoline A (MDL-A) is known to interact with the IL-6 receptor on the surface of the cell and prevent this signaling event. The disclosed compounds can provide more potent and selective derivatives which can be useful therapeutic agents for the treatment of prostate cancer. Disclosed herein are compounds that bind to IL-6 and/or gp130 and inhibit STAT3 phosphorylation.

The disclosed compounds can be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

The pharmaceutical compositions and methods of the present invention can further comprise other therapeutically active compounds as noted herein which are usually applied in the treatment of the above mentioned pathological conditions.

1. Treatment Methods

The compounds disclosed herein are useful for treating, preventing, ameliorating, controlling or reducing the risk of a variety of disorders wherein the patient or subject would benefit from inhibition or negative modulation of a IL6-mediated STAT3 phosphorylation activity. In one aspect, a treatment can include selective inhibition of IL6 mediated signaling pathway to an extent effective to STAT3 phosphorylation. Thus, a disorder can be associated with STAT3 activity, e.g. an immune disorder or a disorder of uncontrolled cellular proliferation. In one aspect, provided is a method of treating or preventing a disorder in a subject comprising the step of administering to the subject at least one disclosed compound; at least one disclosed pharmaceutical composition; and/or at least one disclosed product in a dosage and amount effective to treat the disorder in the subject.

Also provided is a method for the treatment of one or more disorders, for which inhibition of IL6-mediated STAT3 phosphorylation activity is predicted to be beneficial, in a subject comprising the step of administering to the subject at least one disclosed compound; at least one disclosed pharmaceutical composition; and/or at least one disclosed product in a dosage and amount effective to treat the disorder in the subject.

Also provided is a method for the treatment of one or more disorders, for which inhibition of homodimerization of IL6-IL6R-GP130 heterotrimer activity is predicted to be beneficial, in a subject comprising the step of administering to the subject at least one disclosed compound; at least one disclosed pharmaceutical composition; and/or at least one disclosed product in a dosage and amount effective to treat the disorder in the subject.

Also provided is a method for the treatment of one or more disorders, for which modulation of a Jak2/STAT3 signaling pathway dysfunction is predicted to be beneficial, in a subject comprising the step of administering to the subject at least one disclosed compound; at least one disclosed pharmaceutical composition; and/or at least one disclosed product in a dosage and amount effective to treat the disorder in the subject.

In one aspect, provided is a method for treating a disorder of uncontrolled cellular proliferation, comprising: administering to a subject at least one disclosed compound; at least one disclosed pharmaceutical composition; and/or at least one disclosed product in a dosage and amount effective to treat the disorder in the subject. In a further aspect, provided is a method for treating or preventing an immune disorder, comprising: administering to a subject at least one disclosed compound; at least one disclosed pharmaceutical composition; and/or at least one disclosed product in a dosage and amount effective to treat the disorder in the subject. Also provided is a method for the treatment of a disorder in a mammal comprising the step of administering to the mammal at least one disclosed compound, composition, or medicament.

The invention is directed at the use of described chemical compositions to treat diseases or disorders in patients (preferably human) wherein inhibition of IL6-mediated STAT3 phosphorylation activity would be predicted to have a therapeutic effect, such as disorders of uncontrolled cellular proliferation (e.g. cancers) and immune disorders such as inflammatory bowel disease or other chronic inflammatory diseases involving an IL6 dysfunction, by administering one or more disclosed compounds or products.

The compounds disclosed herein are useful for treating, preventing, ameliorating, controlling or reducing the risk of a variety of disorders of uncontrolled cellular proliferation. In one aspect, the disorder of uncontrolled cellular proliferation is associated with STAT3 dysfunction. In a further aspect, the histone demethylase dysfunction is disregulation of the homodimerization of IL6-IL6R-GP130 heterotrimer. In a still further aspect, the histone demethylase dysfunction is disregulation of the IL6-mediated STAT3 phosphorylation activity. In an even further aspect, the histone demethylase dysfunction is disregulation of the a Jak2/STAT3 signaling pathway.

Also provided is a method of use of a disclosed compound, composition, or medicament. In one aspect, the method of use is directed to the treatment of a disorder. In a further aspect, the disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

Examples of disorders associated with a histone demethylase dysfunction include a disorder of uncontrolled cellular proliferation. In a yet further aspect, the disorder of uncontrolled cellular proliferation is cancer. In a yet further aspect, the cancer is a leukemia. In an even further aspect, the cancer is a sarcoma. In a still further aspect, the cancer is a solid tumor. In a yet further aspect, the cancer is a lymphoma.

It is understood that cancer refer to or describe the physiological condition in mammals that is typically characterized by unregulated cell growth. The cancer may be multi-drug resistant (MDR) or drug-sensitive. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

In various aspects, further examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma. In a still further aspect, the cancer is selected from chronic lymphocytic leukemia, small lymphocytic lymphoma, B-cell non-Hodgkin lymphoma, and large B-cell lymphoma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In one aspect, the cancer can be a cancer selected from cancers of the blood, brain, genitourinary tract, gastrointestinal tract, colon, rectum, breast, kidney, lymphatic system, stomach, lung, pancreas, and skin. In a further aspect, the cancer is selected from prostate cancer, glioblastoma multiforme, endometrial cancer, breast cancer, and colon cancer. In a further aspect, the cancer is selected from a cancer of the breast, ovary, prostate, head, neck, and kidney. In a still further aspect, the cancer is selected from cancers of the blood, brain, genitourinary tract, gastrointestinal tract, colon, rectum, breast, livery, kidney, lymphatic system, stomach, lung, pancreas, and skin. In a yet further aspect, the cancer is selected from a cancer of the lung and liver. In an even further aspect, the cancer is selected from a cancer of the breast, ovary, testes and prostate In a still further aspect, the cancer is a cancer of the breast. In a yet further aspect, the cancer is a cancer of the ovary. In an even further aspect, the cancer is a cancer of the prostate. In a still further aspect, the cancer is a cancer of the testes.

In various aspects, disorders associated with a histone demethylase dysfunction include neurodegenerative disorders. In a further aspect, the neurodegenerative disease is selected from Alzheimer's disease, Parkinson's disease, and Huntington's disease.

The compounds are further useful in a method for the prevention, treatment, control, amelioration, or reduction of risk of the diseases, disorders and conditions noted herein. The compounds are further useful in a method for the prevention, treatment, control, amelioration, or reduction of risk of the aforementioned diseases, disorders and conditions in combination with other agents.

The present invention is further directed to administration of a inhibitor of homodimerization of IL6-IL6R-GP130 heterotrimer for improving treatment outcomes in the context of disorders of uncontrolled cellular proliferation, including cancer. That is, in one aspect, the invention relates to a cotherapeutic method comprising the step of administering to a mammal an effective amount and dosage of at least one compound of the invention in connection with cancer therapy.

The present invention is further directed to administration of a inhibitor of IL6-mediated STAT3 phosphorylation activity for improving treatment outcomes in the context of disorders of uncontrolled cellular proliferation, including cancer. That is, in one aspect, the invention relates to a cotherapeutic method comprising the step of administering to a mammal an effective amount and dosage of at least one compound of the invention in connection with cancer therapy.

In a further aspect, administration improves treatment outcomes in the context of cancer therapy. Administration in connection with cancer therapy can be continuous or intermittent. Administration need not be simultaneous with therapy and can be before, during, and/or after therapy. For example, cancer therapy can be provided within 1, 2, 3, 4, 5, 6, 7 days before or after administration of the compound. As a further example, cancer therapy can be provided within 1, 2, 3, or 4 weeks before or after administration of the compound. As a still further example, cognitive or behavioral therapy can be provided before or after administration within a period of time of 1, 2, 3, 4, 5, 6, 7, 8, 9, or 10 half-lives of the administered compound.

In one aspect, the disclosed compounds can be used in combination with one or more other drugs in the treatment, prevention, control, amelioration, or reduction of risk of diseases or conditions for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly.

Accordingly, the pharmaceutical compositions include those that contain one or more other active ingredients, in addition to a compound of the present invention.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1; 1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

Accordingly, the subject compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

In one aspect, the compound can be employed in combination with anti-cancer therapeutic agents. In a further aspect, the anti-cancer therapeutic agent is selected from 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, DepoCyt™, Dexamethasone, Dexamethasone Acetate Dexamethasone Sodium Phosphate Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard ™, Ellence™, Eloxatin™, Elspar®, Emcyt®, Epirubicin, Epoetin Alfa, Erbitux, Erlotinib, Erwinia L-asparaginase, Estramustine, EthyolEtopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, GemzarGleevec™, Gliadel® Wafer, GM-CSF, Goserelin, Granulocyte—Colony Stimulating Factor, Granulocyte Macrophage Colony Stimulating Factor, Halotestin®, Herceptin®, Hexadrol, Hexylen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetanldamycin®, Idarubicin, Ifex®, IFN-alphafosfamide, IL-11 IL-2Imatinib mesylate, Imidazole Carboxamide Interferon alfa, Interferon Alfa-2b (PEG Conjugate), Interleukin-2, Interleukin-11, Intron A® (interferon alfa-2b)Iressa®, Irinotecan, Isotretinoin, Ixabepilone, Ixempra™, K, Kidrolase (t), L, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred ®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine Mutamycin ®, Myleran®, Mylocel™, Mylotarg®, N, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, O, Octreotide, Octreotide acetate, Ofatumumab, Oncospar®, Oncovin®, Ontak®, Onxal™, Oprelvekin, Orapred®, Orasone ®, Oxaliplatin, P, Paclitaxel, Paclitaxel Protein-bound, Pamidronate, Panitumumab, Panretin ®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a)Romiplostim, Rubex®, Rubidomycin hydrochloride, S, Sandostatin®, Sandostatin LAR ®, Sargramostim, Solu-Cortef®, Solu-Medrol®, Sorafenib, SPRYCEL™, STI-571, Streptozocin, SU11248, Sunitinib, Sutent®, T, Tamoxifen, Tarceva®, Targretin®, Tasigna ®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, V, VCR, Vectibix™, Velban®, Velcade®, VePesid®, Vesanoid®, Viadur ™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, X, Xeloda®, Z, Zanosar®, Zevalin™, Zinecard®, Zoladex®, Zoledronic acid, Zolinza, Zometa®.

In another aspect, the subject compounds can be administered in combination with 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, DepoCyt™, Dexamethasone, Dexamethasone Acetate Dexamethasone Sodium Phosphate Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard ™, Ellence™, Eloxatin™, Elspar®, Emcyt®, Epirubicin, Epoetin Alfa, Erbitux, Erlotinib, Erwinia L-asparaginase, Estramustine, EthyolEtopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, GemzarGleevec™, Gliadel® Wafer, GM-CSF, Goserelin, Granulocyte—Colony Stimulating Factor, Granulocyte Macrophage Colony Stimulating Factor, Halotestin®, Herceptin®, Hexadrol, Hexylen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetanldamycin®, Idarubicin, Ifex®, IFN-alphafosfamide, IL-11 IL-2Imatinib mesylate, Imidazole Carboxamide Interferon alfa, Interferon Alfa-2b (PEG Conjugate), Interleukin-2, Interleukin-11, Intron A® (interferon alfa-2b)Iressa®, Irinotecan, Isotretinoin, Ixabepilone, Ixempra™, K, Kidrolase (t), L, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred ®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine Mutamycin ®, Myleran®, Mylocel™, Mylotarg®, N, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, 0, Octreotide, Octreotide acetate, Ofatumumab, Oncospar®, Oncovin®, Ontak®, Onxal™, Oprelvekin, Orapred®, Orasone ®, Oxaliplatin, P, Paclitaxel, Paclitaxel Protein-bound, Pamidronate, Panitumumab, Panretin ®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a)Romiplostim, Rubex®, Rubidomycin hydrochloride, S, Sandostatin®, Sandostatin LAR ®, Sargramostim, Solu-Cortef®, Solu-Medrol®, Sorafenib, SPRYCEL™, STI-571, Streptozocin, SU11248, Sunitinib, Sutent®, T, Tamoxifen, Tarceva®, Targretin®, Tasigna ®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, V, VCR, Vectibix™, Velban®, Velcade®, VePesid®, Vesanoid®, Viadur ™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, X, Xeloda®, Z, Zanosar®, Zevalin™, Zinecard®, Zoladex®, Zoledronic acid, Zolinza, Zometa®

In another aspect, the subject compound can be used in combination with 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, DepoCyt™, Dexamethasone, Dexamethasone Acetate Dexamethasone Sodium Phosphate Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard ™, Ellence™, Eloxatin™, Elspar®, Emcyt®, Epirubicin, Epoetin Alfa, Erbitux, Erlotinib, Erwinia L-asparaginase, Estramustine, EthyolEtopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, GemzarGleevec™, Gliadel® Wafer, GM-CSF, Goserelin, Granulocyte—Colony Stimulating Factor, Granulocyte Macrophage Colony Stimulating Factor, Halotestin®, Herceptin®, Hexadrol, Hexylen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetanldamycin®, Idarubicin, Ifex®, IFN-alphafosfamide, IL-11IL-2Imatinib mesylate, Imidazole Carboxamide Interferon alfa, Interferon Alfa-2b (PEG Conjugate), Interleukin-2, Interleukin-11, Intron A® (interferon alfa-2b)Iressa®, Irinotecan, Isotretinoin, Ixabepilone, Ixempra™, K, Kidrolase (t), L, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred ®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine Mutamycin ®, Myleran®, Mylocel™, Mylotarg®, N, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, 0, Octreotide, Octreotide acetate, Ofatumumab, Oncospar®, Oncovin®, Ontak®, Onxal™, Oprelvekin, Orapred®, Orasone ®, Oxaliplatin, P, Paclitaxel, Paclitaxel Protein-bound, Pamidronate, Panitumumab, Panretin ®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a)Romiplostim, Rubex®, Rubidomycin hydrochloride, S, Sandostatin®, Sandostatin LAR ®, Sargramostim, Solu-Cortef®, Solu-Medrol®, Sorafenib, SPRYCEL™, STI-571, Streptozocin, SU11248, Sunitinib, Sutent®, T, Tamoxifen, Tarceva®, Targretin®, Tasigna ®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, V, VCR, Vectibix™, Velban®, Velcade®, VePesid®, Vesanoid®, Viadur ™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, X, Xeloda®, Z, Zanosar®, Zevalin™, Zinecard®, Zoladex®, Zoledronic acid, Zolinza, Zometa®

In the treatment of conditions which require inhibition or negative modulation of IL6-mediated STAT3 phosphorylation activity, an appropriate dosage level will generally be about 0.01 to 1000 mg per kg patient body weight per day which can be administered in single or multiple doses. Preferably, the dosage level will be about 0.1 to about 250 mg/kg per day; more preferably about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Thus, in one aspect, the invention relates to methods for inhibiting or negatively modulating IL6-mediated STAT3 phosphorylation activity in at least one cell, comprising the step of contacting the at least one cell with at least one compound of the invention, in an amount effective to inhibit or negatively modulate IL6-mediated STAT3 phosphorylation activity in the at least one cell. In a further aspect, the cell is mammalian, for example human. In a further aspect, the cell has been isolated from a subject prior to the contacting step. In a further aspect, contacting is via administration to a subject.

a. Treatment of a Disorder Associated IL6 Dysfunction

In one aspect, the invention relates to a method for the treatment of a disorder associated with an IL6 dysfunction in a mammal comprising the step of administering to the mammal a therapeutically effective amount of at least one compound having a structure represented by a formula:

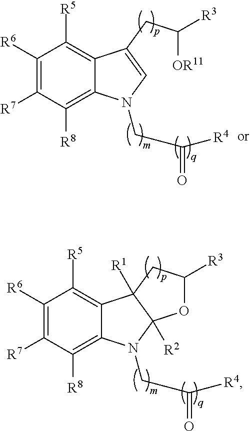

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

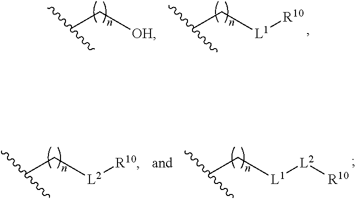

wherein L¹ is —O— or —NH—; wherein L² is —CH₂ or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C₁-C₆ haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

In a further aspect, the compound administered is a disclosed compound or a product of a disclosed method of making a compound.

In one aspect, the mammal is a human. In a further aspect, the mammal has been diagnosed with a need for treatment of the disorder prior to the administering step. In a further aspect, the method further comprises the step of identifying a mammal in need of treatment of the disorder.

In a further aspect, the IL6 dysfunction is associated with activation of the Jak2/STAT3 pathway.

In a further aspect, the disorder is an inflammatory disease or an autoimmune disease. In a still further aspect, the disorder is an inflammatory disease. In a yet further aspect, the inflammatory disease is an acute inflammatory disease. In an even further aspect, the inflammatory disease is a chronic inflammatory disease. In a still further aspect, the inflammatory disease is selected from psoriasis, Alzheimer's disease, rheumatoid arthritis, systemic onset juvenile idiopathic arthritis, hypergammaglobulinemia, Crohn's disease, ulcerative colitis, systemic lupus erythematosus (SLE), multiple sclerosis, Castleman's disease, IgM gammopathy, cardiac myxoma, asthma, allergic asthma, autoimmune insulin-dependent diabetes mellitus, chronic obstructive pulmonary disease, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

In a further aspect, the disorder is an autoimmune disease. In a still further aspect, the autoimmune disorder is selected from alopecia greata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigoid, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjögren's syndrome, stiff-man syndrome, systemic lupus erythematosus, Takayasu arteritis, temporal arteritis/giant cell arteritis, ulcerative colitis, uveitis, vitiligo and Wegener's granulomatosis.

In a further aspect, the disorder is selected from sepsis, bone resorption, osteoporosis, and cachexia.

In a further aspect, the disorder is cancer. In a still further aspects, the disorder is a cancer selected from multiple myeloma disease (MM), renal cell carcinoma (RCC), plasma cell leukaemia, lymphoma, B-lymphoproliferative disorder (BLPD), renal cell carcinoma, breast cancer, prostate cancer, pancreatic cancer, lung cancer, gastric cancer, and colorectal cancer. In a yet further aspect, the cancer is selected from breast cancer, prostate cancer, pancreatic cancer, lung cancer, gastric cancer, and colorectal cancer. In an even further aspect, the cancer is prostate cancer. In a still further aspect, the cancer is breast cancer. In a yet further aspect, the cancer is pancreatic cancer. In an even further aspect, the cancer is lung cancer. In a still further aspect, the cancer is gastric cancer. In a yet further aspect, the cancer is colorectal cancer.

b. Treatment of a Disorder of Uncontrolled Cellular Proliferation

In one aspect, the invention relates to a method for the treatment of a disorder of uncontrolled cellular proliferation associated with STAT3 dysfunction in a mammal comprising the step of administering to the mammal a therapeutically effective amount of at least one compound having a structure represented by a formula:

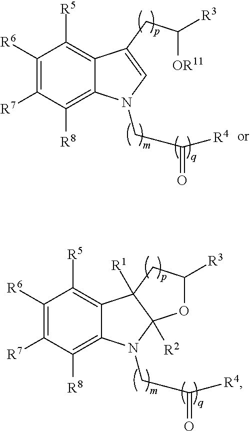

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

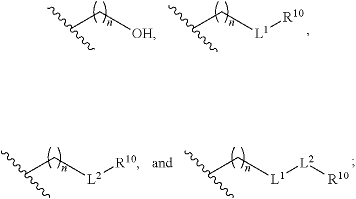

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

In a further aspect, the compound administered is a disclosed compound or a product of a disclosed method of making a compound.

In a further aspect, the mammal is human. In a still further aspect, the mammal has been diagnosed with a need for treatment of the disorder prior to the administering step. In a yet further aspect, the method further comprises the step of identifying a mammal in need of treatment of the disorder.

In a further aspect, the disorder of uncontrolled cellular proliferation is associated with a dysfunction of activation of the Jak2/STAT3 pathway.

In a further aspect, the disorder of uncontrolled cellular proliferation is cancer. In a further aspect, the disorder is cancer. In a still further aspects, the disorder is a cancer selected from multiple myeloma disease (MM), renal cell carcinoma (RCC), plasma cell leukaemia, lymphoma, B-lymphoproliferative disorder (BLPD), renal cell carcinoma, breast cancer, prostate cancer, pancreatic cancer, lung cancer, gastric cancer, and colorectal cancer. In a yet further aspect, the cancer is selected from breast cancer, prostate cancer, pancreatic cancer, lung cancer, gastric cancer, and colorectal cancer. In an even further aspect, the cancer is prostate cancer. In a still further aspect, the cancer is breast cancer. In a yet further aspect, the cancer is pancreatic cancer. In an even further aspect, the cancer is lung cancer. In a still further aspect, the cancer is gastric cancer. In a yet further aspect, the cancer is colorectal cancer.

c. Treatment of an Immune Disorder

In one aspect, the invention relates to a method for the treatment of an immune disorder associated with a STAT3 dysfunction in a mammal comprising the step of administering to the mammal a therapeutically effective amount of at least one compound having a structure represented by a formula:

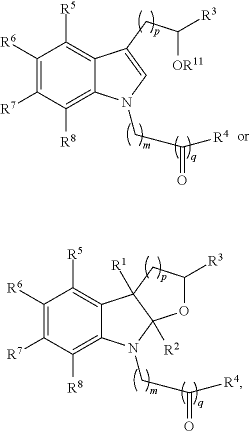

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

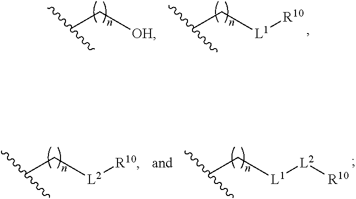

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

In a further aspect, the compound administered is a disclosed compound or a product of a disclosed method of making a compound.

In a further aspect, the mammal is human. In a still further aspect, the mammal has been diagnosed with a need for treatment of the disorder prior to the administering step. In a yet further aspect, the method further comprises the step of identifying a mammal in need of treatment of the disorder.

In a further aspect, the disorder of uncontrolled cellular proliferation is associated with a dysfunction of activation of the Jak2/STAT3 pathway.

In a further aspect, the disorder is an inflammatory disease or an autoimmune disease. In a still further aspect, the disorder is an inflammatory disease. In a yet further aspect, the inflammatory disease is an acute inflammatory disease. In an even further aspect, the inflammatory disease is a chronic inflammatory disease. In a still further aspect, the inflammatory disease is selected from psoriasis, Alzheimer's disease, rheumatoid arthritis, systemic onset juvenile idiopathic arthritis, hypergammaglobulinemia, Crohn's disease, ulcerative colitis, systemic lupus erythematosus (SLE), multiple sclerosis, Castleman's disease, IgM gammopathy, cardiac myxoma, asthma, allergic asthma, autoimmune insulin-dependent diabetes mellitus, chronic obstructive pulmonary disease, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

In a further aspect, the disorder is an autoimmune disease. In a still further aspect, the autoimmune disorder is selected from alopecia greata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigoid, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjögren's syndrome, stiff-man syndrome, systemic lupus erythematosus, Takayasu arteritis, temporal arteritis/giant cell arteritis, ulcerative colitis, uveitis, vitiligo and Wegener's granulomatosis.

In a further aspect, the disorder is inflammatory bowel disease.

d. Inhibition of IL6 Mediated Activation of the Jak2/Stat3 Pathway

In one aspect, the invention relates to a method for inhibition of IL6 mediated activation of the Jak2/STAT3 pathway in a mammal comprising the step of administering to the mammal a therapeutically effective amount of at least one compound having a structure represented by a formula:

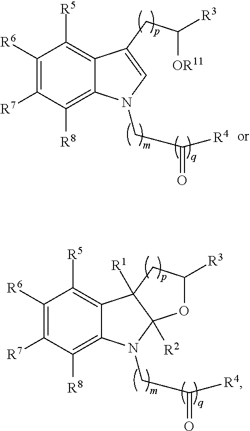

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

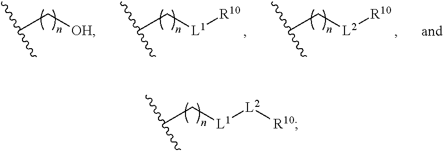

wherein L¹ is —O— or —NH—; wherein L² is —CH₂ or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, (C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

In a further aspect, the compound administered is a disclosed compound or a product of a disclosed method of making a compound.

In a further aspect, the mammal is human. In a still further aspect, the mammal has been diagnosed with a need for inhibition of Jak2/STAT3 pathway prior to the administering step. In a yet further aspect, the method further comprises the step of identifying a mammal in need of inhibition of Jak2/STAT3 pathway.

In a further aspect, the disorder of uncontrolled cellular proliferation is associated with a dysfunction of activation of the Jak2/STAT3 pathway.

In a further aspect, the Jak2/STAT3 pathway is activated by homodimerization of a IL6-IL6R-gp130 heterotrimer.

e. Inhibition of Homodimerization of a IL6-IL6R-gp130 Heterotrimer

In one aspect, the invention relates to a method for inhibition of homodimerization of a IL6-IL6R-gp130 heterotrimer in a mammal comprising the step of administering to the mammal a therapeutically effective amount of at least one compound having a structure represented by a formula:

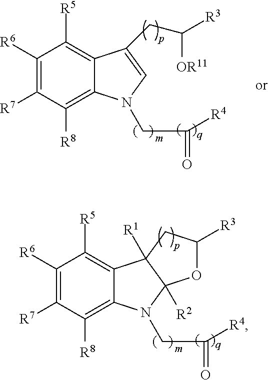

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

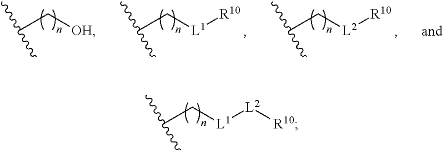

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

In a further aspect, the compound administered is a disclosed compound or a product of a disclosed method of making a compound.

In a further aspect, the mammal is human. In a still further aspect, the mammal has been diagnosed with a need for inhibition of homodimerization of a IL6-IL6R-gp130 heterotrimer prior to the administering step. In a yet further aspect, the method further comprises the step of identifying a mammal in need of inhibition of homodimerization of a IL6-IL6R-gp130 heterotrimer.

f. Inhibition of IL6 Mediated Activation of the Jak2/Stat3 Pathway in at Least One Cell

In one aspect, the invention relates to a method for inhibition of IL6 mediated activation of the Jak2/STAT3 pathway in at least one cell, comprising the step of contacting the at least one cell with an effective amount of at least one compound having a structure represented by a formula:

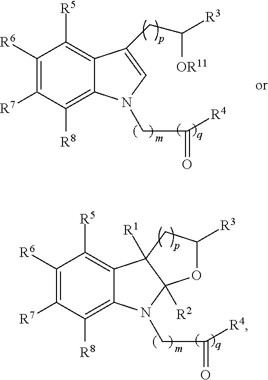

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen

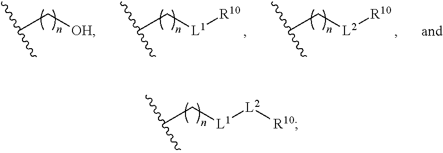

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, OH, NO₂, NR²⁵R²⁶, C1-C6 alkyl, C1-C6 haloalkyl, (C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

In a further aspect, the compound administered is a disclosed compound or a product of a disclosed method of making a compound.

In one aspect, the cell is mammalian. In a further aspect, the cell is human. In a further aspect, the cell has been isolated from a mammal prior to the contacting step.

In a further aspect, contacting is via administration to a mammal. In a further aspect, the mammal has been diagnosed with a need for inhibiting activation of the Jak2/STAT3 pathway activity prior to the administering step. In a further aspect, the mammal has been diagnosed with a need for treatment of a disorder related to activation of the Jak2/STAT3 pathway prior to the administering step.

g. Inhibition of Homodimerization of a IL6-IL6R-gp130 Heterotrimer in at Least One Cell

In one aspect, the invention relates to a method for inhibition homodimerization of a IL6-IL6R-gp130 heterotrimer in at least one cell, comprising the step of contacting the at least one cell with an effective amount of at least one compound having a structure represented by a formula:

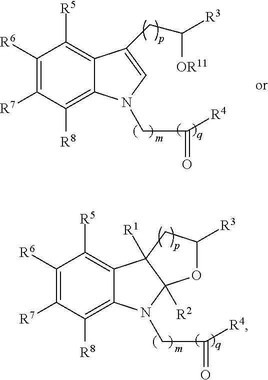

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and OH; wherein R³ is selected from: hydrogen,

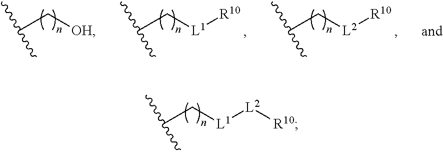

wherein L¹ is —O— or —NH—; wherein L² is —CH₂ or —(C═O); and wherein R¹⁰ is selected from hydrogen, C1-C8 alkyl, C1-C8 alkoxy, NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C₁-C₆ alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof.

In a further aspect, the compound administered is a disclosed compound or a product of a disclosed method of making a compound.

In one aspect, the cell is mammalian. In a further aspect, the cell is human. In a further aspect, the cell has been isolated from a mammal prior to the contacting step.

In a further aspect, contacting is via administration to a mammal. In a further aspect, the mammal has been diagnosed with a need for inhibiting homodimerization of a IL6-IL6R-gp130 heterotrimer prior to the administering step. In a further aspect, the mammal has been diagnosed with a need for treatment of a disorder related to homodimerization of a IL6-IL6R-gp130 heterotrimer prior to the administering step.

2. Manufacture of a Medicament

In one aspect, the invention relates to a method for the manufacture of a medicament for inhibition of IL6-mediated STAT3 phosphorylation activity in a mammal comprising combining a therapeutically effective amount of a disclosed compound or product of a disclosed method with a pharmaceutically acceptable carrier or diluent.

In various aspects, the invention relates to a method for the manufacture of a medicament for inhibition of homodimerization of IL6-IL6R-GP130 heterotrimer activity in a mammal comprising combining a therapeutically effective amount of a disclosed compound or product of a disclosed method with a pharmaceutically acceptable carrier or diluent.

In various aspects, the invention relates to a method for the manufacture of a medicament for treating a cancer comprising combining a therapeutically effective amount of a disclosed compound or product of a disclosed method with a pharmaceutically acceptable carrier or diluent.

In various aspects, the invention relates to a method for the manufacture of a medicament for treating an immune disorder, including an inflammatory disease or an autoimmune disorder, comprising combining a therapeutically effective amount of a disclosed compound or product of a disclosed method with a pharmaceutically acceptable carrier or diluent.

3. Use of Compounds

In one aspect, the invention relates to the use of a disclosed compound or a product of a disclosed method. In a further aspect, a use relates to the manufacture of a medicament for the treatment of a disorder associated with a Jak2/STAT3 signaling pathway dysfunction in a mammal. In a further aspect, the disorder is a disorder of uncontrolled cellular proliferation. In a still further aspect, the disorder is an inflammatory disease. In a still further aspect, the disorder is an autoimmune disorder.

In a further aspect, a use relates to treatment of a disorder uncontrolled cellular proliferation associated with a Jak2/STAT3 signaling pathway dysfunction in a mammal. In a still further aspect, a use relates to treatment of an immune disorder associated with a Jak2/STAT3 signaling pathway dysfunction in a mammal.

In a further aspect, a use relates to inhibition of IL6-mediated STAT3 phosphorylation activity in a mammal. In a further aspect, a use relates to inhibition of homodimerization of IL6-IL6R-GP130 heterotrimer activity in a mammal. In a further aspect, a use relates to a dysfunction in STAT3 phosphorylation regulation in a mammal. In a further aspect, a use relates to homodimerization of IL6-IL6R-GP130 heterotrimer activity in a mammal. In a further aspect, a use relates to homodimerization of IL6-IL6R-GP130 heterotrimer activity in a cell. In a still further aspect, a use relates to IL6-mediated STAT3 phosphorylation activity in a cell. In a still further aspect, a use relates to Jak2/STAT3 signaling pathway activity in a cell.

4. Kits

In one aspect, the invention relates to a kit are kits comprising at least one compound having a structure represented by a formula:

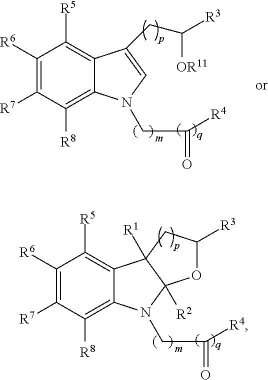

wherein m and n are integers independently selected from 1, 2, 3, 4, 5, and 6; wherein p is an integer selected from 1, 2 and 3; and wherein q is an integer selected from 0 and 1; wherein each of R¹ and R², when present, is independently selected from H and —OH; wherein R³ is selected from: hydrogen,

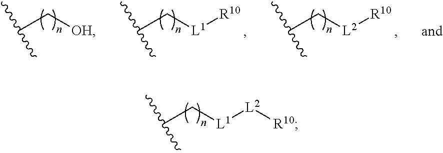

wherein L¹ is —O— or —NH—; wherein L² is —CH₂ or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C₁-C₆ alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, (C1-C6 alkyl)-OH, and C1-C6 alkoxy; and wherein each of R²⁵ and R²⁶ is independently selected from hydrogen and C1-C6 alkyl; wherein R¹¹, when present, is selected from hydrogen and C1-C8 alkyl; or a pharmaceutically acceptable salt, solvate, or polymorph thereof; and one or more of: (a) at least one agent known to increase IL6 activity; (b) at least one agent known to decrease IL6 activity; (c) at least one agent known to treat an immune disorder; (d) at least one agent known to treat a disease of uncontrolled cellular proliferation; or (e) instructions for treating a disorder associated with STAT3 dysfunction.

In a further aspect, the kit comprises a disclosed compound or a product of a disclosed method.

In a further aspect, the at least one compound and the at least one agent are co-formulated. In a still further aspect, the at least one compound and the at least one agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

5. Non-Medical Uses

Also provided are the uses of the disclosed compounds and products as pharmacological tools in the development and standardization of in vitro and in vivo test systems for the evaluation of the effects of inhibition of homodimerization of IL6-IL6R-GP130 heterotrimer related activity in laboratory animals such as cats, dogs, rabbits, monkeys, rats and mice, as part of the search for new therapeutic agents for intervention in dysregulation of IL6 mediated activation of the Jak2/STAT3 pathway.

In various aspects, also provided are the uses of the disclosed compounds and products as pharmacological tools in the development and standardization of in vitro and in vivo test systems for the evaluation of the effects of inhibition of IL6-mediated STAT3 phosphorylation related activity in laboratory animals such as cats, dogs, rabbits, monkeys, rats and mice, as part of the search for new therapeutic agents for intervention in dysregulation of IL6 mediated activation of the Jak2/STAT3 pathway.

### H. REFERENCES

(1) Smith, P. C., Hobisch, A., Lin, D.-L., Culig, Z., and Keller, E. T. (2001) Interleukin-6 and prostate cancer progression. Cytokine and Growth Factor Rev., 12: 33-40.
(2) Kishimoto, T. (2005) Interleukin-6: from basic science to medicine—40 years in immunology. Annu. Rev. Immunol., 23: 1-21.
(3) Siegall, C. B., Schwab, G., Nordan, R. P., FitzGerald, D. J., and Pastan, I.
(1990) Expression of the interleukin 6 receptor and interleukin 6 in prostate carcinoma cells. Cancer Res., 50: 7786-7788.
(4) Siegsmund, M. J., Yamazaki, H., and Pastan, I. (1994) Interleukin 6 receptor mRNA in prostate carcinomas and benign prostate hyperplasia. J. Urol., 151: 1396-1399.
(5) Hobisch, A., Rogatsch, H., Hittmair, A., Fuchs, D., Bartsch, G., Klocker, H., and Culig, Z. (2000) Immunohistochemical localization of interleukin-6 and its receptor in benign, premalignant and malignant prostate tissue. J. Pathol., 191: 239-244.
(6) Palmer, J., Hertzog, P. J., and Hammacher, A. (2004) Differential expression and effects of gp130 cytokines and receptros in prostate cancer cells. Int. J. Biochem. Cell. Biol. 36: 2258-2269.
(7) Lou, W., Ni, Z., Dyer, K., Tweardy, D. J., and Gao, A. C. (2000) Interleukin-6 induces prostate cancer cell growth by activation of Stat3 signaling pathway. Prostate 42: 239-242.
(8) Culig, Z., Steiner, H., Bartsch, G., and Hobisch, A. (2005) Interleukin-6 regulation of prostate cancer cell growth. J. Cell. Biochem., 95: 497-505.
(9) Lee, S. O., Chun, J. Y., Nadiminty, N., Lou, W., and Gao, A. C. (2007) Interleukin-6 undergoes transition from growth inhibitor associated with neuroendocrine differentiation to stimulator accompanied by androgen receptor activation during LNCaP prostate cancer cell progression. Prostate, 67: 764-773.
(10) Schindler, C. and Darnell, J. E., Jr. (1995) Transcriptional response to polypeptide ligands: The JAK/STAT pathway. Ann. Rev. Biochem., 64: 621-651.
(11) Zhong, Z., Wen, Z., and Darnell, J. E., Jr. (1994) Stat3: a STAT family member activated by tyrosine phosphorylation in response to epidermal growth factor and interleukin-6. Science, 264: 95-98.
(12) Mora, L. B., Buettner, R., Seigne, J., Diaz, J., Ahmad, N., Garcia, R., Bowman, T., Falcone, R., Fairclough, R., Cantor, A., Muro-Cacho, C., Livingston, S., Karras, J., Pow-Sang, J., and Jove, R. (2002) Constitutive activation of Stat3 in human prostate tumors and cell lines: direct inhibition of Stat3 signaling induces apoptosis of prostate cancer cells. Cancer Res., 62: 6659-6666.
(13) Barton, B. E. (2005) interleukin-6 and new strategies for the treatment of cancer, hyperproliferative diseases and paraneoplastic syndromes. Expert Opin. Ther. Targets, 9: 737-752.
(14) Barton, B. E., Karras, J. G., Murphy, T. F., Barton, A., and Huang, H. F. (2004) Signal transducer and activator of transcription 3 (STAT3) activation in prostate cancer: Direct STAT3 inhibition induces apoptosis in prostate cancer lines. Mol. Cancer. Ther., 3: 11-20.
(15) Borsellino, N., Bonavida, B., Ciliberto, G., Toniatti, C., Travali, S., and D'Allesandro, N. (1999) Blocking signaling through the gp130 receptor chain by interleukin-6 and oncostatin M inhibits PC-3 cell growth and sensitizes the tumor cells to etoposide and cisplatnin-mediated cytotoxicity. Cancer, 85: 134-144.
(16) Smith, P. C. and Keller, E. T. (2001) Anti-interleukin-6 monoclonal antibody induces regression of human prostate cancer xenografts in nude mice. Prostate 48: 47-53.
(17) Selander, K. S., Li, L., Watson, L., Merrill, M., Dahmen, H., Heinrich, P. C., Muller-Newen, G., and Harris, K. W. (2004) Inhibition of gp130 signaling in breast cancer blocks constitutive activation of stat3 and inhibits in vivo malignancy. Cancer Res., 64: 6924-6933.
(18) Hayashi, M., Kim, Y. P., Takamatsu, S., Enomoto, A., Shinose, M., Takahasi, Y., Tanaka, H., Komiyama, K., and Omura, S. (1996) Madindoline, a novel inhibitor of IL-6 activity from Streptomyces sp. K₉₃-0711.1. Taxonomy, fermentation, isolation and biological activities. J. Antibiot. 49: 1091-1095.
(19) Hayashi, M., Rho, M.-C., Enomoto, A., Fukami, A., Kim, Y.-P., Kikuchi, Y., Sunazuka, T., Hirose, T., Komiyama, K. and Omura, S. (2002) Suppression of bone resorption by madindoline A, a novel nonpeptide antagonist to gp130. Proc. Natl. Acad. Sci. (USA), 99, 14728-14733.
(20) Yamamoto, D., Sunazuka, T., Hirose, T., Kojima, N., Kajim E., and Omura, S. (2006) Design, synthesis, and biological activities of madindoline analogues. Bioorg. Med. Chem. Lett., 16: 2807-2811.
(21) Saleh, A. Z. M., Greenman, K. L., Billings, S., Van Vranken, D. L. and Krolewski, J. J. (2005) Binding of madindoline A to the extracellular domain of gp130. Biochem., 44: 10822-10827.
(22) Wan, L. and Tius, M. A. (2007) Synthesis of (+)-madindoline A and (+)-madindoline B. Org. Lett. 9: 647-650.
(23) Huey, R., G. M. Morris, A. J. Olson, and D. S. Goodsell. (2007) A semiempirical free energy force field with charge-based desolvation. J Comput Chem 28: 1145-52.
(24) Boulanger, M. J., Chow, D.C., Brevnova, E. E., and Garcia, K. C. (2003) Hexameric Structure and Assembly of the Interleukin-6/IL-6 alpha-Receptor/gp130 Complex Science, 300: 2101-2104.
(25) AlleGrow (Boston De Novo, LLC) through private communications.
(26) CombiGlide (Schrodinger, LLC) with academic licensing.
(27) Hosokawa, S., Sekiguchi, K., Enemoto, M., and Kobayashi, S. (2000) Novel stereoselective constructuion of a quaternary carbon: application to synthesis of the cyclopentenedione moiety of madindolines. Tet. Lett., 41: 6429-6433.
(28) Hosokawa, S., Sekiguchi, K., Hayase, K., Hirakawa, Y., and Kobayashi, S. (2000) Total synthesis of madindoline A. Tet. Lett., 41: 6435-6439.
(29) Hou, D.-R., Wang, M.-S., Chung, M.-W., Hsieh, Y.-D, and Tsai, H.-H. G. (2007) Formation of 4,5,6,7-tetrahydro-isoindoles by palladium-catalyzed hydride reduction. J. Org. Chem., 72: 9231-9239.
(30) Sunazuka, T., Hirose, T., Shirahata, T., Harigaya, Y., Hayashi, M., Komiyama, K., Omura, S. (2000) Total Synthesis of (+)-madindoline A and (−)-madindoline B, potent, selective inhibitors of interleukin 6. Determination of the relative and absolute configurations. J. Am. Chem. Soc., 122: 2122-2123.
(31) Sunazuka, T., Shirahata, T., Tsuchiya, S., Hirose, T., Mori, R., Harigaya, Y., Kuwajima, I., Omura, S. (2005) A concise stereoselective route to the indoline spiroaminal framework of neoxaline and oxaline. Org. Lett., 7: 941-943.
(32) Jogireddy, R., Maier, M. E. (2006) Synthesis of luminacin D. J. Org. Chem., 71: 6999-7006.
(33) Aoki, Y., Feldman, G., and Tosato, G. (2003) Inhibition of STAT3 signaling induces apoptosis and decreases survivin expression in primary effusion lymphoma. Blood, 101: 1535-1542.
(34) Real, P., Sierra, A., De Juan, A., Segovia, J., Lopez-Vega, J., and Fernandez-Luna, J. (2002) Resistance to chemotherapy via Stat3-dependent overexpression of Bcl-2 in metastatic breast cancer cells. Oncogene, 21: 7611-7618.
(35) Bromberg, J., Wrzeszcznska, M., Devgan, G., Zhao, Y., Pestell, R., Albanese, C., and Darnell, J. J. (1999) STAT 3 as an Oncogene. Cell, 98: 295-303.
(36) Wei, D., Le, X., Zheng, L., Wang, L., Frey, J., Gao, A., Peng, Z., Huang, S., Xiong, H., Abbruzzese, J., and Xie, K. (2003) Stat3 activation regulates the expression of vascular endothelial growth factor and human pancreatic cancer angiogenesis and metastasis. Oncogene, 22: 319-329.
(37) Wei, L., Kuo, M., Chen, C., Chou, C., Lai, K., Lee, C., and Hsieh, C. (2003) Interleukin-6 promotes cervical tumor growth by VEGF-dependent angiogenesis via a STAT3 pathway. Oncogene, 22: 1517-1527.
(38) Dechow, T. N., Pedranzini, L., Leitch, A., Leslie, K., Gerald, W. L., Linkov, I., and Bromberg, J. F. (2004) Requirement of matrix metalloproteinase-9 for the transformation of human mammary epithelial cells by Stat3-C. Proc. Natl. Acad. Sci. USA, 101: 10602-10607.
(39) Song, H., Wang, R., Wang, S., and Lin, J. (2005) A low-molecular-weight compound discovered through virtual database screening inhibits Stat3 function in breast cancer cells. Proc Natl Acad Sci USA, 102: 4700-4705.
(40) Niu, G., Heller, R., Catlett-Falcone, R., Coppola Jaroszeski, M., Dalton, W., Jove. R, and Yu, Y. (1999) Gene Therapy with Dominant-Negative STAT 3 Suppresses Growth of the Murine Melanoma B16 Tumor in Vivo. Cancer Res., 59: 5059-5063.
(41) Burke, W., Jin, X., Liu, R., Huang, M., Reynolds, R. K., and Lin, J. (2001) Inhibition of constitutively active Stat3 pathway in ovarian and breast cancer cells. Oncogene, 20: 7925-7934.
(42) Catlett-Falcone, R., Landowski, T. H., Oshiro, M. M., Turkson, J., Levitzki, A., Savino, R., Ciliberto, G., Moscinski, L., Fernandez-Luna, J. L., Nunez, G., Dalton, W. S., and Jove, R. (1999) Constitutive activation of Stat3 signaling confers resistance to apoptosis in human U266 myeloma cells. Immunity, 10: 105-115.
(43) Bowman, T., Broome, M. A., Sinibaldi, D., Wharton, W., Pledger, W. J., Sedivy, J. M., Irby, R., Yeatman, T., Courtneidge, S. A., and Jove, R. (2001) Stat3-mediated Myc expression is required for Src transformation and PDGF-induced mitogenesis. Proc. Natl. Acad. Sci. USA, 98: 7319-7324.
(44) Kulp, S. K., Yang, Y.-T., Hung, C.-C., Chen, K.-F., Lai, J.-P., Tseng, P.-H., Fowble, J. W., Ward, P. J., and Chen, C.-S. (2004) 3-Phosphoinositide-dependent protein kinase-1/Akt signaling represents a major cyclooxygenase-2-independent target for celecoxib in prostate cancer cells. Cancer Res., 64: 1444-1451.
(45) Shiau, C.-W., Yang, C.-C., Kulp, S. K., Chen, K.-F., Chen, C.-S., Huang, J.—W., and Chen, C.-S. (2005) Thiazolidenediones mediate apoptosis in prostate cancer cells in part through inhibition of Bcl-xL/Bcl-2 functions independently of PPARγ. Cancer Res., 65: 1561-1569.
(46) Kulp, S. K., Chen, C.-S., Wang, D.-S., Chen, C.-Y., and Chen, C.-S. (2006) Antitumor effects of a novel phenylbutyrate-based histone deacetylase inhibitor, (S)-DAC-42, in prostate cancer. Clin. Cancer Res., 12: 5199-5206.
(47) Lu, Y.-S., Kashida, Y., Kulp, S. K., Wang, Y.-C., Wang, D., Hung, J.-H., Tang, M., Lin, Z.-Z., Chen., T.-J., Cheng, A.-L., Chen, C.-S. (2007) Efficacy of a novel histone deacetylase inhibitor in murine models of hepatocellular carcinoma. Hepatology, 46: 1119-1130.
(48) Weng, J.-R., Tsai, C.-H., Kulp, S. K., Wang, D., Lin, C.-H., Yang, H.-C., Ma, Y., Sargeant, A., Chiu, C.-F., Tsai, M.-H., and Chen, C.-S. (2007) A potent indole-3-carbinol-derived antitumor agent with pleiotropic effects on multiple signaling pathways in prostate cancer cells. Cancer Res., 67: 7815-7824.
(49) Wang, Y. C., Kulp, S. K., Wang, D., Yang, C.-C., Sargeant, A. M., Hung, J.—H., Kashida, Y., Yamaguchi, M., Chang, G.-D., and Chen, C.-S. (2008) Targeting endoplasmic reticulum stress and Akt with OSU-03012 and gefitinib or erlotinib to overcome resistance to epidermal growth factor receptor inhibitors. Cancer Res., 68: 2820-2830.
(50) Weng, S.-C., Kashida, Y., Kulp, S. K., Wang, D., Brueggemeier, R. W., Shapiro, C. L., and Chen, C.-S. (2008) Sensitizing estrogen receptornegative breast cancer cells to tamoxifen with OSU-03012, a novel celecoxib-derived phosphoinositide-dependent protein kinase-1/Akt signaling inhibitor. Mol. Cancer. Ther., 7: 800-808.
(51) Sirotnak, F. M., Zakowski, M. F., Miller, V. A., Scher, H. I., and Kris, M. G. (2000) Efficacy of cytotoxic agents against human tumor xenografts is markedly enhanced by coadministration of ZD1839 (Iressa), an inhibitor of EGFR tyrosine kinase. Clin. Cancer Res., 6: 4885-4892.
Chatelier, R. C. et al A general method to recondition and reuse BIAcore sensor chip fouled by covalently immobilized protein/peptide. Anal. Biochem, 1995, 229 112-118.
Fivash, M.; Towler, E. M.; Fisher, R. J. BIAcore for macromolecular interaction. Curr Opin Biotechnol. 1998, 9(1), 97-101.
Saleh, A. Z. M.; Greenman, K. L.; Billings, S.; Van Vranken, D. L.; Krolewski, J. J. Binding of madindoline A to the extracellular domain of gp130 Biochemistry 2005, 44, 10822-10827.
QikProp, version 3.4, Schrodinger, LLC, New York, N.Y., 2011.

### I. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Several methods for preparing the compounds of this invention are illustrated in the following Examples. Starting materials and the requisite intermediates are in some cases commercially available, or can be prepared according to literature procedures or as illustrated herein.

The following exemplary compounds of the invention were synthesized. The Examples are provided herein to illustrate the invention, and should not be construed as limiting the invention in any way. The Examples are typically depicted in free base form, according to the IUPAC naming convention.

As indicated, some of the Examples were obtained as racemic mixtures of one or more enantiomers or diastereomers. The compounds may be separated by one skilled in the art to isolate individual enantiomers. Separation can be carried out by the coupling of a racemic mixture of compounds to an enantiomerically pure compound to form a diastereomeric mixture, followed by separation of the individual diastereomers by standard methods, such as fractional crystallization or chromatography. A racemic or diastereomeric mixture of the compounds can also be separated directly by chromatographic methods using chiral stationary phases.

### 1. Preparation of Benzyl Subunit 8

The overall synthetic was as shown below.

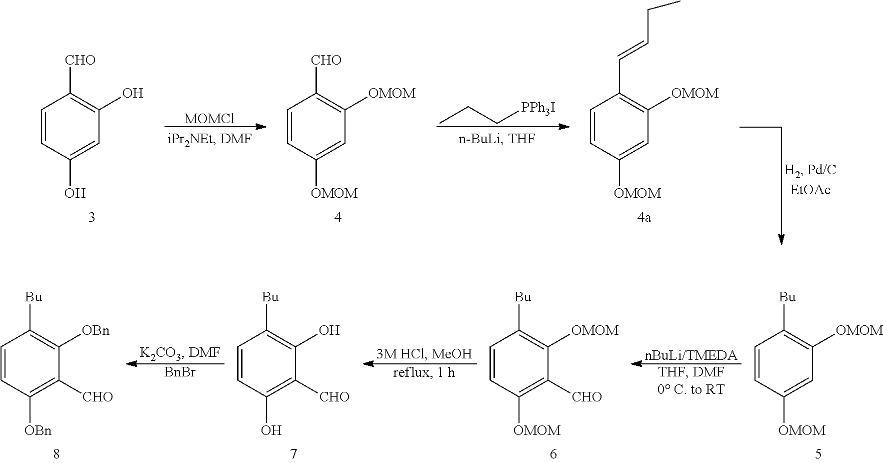

### a. Preparation of 2,4-Bis(Methoxymethoxy)Benzaldehyde (4)

2,4 Dihydroxybenzaldehyde (500 mg, 3.62 mmol) dissolved in DMF (10 mL) was treated with Hunig's base (2.5 mL, 14.48 mmol) dropwise followed by chloromethyl methyl ether (1.1 mL, 14.48 mmol) at room temperature. After 5 h, the reaction mixture was poured on to cold water and extracted with EtOAc (3×10 mL). The combined organic layer were washed with 5% NaOH solution followed by brine. The organic layers were dried over Na₂SO₄ and concentrated. The crude product was purified by flash column chromatography to provide 4 (748 mg, 91% yield) as a colorless oil.^{[1] 1}H-NMR (300 MHz, CDCl₃) δ 10.34 (s, 1H), 7.81 (d, J=8.3 Hz, 1H), 6.83 (d, J=2.1 Hz, 1H), 6.74 (dd, J=2.4, 8.7 Hz, 1H), 5.28 (s, 2H), 5.21 (s, 1H), 3.52 (s, 3H), 3.48 (s, 3H). ¹³C-NMR (100 MHz, CDCl₃) δ 188.1, 163.4, 161.1, 129.9, 120.0, 109.2, 102.4, 94.5, 94.0, 56.3, 56.2. HRMS-TOF m/z (M+Na)⁺ calcd 249.0739. found 249.0724.

### b. Preparation of (2,4-Bis(Methoxymethoxy)-1-(But-1-Enyl)Benzene (4A)

A suspension of CH₃CH₂CH₂P(Ph)₃I (2.14 g, 3.30 mmol) in THF (16 mL) was treated with n-BuLi 2.5 M in hexanes (1.98 mL, 4.95 mmol) drop wise at 0° C. After stirring for 30 min, 2,4-Bis(methoxymethoxy)benzaldehyde 4 (748 mg, 3.30 mmol)pre-dissolved in THF (9 mL) was added drop wise. The resulting reaction mixture was stirred for 5 h at room temperature and then quenched with water and extracted with ether (4×10 mL). The combined organic layer was washed with brine, dried over Na₂SO₄ and concentrated under reduced pressure. The crude product was purified by flash column chromatography to provide 4a (815 mg, 98% yield) as a colorless oil. IR (film): □ₘₐₓ) 2950, 2911, 1604, 1496, 1265, 1211, 1157, 1076, 1006, 782 cm-1. ¹H NMR (400 MHz, CDCl₃) (observed as mixture of E/Z 1.3:1) δ 7.35 (d, J=8.5 Hz, 1H), 7.16 (d, J=8.4 Hz, 1H), 6.82 (d, J=2.4 Hz, 1H), 6.78 (d, J=2.4 Hz, 1H), 6.68 (ddd, J=8.3, 5.5, 2.4 Hz, 2H), 6.63 (d, J=16.0 Hz, 2H), 6.43 (d, J=11.6 Hz, 1H), 6.14 (dt, J=15.9, 6.6 Hz, 1H), 5.65 (dt, J=11.5, 7.3 Hz, 1H), 5.18 (s, 2H), 5.17 (s, 2H), 5.16 (s, 2H), 5.15 (s, 2H), 3.50 (s, 3H), 3.49 (s, 3H), 3.48 (s, 3H), 3.47 (s, 3H), 2.33 2.16 (m, 5H), 1.09 (t, J=7.5 Hz, 3H), 1.04 (t, J=7.5 Hz, 3H). ¹³C-NMR (100 MHz, CDCl₃) δ 157.1, 157.0, 155.5, 154.7, 133.8, 131.8, 130.4, 126.8, 123.0, 122.7, 121.9, 121.3, 109.3, 108.4, 103.8, 103.7, 94.8, 94.7, 94.5, 56.1(2), 56.0(2), 37.2, 27.8, 26.4, 22.7, 21.9, 14.4, 14.1, 13.8. HRMS-TOF m/z (M+Na)⁺ calcd 275.1259. found 275.1232.

### c. Preparation of (2,4-Bis(Methoxymethoxy)-1-(Butyl)Benzene(5)

The olefin 4a (815 mg, 3.23 mmol) in dry EtOAc (15 mL) was stirred with 10% Pd/C (82 mg, 10 mol %) under a balloon atmosphere of hydrogen gas for 16 h. The reaction mixture was then filtered through Celite and washed with EtOAc (3×20 mL). The filtrate was concentrated and the crude product was purified by flash chromatography to give 5 (817.8 mg, quantitative) as colorless oil. ¹H-NMR (300 MHz, CDCl₃) δ 7.03 (d, J=8.3 Hz, 1H), 6.77 (d, J=2.4 Hz, 1H), 6.63 (dd, d, J=2.4, 8.1 Hz, 1H), 5.17 (s, 2H), 5.13 (s, 1H), 3.48 (s, 6H), 2.56 (t, J=7.8 Hz 2H), 1.55 (quin, 2H), 1.36 (m, 2H), 0.92 (t, J=7.2 Hz, 3H). ¹³C-NMR (100 MHz, CDCl₃) δ 156.2, 155.6, 130.1, 125.4, 108.5, 103.3, 94.6, 94.4, 55.9, 55.9, 32.4, 29.3, 22.5, 13.9. HRMS-TOF m/z (M+Na)⁺ calcd 277.1416. found 277.1390.

### d. Preparation of 3-Butyl-2,6-Bis(Methoxymethoxy)Benzaldehyde (6)

A stirred solution of resorcinol derivative 5 (900 mg, 3.53 mmol) and TMEDA (589 mL, 3.89 mmol) in dry ether (12 mL) was treated with n-BuLi [2.5 M in hexanes] (1.55 mL, 3.85 mmol) dropwise at 0° C. under argon. The reaction mixture was stirred for 2 h at the same temperature and then allowed to warm to room temperature. DMF (0.6 mL 7.78 mmol) was then added and stirring continued for 4 h. The reaction mixture was poured into a separatory funnel containing water and extracted with ether (3×10 mL). The combined organic layers were washed with water, saturated aqueous NH₄Cl and brine, dried over Na₂SO₄ and concentrated under reduced pressure. The crude product was purified by flash column chromatography to afford the aldehyde 6 (732 mg, 68% yield) as colorless liquid. ¹H-NMR (300 MHz, CDCl₃) δ 10.64 (s, 1H), 7.33 (d, J=8.7 Hz, 1H), 6.93 (d, J=8.7 Hz, 1H), 5.24 (s, 2H), 5.04 (s, 2H), 3.58 (s, 3H), 3.50 (s, 3H), 2.63 (t, J=7.8 Hz, 2H), 1.57 (quin, 2H), 1.35 (m, 2H) 0.93 (t, J=7.2 Hz, 3H). ¹³C-NMR (100 MHz, CDCl₃) δ 189.6, 158.4, 156.9, 136.1, 130.3, 119.1, 110.7, 101.8, 94.9, 57.5, 56.4, 32.5, 29.2, 22.6, 13.9. HRMS-TOF m/z (M+Na)⁺ calcd 305.1365. found 305.1348.

### e. Preparation of 3-Butyl-2,6-Dihydroxybenzaldehyde (7)

A stirred solution of 3-butyl-2,6-dihydroxy-benzaldehyde (428 mg, 1.52 mmol) in MeOH (12 mL) was treated with 3M HCl (4 mL) and refluxed for 1 h. The reaction mixture was then cooled and concentrated to remove MeOH. The residue was redissolved in EtOAc and washed with water followed by brine. The organic layer was dried over Na₂SO₄ and concentrated under reduced pressure to provide pure 7 (243 mg, 83% yield). ¹H-NMR (400 MHz, CDCl₃) δ 10.37 (s, 1H), 7.18 (d, J=8.2 Hz, 1H), 6.26 (d, J=8.2 Hz, 1H), 2.25 (t, J=7.8 Hz, 1H), 1.50-1.56 (m, 2H), 1.33-1.38 (m, 2H), 0.93 (t, J=7.4 Hz, 3H). ¹³C-NMR (100 MHz, CDCl₃) δ 194.3, 138.6, 109.9, 31.7, 28.2, 22.4, 13.9.

### f. Preparation of 2,6-Bis(Benzyloxy)-3-Butylbenzaldehyde (8)

Dihydroxy derivative 7 (700 mg, 2.37 mmol) in DMF (24 mL) was treated with potassium carbonate (1.31 g, 9.54 mmol), benzyl bromide (1.22 g, 7.11 mmol) and 10 uL water. The resulting reaction mixture was heated to 50° C. for 16 h, after which it was quenched with cold water and extracted with Hexane:EtOAc (1:1, 3×20 mL). The combined organic layers were washed with brine, dried over Na₂SO₄ and concentrated under reduced pressure. The crude product was purified by flash column chromatography to provide the aldehyde (1.34 g, quantitative) as a white solid. ¹H-NMR (400 MHz, CDCl₃) δ 10.58 (s, 1H), 6.78 (d, J=8.0 Hz, 1H), 6.31 (d, J=8.0 Hz, 1H), 5.17 (s, 2H), 4.93 (s, 2H), 2.55 (t, J=8.0 Hz, 2H), 1.51 (quin, 2H), 1.30 (sex, 2H), 0.88 (t, J=4.0 Hz, 3H). ¹³C-NMR (100 MHz, CDCl₃) δ 189.6, 159.8, 158.5, 137.0, 136.3, 136.2, 129.6, 128.7, 128.5, 128.3, 128.2, 128.1, 127.2, 119.3, 108.8, 77.4, 70.8, 32.8, 28.8, 22.6, 13.9. HRMS-TOF m/z (M+Na)⁺ calcd 397.1780. found 397.1767.

### 2. Preparation of Hydroxyfuroindoline Subunit 12

The overall synthetic scheme was as shown below.

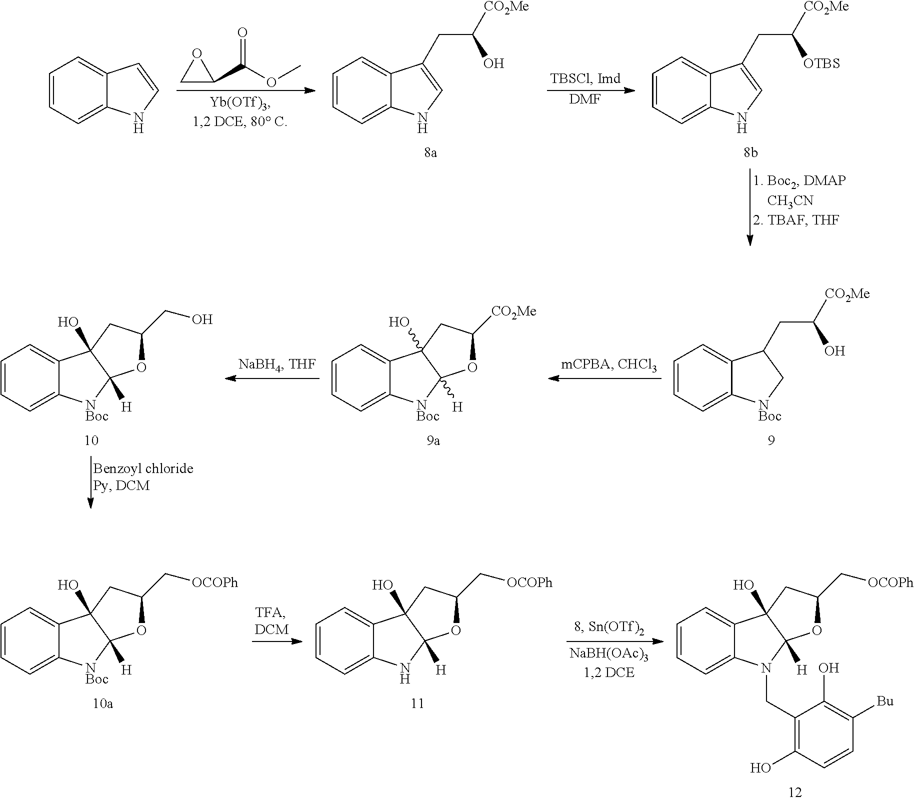

### a. Preparation of (S)-Methyl 2-Hydroxy-3-(1H-Indol-3-Yl)Propanoate (8A)

Yb(OTf)₃ (909 mg, 1.46 mmol) was added to a solution of indole (3.42 g, 29.19 mmol) and methyl (2S)-glycidate (1.49 g, 14.57 mmol) in 1,2 dichloroethane (30 mL) at room temperature. The reaction mixture was warmed to 80° C., stirred for 3 h, and then cooled to room temperature. The resulting reaction mixture was quenched with sat. aq. Na₂CO₃ and acidified with 1 N HCl. The aqueous layer was extracted with CHCl₃ (3×20 mL). The combined organic extracts were washed with brine, dried over Na₂SO₄, filtered and evaporated under reduced pressure. The crude product was purified by flash chromatography to provide the product 8a (2.04 g, 64% yield) as colorless solid. m.p. 62-63° C.; [α]_{D}²²-24.8 (c=1.02, CHCl₃).¹H-NMR (400 MHz, CDCl₃) 6 ¹H NMR (400 MHz, CDCl₃) δ 8.13 (s, 1H), 7.62 (d, J=7.8 Hz, 1H), 7.34 (d, J=8.0 Hz, 1H), 7.20 (t, J=7.4 Hz, 1H), 7.13 (t, J=7.4 Hz, 1H), 7.06 (d, J=1.9 Hz, 1H), 4.54 (dd, J=9.5, 4.9 Hz, 1H), 3.72 (s, 3H), 3.31 (dd, J=14.8, 4.3 Hz, 1H), 3.20 (dd, J=14.8, 6.2 Hz, 1H), 2.83 (d, J=5.9 Hz, 1H). ¹³C-NMR (100 MHz, CDCl₃) δ 174.7, 136.0, 127.5, 123.2, 122.0, 119.4, 118.7, 111.1, 109.9, 70.7, 52.4, 30.2. HRMS-TOF m/z (M+Na)⁺ calcd for 242.0793. found 242.0775.

### b. Preparation of (S)-Methyl 2-((Tert-Butyldimethylsilyl)Oxy)-3-(1H-Indol-3-Yl)Propanoate (8B)

To a solution of (S)-methyl 2-hydroxy-3-(1H-indol-3-yl)propanoate (3.31 g, 15.10 mmol) dissolved in anhydrous DMF (21 mL) was added imidazole (2.05 g, 30.20 mmol) followed by TBSCl (2.50 g, 16.61 mmol) in one portion at room temperature. After stirring for 16 h, the reaction mixture was quenched with cold water and extracted with 1:1 Hexane/EtOAc (4×50 mL). The combined organic layer was washed with brine, dried over Na₂SO₄, filtered and evaporated under reduced pressure. The crude product was purified by flash chromatography to provide 8b (4.25 g, 85% yield) as a white solid. m.p. 78-80° C.; [α]_{D}²²-5.4 (c=1.0, CHCl₃). ¹H NMR (400 MHz, CDCl₃) δ 8.03 (s, 1H), 7.62 (d, J=7.8 Hz, 1H), 7.34 (d, J=8.0 Hz, 1H), 7.21-7.14 (m, 1H), 7.14-7.08 (m, 1H), 7.07 (d, J=2.2 Hz, 1H), 4.48 (dd, J=7.8, 4.7 Hz, 1H), 3.69 (s, 3H), 3.26 (dd, J=14.4, 4.7 Hz, 1H), 3.11 (dd, J=14.4, 7.8 Hz, 1H), 0.81 (s, 9H), −0.11 (s, 3H), −0.18 (s, 3H). ¹³C-NMR (100 MHz, CDCl₃) δ 173.9, 136.0, 127.5, 123.2, 121.8, 119.3, 118.7, 111.2, 111.0, 72.8, 51.8, 31.2, 25.6 (3C), 18.2, −5.4, −5.4. HRMS-TOF m/z (M+Na)⁺ calcd M+Na 356.1658. found 356.1654.

### c. Preparation of (S)-Tert-Butyl 3-(2-Hydroxy-3-Methoxy-3-Oxopropyl)-1H-Indole-1-Carboxylate (9)

Boc₂O (4.64 g, 21.27 mmol) and DMAP (386 mg, 1.77 mmol) were added to a stirred solution of (S)-methyl 2-((tert-butyldimethylsilyl)oxy)-3-(1H-indol-3-yl)propanoate 8b (6.0 g, 17.72 mmol) in acetonitrile (37.5 mL) at room temperature. After stirring for 4 h, the reaction mixture was quenched with water and extracted with CHCl₃ (3×30 mL).The combined organic layer was washed with brine, dried over Na₂SO₄, filtered and concentrated under reduced pressure. The crude was taken forward to the next step without purification. A stirred solution of crude (S)-tert-butyl 3-(2-((tert-butyldimethylsilyl)oxy)-3-methoxy-3-oxopropyl)-1H-indole-1-carboxylate from the previous step in anhydrous THF (25 mL) under argon was treated with TBAF (1.0 M in THF, 17.9 mL) at room temperature. After stirring for 30 min, the reaction mixture was diluted with CHCl₃ and water. The aqueous layer was extracted with CHCl₃ and the combined organic layer was washed with brine, dried over Na₂SO₄, filtered and concentrated under reduced pressure. The crude product was purified by flash chromatography to provide 9 (5.64 g, quantitative yield) as an off-white solid. [α]_{D}22-2.8 (c=1.0, CHCl₃). ¹H-NMR (400 MHz, CDCl₃) δ 8.11 (s, 1H), 7.54 (ddd, J=7.7, 1.2, 0.7 Hz, 1H), 7.49 (s, 1H), 7.31 (ddd, J=8.3, 7.3, 1.2 Hz, 1H), 7.23 (ddd, J=7.7, 7.2, 1.1 Hz, 1H), 4.53 (t, J=5.3 Hz, 1H), 3.74 (s, 3H), 3.23 (ddd, J=14.8, 4.3, 1.0 Hz, 1H), 3.10 (ddd, J=14.8, 6.4, 0.8 Hz, 1H), 2.85 (s, 1H), 1.66 (s, 9H). ¹³C-NMR (100 MHz, CDCl₃) δ 174.5, 130.5, 124.4, 122.4, 118.9, 115.2, 114.9, 83.6, 71.8, 70.2, 52.6, 29.9, 28.2. HRMS-TOF m/z (M+Na)⁺ calcd for 342.1317. found 342.1301.

### d. Preparation of (2S)-8-Tert-Butyl 2-Methyl 3A-Hydroxy-3,3A-Dihydro-2H-Furo[2,3-B]Indole-2,8(8AH)-Dicarboxylate (9A)

m-CPBA [75% in water (8.24 g, 35.6 mmol)] was added portion wise to a stirred solution of (S)-tert-butyl 3-(2-hydroxy-3-methoxy-3-oxopropyl)-1H-indole-1-carboxylate (5.69 g, 17.8 mol) in chloroform (150 mL) at 0° C. under agron. The reaction mixture was allowed to warm to room temperature. After stirring for 5 h, the reaction mixture was quenched with 0.5 M sodium thiosulfate (5 mL). The resulting reaction mixture was partitioned between CHCl₃ and saturated NaHCO₃ solution. The aqueous layer was extracted with chloroform (3×20 mL). The combined organic layers were dried over sodium sulfate and concentrated. The crude product was purified by flash chromatography to provide 9a(mixture of diastereomers, 3.94 g, 62% yield) as a foamy solid. HRMS-TOF m/z (M+Na)⁺ calcd 358.1267 for found 358.1253.

### e. Preparation of (2S)-Tert-Butyl 3A-Hydroxy-2-(Hydroxymethyl)-3,3A-Dihydro-2H-Furo[2,3-B]Indole-8(8AH)-Carboxylate (10)

Sodium borohydride (1.25 g, 33.24 mmol) was added portion wise to the solution of (2S)-8-tert-butyl 2-methyl 3a-hydroxy-3,3a-dihydro-2H-furo[2,3-b]indole-2,8(8aH)-dicarboxylate (3.94 g, 11.08 mmol) in anhydrous THF (35 mL) under argon at room temperature. After stirring for 24 h at room temperature, the reaction mixture was cooled to 0° C. then added water cautiously followed by saturated NH₄Cl solution. After stirring for 10 min, the solution was extracted with EtOAc (3×20 mL). The combined organic layers were washed with saturated aqueous NH₄Cl, brine, dried over sodium sulfate and concentrated. The crude product was purified by flash chromatography afforded the product 10 (mixture of diastereoisomers, 2.29 g, 67% yield) as a thick oil. HRMS-TOF m/z (M+Na)⁺ calcd 330.1317. found 330.1297.

### f. Preparation of (2S,3AR)-T-Butyl 2-((Benzoyloxy)Methyl)-3A-Hydroxy-3,3A-Dihydro-2H-Furo[2,3-B]Indole-8(8AH)-Carboxylate (10A)

Benzoyl chloride (187.1 mg, 1.33 mmol) and pyridine (105.3 mg, 1.33 mmol) were added dropwise sequentially to a stirred solution of (2S)-tert-butyl 3a-hydroxy-2-(hydroxymethyl)-3,3a-dihydro-2H-furo[2,3-b]indole-8(8aH)-carboxylate (315 mg, 1.02 mmol) in anhydrous dichloromethane (6 mL) at room temperature under argon. The reaction mixture was stirred overnight and then quenched with water. The reaction mixture was extracted with dichloromethane (3×10 mL). The combined organic layers were washed with brine, dried over sodium sulfate and concentrated. The crude product was purified by flash chromatography to provide the desired benzoylated diastereomer (A) (242 mg, 57% yield) as a white solid and diastereomer (B) (157 mg, 37% yield) as a thick oil. A: 76-79° C. [α]_{D}²²-12.95° (c=3.43, CHCl₃).¹H NMR (400 MHz, CDCl₃) δ 8.04 (d, J=7.3 Hz, 2H), 7.55 (t, J=7.4 Hz, 1H), 7.42 (t, J=7.9 Hz, 3H), 7.33 (t, J=7.4 Hz, 1H), 7.08 (t, J=7.5 Hz, 1H), 5.90 (s, 1H), 4.43 (qd, J=11.9, 4.5 Hz, 2H), 4.08 (dt, J=9.2, 5.4 Hz, 1H), 2.55-2.39 (m, 2H), 1.56 (s, 9H). ¹³C-NMR (100 MHz, CDCl₃) δ 170.9, 166.3, 152.2, 133.6, 133.1, 130.7, 130.1, 129.8, 129.7, 129.2, 128.4, 128.3, 123.4, 115.1, 98.4, 65.3, 42.5, 28.3. HRMS-TOF m/z (M+Na)⁺ calcd 434.1580. found 434.1560.

### g. Preparation of ((2S,3AR)-3A-Hydroxy-3,3A,8,8A-Tetrahydro-2H-Furo[2,3-B]Indol-2-Yl)Methyl Benzoate (11)

Trifluoroacetic acid (0.032 mL, 0.41 mmol) was added dropwise to a stirred solution of (2S,3aR)-tert-butyl 2-((benzoyloxy)methyl)-3a-hydroxy-3,3a-dihydro-2H-furo[2,3-b]indole-8(8aH)-carboxylate (100.7 mg, 0.24 mmol) in anhydrous dichloromethane (3 mL) at room temperature under argon. The reaction mixture was stirred overnight. Additional trifluoroacetic acid (0.032 mL, 0.41 mmol) was added and stirred for 4 h. The reaction mixture was diluted with ether, treated with 5% NaOH solution to pH 9, and then extracted with ether (3×5 mL). The combined organic layers were dried over sodium sulfate and concentrated. The crude product was purified by flash chromatography to provide 11 (54 mg, 73% yield) as thick oil. [α]_{D}²²-83.9° (c=1, CHCl₃). ¹H-NMR (400 MHz, CDCl₃) δ8.06 (d, J=7.6 Hz, 2H), 7.56 (t, J=7.4 Hz, 1H), 7.43 (t, J=7.7 Hz, 2H), 7.33 (d, J=7.4 Hz, 1H), 7.18 (t, J=7.7 Hz, 1H), 6.83 (t, J=7.4 Hz, 1H), 6.64 (d, J=7.9 Hz, 1H), 5.52 (s, 1H), 4.65 (s, 1H), 4.52 (dd, J=11.8, 3.3 Hz, 1H), 4.36 (dd, J=11.9, 5.9 Hz, 1H), 2.51 (dd, J=12.1, 4.9 Hz, 1H), 2.40 (dd, J=11.5 Hz, 1H), 2.27 (s, 1H). ¹³C-NMR (100 MHz, CDCl₃) δ: 166.4, 149.5, 133.1, 130.6, 129.8, 129.7, 128.4, 124.1, 119.7, 109.6, 99.3, 89.1, 65.8, 42.6. HRMS-TOF m/z (M+Na)⁺ calcd 334.1055. found 334.1032.

### h. Preparation of ((2S,3AR)-8-(2,6-Bis(Benzyloxy)-3-Butylbenzyl)-3A-Hydroxy-3,3A,8,8A-Tetrahydro-2H-Furo[2,3-B]Indol-2-Yl)Methyl Benzoate (12)

((2S,3aR)-3a-hydroxy-3,3a,8,8a-tetrahydro-2H-furo[2,3-b]indol-2-yl)methyl benzoate 11 (50.7 mg, 0.16 mmol), 2,6-bis(benzyloxy)-3-butylbenzaldehyde 8(64.1 mg, 0.17) and 4 A molecular sieves (122 mg) in 1,2-dichloroethane (3.8 mL) were treated with Sn(OTf)₂ (13.5 mg, 0.035 mmol) and NaBH(OAc)₃ (36.2 mg, 0.17 mmol) at 0° C. The reaction mixture was gradually warmed up to room temperature. After stirring for 4 h at room temperature the reaction was quenched by adding 0.2 mL cold sat. aq. NaHCO₃ solution and extracted with dichloromethane (3×5 mL). The combined organic layers were dried over sodium sulfate and concentrated under reduced pressure. The crude product was purified by flash chromatography to provide 12 (49.4 mg, 45% yield) as a thick oil. [α]_{D}²² −25.3° (c=2.48, CHCl₃). ¹H-NMR (400 MHz, CDCl₃) δ 8.01-7.92 (m, 2H), 7.56-7.50 (m, 1H), 7.46 (d, J=9.0 Hz, 2H), 7.38 (t, J=7.9 Hz, 2H), 7.35-7.30 (m, 2H), 7.30-7.26 (m, 5H), 7.18 (d, J=7.4 Hz, 1H), 7.10-7.02 (m, 2H), 6.72-6.63 (m, 3H), 5.43 (s, 1H), 5.05 (s, 2H), 4.90 (dd, J=32.5, 11.2 Hz, 2H), 4.60 (dd, J=34.7, 13.9 Hz, 2H), 4.39 (dd, J=11.7, 3.6 Hz, 1H), 4.29 (dd, J=11.8, 5.7 Hz, 1H), 4.09-4.01 (m, 1H), 2.72-2.60 (m, 1H), 2.58-2.48 (m, 1H), 2.40 (dd, J=11.8, 4.6 Hz, 1H), 2.27 (t, J=11.5 Hz, 1H), 1.74 (s, 1H), 1.64-1.51 (m, 2H), 1.34 (dt, J=14.9, 7.3 Hz, 2H), 0.89 (d, J=7.4 Hz, 3H). ¹³C-NMR (100 MHz, CDCl₃) δ 166.3, 156.8, 156.4, 150.7, 137.3, 136.9, 132.9, 130.4, 129.9, 129.7, 129.5, 129.4, 128.5, 128.4, 128.2, 127.9, 127.7, 127.4, 123.5, 119.7, 117.3, 107.9, 107.2, 102.5, 87.2, 76.4, 76.1, 70.2, 65.9, 42.8, 38.6, 32.9, 29.3, 25.6, 22.6, 13.9. HRMS-TOF m/z (M+Na)⁺ calcd 692.2988 found 692.2969.

### 3. Preparation of MDL-5 (((2S,3AR)-8-(3-butyl-2,6-Dihydroxybenzyl)-3A-Hydroxy-3,3A,8,8A-Tetrahydro-2H-Furo[2,3-B]Indol-2-Yl)Methyl Benzoate) and MDL-16 ((S)-3-(1-(3-Butyl-2,6-Dihydroxybenzyl)-1H-Indol-3Yl)-2-Hydroxypropyl Benzoate)

The overall synthetic scheme was as shown below.

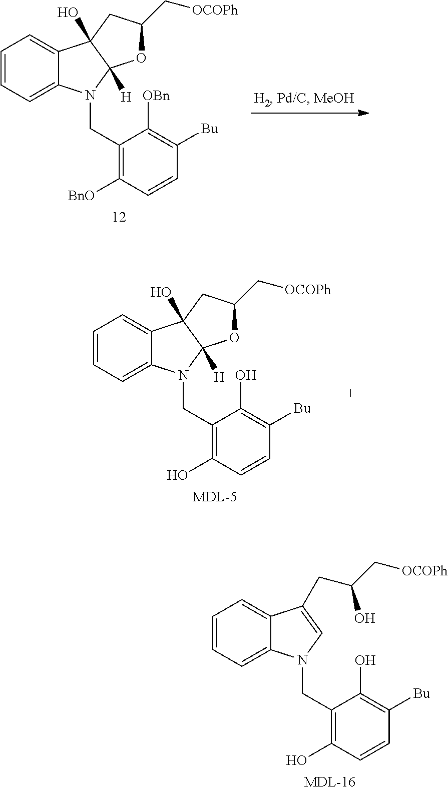

Benzyloxy derivative 12 (149 mg, 0.22 mmol) in MeOH (5 mL) was stirred with 10% Pd/C (40 mg, 20 mol %) under a balloon atmosphere of hydrogen gas for 28 h. The reaction mixture was filtered through Celite washed with ethyl acetate and concentrated under reduced pressure. The crude product was purified by flash chromatography to provide MDL-5 (42 mg) as a solid and MDL-16 (19.2 mg) as a thick colorless oil. MDL-5: m.p 64-68; [α]_{D}²² −121.1° (c=1, CHCl₃). ¹H-NMR (400 MHz, CDCl₃) δ: 8.13 (d, J=7.5 Hz, 2H), 7.57 (t, J=7.4 Hz, 1H), 7.45 (t, J=7.6 Hz, 2H), 7.29 (t, J=7.6 Hz, 1H), 7.19 (t, J=7.7 Hz, 1H), 6.91 (d, J=8.2 Hz, 1H), 6.83 (t, J=7.4 Hz, 1H), 6.78 (d, J=7.9 Hz, 1H), 6.74 (s, 1H), 6.37 (d, J=8.2 Hz, 1H), 6.21 (s, 1H), 5.35 (s, 1H), 4.63 (d, J=14.3 Hz, 1H), 4.55 (dd, J=11.7, 2.4 Hz, 1H), 4.41-4.29 (m, 2H), 4.25 (m, 1H), 2.64 (s, 1H), 2.54 2.44 (m, 3H), 2.34 (t, J=11.5 Hz, 1H), 1.51 (quin, 2H), 1.36-1.24 (m, 2H), 0.88 (t, J=7.3 Hz, 3H). ¹³C-NMR (100 MHz, CDCl₃) δ: 166.2, 154.2, 153.2, 150.1, 133.2, 130.9, 130.5, 129.9, 129.7, 129.5, 128.4, 123.7, 122.2, 119.9, 110.1, 108.9, 108.2, 104.2, 87.2, 65.7, 42.4, 41.2, 32.2, 29.3, 22.4, 13.9. HRMS-TOF m/z (M+Na)⁺ calcd 512.2049. found 512.2030. MDL-16: [α]_{D}²² −3.48° (c=1, CHCl₃). ¹H-NMR (400 MHz, CDCl₃) δ 8.04 (d, J=7.2 Hz, 2H), 7.70 (d, J=8.2 Hz, 1H), 7.56 (t, J=7.5 Hz, 2H), 7.44 (q, J=7.7 Hz, 3H), 7.22 (s, 1H), 7.18 (dd, J=15.4, 7.9 Hz, 1H), 7.08 (t, J=7.5 Hz, 1H), 6.83 (d, J=8.2 Hz, 1H), 6.31 (d, J=8.2 Hz, 1H), 5.70 (s, 1H), 5.33 (s, 2H), 5.19 (s, 1H), 4.39 (t, J=7.6 Hz, 1H), 4.28 (t, J=6.3 Hz, 2H), 3.01 (ddd, J=35.0, 20.4, 14.3 Hz, 2H), 2.45 (t, J=7.5 Hz, 2H), 2.33 (s, 1H) 1.52 (m, 2H), 1.36 (m, 2H), 0.92 (t, J=7.3 Hz, 3H). ¹³C-NMR (100 MHz, CDCl₃) δ: δ 167.0, 153.5, 153.2, 136.8, 133.2, 129.8, 129.7, 129.5, 128.4, 128.3, 127.9, 121.5, 120.0, 118.9, 118.5, 110.9, 110.4, 108.4, 107.8, 68.3, 39.0, 31.9, 29.6, 29.1, 22.5, 13.9. HRMS-TOF m/z (M+Na)⁺ calcd 496.2100. found 496.2078.

### 4. Compounds

The compounds in Table 4 were prepared using the methods described herein. The table also shows KD data determined using surface plasmon resonance analysis, and calculated binding energy for binding to GP130.

TABLE 4

| | MDL | | SPR data | Binding Energy* |
|---|---|---|---|---|
| No. | number | Structure | (K_{d} μM) | kcal/mol) |
| 1 | MDL-1 | 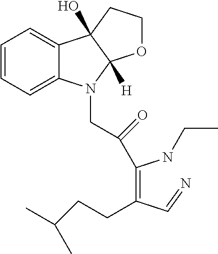 [SMILES: CCN/C(C(=O)CN1c2ccccc2[C@]2(O)CCO[C@H]12)=C(\C=N)CCC(C)C] | n.d. | −7.0 |
| 2 | MDL-2 | 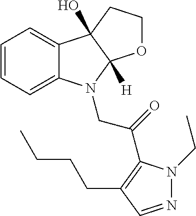 [SMILES: CCCCc1cnn(CC)c1C(=O)CN1c2ccccc2[C@]2(O)CCO[C@H]12] | n.d. | −6.9 |
| 3 | MDL-3 | 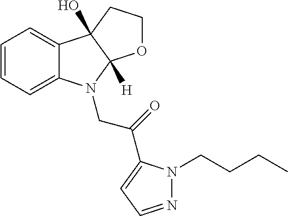 [SMILES: CCCCn1nccc1C(=O)CN1c2ccccc2[C@]2(O)CCO[C@H]12] | n.d. | −6.3 |
| 4 | MDL-4 | 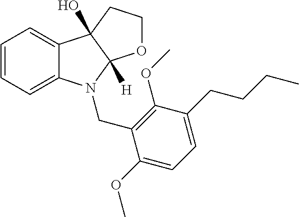 [SMILES: CCCCc1ccc(OC)c(CN2c3ccccc3[C@]3(O)CCO[C@H]23)c1OC] | n.d. | −7.2 |
| 5 | MDL-5 | 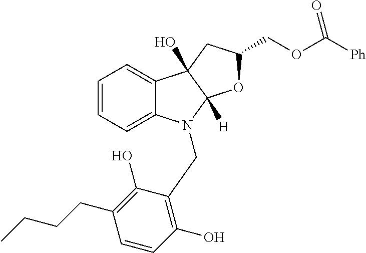 | 36.97 | −9.0 |
| 6 | MDL-6 | 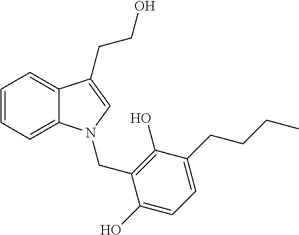 [SMILES: CCCCc1ccc(O)c(Cn2cc(CCO)c3ccccc32)c1O] | 49.50 | −6.5 |
| 7 | MDL-7 | 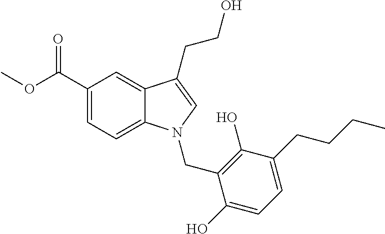 [SMILES: CCCCc1ccc(O)c(Cn2cc(CCO)c3cc(C(=O)OC)ccc32)c1O] | 40.75 | −7.5 |
| 8 | MDL-8 | 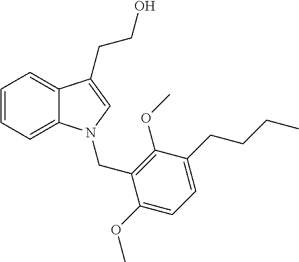 [SMILES: CCCCc1ccc(OC)c(Cn2cc(CCO)c3ccccc32)c1OC] | 41.37 | −6.0 |
| 9 | MDL-16 | 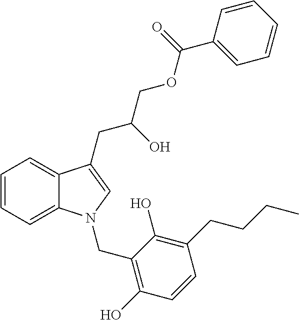 [SMILES: CCCCc1ccc(O)c(Cn2cc(CC(O)COC(=O)c3ccccc3)c3ccccc32)c1O] | 29.00 | −9.2 |
| 10 | MDL-17 | 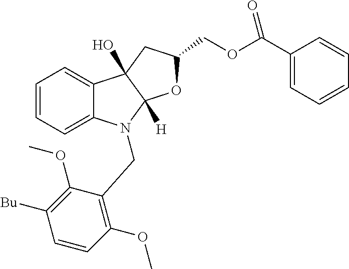 | 42.46 | −6.8 |
| 11 | MDL-18 | 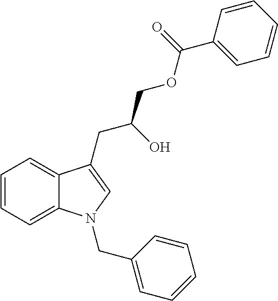 [SMILES: O=C(OC[C@@H](O)Cc1cn(Cc2ccccc2)c2ccccc12)c1ccccc1] | n.d. | n.d. |
| 12 | MDL-21 | 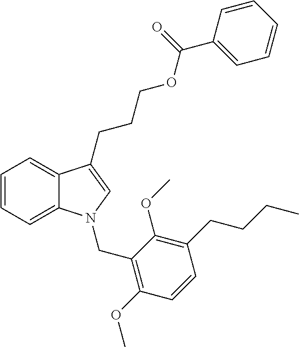 [SMILES: CCCCc1ccc(OC)c(Cn2cc(CCCOC(=O)c3ccccc3)c3ccccc32)c1OC] | n.d. | n.d. |
| 13 | MDL-22 | 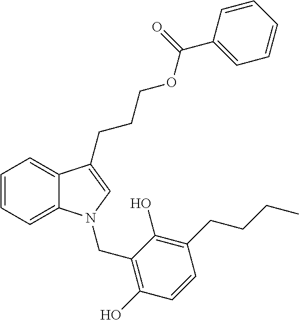 [SMILES: CCCCc1ccc(O)c(Cn2cc(CCCOC(=O)c3ccccc3)c3ccccc32)c1O] | n.d. | n.d. |
| 14 | MDL-23 | 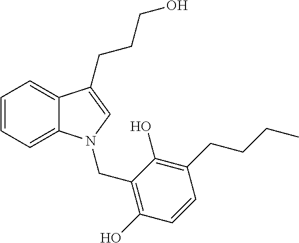 [SMILES: CCCCc1ccc(O)c(Cn2cc(CCCO)c3ccccc32)c1O] | n.d. | n.d. |
| 15 | MDL-24 | 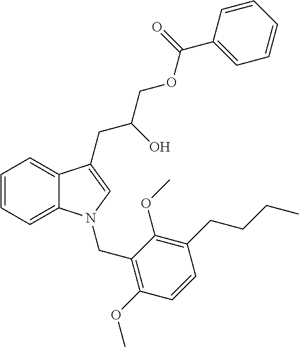 [SMILES: CCCCc1ccc(OC)c(Cn2cc(CC(O)COC(=O)c3ccccc3)c3ccccc32)c1OC] | n.d. | n.d. |
| 16 | MDL-27 | 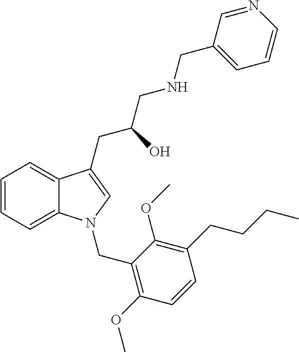 [SMILES: CCCCc1ccc(OC)c(Cn2cc(C[C@H](O)CNCc3cccnc3)c3ccccc32)c1OC] | n.d. | n.d. |
| 17 | MDL-28 | 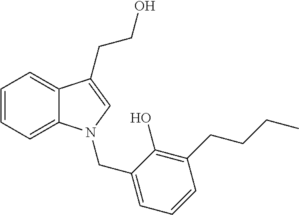 [SMILES: CCCCc1cccc(Cn2cc(CCO)c3ccccc32)c1O] | n.d. | n.d. |
| 18 | MDL-29 | 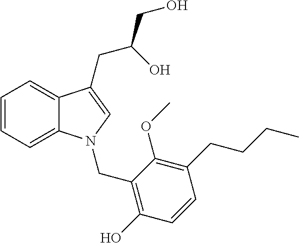 [SMILES: CCCCc1ccc(O)c(Cn2cc(C[C@H](O)CO)c3ccccc32)c1OC] | n.d. | n.d. |
| 19 | MDL-30 | 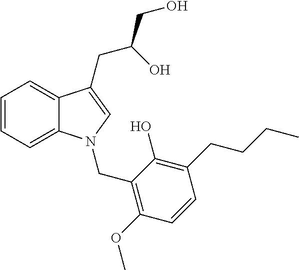 [SMILES: CCCCc1ccc(OC)c(Cn2cc(C[C@H](O)CO)c3ccccc32)c1O] | n.d. | n.d. |

### 5. Molecular Docking Methods

For molecular docking simulations three dimensional structures of GP130 D1, D2 and D3 domains were taken from an X-ray crystal structure of the hexameric assembly of the IL-6/IL-6Rα/GP130 complex (PDBID:1P9M). {Boulanger, 2003 #28} MDL-A was globally docked onto each GP 130 domain separately using AutoDock4.0. {Morris, 1998 #49} {Huey, 2007 #27} During docking simulations MDL-A was kept fully flexible (rotation of bonds) and Gasteiger charges were applied to both the protein and ligand structures. As the binding site for MDL-A was unknown, a grid map of 80×80×75 points with a spacing of 0.375 Å was used, which covered the whole GP130 D1 domain. After the grid box was centered in the domain, grid potential maps were calculated using the module AutoGrid 4.0. The Lamarckian genetic algorithm (LGA), which uses a combination of a genetic algorithm and a local search, was used as the search method. All docked conformations were clustered at RMSD of 1.5 Å.

### 6. Materials: Cell Lines and Culture

LNCaP prostate cancer cells were acquired from American Type Culture Collection. Cells were maintained in RPMI 1640 medium, 1× with 4.5 g/L L-glutamine, supplemented with 10% FBS, 100 U/ml penicillin and 100 mg/ml streptomycin (Invitrogen), in a humidified atmosphere of 5% CO₂ at 37° C.

### 7. Materials: Expression and Purification of GP130

The plasmid expressing gp130-Fc-HA was obtained from Dr. John J. Krolewski (Department of Pathology, University of California, Irvine, Calif.). HEK293T cells were obtained from Dr. Kirk Mykytyn, Department of Pharmacology, OSU). The following antibodies were used in immunoblotting of the recombinant gp130 protein: 1° antibody: Monoclonal antibody HA.11 (Covance), 2° antibody: anti-mouse IgG, HRP-linked Antibody (Cell Signaling Technology). Calcium phosphate transfection kit (Invitrogen), anti-HA affinity matrix and HA peptide (Roche Applied Science).

### 8. Plasmid Purification

Bacteria expressing recombinant gp130 (spotted on filter paper) were obtained from Dr. John J. Krolewski (Department of Pathology, University of California, Irvine, Calif.). Bacteria from the paper were eluted off using SOC (non-selective medium) and colonies were grown by streaking culture on an agar plate. A selective bacterial colony (ampicillin resistant) was grown by picking one colony and growing in LB+100 μg/ml ampicillin. QIAGEN plasmid purification protocol was followed to purify the gp130 plasmid. The purified gp130 plasmid was confirmed by sequencing using primers 5′-ACGCTAGCAGAATCTACAGGTGAAC and 5′-TAGGATCCGCGGCTTCAATTTCTC at The Plant-Microbe Genomics Facilities, OSU. Plasmid concentration (426 mg/ml) was determined by Qubit fluorometer (Invitrogen).

### 9. Expression and Purification of GP130 Protein

HEK293T cells were grown in media containing DMEM+10% fetal bovine serum (FBS),100 U/ml penicillin and 100 mg/ml streptomycin (Invitrogen) in a humidified 37° C. incubator with 5% CO₂. Plasmid DNA expressing gp130-Fc-HA was transfected in the form of calcium phosphate precipitate into HEK293T cells (ten T75 flasks containing 5.5×10⁶ cells). The supernatant was harvested sixty hours post transfection and centrifuged to remove residual cells and adjusted to pH 8.0. The supernatant was passed through a 0.45 μm filter. The gp130-Fc-HA tagged protein was immunoprecipitated using anti-HA affinity matrix (Roche Applied Bioscience).A one milliliter column containing anti-HA affinity matrix was equilibrated with 20 mM Tris-HCl, 0.1M NaCl, 0.1 mM EDTA (pH 8.0). The supernatant was applied to the equilibrated column and the column was washed successively with 20 mM Tris-HCl, 1M NaCl (pH 8.0). Bound protein was eluted with HA peptide dissolved in equilibration buffer. The column was regenerated with 0.1 M glycine (pH 2.0). Fractions containing recombinant protein were pooled, dialyzed against HBS buffer [10 mM HEPES (pH 7.4), 0.15 M NaCl, 3 mM EDTA, 0.005% P20] and concentrated by Centricon 30 (Amicon) centrifugation. Protein concentration was determined using Bradford protein assay reagent (Pierce), with bovine serum albumin (BSA, Fisher Scientific) as a standard. The purified recombinant gp130 protein was analyzed using SDS-PAGE and immunoblotting.

### 10. Immunoblotting of GP130

Purified gp130-Fc-HA protein was incubated with reduced loading buffer for 5 minutes at 95° C. and electrophoresed on 10% acrylamide-SDS gel in 1×TGS buffer (0.025 M Tris, 0.192 M glycine, 0.1% SDS). Proteins were electrotransferred to PVDF membranes; the transfers were carried out under 100 V, 350 mA for one hour in a cooled reservoir containing 25 mmol/L Tris, 192 mmol/L glycine and 20% methanol (pH 8.3) transfer buffer. The membranes were then removed and placed in Ponceau S staining solution (0.5% Ponceau 5 and 1% glacial acetic acid in water). The membrane was subsequently washed and blocked with 5% nonfat dry milk in TBST (60 mM Tris-base, 120 mM NaCl, 0.1% Tween-20) for at least one hour. The membrane was incubated with monoclonal antibody HA.11 (Covance) in 5% nonfat dry milk in TBST overnight at 4° C. Blots were then washed three times in TBST and incubated with anti-mouse IgG, HRP-linked secondary antibody (Cell Signaling Technology in 5% nonfat dry milk in TBST for one hour at room temperature. The bound antibody was detected using Enhanced Chemiluminescence detection reagents (Pierce) according to the manufacturer's instructions. Chemiluminescence was visualized using ECL Hyperfilm (Amersham).

### 11. Surface Plasmon Resonance Analysis

Surface plasmon resonance (SPR) analysis was performed using BIAcore T100. Recombinant gp130-Fc-HA was cross-linked to the flow cell on a carboxymethylated dextran matrix of a CM5 sensor chip (BIAcore) using standard amino group coupling methods according to the manufacturer's instructions. Sodium acetate (10 mM, pH 3.5) gave the best immobilization of the protein on the chip surface and unreactive groups on the chip were blocked by ethanolamine according to the manufacturer's instructions. Approximately 5000 resonance units (RU) of gp130-Fc-HA were cross-linked to the flow cell. HBS+1% DMSO were used in all buffers (running and sample) to avoid background response due to differences in buffer refractive index. For the equilibrium analysis, various concentrations of MDL-A, 1, and 2 were injected for a period of 2 min at a flow rate of 30 μl/min, in series, into flow cells containing covalently bound gp130-fc-HA and reference flow cell. Bound ligand (MDL-A, 1, and 2) in each assay was removed by passing 10 mM glycine (pH 2.2) over the chip surface. Biacore T100 Evaluation 2.0 was used for all interaction analyses. The steady-state equilibrium response (RUeq) was determined from the reference-subtracted sensogram. The kinetics parameter was calculated according to a 1:1 Langmuir binding model (A+B [symbol] AB) by direct fitting of ligand binding sensor grams at multiple concentrations. The dissociation equilibrium constant is defined as dissociation constant (K_{D})=dissociation rate constant (k_{d})/association rate constant (kₐ). K_{D} was determined by scattered analysis of equilibrium-state data obtained at different concentrations of analogues.

### 12. Cell Culture and Lysate Preparation for IL-6 Induction of Stat3 Phosphorylation

LNCaP prostate cancer cells were seeded in T75 flasks, grown to 50% confluence and serum-starved the following night. The cells were left untreated or were treated with MDL-A (100-400 μM), 1, 2 (5-40 μM) or DMSO in the absence of FBS. After 4 hours, the cells were stimulated with 12.5 ng/ml IL-6 (Cell Signaling Technology). The cells were then harvested at 30 minutes and washed with PBS and lysed in cold M-PER lysis buffer (Pierce) containing protease inhibitors (Sigma) and phosphatase inhibitors (Roche). Lysates were centrifuged at 14,000 rpm for 15 minutes at 4° C. and supernatants were collected and stored at −20° C. until further use.

### 13. Immunoblotting Methods

Cell lysates were assayed for their protein concentrations using the Bradford protein assay reagent (Pierce), using BSA (Fisher Scientific) as the standard. Lysate samples were adjusted for the same protein concentration, approximately 5 μg of sample protein was loaded into each of the appropriate lanes. Protein samples were separated by SDS-PAGE and electrotransferred to PVDF membranes. The membranes were washed and blocked with 5% BSA in TBST (60 mM Tris-base, 120 mM NaCl, 0.1% Tween-20) for at least one hour and incubated with anti-phospho-STAT3 (p-Tyr705) antibody, anti-STAT3 antibody or anti-GAPDH antibody (all from Cell Signaling Technology) in 5% BSA in TBST overnight at 4° C. Blots were then washed three times in TBST and incubated with a horseradish peroxidase-conjugated goat anti-rabbit IgG secondary antibody (Cell Signaling Technology) in 5% BSA in TBST for 1 hour at room temperature. The bound antibody was detected using Enhanced Chemiluminescence detection reagents (Pierce) according to the manufacturer's instructions and chemiluminescence visualized using ECL Hyperfilm (Amersham).

### 14. Computational Analysis and Structure Based Design

In order to design potent, specific, synthetically tractable and drug-like small molecules, a new strategy is urgently needed. As described herein, MDL-A was globally docked computationally to the extracellular D1, D2 and D3 modular domains of GP130, as the D2 and D3 domains are responsible for heterotrimer formation and the D1 domain is responsible for trimer homodimerization. It was determined that MDL-A binds to the D1 domain and not the D2 or D3 domains, thus confirming that the compound disrupts GP130 dimerization. The docking free energy is −6.4 Kcal/mol, close to the experimental −5.0 Kcal/mol, which is considered as weak binding (binding free energies estimated from current docking programs have a 2-3 Kcal/mol standard deviation). In this case, AutoDock4²³ was used to carry out the docking simulation using a GP130 structure from the crystal structure of the heterohexamer of IL-6/IL-6R/GP130 (PDB ID: 1P9M).²⁴ FIG. 4 shows its binding mode to the D1 domain and how it prevents IL-6 binding to GP130, thus disabling the functional dimerization of the heterotrimers (see Figure legend for details). With the structural binding model established, our aim is to use a structure-based computation-assisted approach to design more potent and specific, drug-like synthetic small molecules to mimic the MDL-A interactions with GP130 and additional interactions utilizing the extra binding subpocket.

Analysis of the structure of madindoline A (MDL-A) and the computational model of its binding to the gp130 D1 extracellular domain has highlighted key structural features. To design novel derivatives with increased potency and selectivity, modifications through structure-based strategy can be used. For the start, two optimizations were addressed: a) improved synthetic efficiency. Fragment-based design methods were used to search for new fragments to replace the pentendione ring. With AlleGrow²⁵, hydroxylbenzyl and pyrazole rings were identified (see FIG. 6). b) improved potency/selectivity via targeting additional D1 domain binding subpocket. As shown in both FIGS. 4 and 5, additional fragments can be designed to bind to the extra subpocket. CombiGlide²⁶ was used to search a fragment library with 6000 fragments and came up with several options. FIGS. 5 and 7 show two possible choices. As shown in FIG. 5, the optimized analogues bind exactly as MDL-A with all its binding features preserved, except that the “southern” half of the molecules is easier to be synthesized and the extra subpocket is occupied plus additional hydrogen bond to G1n78 side chain. With hydroxybenzyl binding to the extra subpocket and the benzyl- and pyrazole-substituted “southern” half (see FIG. 7, compounds C and G), the binding free energies are −8.2 Kcal/mol and −8.6 Kcal/mol, respectively. These translate to 21- and 41-fold stronger affinity to GP130 compared to MDL-A, respectively. The analogues, therefore, can feature the addition of functional groups to the “northern” hydroxyfuroindoline portion of the molecule and/or replacement of the “southern” pentendione ring with benzyl or 5-acylpyrazole derivatives.

The computational design of compounds has been carried out through docking and analysis of the gp130 model with a variety of possible substrates based on pyrazole and benzyl modifications to madindoline A. This has been carried out using both Glide and AutoDock. Protein dynamics simulations have demonstrated that the docking site of madindoline A is somewhat flexible. When examined over a period of time, the hydroxyfuroindoline portion of madindoline A is found to “fly out” of the binding pocket, indicating a weaker binding interaction. The cyclopentendione ring containing the butyl chain, however, is held relatively tightly in place. This indicates that this is a stronger interaction and can be involved in binding. This data also supports the results found by the group at UC Irvine (1), which suggested that the hydroxyfuroindoline portion of madindoline A was not capable of binding to gp130 on its own. Finally, the docking models shows that taking advantage of an additional hotspot on gp130 by adding a substituent to the hydroxyfuroindoline ring should increase interactions with the protein and hold this portion of the molecule more tightly to the protein binding pocket. This finding was validated with the synthesis and biological testing of MDL-5.

Computational design showed the synthesis of two key classes of analogues (FIG. 6) to simplify the lower half of the natural product while retaining binding affinity. An example of the systematic modification of the top half of the molecule is shown in FIG. 7. The order of synthesis can proceed from left to right in each series as complexity increases with the addition of each substituent. In addition to the eight analogues shown in FIG. 7, additional analogues can be designed and synthesized. The synthetic approach can rely upon disconnection of the molecules into two halves. This is illustrated for the synthesis of a selected pyrazole analogue in FIG. 8. One bond forming reaction is the alkylation of the hydroxyfuroindoline nitrogen with an activated alkylhalide. Although there is some precedent for the alkylation of indolines with activated halides,²⁹ model studies have been carried out to determine the feasibility of this strategy. For example, alkylation of hydroxyfuroindoline 2 with t-butyl bromoacetate in DMF with potassium carbonate provided the desired N-alkylated product in 51% yield. Additionally, alkylation with benzyl bromide can also be used.

Retrosynthetically, the approach to generate analogues relied upon disconnection of the molecules into two halves, the hydroxyfuroindoline portion and a “southern” pyrazole or benzyl-containing portion. An efficient synthesis of the “northern” hydroxyfuroindoline portion of madindoline employing the Sharpless epoxidation and concomitant cyclization of tryptophol has been reported by Smith and coworkers (2). This strategy was employed for some of the analogues synthesized

The pyrazole fragments were synthesized as described in Scheme 1 below. Pyrazole was readily alkylated using an alkyl iodide, either iodoethane or iodobutane. The alkylated pyrazoles were then formylated at the C4 position using Vilsmeier-Haack conditions. At this stage, Wittig olefination of the aldehyde and hydrogenation of the resulting olefin was employed to introduce the alkyl chain. The pyrazole could then be functionalized regioselectively at the C5 position upon treatment with n-butyllithium and acetaldehyde. The resulting alcohol was subsequently oxidized to the methyl ketone with PDC. Bromination of the ketone could then be accomplished with pyrrolidine tribromide, providing the substrate for alkylation with the hydroxyfuroindoline. Conversely, attempts to directly introduce the brominated acetyl group using bromoacetyl bromide resulted in only poor yields of the desired bromide.

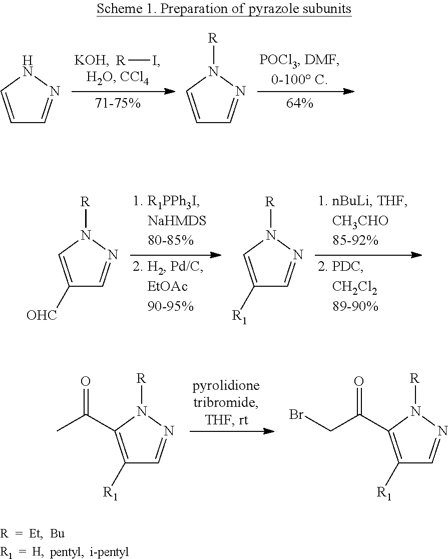

The synthesis of the benzyl fragment required for the second series of analogues is shown in Scheme 2 below. In this case, the synthesis began with protection of 2,4-dihydroxybenzaldehyde as the methoxymethyl (MOM) ether derivative. Conversion of the aldehyde to the styrene derivative via Wittig olefination and subsequent hydrogenation produced the resorcinol derivative in a manner analogous to the introduction of the pyrazole alkyl chain. This compound could then be formylated by lithiation and trapping of the anion with DMF. This aldehyde would serve as the substrate for a reductive amination reaction with the hydroxyfuroindoline. Alternatively, reduction of this aldehyde to the primary alcohol followed by bromination of the resultant alcohol gave rise to the corresponding bromide which could be employed in an alkylation reaction (again similar to the plan for the pyrazoles, above).

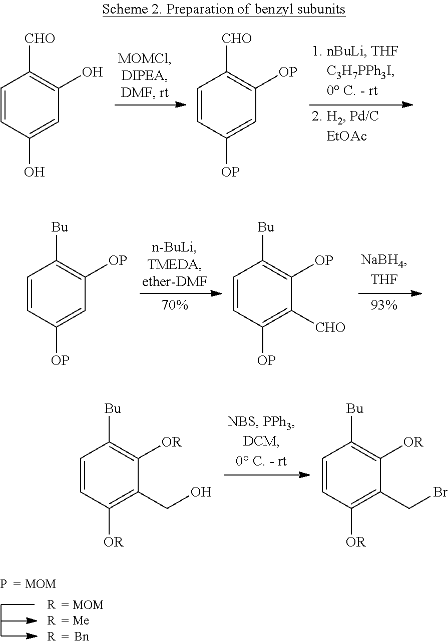

The combination of the HFI unit and the “southern” pyrazole half, was initiated by alkylation of aryl halide in the case of MDL-1-MDL-3 (Scheme 3 below), albeit in relatively low yield. In the case of the benzyl analogues, this was initially accomplished via reductive amination of the benzaldehyde with the aniline nitrogen of the HFI moiety. Not surprisingly, however, the acidic conditions caused the tricyclic ring system to open up and upon elimination of the hydroxyl group, also provided the corresponding tryptophol derivatives (e.g., MDL-8, Scheme 4). The major obstacle in the synthesis of the benzyl analogues, however, has been the late-stage deprotection of the phenols. Me and Bn protecting groups have also been explored.

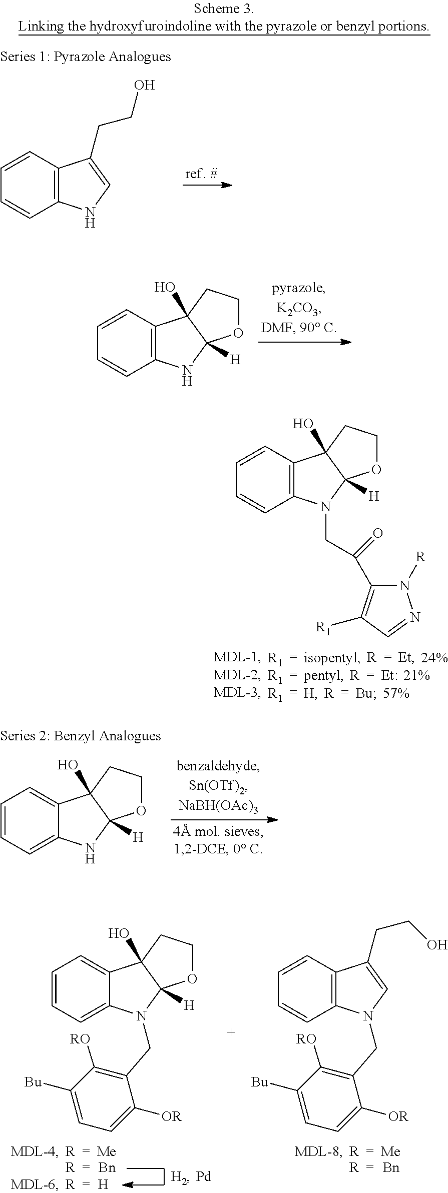

In order to test the hypothesis that adding substituents to the hydroxyfuroindoline ring would improve gp130 binding, synthesized MDL-5 was synthesized. This was accomplished by using ethyl glycidate to introduce a stereogenic center. In this case, however, Sharpless epoxidation failed to provide the desired product in sufficient yield. Alternatively, the epoxidation was carried out using mCPBA, resulting in the production of 2 diastereomeric products. Upon reduction, acylation, and deprotection of the indole nitrogen, these products were separable by column chromatography. Finally, alkylation and hydrogenation provided MDL-5.

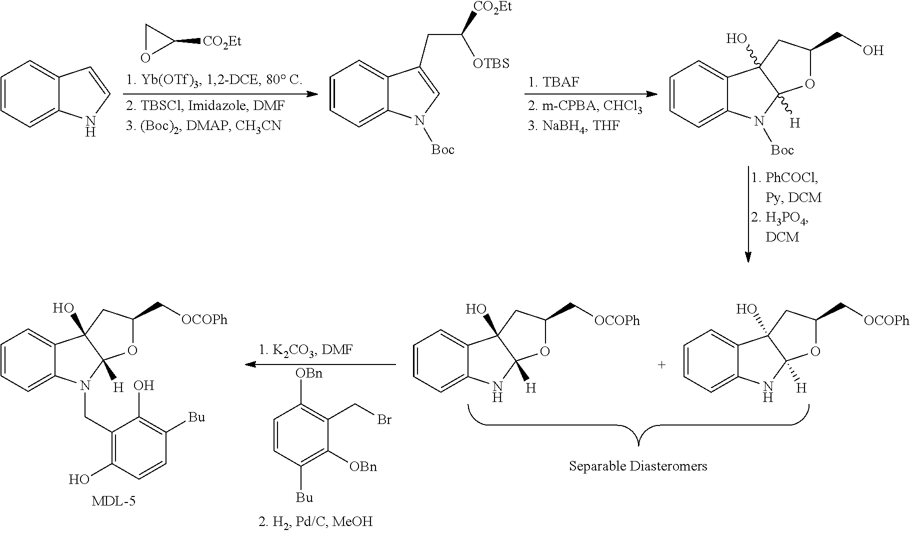

### 15. Purification of GP130 Extracellular Domain

MDL-A has been reported to show direct binding at extracellular domain of gp130 by Saleh, et. al.³ To determine whether our designed MDL-A analogues binds directly the extracellular domain of gp130, we purified recombinant gp130 protein (gp130-Fc-HA) by expressing in HEK293T cells and purified by protein A affinity chromatography from the medium of transfected cells, as described in method section (FIG. 13 A). Immunoblotting with an anti-HA antibody confirmed that the major species corresponded to the predicted gp130 protein (FIG. 13B).

### 16. Direct Binding Measurements of Disclosed Compounds to GP130 Protein Using Surface Plasmon Resonance Technique

To examine the direct binding and calculation of equilibrium dissociation constant (K_{D}) of MDL-A analogues with gp130 extracellular domain, surface plasmon resonance analysis was done. Protein was covalently cross linked to the dextran matrix of the biosensor chip CM5. Successively, various concentrations of MDL-A and MDL-A analogues were injected into the flow cells containing bound protein and no protein (reference). Interactions were monitored in real time and K_{D} values were calculated by reference-substrated sensogram. K_{D} values were calculated using binding affinity analysis program in Biacore evaluation software version 2.0 (shown in Table 4).

TABLE 4

| | AutoDock's Binding | |
|---|---|---|
| MDL-Analogues | Energy (Kcal/mol) | K_{D} (μM) |
| MDL-A | −6.0 | 288 |
| MDL-4 | −7.2 | N/A |
| MDL-5 | −9.0 | 36.97 |
| MDL-6 | −6.5 | 49.5 |
| MDL-7 | −7.5 | 40.75 |
| MDL-8 | −6.0 | 41.37 |

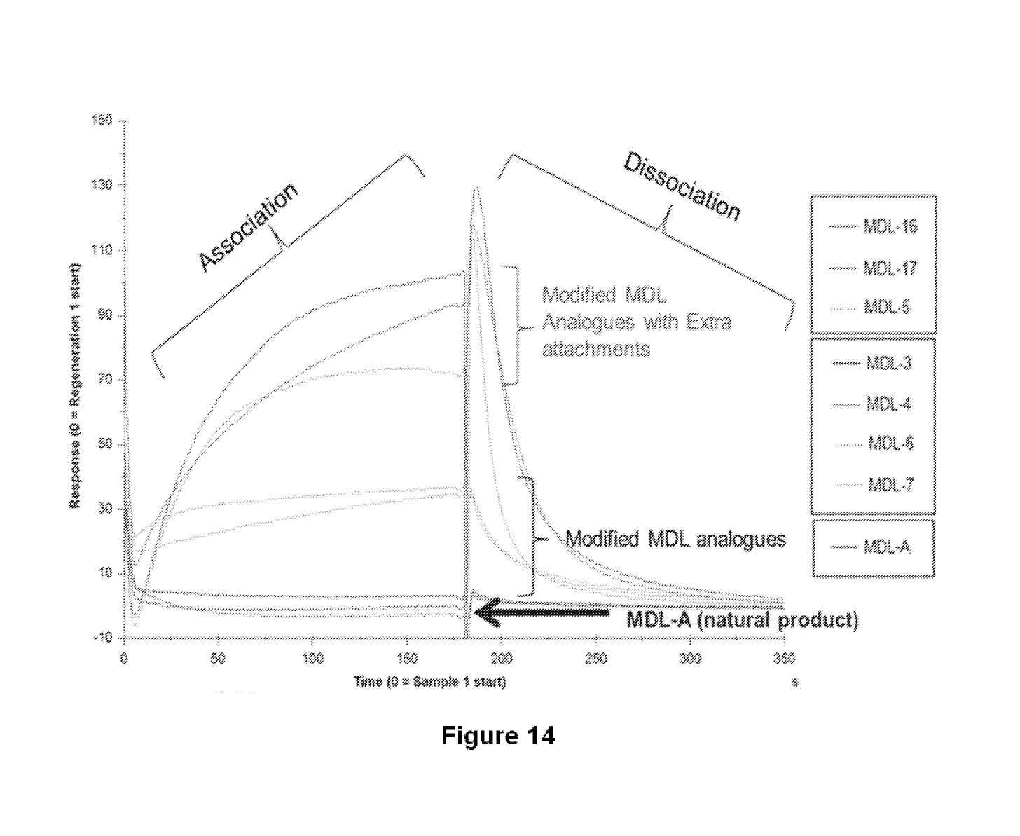

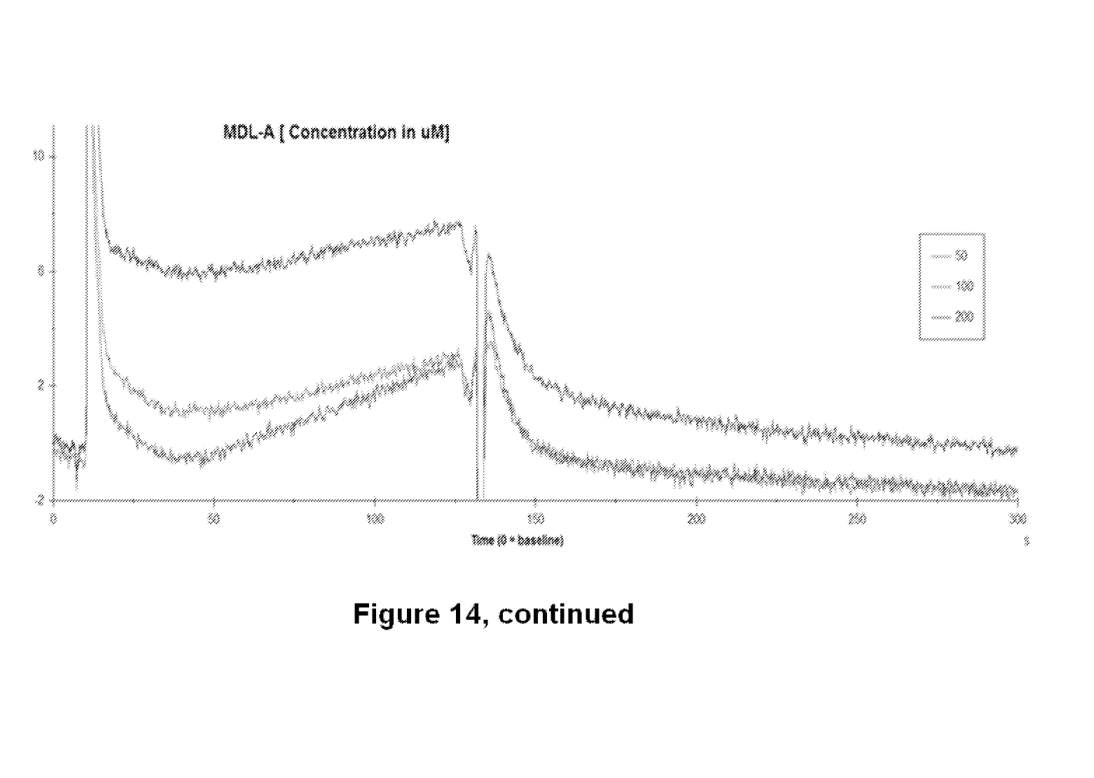

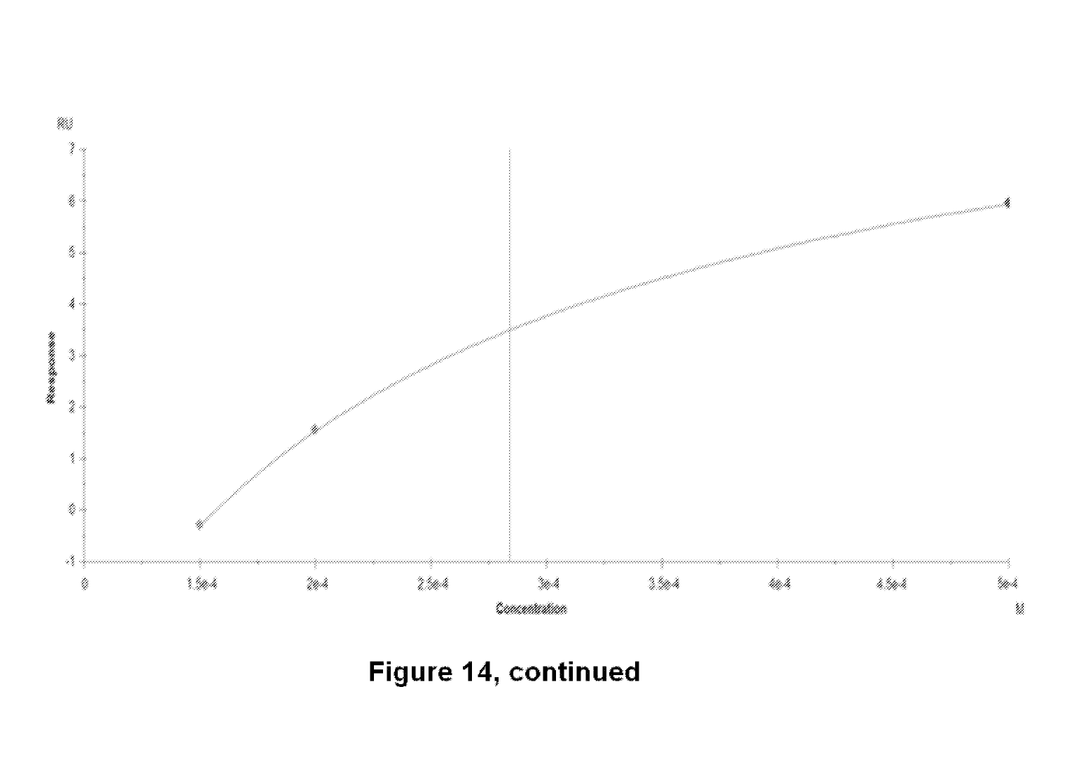

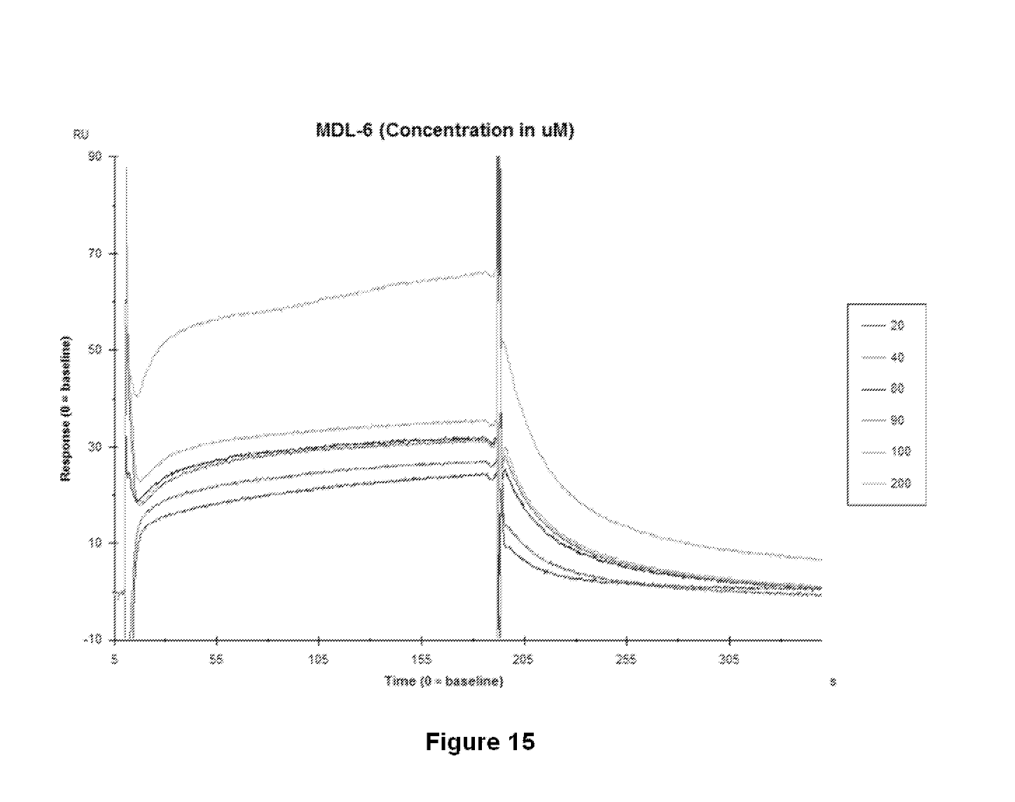

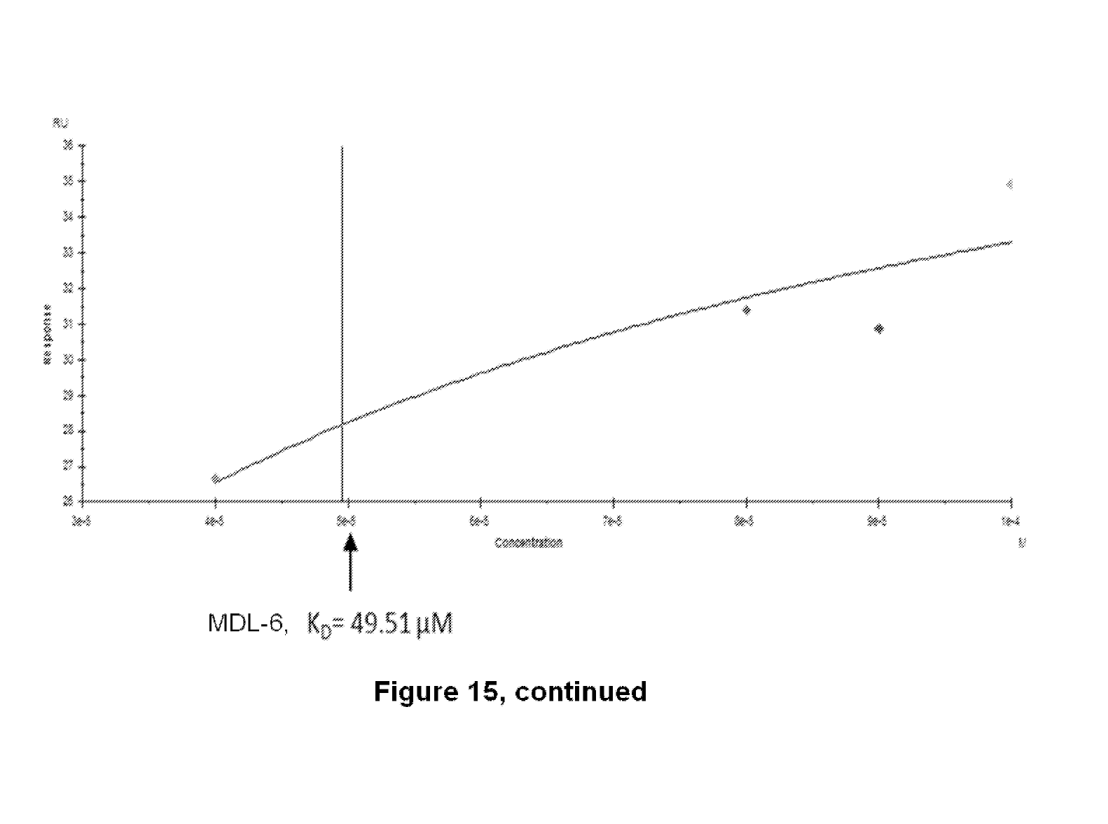

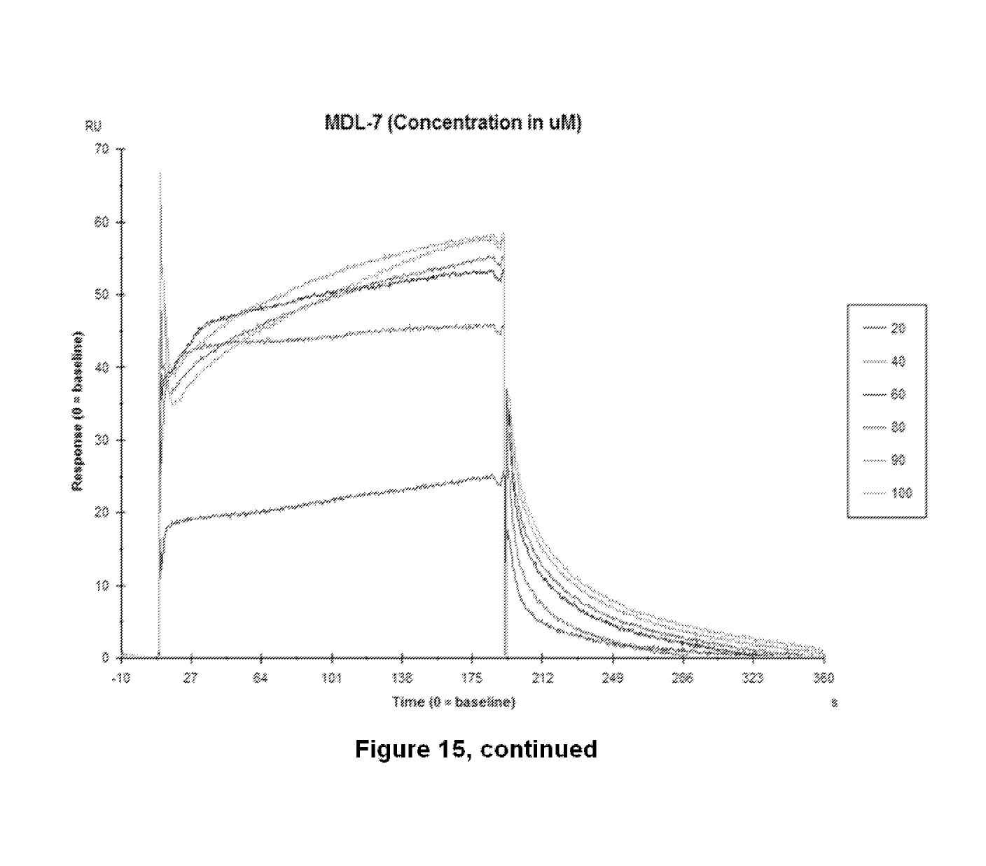

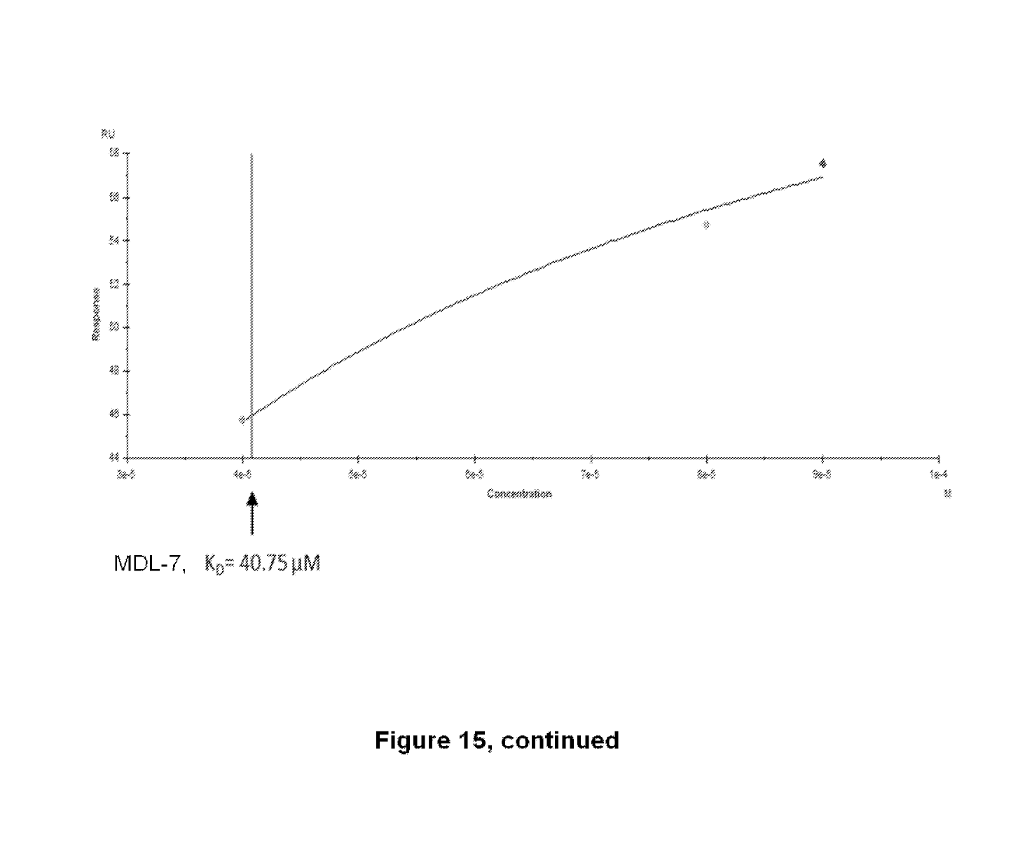

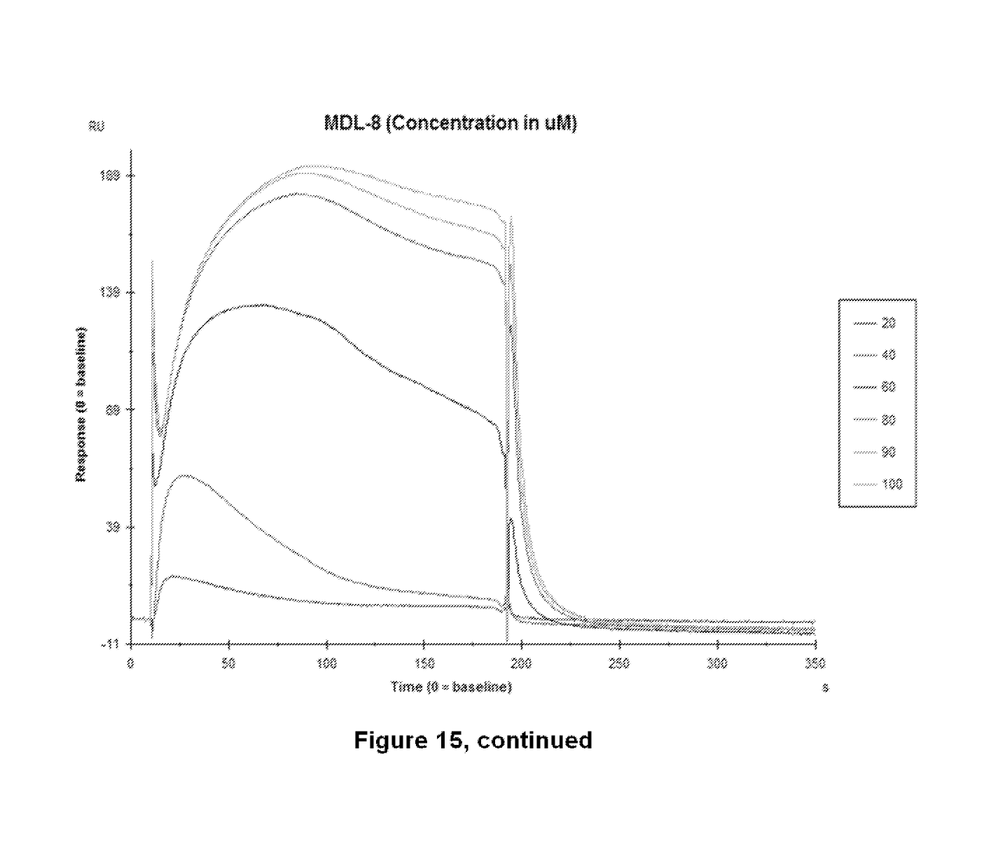

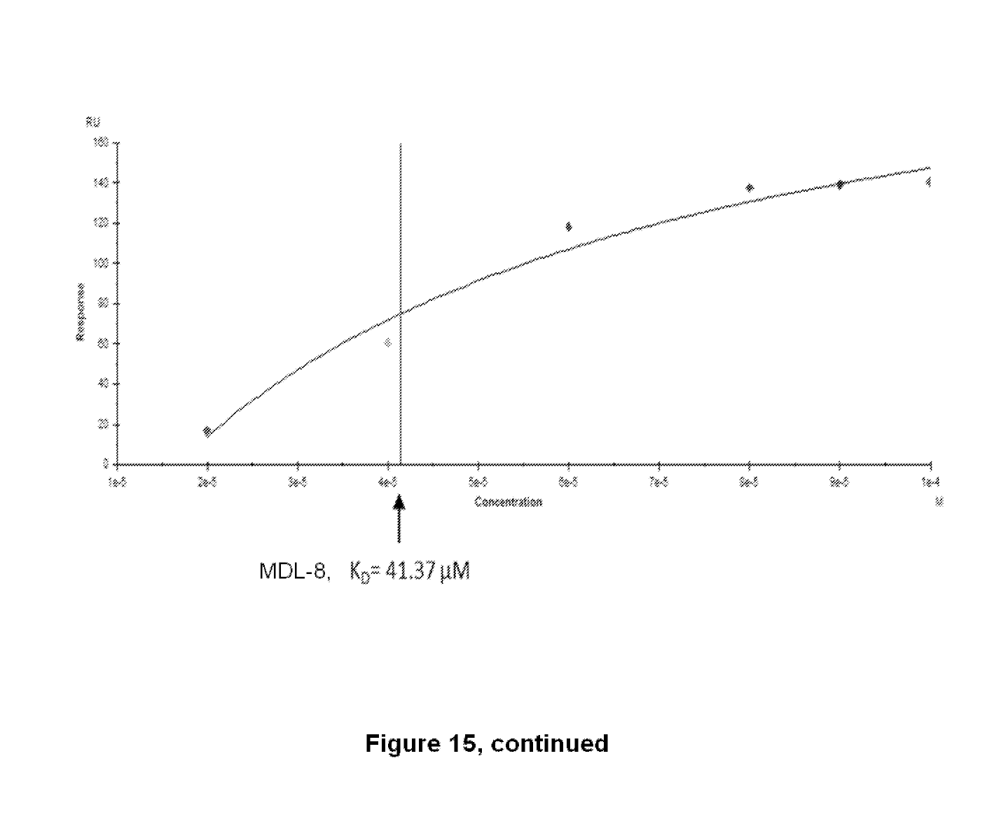

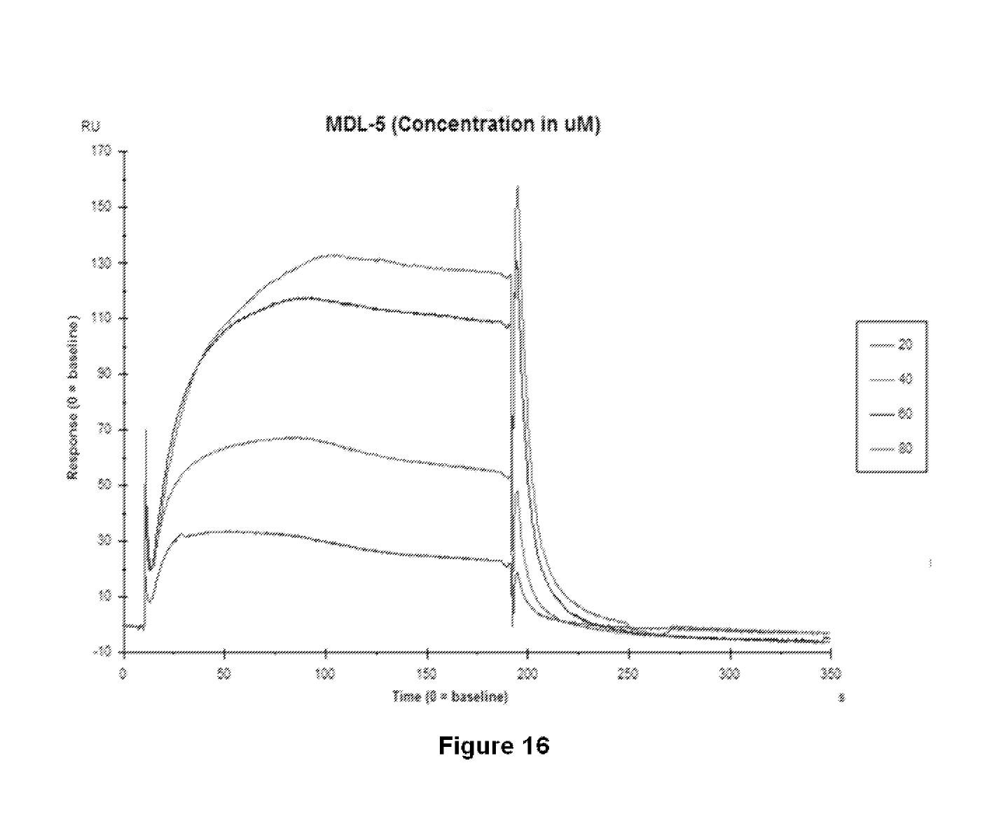

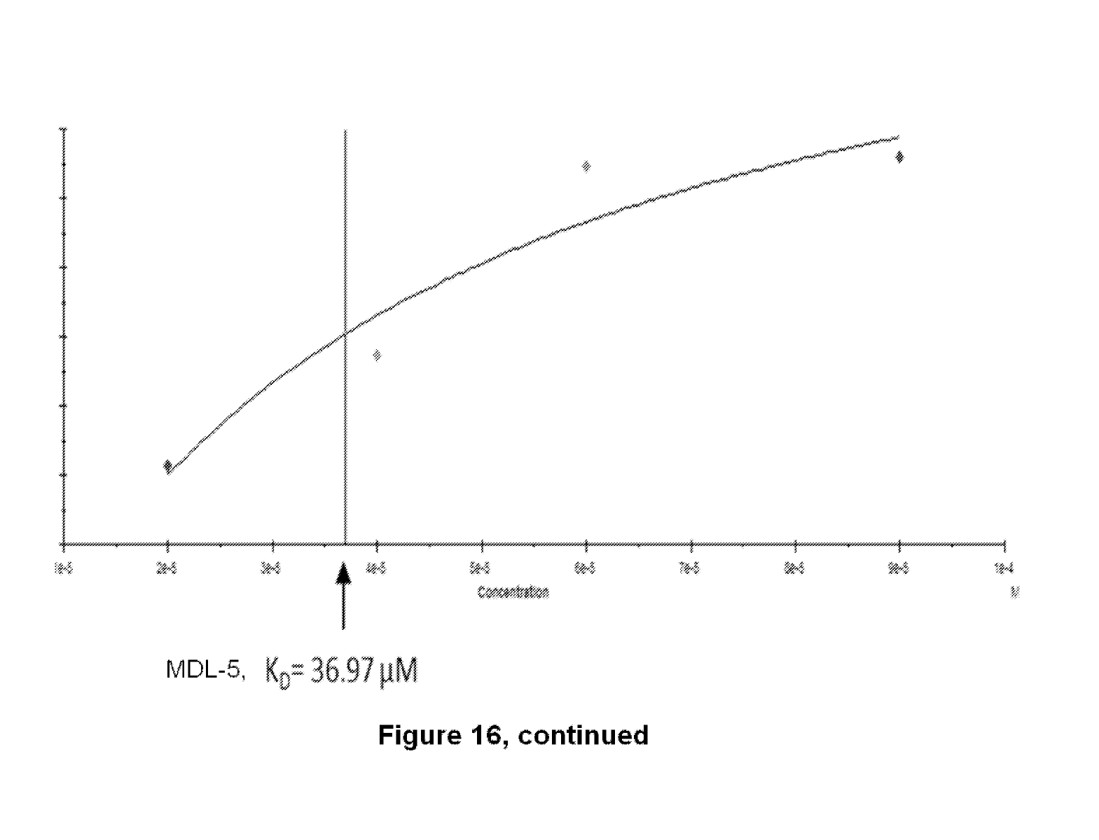

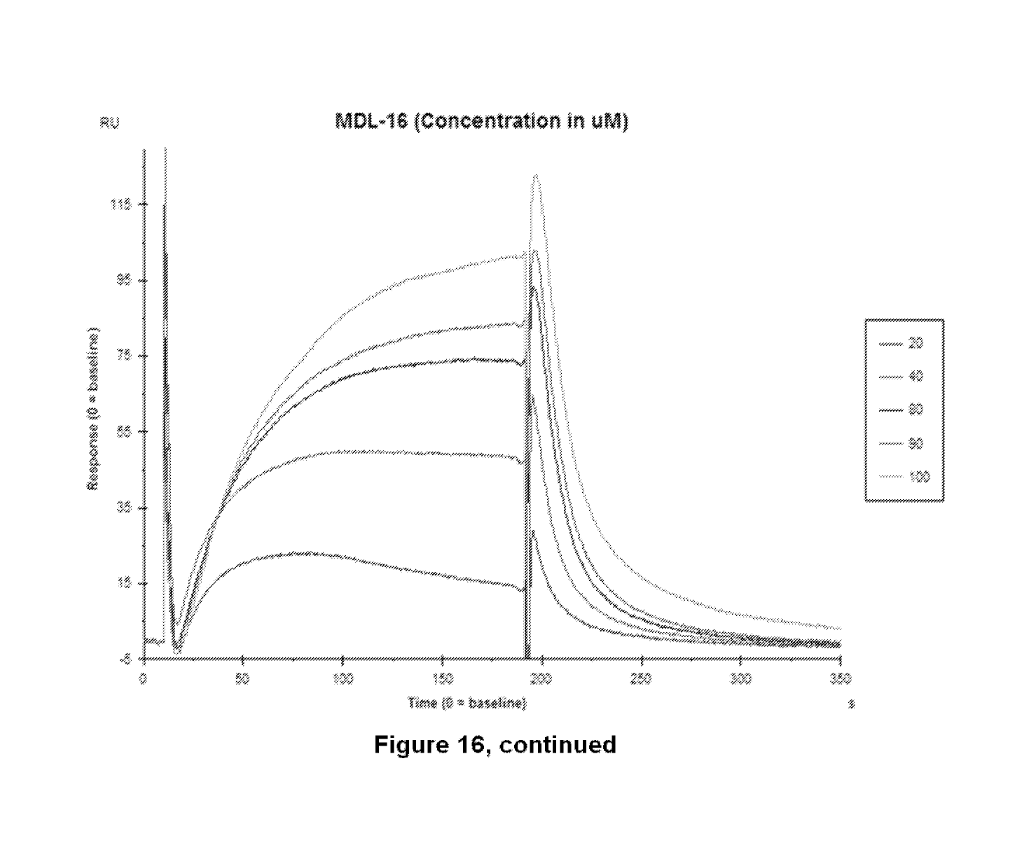

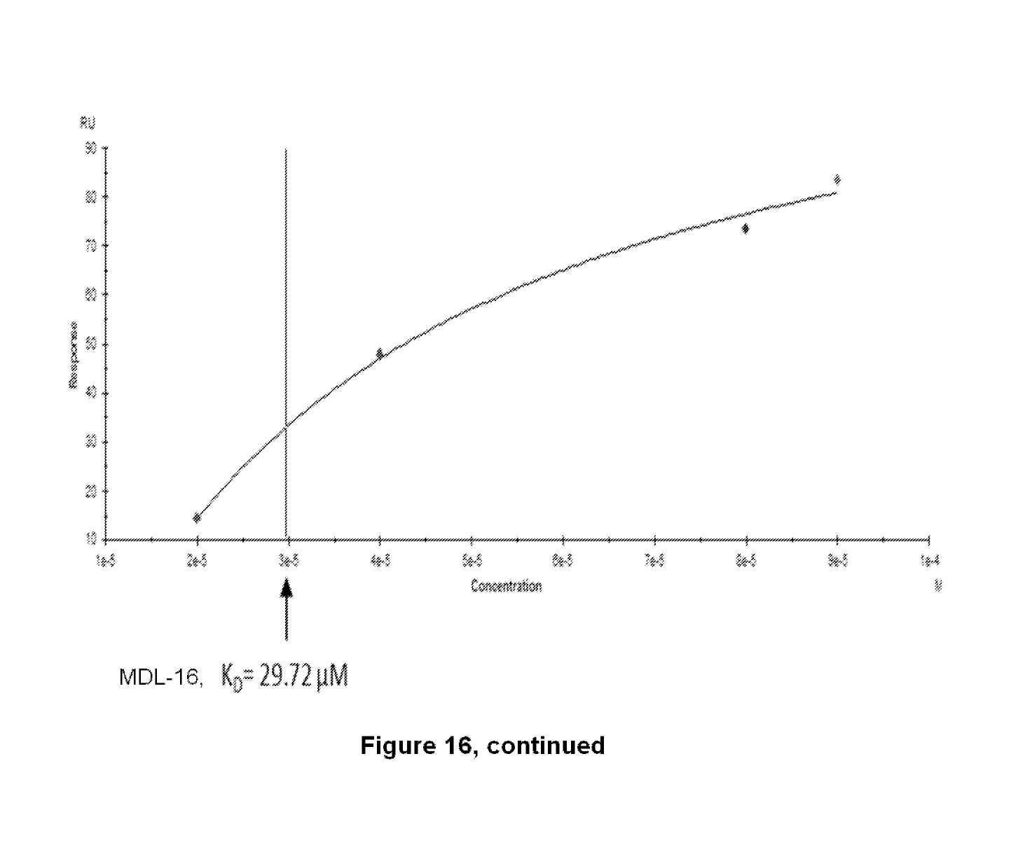

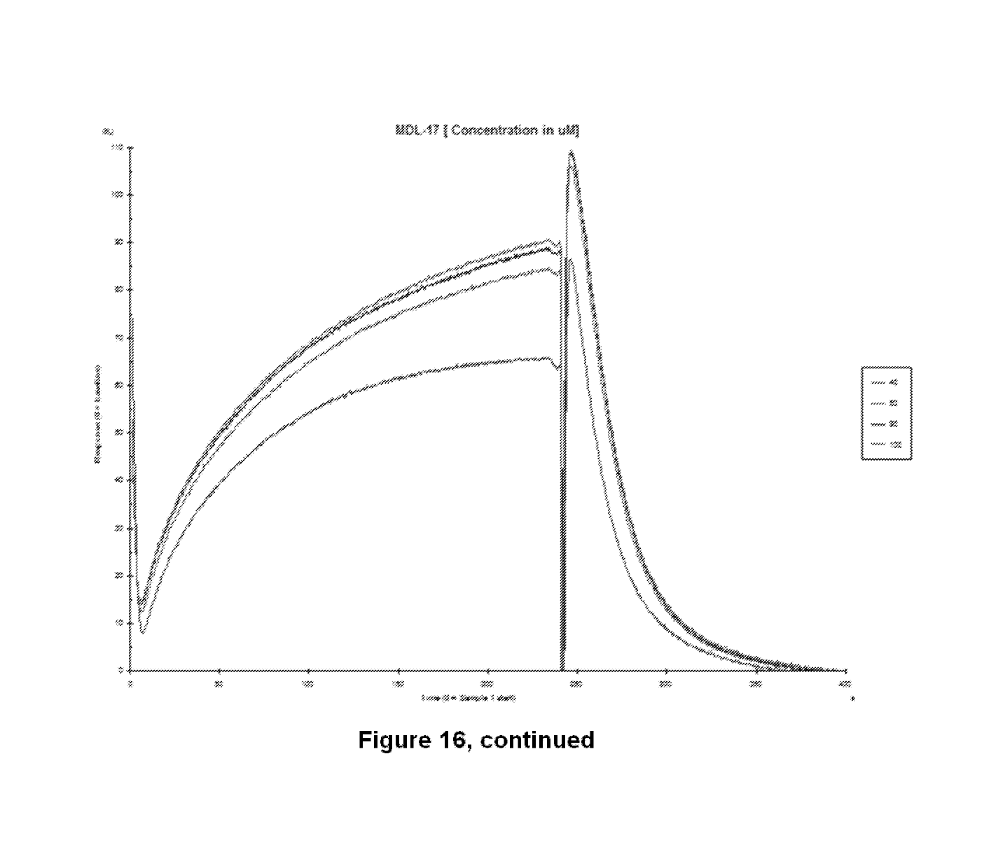

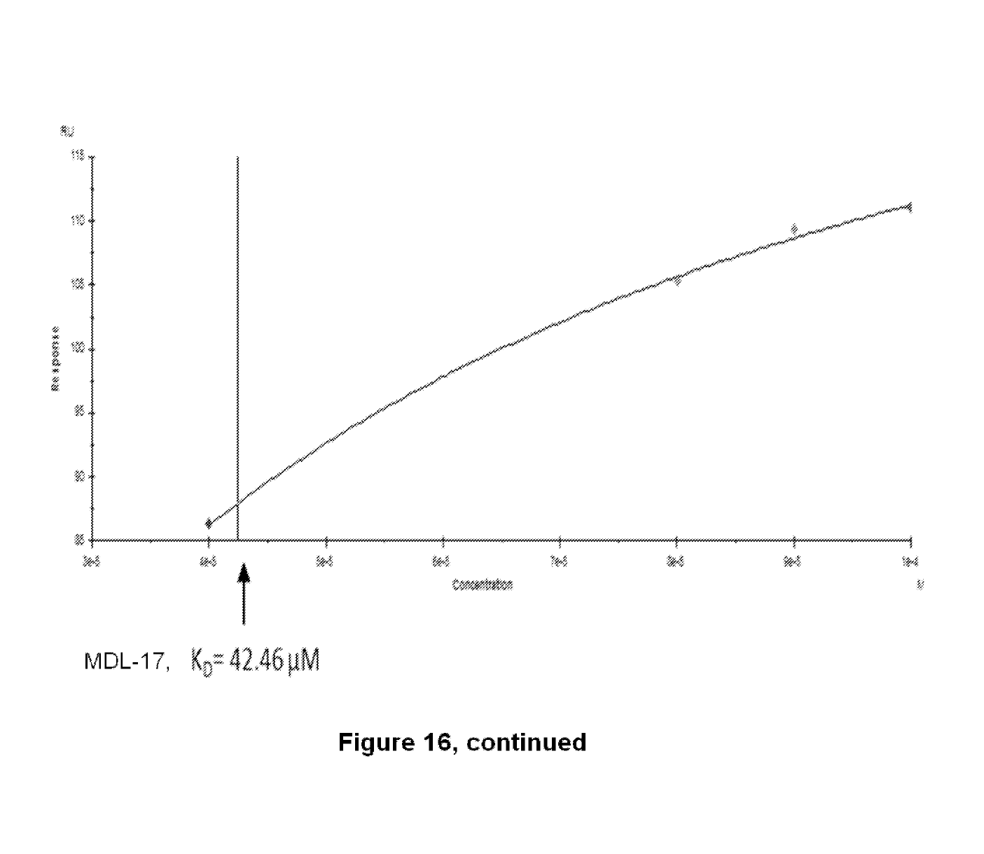

The SPR data (shown in FIGS. 14, 15 and 16), confirmed binding of designed analogues to gp130 extracellular domain with stronger affinity than MDL-A. Analogues with extra attachment group (MDL-5, MDL-16 and MDL-17) showed stronger binding than analogues without extra attachments (MDL-3, MDL-4, MDL-6, MDL-7 and MDL-8), data shown in Table 1a. Among all analogues MDL-16 showed strongest binding affinity (K_{D}=29.72 μM). K_{D} of MDL-A was about 300 μM which is consistent with the previously reported K_{D} of MDL-A by Saleh, et al.³

### 17. Inhibition of Stat3 Phosphorylation by MDL-A and MDL-A Analogues

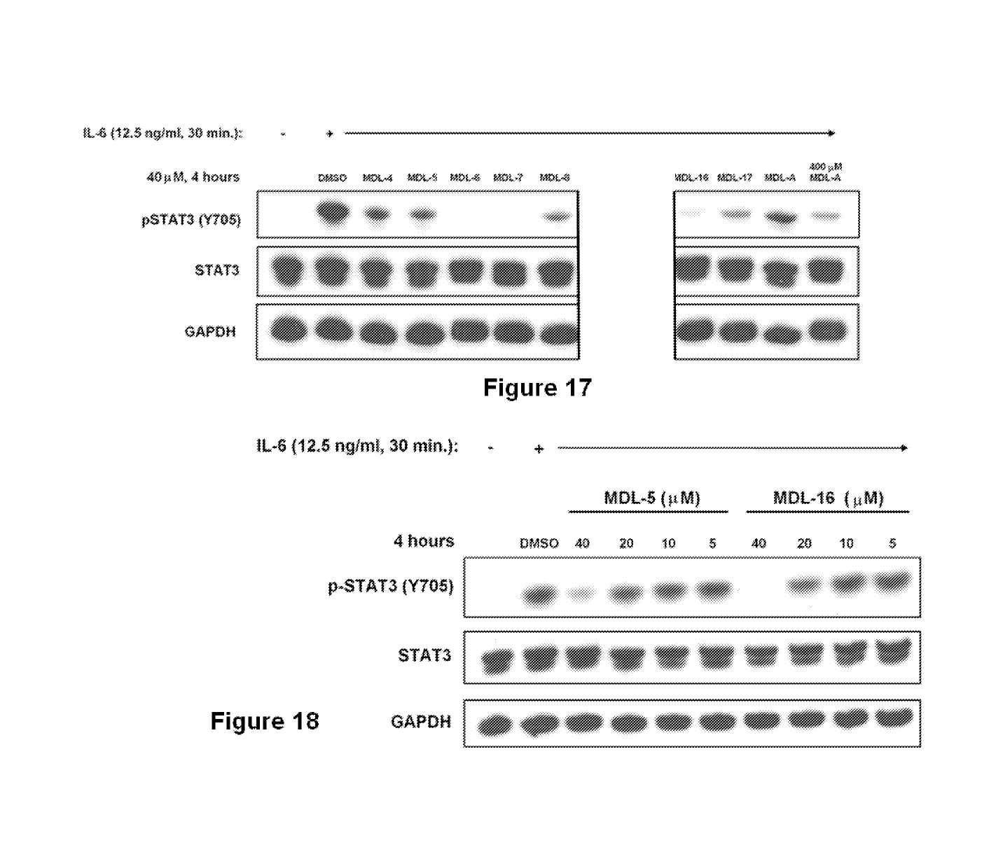

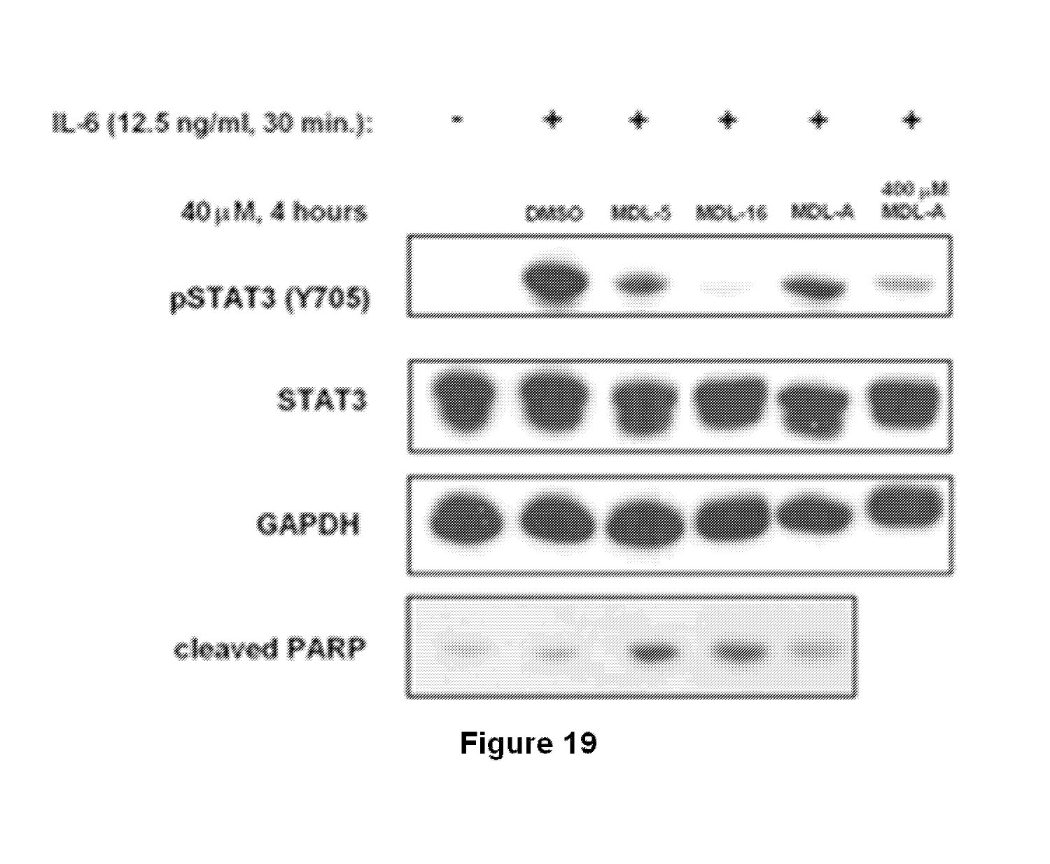

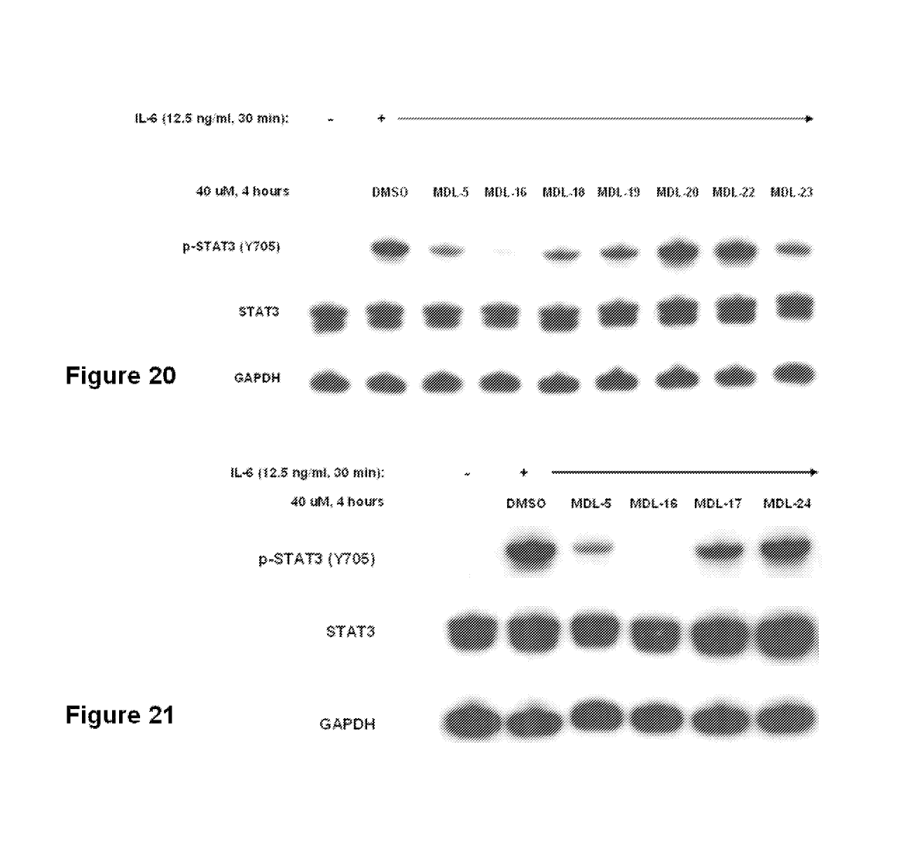

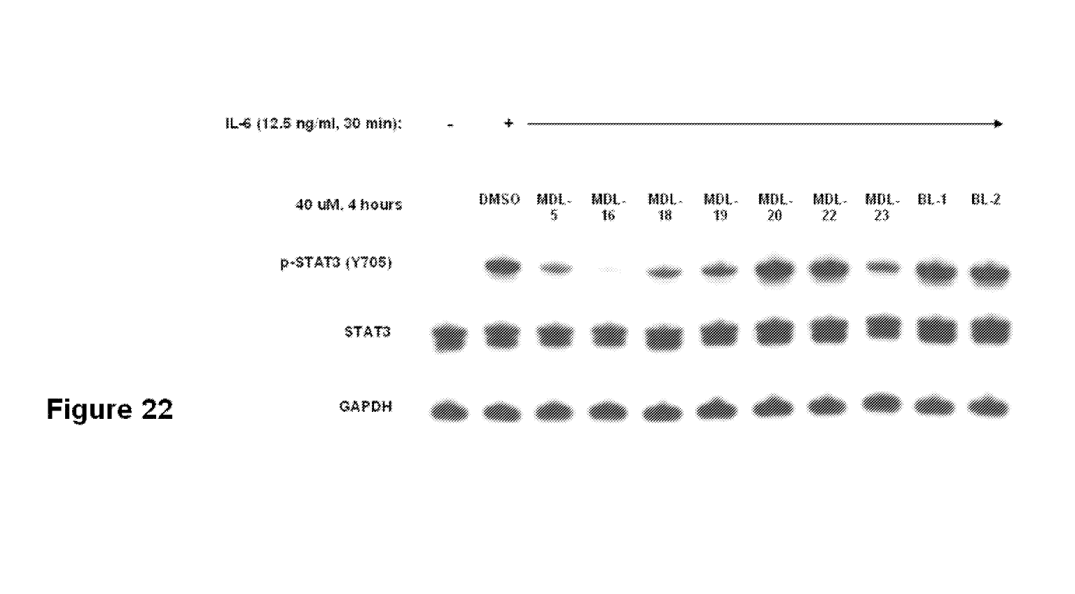

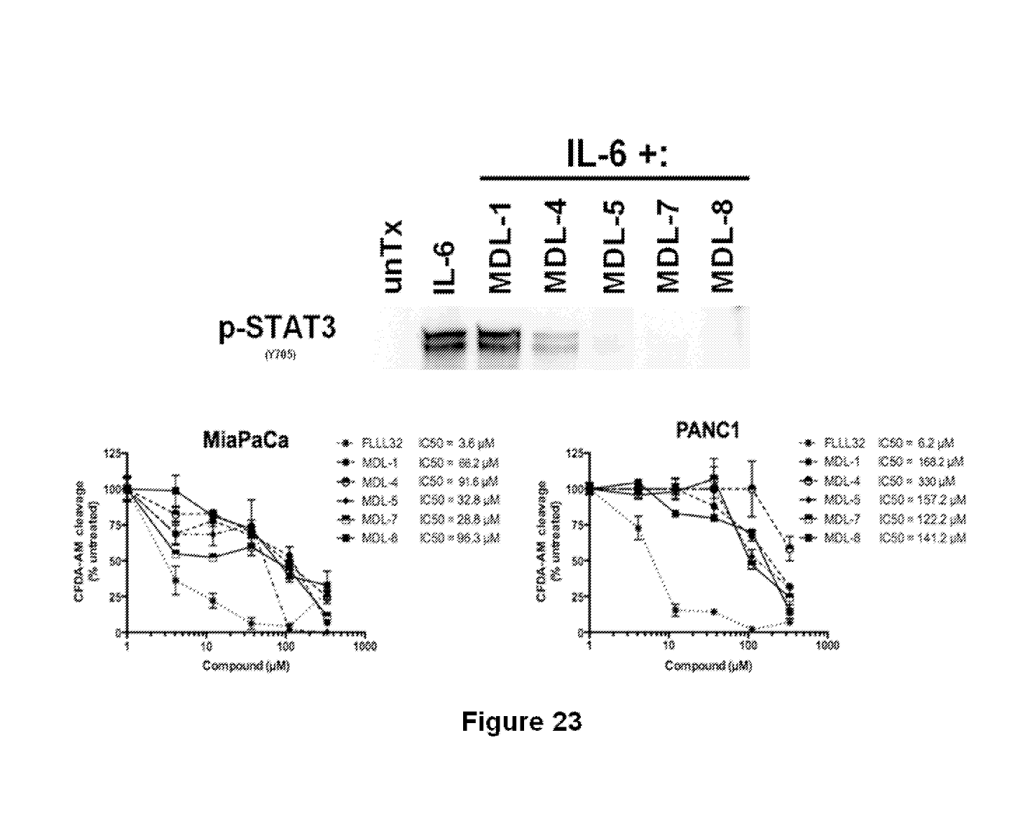

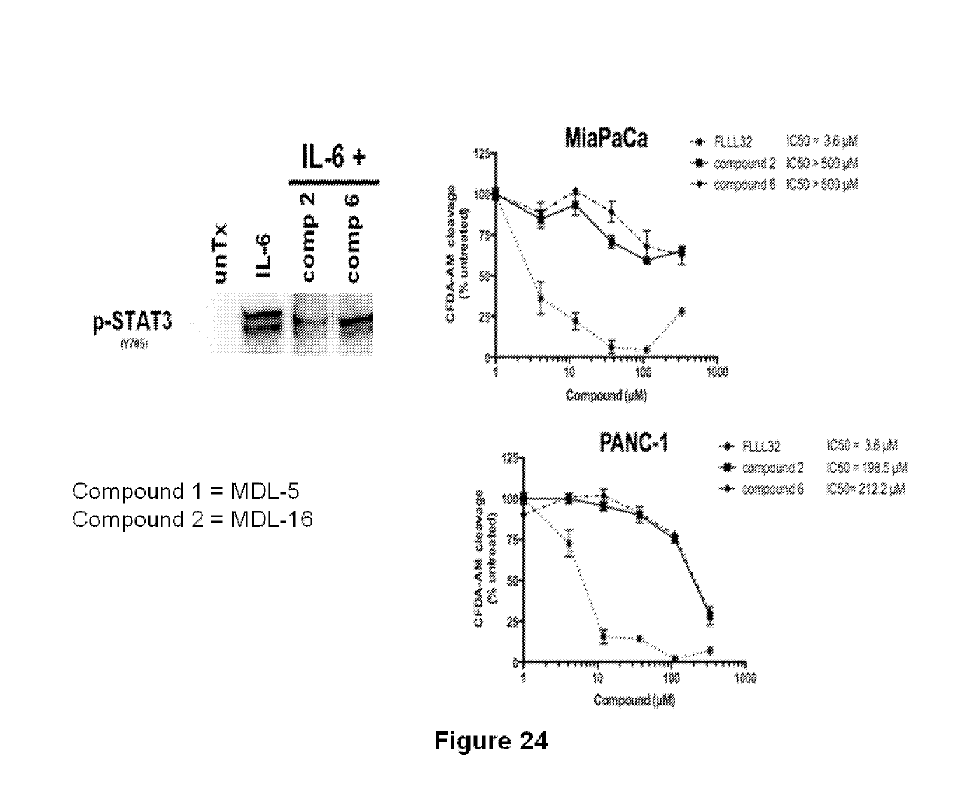

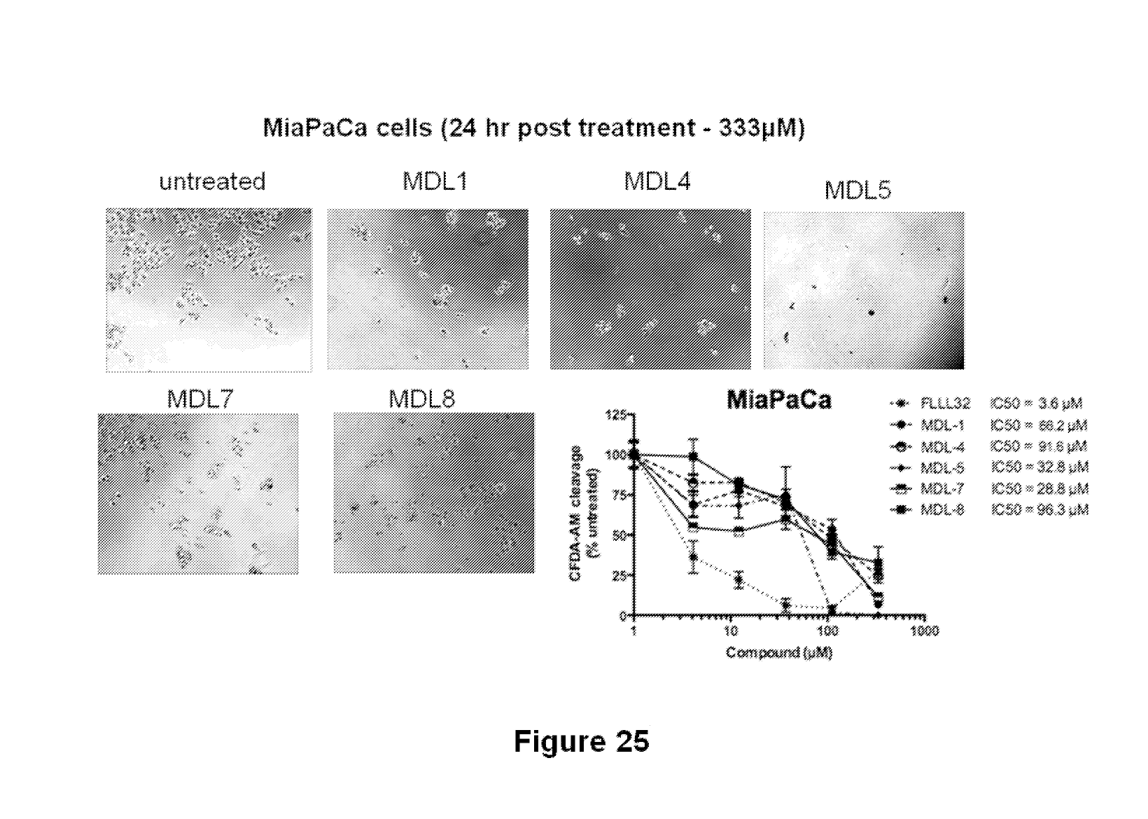

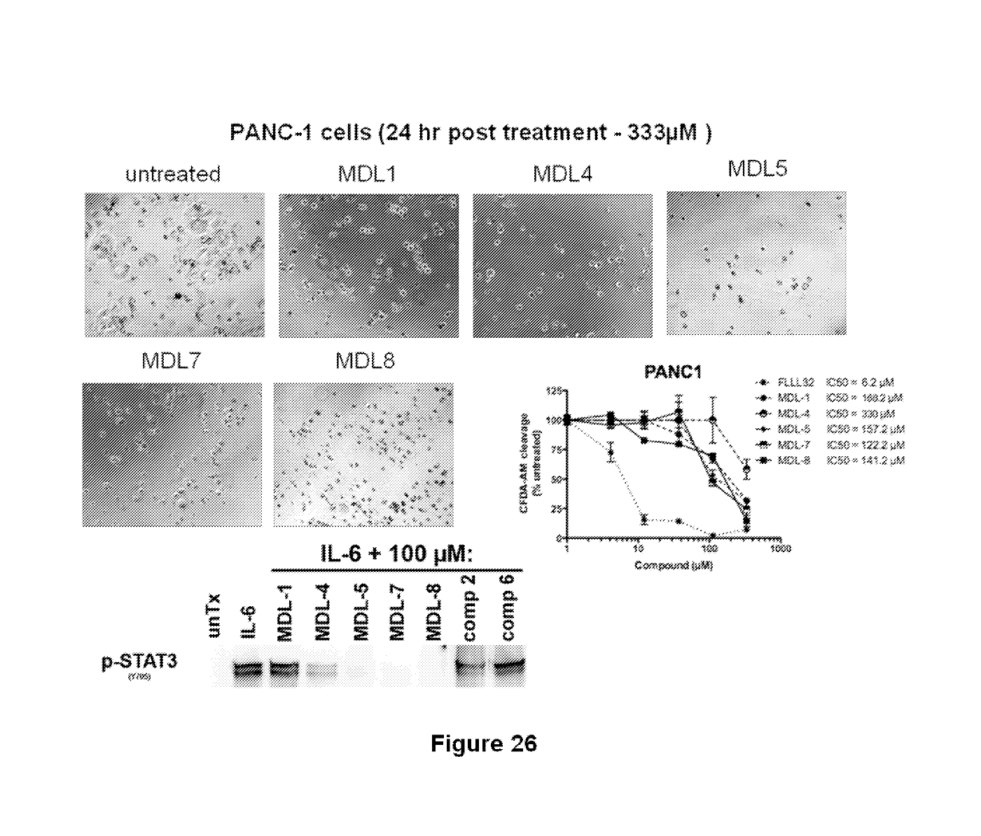

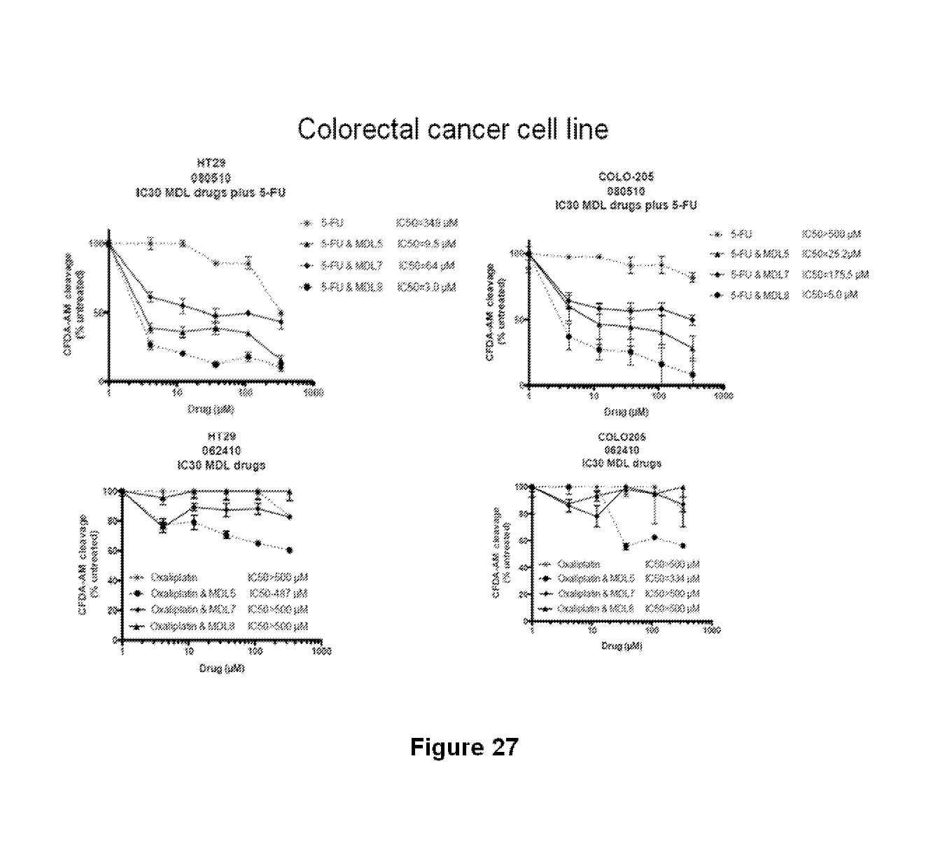

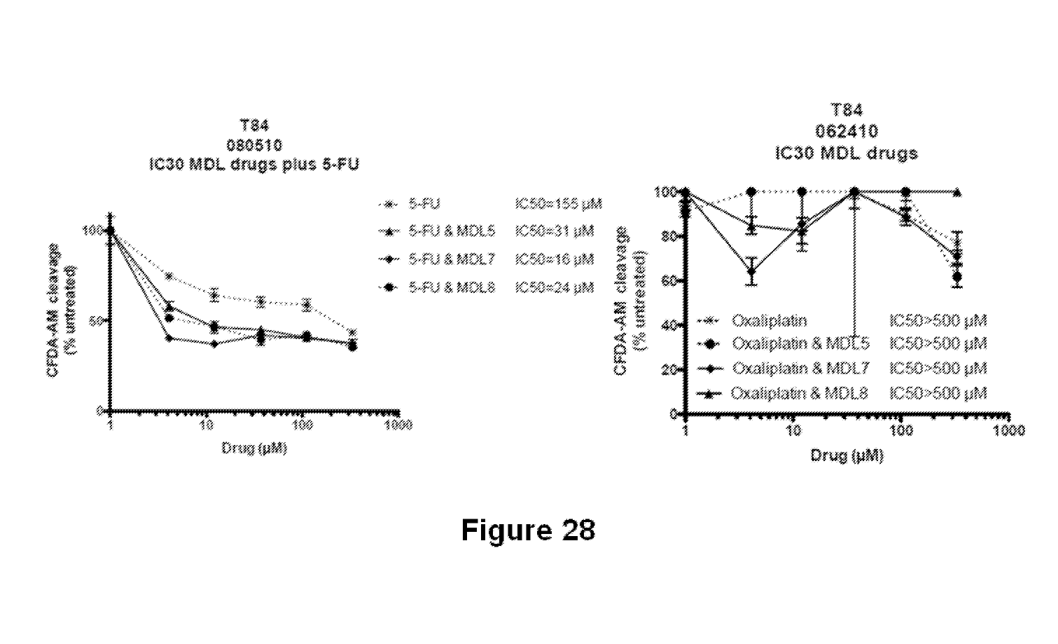

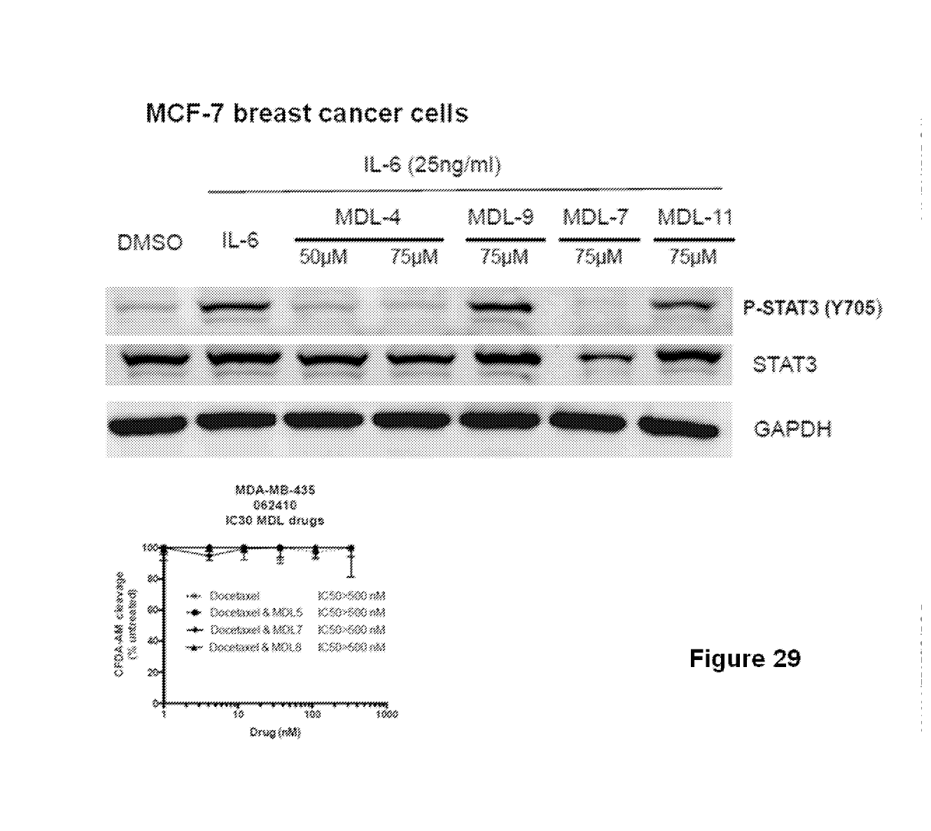

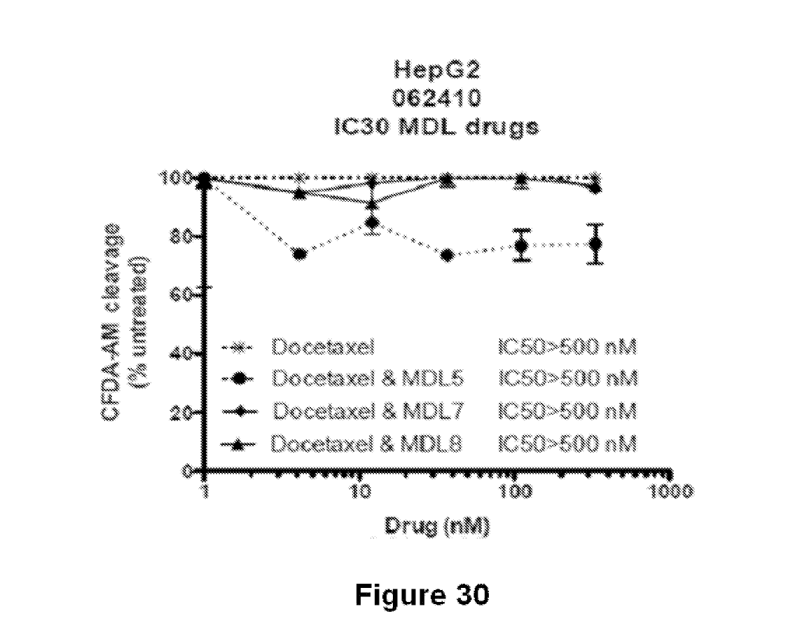

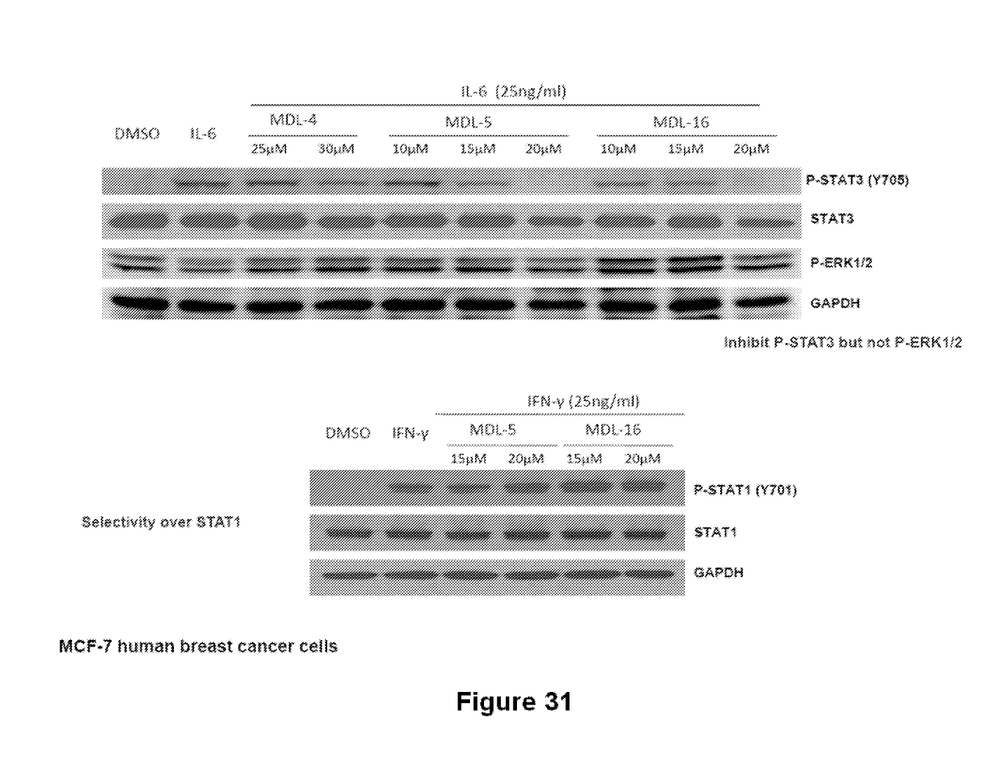

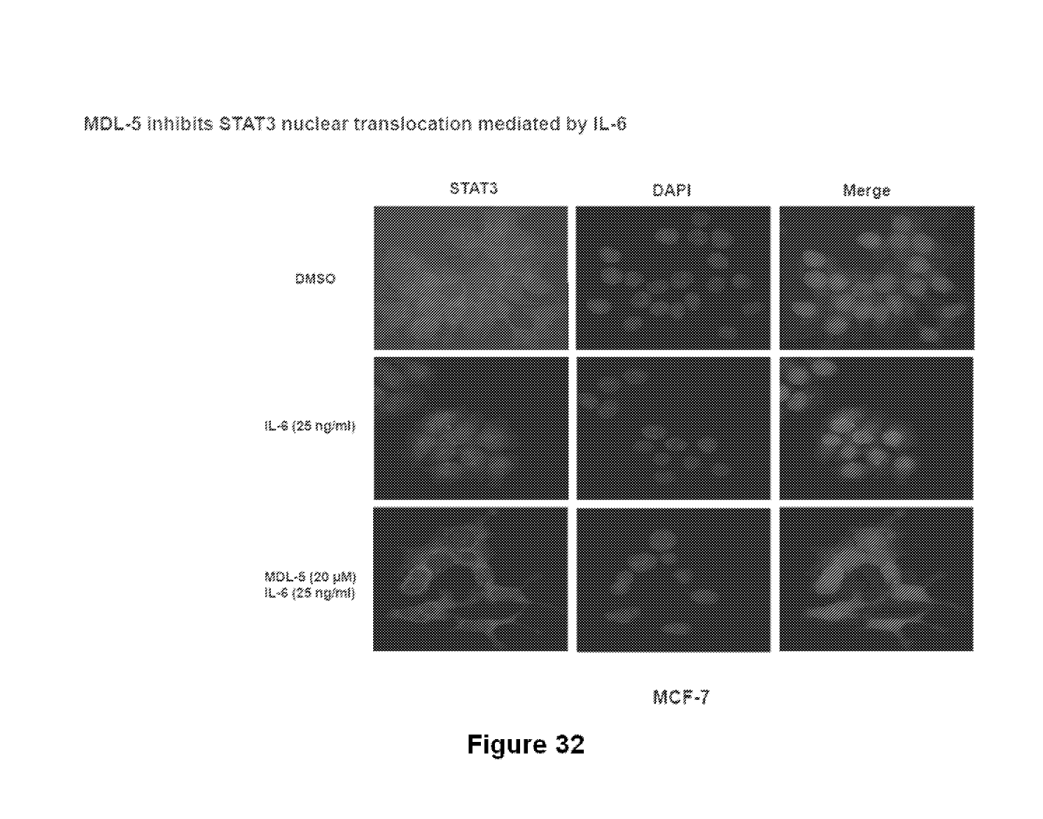

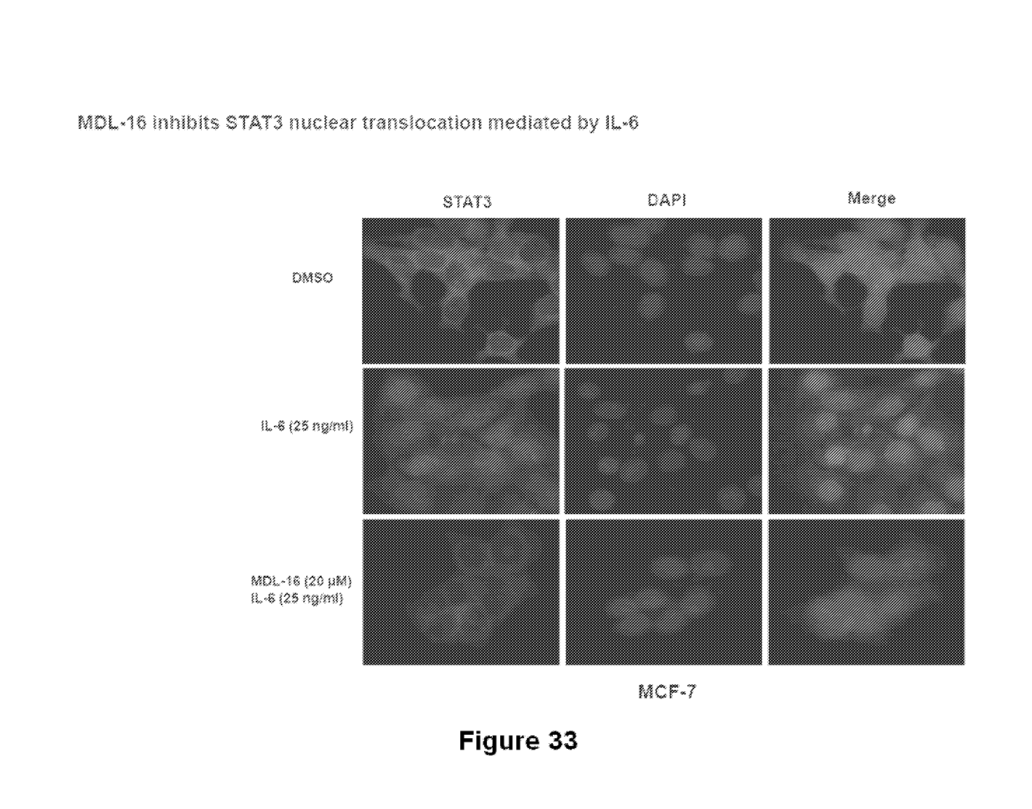

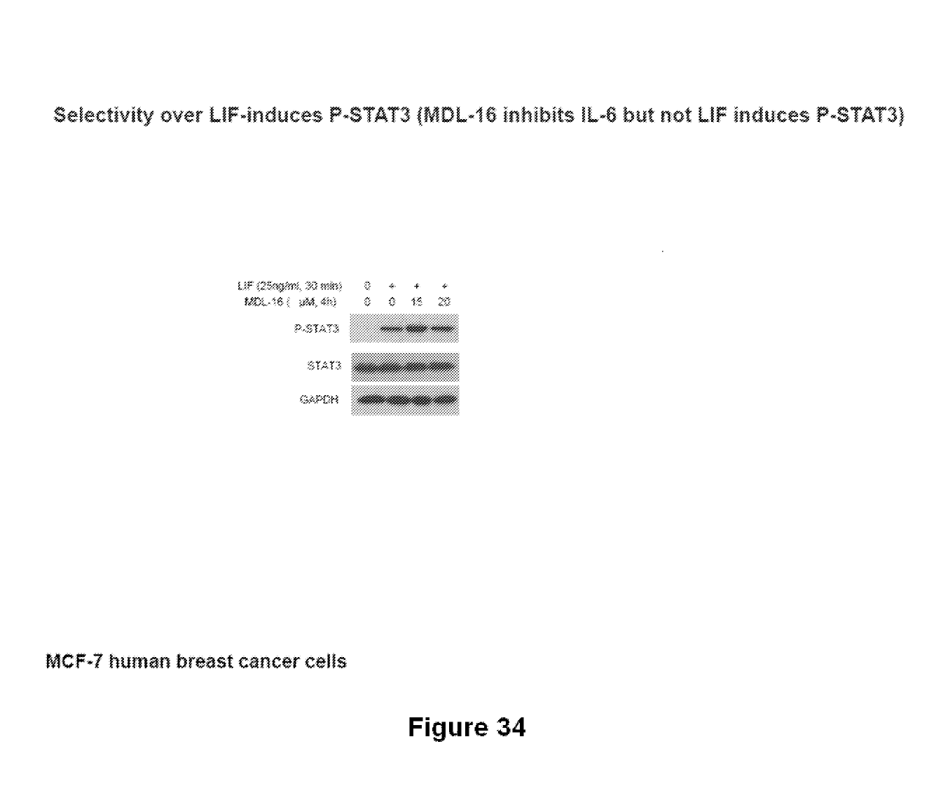

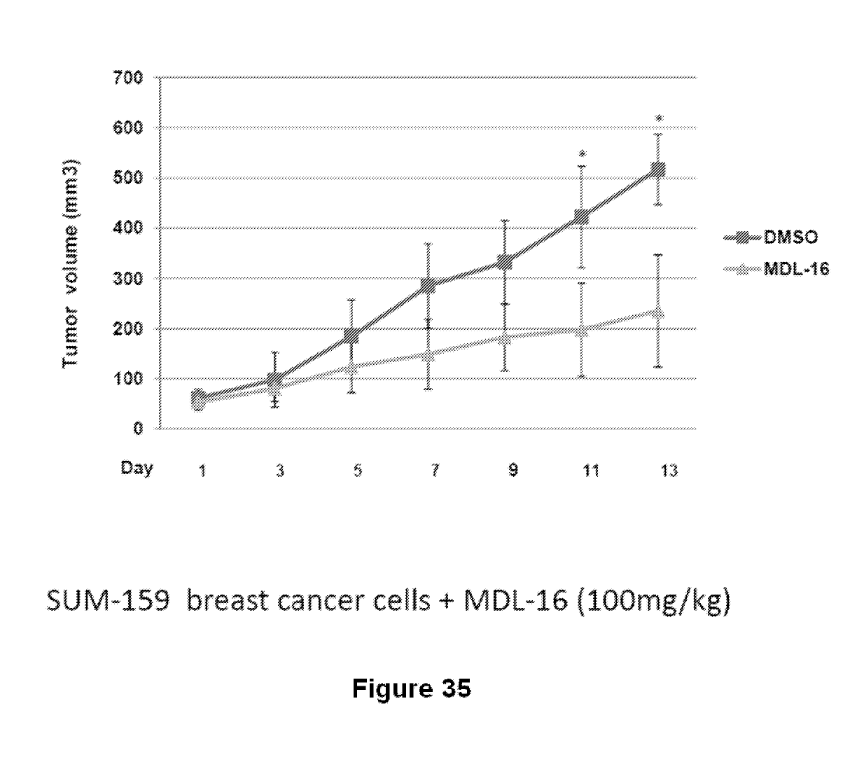

MDL-A and MDL-A analogues inhibit Stat3 phosphorylation induced by IL6 (FIG. 17). In addition, the analogues are significantly more potent than MDL-A in terms of inhibition of Stat3 phosphorylation. Both MDL-5 and 16 showed a dose dependent inhibition of Stat3 phosphorylation. At 40 μM, both MDL-16 completely inhibits Stat3 phosphorylation induced by IL-6 (12.5 ng/ml, 30 min). It is slightly more potent than MDL-5 (FIG. 18) and is directly correlated with the K_{D} values. In addition, all the compounds induced apoptosis in LNCap cells.

### 18. In Vitro Studies Methods

MDL-A has been reported to show direct binding at the extracellular domain of gp130 by Saleh and coworkers (1). The same type of direct binding assay as described elsewhere herein were used to assess the binding of analogues. Therefore, recombinant gp130 protein (gp130-Fc-HA) was purified by expressing in HEK293T cells and purified by protein A affinity chromatography from the medium of transfected cells. Immunoblotting with an anti-HA antibody confirmed that the major species corresponded to the predicted gp130 protein (FIG. 13B).

To examine the direct binding and calculate the equilibrium dissociation constant (KD) of MDL-A analogues with gp130 extracellular domain, surface plasmon resonance analysis was performed. Surface plasmon resonance (SPR) analysis was performed using a BIAcore T100. The protein was covalently cross linked to the dextran matrix of the biosensor chip CM5. Successively, various concentrations of MDL-A and MDL-A analogues were injected into the flow cells containing bound protein and no protein. Interactions were monitored in real time and KD values were calculated by reference-substrate sensogram. KD values (Table 1a above) were calculated using binding affinity analysis program in Biacore evaluation software version 2.0.

MDL-A and MDL-A analogues also inhibit Stat3 phosphorylation induced by IL-6 (FIG. 17). Furthermore, the analogues are significantly more potent than MDL-A in terms of inhibition of Stat3 phosphorylation. At 40 mM, MDL-6 and MDL-7 appear to completely inhibit Stat3 phosphorylation induced by IL-6 (12.5 ng/ml, 30 min). In the LnCap cells they appear to be slightly more potent than MDL-5 (FIG. 18) and the level of pSTAT3 observed is at least somewhat correlated with the KD values. MDL-5, however, demonstrates a dose dependent inhibition of Stat3 phosphorylation and the pSTAT3 levels appear to vary based on the cell line.

### 19. Prophetic In Vivo Anti-Tumor Effects: Cell-Line Xenograft Model

The following example of the in vivo effect of the disclosed compounds are prophetic. Generally agents which modulate the regulation of chromatin, including histone demethylase inhibitors, display efficacy in preclinical models of cancer. In vivo effects of the compounds described in the preceding examples are expected to be shown in various animal models of cancer known to the skilled person, such as tumor xenograft models. These models are typically conducted in rodent, most often in mouse, but may be conducted in other animal species as is convenient to the study goals. Compounds, products, and compositions disclosed herein are expected to show in vivo effects in various animal models of cancer known to the skilled person, such as mouse tumor xenograft models.

In vivo effects of compounds can be assessed with in a mouse tumor xenograft study, one possible study protocol is described herein. Briefly, cells (2 to 5×10⁶ in 100 mL culture media) were implanted subcutaneously, e.g. by subcutaneous injection, in the right hind flank of athymic nu/nu nude mice (5 to 6 weeks old, 18-22 g). For test compounds of the present invention, a typical cell-line used for the tumor xenograft study would be prostate cancer cell-lines such as LNCAP, PC3, or DU145 or breast cancer cell-lines such as MDA-MB-231, SUM-159, and SK-BR-3. The cells are cultured prior to harvesting for this protocol as described herein.

Following implantation, the tumors are allowed to grow to about 100 mm³, typically about 6-18 days post-implantation, before the animals are randomized into treatment groups (e.g. vehicle, positive control and various dose levels of the test compound); the number of animals per group is typically 8-12. Day 1 of study corresponds to the day that the animals receive their first dose. The efficacy of a test compound can be determined in studies of various length dependent upon the goals of the study. Typical study periods are for 14, 21 and 28-days. The dosing frequency (e.g. whether animals are dosed with test compound daily, every other day, every third day or other frequencies) is determined for each study depending upon the toxicity and potency of the test compound. A typical study design would involve dosing daily (M-F) with the test compound with recovery on the weekend. Throughout the study, tumor volumes and body weights are measured twice a week. At the end of the study the animals are euthanized and the tumors harvested and frozen for further analysis. Alternatively, tumors may be processed immediately for analysis, e.g. fixed in buffered-formalin, paraffin embedded, and sectioned for hematoxylin/eosin staining and further immunohistochemical analysis for desired oncology markers.

For example, it is anticipated that one or more disclosed compounds, or a pharmaceutically acceptable salt, solvate, polymorph, hydrate and the stereochemically isomeric form thereof, are expected to show such in vivo effects. That is, one or more disclosed compounds having a structure represented by a formula: are expected to show such in vivo effects:

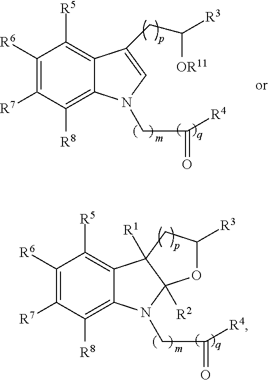

wherein m and n are integers independently selected from 1, 2, 3, 4, 5, and 6; wherein p is an integer selected from 1, 2 and 3; and wherein q is an integer selected from 0 and 1; wherein each of R¹ and R² is independently selected from H and —OH; wherein R³ is selected from: hydrogen,

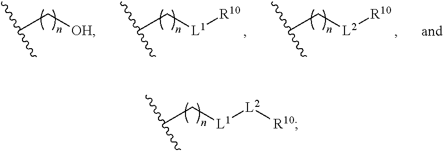

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 haloalkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹ is selected from hydrogen and C1-C8 alkyl; or a pharmaceutically acceptable salt, solvate, or polymorph thereof.

### 20. Prophetic In Vivo Anti-Tumor Effects: Tumor Graft Model

Alternatively, it can be desirable to assess the in vivo efficacy of the disclosed compounds in a tumor explant or tumor graft animal models (e.g. see Rubio-Viqueira B., et al. Clin Cancer Res. (2006) 12:4652-4661; Fiebig, H. H., Maier, A. and Burger, A. M. Eur. J. Canc. (2004) 40:802-820; and DeRose, Y. S., et al. “Patient-derived tumor grafts authentically reflect tumor pathology, growth, metastasis and disease outcomes.” (2011) Nat. Med., in press). These models can provide higher quality information on in vivo effects of therapeutic compounds. It is believed tumor graft models are more authentic in vivo models of many types of cancer, e.g. human breast cancer, with which to examine the biology of tumors and how they metastasize. Engraftment of actual patient tumor tissues into immunodeficient mice (termed ‘tumor grafts’) provides improvement over implantation of cell lines, in terms of phenocopying human tumors and predicting drug responses in patients (Clarke, R. Breast Cancer Res (2009) 11 Suppl 3, S22; Press, J. Z., et al. Gynecol Oncol (2008) 110:56-264; Kim, M. P., et al. Nat Protoc (2009) 4:670-1680; Daniel, V. C., et al. Cancer Res (2009) 69:3364-3373; and Ding, L., et al. Nature (2010) 464:999-1005).

Briefly, tissue samples will be collected from informed, consented patients under an approved IRB protocol. Samples will be collected and deidentified before being obtained for implantation. It is anticipated that all primary tumors will be from individuals that had not received chemotherapy prior to tissue collection, and all metastatic effusions will be from individuals that had been treated with chemotherapy, hormone therapy, and/or radiation therapy. All animal studies will be subject to an Institutional Animal Care and Use Committee review and approval. It is anticipated that a minimum of three mice per experimental group will be used, and only female mice will be used for studies involving breast cancer tumors. A single fragment of fresh or frozen tumor (˜8 mm3), or about 10⁶ cells in matrigel, is implanted into cleared inguinal mammary fat pads of 3-4 week old female NOD/SCID mice. At the same, interscapular estrogen pellets are subcutaneously implanted in mice with ER+ tumors. Tumor growth is measured weekly using calipers. When tumors reach about 150-2,000 mm³, the mice are euthanized, and tissue fragments are re-transplanted into another cohort of mice, frozen for later use, and/or analyzed for histology, gene expression, and DNA copy number. Tumor volumes are calculated using the formula 0.5×length×(width)². For experiments to determine estrogen dependence, ER⁺ tumors are implanted into mice as described above, in the presence or absence of intrascapular estrogen pellets and with or without a concurrent surgical procedure to remove the ovaries, which is performed according to standard methods.

Freshly harvested tumor tissues from patients or mice are cut into 8 mm3 pieces and stored in liquid nitrogen, in a solution of 95% FBS and 5% DMSO for later implantation. Alternatively, the tissue is digested with collagenase solution (1 mg/ml collagenase [Type IV, Sigma] in RPMI 1640 supplemented with 2.5% FBS, 10 mM HEPES, 10 μg/mL penicillin-streptomycin) at 37° C. for 40-60 min, while shaking at 250 rpm. Digested tissue is strained to remove debris and washed in human breast epithelial cell (HBEC) medium (DMEM F/12 supplemented with 10 mM HEPES, 5% FBS, 1 mg/mL BSA, 0.5 μg/mL hydrocortisone, 50 μg mL Gentamycin, 1 μg/mL ITS-X100) three times. The pellet is resuspended in freezing medium (5% FBS and 10% DMSO in HBEC medium) and stored in liquid nitrogen.

To assess the effect of a disclosed compound, tumors in mice are allowed to grow to about 100 mm³, typically about 6-18 days post-implantation, before the animals are randomized into treatment groups (e.g. vehicle, positive control and various dose levels of the test compound); the number of animals per group is typically 8-12. Day 1 of study corresponds to the day that the animals receive their first dose. The efficacy of a test compound can be determined in studies of various length dependent upon the goals of the study. Typical study periods are for 14, 21 and 28-days. The dosing frequency (e.g. whether animals are dosed with test compound daily, every other day, every third day or other frequencies) is determined for each study depending upon the toxicity and potency of the test compound. A typical study design would involve dosing daily (M-F) with the test compound with recovery on the weekend. Throughout the study, tumor volumes and body weights are measured twice a week. At the end of the study the animals are euthanized and the tumors harvested and frozen for further analysis. Alternatively, tumors may be processed immediately for analysis, e.g. fixed in buffered-formalin, paraffin embedded, and sectioned for hematoxylin/eosin staining and further immunohistochemical analysis for desired oncology markers.

For example, it is anticipated that one or more disclosed compounds, or a pharmaceutically acceptable salt, solvate, polymorph, hydrate and the stereochemically isomeric form thereof, are expected to show such in vivo effects. That is, one or more disclosed compounds having a structure represented by a formula: are expected to show such in vivo effects:

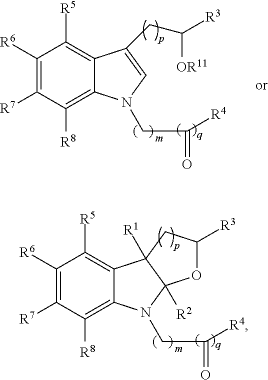

wherein m and n are integers independently selected from 1, 2, 3, 4, 5, and 6; wherein p is an integer selected from 1, 2 and 3; and wherein q is an integer selected from 0 and 1; wherein each of R¹ and R² is independently selected from H and —OH; wherein R³ is selected from: hydrogen,

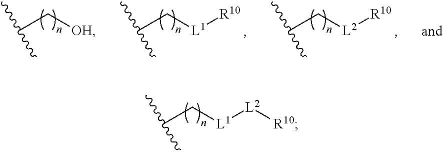

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 haloalkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹ is selected from hydrogen and C1-C8 alkyl; or a pharmaceutically acceptable salt, solvate, or polymorph thereof.

### 21. Prophetic Pharmaceutical Composition Examples

“Active ingredient” as used throughout these examples relates to one or more disclosed compounds having a structure represented by a formula:

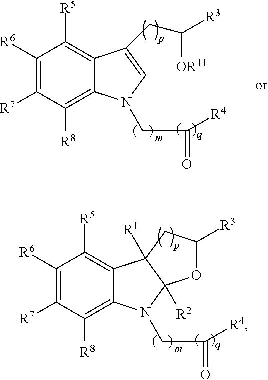

wherein m and n are integers independently selected from 1, 2, 3, 4, 5, and 6; wherein p is an integer selected from 1, 2 and 3; and wherein q is an integer selected from 0 and 1; wherein each of R¹ and R² is independently selected from H and —OH; wherein R³ is selected from: hydrogen,

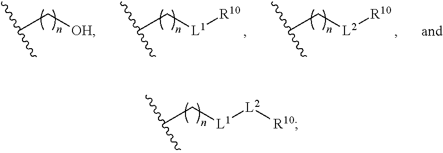

wherein L¹ is —O— or —NH—; wherein L² is —CH₂— or —(C═O)—; and wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹; wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²¹ and R²² is independently selected from hydrogen and C1-C6 alkyl; wherein R⁴ is selected from C1-C8 alkyl, C1-C8 haloalkyl, C1-C8 alkoxy, —NR²³R²⁴, —O—Ar², —NH—Ar², —O-Cy², and —NH-Cy²; wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy; wherein each of R²³ and R²⁴ is independently selected from hydrogen and C1-C6 alkyl; wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and wherein each of R²⁵ and R²⁶ is independently selected from hydrogen and C1-C6 alkyl; wherein R¹¹ is selected from hydrogen and C1-C8 alkyl; or a pharmaceutically acceptable salt, solvate, or polymorph thereof. The following examples of the formulation of the compounds of the present invention in tablets, suspension, injectables and ointments are prophetic.

Typical examples of recipes for the formulation of the invention are as given below. Various other dosage forms can be applied herein such as a filled gelatin capsule, liquid emulsion/suspension, ointments, suppositories or chewable tablet form employing the disclosed compounds in desired dosage amounts in accordance with the present invention. Various conventional techniques for preparing suitable dosage forms can be used to prepare the prophetic pharmaceutical compositions, such as those disclosed herein and in standard reference texts, for example the British and US Pharmacopoeias, Remington's Pharmaceutical Sciences (Mack Publishing Co.) and Martindale The Extra Pharmacopoeia (London The Pharmaceutical Press).

The disclosure of this reference is hereby incorporated herein by reference.

a. Pharmaceutical Composition for Oral Administration

A tablet can be prepared as follows:

| Component | Amount | |
|---|---|---|
| Active ingredient | 10 to 500 | mg |
| Lactose | 100 | mg |
| Crystalline cellulose | 60 | mg |
| Magnesium stearate | 5 | |
| Starch (e.g. potato starch) | Amount necessary | |
| | to yield total weight | |
| | indicated below | |
| Total (per capsule) | 1000 | mg |

Alternatively, about 100 mg of a disclosed compound, 50 mg of lactose (monohydrate), 50 mg of maize starch (native), 10 mg of polyvinylpyrrolidone (PVP 25) (e.g. from BASF, Ludwigshafen, Germany) and 2 mg of magnesium stearate are used per tablet. The mixture of active component, lactose and starch is granulated with a 5% solution (m/m) of the PVP in water. After drying, the granules are mixed with magnesium stearate for 5 min. This mixture is moulded using a customary tablet press (e.g. tablet format: diameter 8 mm, curvature radius 12 mm). The moulding force applied is typically about 15 kN.

Alternatively, a disclosed compound can be administered in a suspension formulated for oral use. For example, about 100-5000 mg of the desired disclosed compound, 1000 mg of ethanol (96%), 400 mg of xanthan gum, and 99 g of water are combined with stirring. A single dose of about 10-500 mg of the desired disclosed compound according can be provided by 10 ml of oral suspension.

In these Examples, active ingredient can be replaced with the same amount of any of the compounds according to the present invention, in particular by the same amount of any of the exemplified compounds. In some circumstances it may be desirable to use a capsule, e.g. a filled gelatin capsule, instead of a tablet form. The choice of tablet or capsule will depend, in part, upon physicochemical characteristics of the particular disclosed compound used.

Examples of alternative useful carriers for making oral preparations are lactose, sucrose, starch, talc, magnesium stearate, crystalline cellulose, methyl cellulose, hydroxypropyl cellulose, hydroxypropylmethyl cellulose, carboxymethyl cellulose, glycerin, sodium alginate, gum arabic, etc. These alternative carriers can be substituted for those given above as required for desired dissolution, absorption, and manufacturing characteristics.

The amount of a disclosed compound per tablet for use in a pharmaceutical composition for human use is determined from both toxicological and pharmacokinetic data obtained in suitable animal models, e.g. rat and at least one non-rodent species, and adjusted based upon human clinical trial data. For example, it could be appropriate that a disclosed compound is present at a level of about 10 to 1000 mg per tablet dosage unit.

b. Pharmaceutical Composition for Injectable Use

A parenteral composition can be prepared as follows:

| Component | Amount* | |
|---|---|---|
| Active ingredient | 10 to 500 | mg |
| Sodium carbonate | 560 | mg* |
| Sodium hydroxide | 80 | mg* |
| Distilled, sterile water | Quantity sufficient | |
| | to prepare total volumen | |
| | indicated below. | |
| Total (per capsule) | 10 | ml per ampule |

*Amount adjusted as required to maintain physiological pH in the context of the amount of active ingredient, and form of active ingredient, e.g. a particular salt form of the active ingredient.

Alternatively, a pharmaceutical composition for intravenous injection can be used, with composition comprising about 100-5000 mg of a disclosed compound, 15 g polyethylenglycol 400 and 250 g water in saline with optionally up to about 15% Cremophor EL, and optionally up to 15% ethyl alcohol, and optionally up to 2 equivalents of a pharmaceutically suitable acid such as citric acid or hydrochloric acid are used. The preparation of such an injectable composition can be accomplished as follows: The disclosed compound and the polyethylenglycol 400 are dissolved in the water with stirring. The solution is sterile filtered (pore size 0.22 μm) and filled into heat sterilized infusion bottles under aseptic conditions. The infusion bottles are sealed with rubber seals.

In a further example, a pharmaceutical composition for intravenous injection can be used, with composition comprising about 10-500 mg of a disclosed compound, standard saline solution, optionally with up to 15% by weight of Cremophor EL, and optionally up to 15% by weight of ethyl alcohol, and optionally up to 2 equivalents of a pharmaceutically suitable acid such as citric acid or hydrochloric acid. Preparation can be accomplished as follows: a desired disclosed compound is dissolved in the saline solution with stirring. Optionally Cremophor EL, ethyl alcohol or acid are added. The solution is sterile filtered (pore size 0.22 μm) and filled into heat sterilized infusion bottles under aseptic conditions. The infusion bottles are sealed with rubber seals.

In this Example, active ingredient can be replaced with the same amount of any of the compounds according to the present invention, in particular by the same amount of any of the exemplified compounds.

The amount of a disclosed compound per ampule for use in a pharmaceutical composition for human use is determined from both toxicological and pharmacokinetic data obtained in suitable animal models, e.g. rat and at least one non-rodent species, and adjusted based upon human clinical trial data. For example, it could be appropriate that a disclosed compound is present at a level of about 10 to 1000 mg per tablet dosage unit.

Carriers suitable for parenteral preparations are, for example, water, physiological saline solution, etc. which can be used with tris(hydroxymethyl)aminomethane, sodium carbonate, sodium hydroxide or the like serving as a solubilizer or pH adjusting agent. The parenteral preparations contain preferably 50 to 1000 mg of a disclosed compound per dosage unit.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 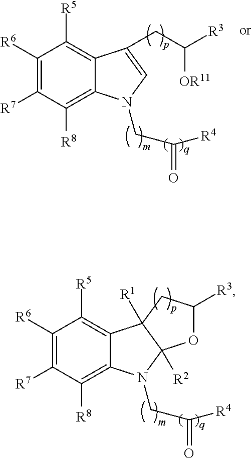
wherein m and n are integers independently selected from 1, 2, 3, 4, 5, and 6; wherein p is an integer selected from 1, 2 and 3; and wherein q is an integer selected from 0 and 1;
wherein each of R¹ and R², when present, is independently selected from H and —OH;
wherein R³ is selected from:
hydrogen, 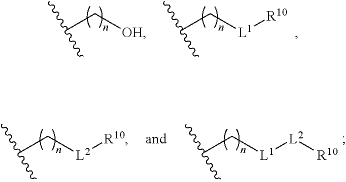
wherein L¹ is —O— or —NH—;
wherein L² is CH₂ or (C═O); and
wherein R¹⁰ is selected from hydrogen, C1-C8 alkyl, C1-C8 alkoxy, —NR²¹R²², —O—Ar¹, —NH—Ar¹, —O—Cy¹, and —NH—Cy¹;
wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy;
wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy;
wherein each of R²¹ and R²² is independently selected from hydrogen and C1-C6 alkyl;
wherein R⁴ is selected from C1-C8 alkyl, C1-C8 alkoxy, —NR²³R²⁴, —O—Ar², —NH—Ar², —O-Cy², and —NH-Cy²;
wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy;
wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy;
wherein each of R²³ and R²⁴ is independently selected from hydrogen and C1-C6 alkyl;
wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and
wherein each of R²⁵ and R²⁶ is independently selected from hydrogen and C1-C6 alkyl;
wherein R¹¹, when present, is selected from hydrogen and C1-C8 alkyl;
or a pharmaceutically acceptable salt, solvate, or polymorph thereof.

2. The compound of claim 1, wherein m is 1; wherein q is 0; and wherein R⁴ is phenyl.

3. The compound of claim 2, wherein R⁴ is: 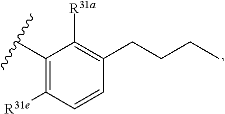
wherein each of R^{31a} and R^{31e} is independently selected from —F, —OH, —NH₂, —NHCH₃, —CH₂F, —CHF₂, —CF₃, and —OCH₃.

4. The compound of claim 1, wherein R¹⁰ is phenyl substituted with, 1, 2, or 3 groups independently selected from —F, —OH, —NH₂, —NHCH₃, —CH₂F, —CHF₂, —CF₃, and —OCH₃.

5. The compound of claim 1, wherein R³ is 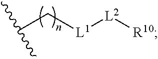
and wherein L¹ is —O—.

6. The compound of claim 1, wherein each of R⁵, R⁶, R⁷, and R⁸ is hydrogen.

7. The compound of claim 1, wherein each of R⁵, R⁶, and R⁸ is hydrogen, and R⁸ is —CH₂OH.

8. The compound of claim 1, having a structure represented by a formula:

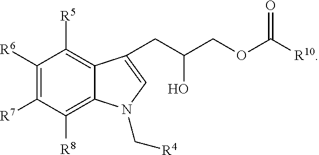

9. The compound of claim 1, having a structure represented by a formula: 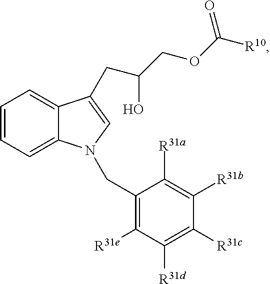
wherein each of R^{31a}, R^{31b}, R^{31c}, R^{31d}, and R^{31e} is independently selected from hydrogen, —F, —OH, —NH₂, —NHCH₃, —NHCH₂CH₃, methyl, —CH₂F, —CHF₂, —CF₃, and —OCH₃, provided that at least two of R^{31a}, R^{31b}, R^{31c}, R^{31d}, and R^{31e} are hydrogen.

10. The compound of claim 1, having a structure represented by a formula: 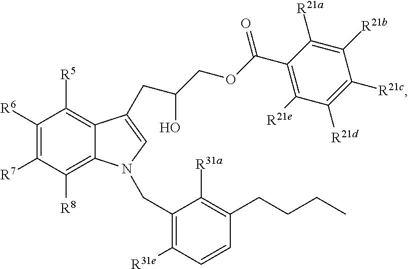
wherein each of R^{21a}, R^{21b}, R^{21c}, R^{21d}, and R^{21e} is independently selected from hydrogen, —F, —OH, —NH₂, —NHCH₃, —NHCH₂CH₃, methyl, —CH₂F, —CHF₂, —CF₃, and —OCH₃, provided that at least two of R^{21a}, R^{21b}, R^{21c}, R^{21d}, and R^{21e} are hydrogen; and wherein each of R^{31a} and R^{31e} is independently selected from hydrogen, —F, —OH, —NH₂, —NHCH₃, —NHCH₂CH₃, methyl, —CH₂F, —CHF₂, —CF₃, and —OCH₃.

11. A method for the treatment of a disorder associated with an IL6 dysfunction in a mammal comprising the step of administering to the mammal a therapeutically effective amount of at least one compound having a structure represented by a formula: 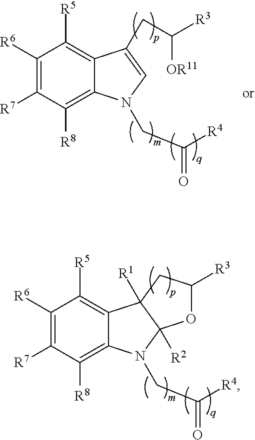
wherein m and n are integers independently selected from 1, 2, 3, 4, 5, and 6; wherein p is an integer selected from 1, 2 and 3; and wherein q is an integer selected from 0 and 1;
wherein each of R¹ and R², when present, is independently selected from H and —OH;
wherein R³ is selected from:
hydrogen, 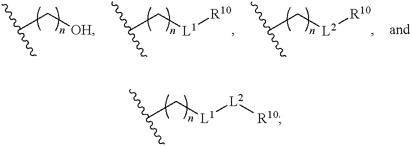
wherein L¹ is —O— or —NH—;
wherein L² is —CH₂— or —(C═O)—; and
wherein R¹⁰ is selected from hydrogen, C1-C8 alkyl, C1-C8 alkoxy, —NR²¹R²², —O—Ar¹, —NH—Ar¹, —O-Cy¹, and —NH-Cy¹;
wherein Ar¹ is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy;
wherein Cy¹ is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy;
wherein each of R²¹ and R²² is independently selected from hydrogen and C1-C6 alkyl;
wherein R⁴ is selected from C1-C8 alkyl, C1-C8 alkoxy, —NR²³R²⁴, —O—Ar², —NH—Ar², —O-Cy², and —NH-Cy²;
wherein Ar² is phenyl or heteroaryl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy;
wherein Cy² is C3-C6 cycloalkyl or C2-C5 heterocycloalkyl, and substituted with 0, 1, 2, or 3 groups independently selected from halogen, —OH, —NO₂, —NH₂, —NHCH₃, —N(CH₃)₂, —NHCH₂CH₃, —N(CH₂CH₃)₂, —N(CH₃)(CH₂CH₃), C1-C6 alkyl, C1-C6 haloalkyl, and C1-C6 alkoxy;
wherein each of R²³ and R²⁴ is independently selected from hydrogen and C1-C6 alkyl;
wherein each of R⁵, R⁶, R⁷, and R⁸ is independently selected from hydrogen, halogen, —OH, —NO₂, —NR²⁵R²⁶, C1-C6 alkyl, C1-C6 haloalkyl, —(C1-C6 alkyl)-OH, and C1-C6 alkoxy; and
wherein each of R²⁵ and R²⁶ is independently selected from hydrogen and C1-C6 alkyl;
wherein R¹¹, when present, is selected from hydrogen and C1-C8 alkyl;
or a pharmaceutically acceptable salt, solvate, or polymorph thereof.

12. The method of claim 11, wherein the mammal is a human.

13. The method of claim 11, wherein the mammal has been diagnosed with a need for treatment of the disorder prior to the administering step.

14. The method of claim 11, further comprising the step of identifying a mammal in need of treatment of the disorder.

15. The method of claim 11, wherein the IL6 dysfunction is associated with activation of the Jak2/STAT3 pathway.

16. The method of claim 11, wherein the disorder is cancer.

17. The method of claim 16, wherein the disorder is a cancer selected from multiple myeloma disease (MM), renal cell carcinoma (RCC), plasma cell leukaemia, lymphoma, B-lymphoproliferative disorder (BLPD), renal cell carcinoma, breast cancer, prostate cancer, pancreatic cancer, lung cancer, gastric cancer, and colorectal cancer.

18. The method of claim 17, wherein the cancer is selected from breast cancer, prostate cancer, pancreatic cancer, lung cancer, gastric cancer, and colorectal cancer.

19. The method of claim 18, wherein the cancer is prostate cancer.

20. The method of claim 18, wherein the cancer is breast cancer.
